SEQUENCE LISTING (380 sequences)

SEQ ID NO: 1 - DNA, 1422 bp, Artificial Sequence
  Description of Artificial Sequence: Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctccgcagccgaacctaaatctagtgacaagactcatacctgccccccttgtccagcaccagaggctgcaggaggaccttccgtgttcctgtttccacccaaaccaaaggatactctgatgatctcccggacacctgaagtcacttgcgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 2 - DNA, 318 bp, Artificial Sequence
  Description of Artificial Sequence: Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaat

SEQ ID NO: 3 - DNA, 357 bp, Artificial Sequence
  Description of Artificial Sequence: Synthetic polynucleotide
caggtgcagctgcagcagtccggagcagagctggctcgaccaggagctagtgtgaaaatgtcctgtaaggcaagcggctacaccttcacacggtataccatgcattgggtgaaacagagacccgggcagggactggaatggatcgggtacattaatcctagccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactgtctggattattggggccaggggactaccctgacagtgagctcc

SEQ ID NO: 4 - DNA, 330 bp, Artificial Sequence
  Description of Artificial Sequence: Synthetic polynucleotide
gcaccagaggctgcaggaggaccttccgtgttcctgtttccacccaaaccaaaggatactctgatgatctcccggacacctgaagtcacttgcgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaag

SEQ ID NO: 5 - DNA, 318 bp, Artificial Sequence
  Description of Artificial Sequence: Synthetic polynucleotide
gggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggc

SEQ ID NO: 6 - DNA, 1347 bp, Artificial Sequence
  Description of Artificial Sequence: Synthetic polynucleotide
caggtccagctggtgcagagcggaggaggagtggtccagccaggacggtctctgagactgagttgcaaggcatcagggtacactttcacccgatataccatgcactgggtgcggcaggcaccagggaaaggactggaatggatcgggtacattaacccttccaggggatacacaaactataatcagaaggtgaaagacaggttcactatcagccgcgataactccaagaataccgcttttctgcagatggactctctgcgccccgaggatacaggcgtgtatttctgcgcacgatactatgacgatcactactgtctggactattggggccaggggactccagtcaccgtgagctccgcctctactaagggacccagtgtgtttccactggctccctctagtaaatccacatctggaggaactgcagctctgggatgcctggtgaaggattacttcccagagcccgtcaccgtgagttggaactcaggagctctgactagcggcgtccatacctttcccgcagtgctgcagtcaagcgggctgtacagcctgtcctctgtggtcacagtgcctagttcaagcctgggaacacagacttatatctgcaacgtgaatcacaagccttctaatactaaagtcgacaagaaagtggaaccaaagagttgtgataaaacccatacatgcccaccttgtcctgcaccagagctgctgggaggaccaagcgtgttcctgtttccacccaagcctaaagacaccctgatgattagccggacccctgaagtcacatgtgtggtcgtggacgtgagccacgaggaccccgaagtcaagttcaactggtacgtggatggcgtcgaggtgcataatgccaagacaaaacctagagaggaacagtacaattcaacctatagggtcgtgagcgtcctgacagtgctgcaccaggactggctgaacgggaaggagtataagtgcaaagtgtccaataaggcactgcccgcccctatcgagaaaaccatttctaaggcaaaaggccagcctagggaaccacaggtctacgtgtatcctccaagccgcgacgagctgacaaagaaccaggtctccctgacttgtctggtgaaaggattttacccaagtgatattgctgtggagtgggaatcaaatggccagcccgaaaacaattataagaccacaccccctgtgctggacagcgatggctccttcgccctggtctccaagctgactgtggataaatctagatggcagcaggggaacgtctttagttgttcagtgatgcatgaggctctgcacaatcattacacccagaagagcctgtccctgtctcccggcaaa

SEQ ID NO: 7 - DNA, 357 bp, Artificial Sequence
  Description of Artificial Sequence: Synthetic polynucleotide
caggtccagctggtgcagagcggaggaggagtggtccagccaggacggtctctgagactgagttgcaaggcatcagggtacactttcacccgatataccatgcactgggtgcggcaggcaccagggaaaggactggaatggatcgggtacattaacccttccaggggatacacaaactataatcagaaggtgaaagacaggttcactatcagccgcgataactccaagaataccgcttttctgcagatggactctctgcgccccgaggatacaggcgtgtatttctgcgcacgatactatgacgatcactactgtctggactattggggccaggggactccagtcaccgtgagctcc

SEQ ID NO: 8 - DNA, 294 bp, Artificial Sequence
  Description of Artificial Sequence: Synthetic polynucleotide
gcctctactaagggacccagtgtgtttccactggctccctctagtaaatccacatctggaggaactgcagctctgggatgcctggtgaaggattacttcccagagcccgtcaccgtgagttggaactcaggagctctgactagcggcgtccatacctttcccgcagtgctgcagtcaagcgggctgtacagcctgtcctctgtggtcacagtgcctagttcaagcctgggaacacagacttatatctgcaacgtgaatcacaagccttctaatactaaagtcgacaagaaagtg

SEQ ID NO: 9 - DNA, 330 bp, Artificial Sequence
  Description of Artificial Sequence: Synthetic polynucleotide
gcaccagagctgctgggaggaccaagcgtgttcctgtttccacccaagcctaaagacaccctgatgattagccggacccctgaagtcacatgtgtggtcgtggacgtgagccacgaggaccccgaagtcaagttcaactggtacgtggatggcgtcgaggtgcataatgccaagacaaaacctagagaggaacagtacaattcaacctatagggtcgtgagcgtcctgacagtgctgcaccaggactggctgaacgggaaggagtataagtgcaaagtgtccaataaggcactgcccgcccctatcgagaaaaccatttctaaggcaaaa

SEQ ID NO: 10 - DNA, 318 bp, Artificial Sequence
  Description of Artificial Sequence: Synthetic polynucleotide
ggccagcctagggaaccacaggtctacgtgtatcctccaagccgcgacgagctgacaaagaaccaggtctccctgacttgtctggtgaaaggattttacccaagtgatattgctgtggagtgggaatcaaatggccagcccgaaaacaattataagaccacaccccctgtgctggacagcgatggctccttcgccctggtctccaagctgactgtggataaatctagatggcagcaggggaacgtctttagttgttcagtgatgcatgaggctctgcacaatcattacacccagaagagcctgtccctgtctcccggc

SEQ ID NO: 11 - DNA, 1353 bp, Artificial Sequence
  Description of Artificial Sequence: Synthetic polynucleotide
gaggtccagctggtcgaatccggaggaggactggtgaagccaggagggagtctgaaactgtcatgcgccgctagcggctataccttcacatcttacgtcatgcactgggtgaggcaggcacctggcaagggactggaatggatcggatatattaacccatacaatgacggcactaagtataacgagaaatttcagggcagagtgaccatcagctccgataagagcatttccacagcttacatggagctgtctagtctgaggagcgaagacaccgccatgtactattgcgctcgggggacctactattacggaacaagagtgttcgattattggggacagggcaccctggtcacagtgtcaagcgcttccacaaaggggccttctgtgtttccactggcaccctcctctaaatctactagtggaggcaccgcagccctgggatgtctggtgaaggactacttcccagagcccgtcacagtgtcatggaacagcggcgcactgactagcggggtccatacctttcctgccgtgctgcagagttcaggcctgtatagcctgagctccgtggtcacagtgccatctagttcactggggactcagacctacatctgcaacgtgaatcacaagccatccaatactaaagtcgacaagaaagtggaacccaagtcttgtgataaaacacatacttgcccaccttgtcctgcaccagagctgctgggaggaccatccgtgttcctgtttccacccaagcctaaagatactctgatgattagtcgcacaccagaagtgacttgcgtggtcgtggacgtgagccacgaggaccccgaagtcaagttcaactggtacgtggacggcgtcgaggtgcataatgccaagaccaaacccagggaggaacagtataatagtacatacagagtcgtgtcagtgctgaccgtcctgcaccaggattggctgaacggcaaggagtacaagtgcaaagtgtccaataaggctctgcccgcacctatcgagaaaaccatttctaaggcaaaagggcagcctcgagaaccacaggtctatgtgctgcctccatcacgggatgagctgacaaagaaccaggtcagcctgctgtgtctggtgaaagggttctacccctctgacatcgctgtggagtgggaaagtaatggacagcctgaaaacaattatctgacttggccccctgtgctggactccgatggatctttctttctgtacagcaagctgaccgtggacaaatcccgatggcagcagggcaacgtcttttcatgtagcgtgatgcatgaggccctgcacaatcattacacccagaagtccctgtctctgagtcccggcaaa

SEQ ID NO: 12 - DNA, 363 bp, Artificial Sequence
  Description of Artificial Sequence: Synthetic polynucleotide
gaggtccagctggtcgaatccggaggaggactggtgaagccaggagggagtctgaaactgtcatgcgccgctagcggctataccttcacatcttacgtcatgcactgggtgaggcaggcacctggcaagggactggaatggatcggatatattaacccatacaatgacggcactaagtataacgagaaatttcagggcagagtgaccatcagctccgataagagcatttccacagcttacatggagctgtctagtctgaggagcgaagacaccgccatgtactattgcgctcgggggacctactattacggaacaagagtgttcgattattggggacagggcaccctggtcacagtgtcaagc

SEQ ID NO: 13 - DNA, 294 bp, Artificial Sequence
  Description of Artificial Sequence: Synthetic polynucleotide
gcttccacaaaggggccttctgtgtttccactggcaccctcctctaaatctactagtggaggcaccgcagccctgggatgtctggtgaaggactacttcccagagcccgtcacagtgtcatggaacagcggcgcactgactagcggggtccatacctttcctgccgtgctgcagagttcaggcctgtatagcctgagctccgtggtcacagtgccatctagttcactggggactcagacctacatctgcaacgtgaatcacaagccatccaatactaaagtcgacaagaaagtg

SEQ ID NO: 14 - DNA, 330 bp, Artificial Sequence
  Description of Artificial Sequence: Synthetic polynucleotide
gcaccagagctgctgggaggaccatccgtgttcctgtttccacccaagcctaaagatactctgatgattagtcgcacaccagaagtgacttgcgtggtcgtggacgtgagccacgaggaccccgaagtcaagttcaactggtacgtggacggcgtcgaggtgcataatgccaagaccaaacccagggaggaacagtataatagtacatacagagtcgtgtcagtgctgaccgtcctgcaccaggattggctgaacggcaaggagtacaagtgcaaagtgtccaataaggctctgcccgcacctatcgagaaaaccatttctaaggcaaaa

SEQ ID NO: 15 - DNA, 318 bp, Artificial Sequence
  Description of Artificial Sequence: Synthetic polynucleotide
gggcagcctcgagaaccacaggtctatgtgctgcctccatcacgggatgagctgacaaagaaccaggtcagcctgctgtgtctggtgaaagggttctacccctctgacatcgctgtggagtgggaaagtaatggacagcctgaaaacaattatctgacttggccccctgtgctggactccgatggatctttctttctgtacagcaagctgaccgtggacaaatcccgatggcagcagggcaacgtcttttcatgtagcgtgatgcatgaggccctgcacaatcattacacccagaagtccctgtctctgagtcccggc

SEQ ID NO: 16 - DNA, 639 bp, Artificial Sequence
  Description of Artificial Sequence: Synthetic polynucleotide
gatattcagatgacccagagcccaagctccctgagtgcctcagtgggcgaccgagtcaccatcacatgctccgcttctagttcagtgtcttacatgaactggtatcagcagactccagggaaggcacccaaacggtggatctacgatacctcaaagctggccagcggagtgccctccagattcagcggctccgggtctggaacagactatacttttaccatcagctccctgcagcctgaggatattgctacttactattgccagcagtggtctagtaatccattcacttttggccaggggaccaagctgcagatcacaaggactgtggccgctcccagcgtcttcatttttccccctagcgacgagcagctgaaatctggcacagccagtgtggtctgtctgctgaacaatttctaccctcgcgaagcaaaggtgcagtggaaagtcgataacgccctgcagagtggcaacagccaggagagcgtgacagaacaggactccaaggattctacttatagtctgtcaagcaccctgacactgtccaaagctgactacgagaagcacaaagtgtatgcatgcgaagtcacccatcagggactgtcctctcctgtgacaaaatcttttaacagaggcgaatgt

SEQ ID NO: 17 - DNA, 318 bp, Artificial Sequence
  Description of Artificial Sequence: Synthetic polynucleotide
gatattcagatgacccagagcccaagctccctgagtgcctcagtgggcgaccgagtcaccatcacatgctccgcttctagttcagtgtcttacatgaactggtatcagcagactccagggaaggcacccaaacggtggatctacgatacctcaaagctggccagcggagtgccctccagattcagcggctccgggtctggaacagactatacttttaccatcagctccctgcagcctgaggatattgctacttactattgccagcagtggtctagtaatccattcacttttggccaggggaccaagctgcagatcaca

SEQ ID NO: 18 - DNA, 321 bp, Artificial Sequence
  Description of Artificial Sequence: Synthetic polynucleotide
aggactgtggccgctcccagcgtcttcatttttccccctagcgacgagcagctgaaatctggcacagccagtgtggtctgtctgctgaacaatttctaccctcgcgaagcaaaggtgcagtggaaagtcgataacgccctgcagagtggcaacagccaggagagcgtgacagaacaggactccaaggattctacttatagtctgtcaagcaccctgacactgtccaaagctgactacgagaagcacaaagtgtatgcatgcgaagtcacccatcagggactgtcctctcctgtgacaaaatcttttaacagaggcgaatgt

SEQ ID NO: 19 - DNA, 654 bp, Artificial Sequence
  Description of Artificial Sequence: Synthetic polynucleotide
gatattcagctgactcagtcacccgctagcctggcagtgagtctgggccagagggccaccatcagctgcaaggcttcacagagcgtcgactacgatggcgacagctacctgaactggtatcagcagatccctgggcagccccctaaactgctgatctacgacgcctctaatctggtgagtggcatccccccacgcttctccggctctgggagtggaactgattttaccctgaacattcaccccgtggagaaggtcgacgccgctacataccattgccagcagtccacagaggacccctggactttcggcgggggaaccaagctggaaatcaaacggacagtggcagccccatccgtcttcatttttcctccatctgacgagcagctgaaatcagggactgctagcgtggtctgtctgctgaacaatttttacccaagagaagcaaaggtgcagtggaaagtcgataacgccctgcagtccggaaattctcaggagagtgtgacagaacaggattcaaaggacagcacttattccctgagctccaccctgacactgtccaaagctgattacgagaagcacaaagtgtatgcatgcgaagtcacccatcagggactgtctagtcccgtgacaaagtctttcaatcgaggcgaatgt

SEQ ID NO: 20 - DNA, 333 bp, Artificial Sequence
  Description of Artificial Sequence: Synthetic polynucleotide
gatattcagctgactcagtcacccgctagcctggcagtgagtctgggccagagggccaccatcagctgcaaggcttcacagagcgtcgactacgatggcgacagctacctgaactggtatcagcagatccctgggcagccccctaaactgctgatctacgacgcctctaatctggtgagtggcatccccccacgcttctccggctctgggagtggaactgattttaccctgaacattcaccccgtggagaaggtcgacgccgctacataccattgccagcagtccacagaggacccctggactttcggcgggggaaccaagctggaaatcaaa

SEQ ID NO: 21 - DNA, 321 bp, Artificial Sequence
  Description of Artificial Sequence: Synthetic polynucleotide
cggacagtggcagccccatccgtcttcatttttcctccatctgacgagcagctgaaatcagggactgctagcgtggtctgtctgctgaacaatttttacccaagagaagcaaaggtgcagtggaaagtcgataacgccctgcagtccggaaattctcaggagagtgtgacagaacaggattcaaaggacagcacttattccctgagctccaccctgacactgtccaaagctgattacgagaagcacaaagtgtatgcatgcgaagtcacccatcagggactgtctagtcccgtgacaaagtctttcaatcgaggcgaatgt

SEQ ID NO: 22 - DNA, 1452 bp, Artificial Sequence
  Description of Artificial Sequence: Synthetic polynucleotide
gatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagcgcagccgaacccaaatcctctgataagacccacacatgccctccatgtccagctcctgaggctgcaggaggaccaagcgtgttcctgtttccccctaaacctaaggacacactgatgatctctcggacacccgaagtcacttgtgtggtcgtgagcgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggcaag

SEQ ID NO: 23 - DNA, 333 bp, Artificial Sequence
  Description of Artificial Sequence: Synthetic polynucleotide
gatattcagctgacacagagtcctgcatcactggctgtgagcctgggacagcgagcaactatctcctgcaaagccagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 24 - DNA, 372 bp, Artificial Sequence
  Description of Artificial Sequence: Synthetic polynucleotide
caggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacaccaactataatggaaagttcaaaggcaaggccacactgactgctgacgagtcaagctccacagcctatatgcagctgtctagtctggcaagcgaggattccgccgtgtacttttgcgctcggagagaaaccacaactgtgggcaggtactattacgctatggactactggggccaggggaccacagtcaccgtgtcaagc

SEQ ID NO: 25 - DNA, 330 bp, Artificial Sequence
  Description of Artificial Sequence: Synthetic polynucleotide
gctcctgaggctgcaggaggaccaagcgtgttcctgtttccccctaaacctaaggacacactgatgatctctcggacacccgaagtcacttgtgtggtcgtgagcgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggcactgccagcccccatcgagaagacaatttccaaagcaaag

SEQ ID NO: 26 - DNA, 318 bp, Artificial Sequence
  Description of Artificial Sequence: Synthetic polynucleotide
ggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgctctggtcagtaaactgactgtggataagtcacggtggcagcagggaaacgtctttagttgttcagtgatgcacgaggcactgcacaatcattacacccagaaaagcctgtccctgtctcccggc

SEQ ID NO: 27 - DNA, 657 bp, Artificial Sequence
  Description of Artificial Sequence: Synthetic polynucleotide
gacattgtgatgacacagtcccctgccactctgagtctgtcaccaggcgagcgggctaccctgagttgcagaagctccaagagcctgcagaacgtgaatggaaacacatacctgtattggttccagcagaaaccaggccagtctccccagctgctgatctacaggatgtcaaatctgaacagcggagtgcctgaccgcttcagcggctccgggtctggaaccgagttcaccctgacaatttctagtctggagcccgaagatttcgcagtctactattgcatgcagcacctggagtatcctatcacctttggcgctgggacaaagctggagatcaagcgaactgtggccgctccatccgtcttcatctttcccccttctgacgagcagctgaagtccggcacagcctctgtggtctgtctgctgaacaatttctaccccagagaagcaaaggtgcagtggaaagtcgataatgccctgcagagtgggaactcacaggagagcgtgactgaacaggactccaaggattctacctatagtctgtcaagcactctgaccctgagcaaagctgactacgagaagcacaaagtgtatgcatgcgaagtcacacatcaggggctgtcctctcccgtgactaaaagctttaatcggggagagtgt

SEQ ID NO: 28 - DNA, 336 bp, Artificial Sequence
  Description of Artificial Sequence: Synthetic polynucleotide
gacattgtgatgacacagtcccctgccactctgagtctgtcaccaggcgagcgggctaccctgagttgcagaagctccaagagcctgcagaacgtgaatggaaacacatacctgtattggttccagcagaaaccaggccagtctccccagctgctgatctacaggatgtcaaatctgaacagcggagtgcctgaccgcttcagcggctccgggtctggaaccgagttcaccctgacaatttctagtctggagcccgaagatttcgcagtctactattgcatgcagcacctggagtatcctatcacctttggcgctgggacaaagctggagatcaag

SEQ ID NO: 29 - DNA, 321 bp, Artificial Sequence
  Description of Artificial Sequence: Synthetic polynucleotide
cgaactgtggccgctccatccgtcttcatctttcccccttctgacgagcagctgaagtccggcacagcctctgtggtctgtctgctgaacaatttctaccccagagaagcaaaggtgcagtggaaagtcgataatgccctgcagagtgggaactcacaggagagcgtgactgaacaggactccaaggattctacctatagtctgtcaagcactctgaccctgagcaaagctgactacgagaagcacaaagtgtatgcatgcgaagtcacacatcaggggctgtcctctcccgtgactaaaagctttaatcggggagagtgt

SEQ ID NO: 30 - DNA, 1344 bp, Artificial Sequence
  Description of Artificial Sequence: Synthetic polynucleotide
caggtccagctggtggaatccggaggaggagtggtccagcctggacgatctctgagactgagttgcgccgcttcagggttcaagtttagcgggtacggaatgcactgggtgaggcaggcaccaggcaaagggctggagtgggtcgccgtgatctggtatgacggcagcaagaagtactatgtcgattctgtgaagggcaggttcaccattagccgcgacaactccaaaaatacactgtacctgcagatgaactccctgagagccgaagacaccgctgtgtactattgcgccaggcagatgggctattggcacttcgatctgtggggacgaggaaccctggtcacagtgagctccgcatctacaaaggggcccagtgtgtttccactggctccctctagtaaatccacttctggaggaaccgcagcactgggatgtctggtgaaggattacttcccagagcccgtcaccgtgagttggaactcaggggctctgacctccggagtccatacatttccagcagtgctgcagtcaagcggcctgtacagcctgtcctctgtggtcactgtgcccagttcaagcctggggactcagacctatatctgcaacgtgaatcacaagccatcaaataccaaagtcgacaagaaagtggaacccaagagctgtgataaaacacatacttgcccaccttgtcctgcaccagagctgctgggaggaccaagcgtgttcctgtttccacccaagcctaaagacactctgatgatttcccggacacccgaagtgacttgcgtggtcgtggacgtgtctcacgaggaccccgaagtcaagttcaactggtacgtggatggcgtcgaggtgcataatgctaagacaaaaccccgagaggaacagtacaattcaacatatcgggtcgtgagcgtcctgactgtgctgcaccaggactggctgaacggcaaggagtataagtgcaaagtgagtaataaggctctgcccgcacctatcgagaaaaccatttctaaggctaaagggcagcctcgcgaaccacaggtctacgtgtatcctccatctcgagacgagctgactaagaaccaggtcagtctgacctgtctggtgaaagggttttaccctagcgatatcgcagtggagtgggaatccaatggacagccagaaaacaattataagaccacaccccctgtgctggacagcgatggcagcttcgcactggtcagtaagctgacagtggataaatcaagatggcagcagggcaacgtctttagttgttcagtgatgcatgaggccctgcacaatcattacactcagaagagcctgtccctgtctcctggcaaa

SEQ ID NO: 31 - DNA, 354 bp, Artificial Sequence
  Description of Artificial Sequence: Synthetic polynucleotide
caggtccagctggtggaatccggaggaggagtggtccagcctggacgatctctgagactgagttgcgccgcttcagggttcaagtttagcgggtacggaatgcactgggtgaggcaggcaccaggcaaagggctggagtgggtcgccgtgatctggtatgacggcagcaagaagtactatgtcgattctgtgaagggcaggttcaccattagccgcgacaactccaaaaatacactgtacctgcagatgaactccctgagagccgaagacaccgctgtgtactattgcgccaggcagatgggctattggcacttcgatctgtggggacgaggaaccctggtcacagtgagctcc

SEQ ID NO: 32 - DNA, 294 bp, Artificial Sequence
  Description of Artificial Sequence: Synthetic polynucleotide
gcatctacaaaggggcccagtgtgtttccactggctccctctagtaaatccacttctggaggaaccgcagcactgggatgtctggtgaaggattacttcccagagcccgtcaccgtgagttggaactcaggggctctgacctccggagtccatacatttccagcagtgctgcagtcaagcggcctgtacagcctgtcctctgtggtcactgtgcccagttcaagcctggggactcagacctatatctgcaacgtgaatcacaagccatcaaataccaaagtcgacaagaaagtg

SEQ ID NO: 33 - DNA, 330 bp, Artificial Sequence
  Description of Artificial Sequence: Synthetic polynucleotide
gcaccagagctgctgggaggaccaagcgtgttcctgtttccacccaagcctaaagacactctgatgatttcccggacacccgaagtgacttgcgtggtcgtggacgtgtctcacgaggaccccgaagtcaagttcaactggtacgtggatggcgtcgaggtgcataatgctaagacaaaaccccgagaggaacagtacaattcaacatatcgggtcgtgagcgtcctgactgtgctgcaccaggactggctgaacggcaaggagtataagtgcaaagtgagtaataaggctctgcccgcacctatcgagaaaaccatttctaaggctaaa

SEQ ID NO: 34 - DNA, 318 bp, Artificial Sequence
  Description of Artificial Sequence: Synthetic polynucleotide
gggcagcctcgcgaaccacaggtctacgtgtatcctccatctcgagacgagctgactaagaaccaggtcagtctgacctgtctggtgaaagggttttaccctagcgatatcgcagtggagtgggaatccaatggacagccagaaaacaattataagaccacaccccctgtgctggacagcgatggcagcttcgcactggtcagtaagctgacagtggataaatcaagatggcagcagggcaacgtctttagttgttcagtgatgcatgaggccctgcacaatcattacactcagaagagcctgtccctgtctcctggc

SEQ ID NO: 35 - DNA, 1356 bp, Artificial Sequence
  Description of Artificial Sequence: Synthetic polynucleotide
caggcttacctgcagcagtccggagcagaactggtccgaccaggagcttccgtgaaaatgtcttgcaaagcaagtggctacactttcaccagctataacatgcactgggtgaaacagacacctcgacagggactggagtggatcggagcaatctacccagggaacggagacactagctataatcagaagtttaaagggaaggctacactgactgtggataagagctcctctactgcatacatgcagctgagttcactgaccagcgaagactccgctgtgtatttctgcgcaagggtggtctactactccaattcttactggtacttcgatgtgtggggcactgggaccacagtcaccgtgagctccgcctcaaccaaaggacctagcgtgttcccactggctccctctagtaagagtacatcaggaggaactgcagctctgggatgtctggtgaaggactacttcccagagcccgtcacagtgtcttggaacagtggggcactgacatctggagtccatacttttcctgccgtgctgcagtcaagcgggctgtacagcctgtcctctgtggtcactgtgccaagttcaagcctgggaacccagacatatatctgcaacgtgaatcacaaaccaagcaataccaaggtcgacaagaaagtggaacccaaatcctgtgataagactcatacctgcccaccttgtcctgcaccagagctgctgggaggaccatccgtgttcctgtttccacccaaacctaaggacaccctgatgatttctagaaccccagaagtcacatgcgtggtcgtggacgtgagccacgaggaccccgaagtcaagtttaactggtacgtggatggcgtcgaggtgcataatgctaaaacaaagccccgggaggaacagtacaactccacctatagagtcgtgtctgtcctgacagtgctgcaccaggactggctgaacgggaaggagtataaatgcaaggtgagcaacaaggcactgcccgcccctatcgagaagacaatttccaaagctaagggacagcctagggaaccacaggtctacgtgctgcctccatctcgcgacgagctgactaaaaaccaggtcagtctgctgtgtctggtgaagggattctatcccagcgatatcgcagtggagtgggaatccaatggccagcctgaaaacaattacctgacctggccccctgtgctggactcagatggcagcttctttctgtatagtaaactgacagtggataagtcacgctggcagcaggggaacgtctttagctgttccgtgatgcatgaggccctgcacaatcattacacccagaaatctctgagtctgtcacccggcaag

SEQ ID NO: 36 - DNA, 366 bp, Artificial Sequence
  Description of Artificial Sequence: Synthetic polynucleotide
caggcttacctgcagcagtccggagcagaactggtccgaccaggagcttccgtgaaaatgtcttgcaaagcaagtggctacactttcaccagctataacatgcactgggtgaaacagacacctcgacagggactggagtggatcggagcaatctacccagggaacggagacactagctataatcagaagtttaaagggaaggctacactgactgtggataagagctcctctactgcatacatgcagctgagttcactgaccagcgaagactccgctgtgtatttctgcgcaagggtggtctactactccaattcttactggtacttcgatgtgtggggcactgggaccacagtcaccgtgagctcc

SEQ ID NO: 37 - DNA, 294 bp, Artificial Sequence
  Description of Artificial Sequence: Synthetic polynucleotide
gcctcaaccaaaggacctagcgtgttcccactggctccctctagtaagagtacatcaggaggaactgcagctctgggatgtctggtgaaggactacttcccagagcccgtcacagtgtcttggaacagtggggcactgacatctggagtccatacttttcctgccgtgctgcagtcaagcgggctgtacagcctgtcctctgtggtcactgtgccaagttcaagcctgggaacccagacatatatctgcaacgtgaatcacaaaccaagcaataccaaggtcgacaagaaagtg

SEQ ID NO: 38 - DNA, 330 bp, Artificial Sequence
  Description of Artificial Sequence: Synthetic polynucleotide
gcaccagagctgctgggaggaccatccgtgttcctgtttccacccaaacctaaggacaccctgatgatttctagaaccccagaagtcacatgcgtggtcgtggacgtgagccacgaggaccccgaagtcaagtttaactggtacgtggatggcgtcgaggtgcataatgctaaaacaaagccccgggaggaacagtacaactccacctatagagtcgtgtctgtcctgacagtgctgcaccaggactggctgaacgggaaggagtataaatgcaaggtgagcaacaaggcactgcccgcccctatcgagaagacaatttccaaagctaag

SEQ ID NO: 39 - DNA, 318 bp, Artificial Sequence
  Description of Artificial Sequence: Synthetic polynucleotide
ggacagcctagggaaccacaggtctacgtgctgcctccatctcgcgacgagctgactaaaaaccaggtcagtctgctgtgtctggtgaagggattctatcccagcgatatcgcagtggagtgggaatccaatggccagcctgaaaacaattacctgacctggccccctgtgctggactcagatggcagcttctttctgtatagtaaactgacagtggataagtcacgctggcagcaggggaacgtctttagctgttccgtgatgcatgaggccctgcacaatcattacacccagaaatctctgagtctgtcacccggc

SEQ ID NO: 40 - DNA, 1356 bp, Artificial Sequence
  Description of Artificial Sequence: Synthetic polynucleotide
gaagtccagctggtcgaatctggaggaggactggtgcagcctggacgatccctgagactgtcttgcgccgctagtggcttcacttttaacgactatgcaatgcactgggtgcgccaggcaccagggaagggactggagtgggtgagcaccatctcctggaacagcggatctattggctatgcagacagcgtgaaaggcaggttcacaatcagtcgcgataacgccaagaaatcactgtacctgcagatgaatagcctgcgagccgaagacacagctctgtactattgcgccaaggatattcagtatgggaactactattacggaatggacgtgtggggccaggggaccacagtcaccgtgagctccgcctcaacaaaggggcccagcgtgtttccactggctccctctagtaaaagtacctcaggcgggacagcagccctgggatgtctggtgaaggattacttcccagagcccgtcaccgtgtcttggaacagtggcgctctgacaagcggggtccatacttttccagcagtgctgcagtcaagcggcctgtattccctgtcctctgtggtcactgtgcccagttcaagcctggggactcagacctacatctgcaacgtgaatcacaagccatctaataccaaagtcgacaagaaagtggaacccaagagttgtgataaaacacatacttgcccaccttgtcctgcaccagagctgctgggaggaccatccgtgttcctgtttccacccaagcctaaagacaccctgatgattagcaggactcccgaagtcacctgcgtggtcgtggacgtgtcccacgaggaccccgaagtcaagttcaactggtacgtggatggcgtcgaggtgcataatgctaagacaaaaccccgagaggaacagtataattccacttaccgggtcgtgtctgtcctgaccgtgctgcaccaggactggctgaacggcaaggagtacaagtgcaaagtgtctaataaggctctgcccgcacctatcgagaaaacaattagcaaggctaaagggcagcctagagaaccacaggtctatgtgctgcctccaagcagggacgagctgactaagaaccaggtctccctgctgtgtctggtgaaagggttctaccctagtgatatcgcagtggagtgggaatcaaatggacagccagaaaacaattatctgacatggccccctgtgctggactcagatggaagcttctttctgtactccaagctgactgtggataaatctcggtggcagcagggcaacgtctttagctgttccgtgatgcatgaggccctgcacaatcattacacccagaagtctctgagtctgtcacctggcaaa

SEQ ID NO: 41 - DNA, 366 bp, Artificial Sequence
  Description of Artificial Sequence: Synthetic polynucleotide
gaagtccagctggtcgaatctggaggaggactggtgcagcctggacgatccctgagactgtcttgcgccgctagtggcttcacttttaacgactatgcaatgcactgggtgcgccaggcaccagggaagggactggagtgggtgagcaccatctcctggaacagcggatctattggctatgcagacagcgtgaaaggcaggttcacaatcagtcgcgataacgccaagaaatcactgtacctgcagatgaatagcctgcgagccgaagacacagctctgtactattgcgccaaggatattcagtatgggaactactattacggaatggacgtgtggggccaggggaccacagtcaccgtgagctcc

SEQ ID NO: 42 - DNA, 294 bp, Artificial Sequence
  Description of Artificial Sequence: Synthetic polynucleotide
gcctcaacaaaggggcccagcgtgtttccactggctccctctagtaaaagtacctcaggcgggacagcagccctgggatgtctggtgaaggattacttcccagagcccgtcaccgtgtcttggaacagtggcgctctgacaagcggggtccatacttttccagcagtgctgcagtcaagcggcctgtattccctgtcctctgtggtcactgtgcccagttcaagcctggggactcagacctacatctgcaacgtgaatcacaagccatctaataccaaagtcgacaagaaagtg

SEQ ID NO: 43 - DNA, 330 bp, Artificial Sequence
  Description of Artificial Sequence: Synthetic polynucleotide
gcaccagagctgctgggaggaccatccgtgttcctgtttccacccaagcctaaagacaccctgatgattagcaggactcccgaagtcacctgcgtggtcgtggacgtgtcccacgaggaccccgaagtcaagttcaactggtacgtggatggcgtcgaggtgcataatgctaagacaaaaccccgagaggaacagtataattccacttaccgggtcgtgtctgtcctgaccgtgctgcaccaggactggctgaacggcaaggagtacaagtgcaaagtgtctaataaggctctgcccgcacctatcgagaaaacaattagcaaggctaaa

SEQ ID NO: 44 - DNA, 318 bp, Artificial Sequence
  Description of Artificial Sequence: Synthetic polynucleotide
gggcagcctagagaaccacaggtctatgtgctgcctccaagcagggacgagctgactaagaaccaggtctccctgctgtgtctggtgaaagggttctaccctagtgatatcgcagtggagtgggaatcaaatggacagccagaaaacaattatctgacatggccccctgtgctggactcagatggaagcttctttctgtactccaagctgactgtggataaatctcggtggcagcagggcaacgtctttagctgttccgtgatgcatgaggccctgcacaatcattacacccagaagtctctgagtctgtcacctggc

SEQ ID NO: 45 - DNA, 639 bp, Artificial Sequence
  Description of Artificial Sequence: Synthetic polynucleotide
cagatcgtcctgtcacagagccccgctatcctgtccgcatctcctggcgagaaggtgaccatgacatgccgagctagctcctctgtctcctacatgcactggtatcagcagaagcccgggagttcacctaaaccatggatctacgccccatcaaacctggctagcggagtgccagcacggttcagtggctcagggagcggaacatcctattctctgactatttctagagtggaggctgaagacgccgctacctactattgccagcagtggtccttcaatccccctacctttggcgcagggacaaagctggagctgaaaaggaccgtggcagcccctagtgtcttcatttttccaccctccgacgaacagctgaagtccggcacagcctctgtggtctgtctgctgaacaatttctacccacgcgaggccaaggtgcagtggaaagtcgataacgctctgcagagtggcaacagccaggagagcgtgactgaacaggactccaaggattctacctatagtctgagctccactctgaccctgagcaaagcagattacgagaagcacaaagtgtatgcctgcgaagtcacacatcagggactgtctagtcctgtgactaaaagctttaacagaggcgaatgt

SEQ ID NO: 46 - DNA, 318 bp, Artificial Sequence
  Description of Artificial Sequence: Synthetic polynucleotide
cagatcgtcctgtcacagagccccgctatcctgtccgcatctcctggcgagaaggtgaccatgacatgccgagctagctcctctgtctcctacatgcactggtatcagcagaagcccgggagttcacctaaaccatggatctacgccccatcaaacctggctagcggagtgccagcacggttcagtggctcagggagcggaacatcctattctctgactatttctagagtggaggctgaagacgccgctacctactattgccagcagtggtccttcaatccccctacctttggcgcagggacaaagctggagctgaaa

SEQ ID NO: 47 - DNA, 321 bp, Artificial Sequence
  Description of Artificial Sequence: Synthetic polynucleotide
aggaccgtggcagcccctagtgtcttcatttttccaccctccgacgaacagctgaagtccggcacagcctctgtggtctgtctgctgaacaatttctacccacgcgaggccaaggtgcagtggaaagtcgataacgctctgcagagtggcaacagccaggagagcgtgactgaacaggactccaaggattctacctatagtctgagctccactctgaccctgagcaaagcagattacgagaagcacaaagtgtatgcctgcgaagtcacacatcagggactgtctagtcctgtgactaaaagctttaacagaggcgaatgt

SEQ ID NO: 48 - DNA, 642 bp, Artificial Sequence
  Description of Artificial Sequence: Synthetic polynucleotide
gaaatcgtcctgacacagtcccctgccactctgagtctgtcaccaggcgagagggctaccctgtcttgccgcgcaagccagtccgtgagctcctacctggcctggtatcagcagaagccagggcaggctcccagactgctgatctacgacgcatccaaccgagcaaccggcatccccgcacggttctctggcagtgggtcaggaacagactttaccctgacaatctctagtctggagcccgaagatttcgctgtgtactattgccagcagaggtctaattggcctatcacctttggccaggggacacggctggagattaagagaactgtggccgctccaagtgtcttcatttttccccctagcgacgaacagctgaaatccggcacagcctctgtggtctgtctgctgaacaatttctacccccgcgaggcaaaggtgcagtggaaagtcgataacgccctgcagagcggcaacagccaggagtctgtgactgaacaggacagtaaggattcaacctatagcctgtcaagcactctgaccctgagcaaagctgattacgagaagcacaaagtgtatgcatgcgaagtcacacatcagggactgtcctctcccgtcactaaaagctttaaccgaggcgaatgt

SEQ ID NO: 49 - DNA, 321 bp, Artificial Sequence
  Description of Artificial Sequence: Synthetic polynucleotide
gaaatcgtcctgacacagtcccctgccactctgagtctgtcaccaggcgagagggctaccctgtcttgccgcgcaagccagtccgtgagctcctacctggcctggtatcagcagaagccagggcaggctcccagactgctgatctacgacgcatccaaccgagcaaccggcatccccgcacggttctctggcagtgggtcaggaacagactttaccctgacaatctctagtctggagcccgaagatttcgctgtgtactattgccagcagaggtctaattggcctatcacctttggccaggggacacggctggagattaag

SEQ ID NO: 50 - DNA, 321 bp, Artificial Sequence
  Description of Artificial Sequence: Synthetic polynucleotide
agaactgtggccgctccaagtgtcttcatttttccccctagcgacgaacagctgaaatccggcacagcctctgtggtctgtctgctgaacaatttctacccccgcgaggcaaaggtgcagtggaaagtcgataacgccctgcagagcggcaacagccaggagtctgtgactgaacaggacagtaaggattcaacctatagcctgtcaagcactctgaccctgagcaaagctgattacgagaagcacaaagtgtatgcatgcgaagtcacacatcagggactgtcctctcccgtcactaaaagctttaaccgaggcgaatgt

SEQ ID NO: 51 - DNA, 1431 bp, Artificial Sequence
  Description of Artificial Sequence: Synthetic polynucleotide
gacatcaaactgcagcagagcggagctgagctggcacgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggccaccctgaccacagataagagctcctctacagcttacatgcagctgagttcactgactagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtgactatgacctgccgcgcctctagttcagtgagctacatgaactggtatcagcagaaatcaggcaccagccccaagagatggatctacgacacatccaaggtcgcttctggggtgccttataggttcagtgggtcaggaagcggcacttcctactctctgaccattagctccatggaggcagaagatgccgctacatactattgtcagcagtggtctagtaatccactgacatttggggccggaactaaactggagctgaaggcagccgaacccaaatcaagcgacaagacacacacttgcccaccttgtccagcaccagaactgctgggaggacctagcgtgttcctgtttccacccaaaccaaaggatacactgatgatcagccggacccctgaggtcacatgcgtggtcgtggacgtgagccacgaggaccccgaagtcaagttcaactggtacgtggacggcgtcgaagtgcataatgccaaaaccaagcctagggaggaacagtacaatagtacttatcgcgtcgtgtcagtcctgaccgtgctgcatcaggattggctgaacgggaaggagtacaaatgcaaggtgtccaacaaggccctgcctgctccaatcgagaagaccatttctaaagcaaagggccagccccgagaacctcaggtctacgtgtatcctccatcccgggacgagctgaccaaaaaccaggtctctctgacatgtctggtgaaggggttttatccatctgatattgctgtggagtgggaaagtaatggacagcccgagaacaattacaagacaactccccctgtgctggactccgatggatctttcgctctggtcagcaaactgacagtggacaagtccagatggcagcagggcaacgtctttagttgttcagtgatgcacgaggcactgcacaatcattacactcagaaaagcctgtccctgtctcccggcaag

SEQ ID NO: 52 - DNA, 357 bp, Artificial Sequence
  Description of Artificial Sequence: Synthetic polynucleotide
gacatcaaactgcagcagagcggagctgagctggcacgaccaggagccagtgtgaaaatgtcatgcaagacaagcggctacaccttcacacggtatactatgcactgggtgaaacagagacccggccaggggctggaatggatcggatatattaacccttcccgaggctacaccaactataatcagaagtttaaagacaaggccaccctgaccacagataagagctcctctacagcttacatgcagctgagttcactgactagtgaggactcagctgtgtactattgcgcaaggtactatgacgatcattactgtctggattattggggacagggcactaccctgactgtcagctcc

SEQ ID NO: 53 - DNA, 318 bp, Artificial Sequence
  Description of Artificial Sequence: Synthetic polynucleotide
gatatccagctgacccagtccccagcaattatgtccgcctctcccggcgagaaagtgactatgacctgccgcgcctctagttcagtgagctacatgaactggtatcagcagaaatcaggcaccagccccaagagatggatctacgacacatccaaggtcgcttctggggtgccttataggttcagtgggtcaggaagcggcacttcctactctctgaccattagctccatggaggcagaagatgccgctacatactattgtcagcagtggtctagtaatccactgacatttggggccggaactaaactggagctgaag

SEQ ID NO: 54 - DNA, 330 bp, Artificial Sequence
  Description of Artificial Sequence: Synthetic polynucleotide
gcaccagaactgctgggaggacctagcgtgttcctgtttccacccaaaccaaaggatacactgatgatcagccggacccctgaggtcacatgcgtggtcgtggacgtgagccacgaggaccccgaagtcaagttcaactggtacgtggacggcgtcgaagtgcataatgccaaaaccaagcctagggaggaacagtacaatagtacttatcgcgtcgtgtcagtcctgaccgtgctgcatcaggattggctgaacgggaaggagtacaaatgcaaggtgtccaacaaggccctgcctgctccaatcgagaagaccatttctaaagcaaag

SEQ ID NO: 55 - DNA, 318 bp, Artificial Sequence
  Description of Artificial Sequence: Synthetic polynucleotide
ggccagccccgagaacctcaggtctacgtgtatcctccatcccgggacgagctgaccaaaaaccaggtctctctgacatgtctggtgaaggggttttatccatctgatattgctgtggagtgggaaagtaatggacagcccgagaacaattacaagacaactccccctgtgctggactccgatggatctttcgctctggtcagcaaactgacagtggacaagtccagatggcagcagggcaacgtctttagttgttcagtgatgcacgaggcactgcacaatcattacactcagaaaagcctgtccctgtctcccggc

SEQ ID NO: 56 - DNA, 1419 bp, Artificial Sequence
  Description of Artificial Sequence: Synthetic polynucleotide
cagatcgtcctgactcagagccccgctattatgtccgcttcccctggagaaaaggtcactatgacttgttccgcctctagttccgtctcctacatgaactggtatcagcagaaatctggaacaagtcccaagcgatggatctacgacacttccaagctggcatctggagtgcctgcccacttccgaggcagcggctctgggacaagttattcactgactatttctggcatggaggccgaagatgccgctacatactattgccagcagtggagctccaacccattcacctttggatgtggcacaaagctggagatcaatggcggaggaggctccggaggaggagggtctggaggaggaggaagtcaggtccagctgcagcagagcggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactgtctggattattgggggcagggaactaccctgaccgtgtctagtgcagccgagcctaaatcaagcgacaagacccatacatgccccccttgtccggcgccagaagctgcaggcggaccaagcgtgttcctgtttccacccaaacctaaggatactctgatgattagccgaactcctgaggtcacctgcgtggtcgtgagcgtgtcccacgaggacccagaagtcaagttcaactggtacgtggatggggtcgaagtgcataatgccaaaaccaagcccagggaggaacagtacaactccacttatcgcgtcgtgtctgtcctgaccgtgctgcaccaggactggctgaatggcaaggagtacaaatgtaaggtctcaaataaggctctgcccgcccctatcgaaaaaactatctcaaaggcaaaaggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 57 - DNA, 318 bp, Artificial Sequence
  Description of Artificial Sequence: Synthetic polynucleotide
cagatcgtcctgactcagagccccgctattatgtccgcttcccctggagaaaaggtcactatgacttgttccgcctctagttccgtctcctacatgaactggtatcagcagaaatctggaacaagtcccaagcgatggatctacgacacttccaagctggcatctggagtgcctgcccacttccgaggcagcggctctgggacaagttattcactgactatttctggcatggaggccgaagatgccgctacatactattgccagcagtggagctccaacccattcacctttggatgtggcacaaagctggagatcaat

SEQ ID NO: 58 - DNA, 357 bp, Artificial Sequence
  Description of Artificial Sequence: Synthetic polynucleotide
caggtccagctgcagcagagcggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactgtctggattattgggggcagggaactaccctgaccgtgtctagt

SEQ ID NO: 59 - DNA, 330 bp, Artificial Sequence
  Description of Artificial Sequence: Synthetic polynucleotide
gcgccagaagctgcaggcggaccaagcgtgttcctgtttccacccaaacctaaggatactctgatgattagccgaactcctgaggtcacctgcgtggtcgtgagcgtgtcccacgaggacccagaagtcaagttcaactggtacgtggatggggtcgaagtgcataatgccaaaaccaagcccagggaggaacagtacaactccacttatcgcgtcgtgtctgtcctgaccgtgctgcaccaggactggctgaatggcaaggagtacaaatgtaaggtctcaaataaggctctgcccgcccctatcgaaaaaactatctcaaaggcaaaa

SEQ ID NO: 60 - DNA, 318 bp, Artificial Sequence
  Description of Artificial Sequence: Synthetic polynucleotide
ggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 61 - DNA, 1419 bp, Artificial Sequence
  Description of Artificial Sequence: Synthetic polynucleotide
cagatcgtcctgactcagagccccgctattatgtccgcaagccctggagagaaagtgactatgacctgttccgcatctagttccgtgtcctacatgaactggtatcagcagaaatctggaacaagtcccaagcgatggatctacgacacttccaagctggcatctggagtgcctgcccacttccgaggcagcggctctgggacaagttattcactgactattagcggcatggaggccgaagatgccgctacatactattgccagcagtggagctccaacccattcacctttggatgtggcacaaagctggagatcaatggcggaggaggctccggaggaggagggtctggaggaggaggaagtcaggtccagctgcagcagtccggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactccctggattattgggggcagggaactaccctgaccgtgtctagtgcagccgagcctaaatcaagcgacaagacccatacatgccccccttgtccggcgccagaagctgcaggcggaccaagtgtgttcctgtttccacccaaacctaaggatactctgatgatttctcgaactcctgaggtcacctgcgtggtcgtgagcgtgtcccacgaggacccagaagtcaagttcaactggtacgtggatggggtcgaagtgcataatgccaaaaccaagcccagggaggaacagtacaactcaacttatcgcgtcgtgtctgtcctgaccgtgctgcaccaggactggctgaatggcaaggagtacaaatgtaaggtctcaaataaggctctgcccgcccctatcgaaaaaactatctctaaggcaaaaggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 62 - DNA, 318 bp, Artificial Sequence
  Description of Artificial Sequence: Synthetic polynucleotide
cagatcgtcctgactcagagccccgctattatgtccgcaagccctggagagaaagtgactatgacctgttccgcatctagttccgtgtcctacatgaactggtatcagcagaaatctggaacaagtcccaagcgatggatctacgacacttccaagctggcatctggagtgcctgcccacttccgaggcagcggctctgggacaagttattcactgactattagcggcatggaggccgaagatgccgctacatactattgccagcagtggagctccaacccattcacctttggatgtggcacaaagctggagatcaat

SEQ ID NO: 63 - DNA, 357 bp, Artificial Sequence
  Description of Artificial Sequence: Synthetic polynucleotide
caggtccagctgcagcagtccggagcagaactggctagaccaggagccagtgtgaaaatgtcatgcaaggccagcggctacacattcactcggtataccatgcattgggtgaaacagagaccaggacagtgtctggagtggatcggctacattaatcccagcagggggtacacaaactacaaccagaagtttaaagacaaggcaaccctgaccaccgataagtctagttcaacagcttatatgcagctgagctccctgacttcagaagacagcgctgtgtactattgcgcacgctactatgacgatcactactccctggattattgggggcagggaactaccctgaccgtgtctagt

SEQ ID NO: 64 - DNA, 330 bp, Artificial Sequence
  Description of Artificial Sequence: Synthetic polynucleotide
gcgccagaagctgcaggcggaccaagtgtgttcctgtttccacccaaacctaaggatactctgatgatttctcgaactcctgaggtcacctgcgtggtcgtgagcgtgtcccacgaggacccagaagtcaagttcaactggtacgtggatggggtcgaagtgcataatgccaaaaccaagcccagggaggaacagtacaactcaacttatcgcgtcgtgtctgtcctgaccgtgctgcaccaggactggctgaatggcaaggagtacaaatgtaaggtctcaaataaggctctgcccgcccctatcgaaaaaactatctctaaggcaaaa

SEQ ID NO: 65 - DNA, 318 bp, Artificial Sequence
  Description of Artificial Sequence: Synthetic polynucleotide
ggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 66 - DNA, 1449 bp, Artificial Sequence
  Description of Artificial Sequence: Synthetic polynucleotide
gatattcagctgacacagagccccgcatccctggccgtgagcctgggacagagagcaactatttcctgcaaagcctcacagagcgtggactatgatggagacagctatctgaactggtaccagcagatcccaggccagccccctaaactgctgatctacgacgccagcaatctggtgtccggcatcccacccaggttcagtggatcaggcagcgggaccgattttacactgaacattcaccctgtcgagaaggtggacgccgctacctaccattgccagcagtccacagaggacccctggactttcggatgtggcaccaaactggaaatcaagggcgggggaggctcaggaggaggagggagcggaggaggaggcagccaggtgcagctgcagcagagcggagcagaactggtccgacctggaagctccgtgaaaatttcttgcaaggccagtggctatgctttttctagttactggatgaattgggtgaagcagcgaccaggacagtgtctggagtggatcgggcagatttggcctggggatggagacaccaactataatggaaagttcaaaggcaaggcaactctgaccgccgacgaatcaagctccacagcttatatgcagctgtctagtctggctagtgaggattcagcagtgtacttttgcgcccggagagaaaccacaactgtgggcagatactattacgcaatggactactggggccaggggaccacagtcaccgtgtcaagcgcagccgagcccaaatcctctgataagacacacacttgccctccatgtccggcgccagaagctgcaggcggaccttccgtgttcctgtttccccctaaaccaaaggacactctgatgatctctcgcactccagaggtcacctgcgtggtcgtgtccgtgtctcacgaggaccccgaagtcaaattcaactggtatgtggacggggtcgaagtgcataatgccaaaacaaagcctagggaggaacagtataactctacataccgcgtcgtgagtgtcctgactgtgctgcatcaggattggctgaatggcaaggagtacaaatgtaaggtctcaaataaggctctgcccgcccctatcgaaaaaactatctctaaagctaaaggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 67 - DNA, 333 bp, Artificial Sequence
  Description of Artificial Sequence: Synthetic polynucleotide
gatattcagctgacacagagccccgcatccctggccgtgagcctgggacagagagcaactatttcctgcaaagcctcacagagcgtggactatgatggagacagctatctgaactggtaccagcagatcccaggccagccccctaaactgctgatctacgacgccagcaatctggtgtccggcatcccacccaggttcagtggatcaggcagcgggaccgattttacactgaacattcaccctgtcgagaaggtggacgccgctacctaccattgccagcagtccacagaggacccctggactttcggatgtggcaccaaactggaaatcaag

SEQ ID NO: 68 - DNA, 372 bp, Artificial Sequence
  Description of Artificial Sequence: Synthetic polynucleotide
caggtgcagctgcagcagagcggagcagaactggtccgacctggaagctccgtgaaaatttcttgcaaggccagtggctatgctttttctagttactggatgaattgggtgaagcagcgaccaggacagtgtctggagtggatcgggcagatttggcctggggatggagacaccaactataatggaaagttcaaaggcaaggcaactctgaccgccgacgaatcaagctccacagcttatatgcagctgtctagtctggctagtgaggattcagcagtgtacttttgcgcccggagagaaaccacaactgtgggcagatactattacgcaatggactactggggccaggggaccacagtcaccgtgtcaagc

SEQ ID NO: 69 - DNA, 330 bp, Artificial Sequence
  Description of Artificial Sequence: Synthetic polynucleotide
gcgccagaagctgcaggcggaccttccgtgttcctgtttccccctaaaccaaaggacactctgatgatctctcgcactccagaggtcacctgcgtggtcgtgtccgtgtctcacgaggaccccgaagtcaaattcaactggtatgtggacggggtcgaagtgcataatgccaaaacaaagcctagggaggaacagtataactctacataccgcgtcgtgagtgtcctgactgtgctgcatcaggattggctgaatggcaaggagtacaaatgtaaggtctcaaataaggctctgcccgcccctatcgaaaaaactatctctaaagctaaa

SEQ ID NO: 70 - DNA, 318 bp, Artificial Sequence; identical to SEQ ID NO: 60
  Description of Artificial Sequence: Synthetic polynucleotide

SEQ ID NO: 71 - DNA, 1452 bp, Artificial Sequence
  Description of Artificial Sequence: Synthetic polynucleotide
gacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcttatatgcagctgtctagtctggccagcgaggattccgctgtctacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaagggccagcctcgagaaccacaggtgtatacatacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgacagtggataagtcacggtggcagcagggaaacgtctttagttgttcagtgatgcacgaggccctgcacaatcattacactcagaaaagcctgtccctgtctcccggcaag

SEQ ID NO: 72 - DNA, 333 bp, Artificial Sequence
  Description of Artificial Sequence: Synthetic polynucleotide
gacattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 73 - DNA, 372 bp, Artificial Sequence
  Description of Artificial Sequence: Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacactaactataatggaaagttcaaaggcaaggctacactgactgcagacgagtcaagctccaccgcttatatgcagctgtctagtctggccagcgaggattccgctgtctacttttgcgcacggagagaaaccacaactgtgggcaggtactattacgcaatggactactggggccaggggaccacagtcaccgtgtcaagc

SEQ ID NO: 74 - DNA, 330 bp, Artificial Sequence
  Description of Artificial Sequence: Synthetic polynucleotide
gcacctgagctgctgggaggaccaagcgtgttcctgtttccacctaaacctaaggacaccctgatgatctctcggacacccgaagtcacttgtgtggtcgtggatgtgagccacgaggaccctgaagtcaaattcaactggtacgtggatggcgtcgaggtgcataatgccaaaacaaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagaccatttccaaagctaag

SEQ ID NO: 75 - DNA, 318 bp, Artificial Sequence
  Description of Artificial Sequence: Synthetic polynucleotide
ggccagcctcgagaaccacaggtgtatacatacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgacagtggataagtcacggtggcagcagggaaacgtctttagttgttcagtgatgcacgaggccctgcacaatcattacactcagaaaagcctgtccctgtctcccggc

SEQ ID NO: 76 - DNA, 1431 bp, Artificial Sequence
  Description of Artificial Sequence: Synthetic polynucleotide
gatattaagctgcagcagagcggagctgagctggcacgaccaggagccagtgtgaaaatgtcatgcaagaccagcggctacacattcactcggtatacaatgcactgggtgaagcagagaccaggacagggactggaatggatcggatatattaacccttcccgaggctacaccaactataatcagaagtttaaagacaaggccactctgaccacagataagagctcctctaccgcttacatgcagctgagttcactgacaagtgaggactcagctgtgtactattgcgcaaggtactatgacgatcattactgtctggattattggggacagggcactaccctgactgtcagctccgtggaaggagggagcggaggctccggaggatctggcgggagtggaggcgtggacgatatccagctgacccagtccccagcaattatgtccgcctctcccggcgagaaagtcaccatgacatgccgcgcttctagttcagtgagctacatgaactggtatcagcagaaatcaggcactagccccaagagatggatctacgacacctccaaggtcgcatctggggtgccttataggttcagtgggtcaggaagcggcacctcctactctctgacaattagctccatggaggcagaagatgccgctacctactattgtcagcagtggtctagtaatccactgacttttggggccggaaccaaactggagctgaaggcagccgaacccaaatcaagcgacaagactcacacctgccccccttgtccagcacccgaactgctggggggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgctaaaactaagcctagggaggaacagtacaatagtacatatagagtcgtgtcagtgctgaccgtcctgcatcaggattggctgaacgggaaggagtacaaatgcaaggtgtccaacaaggccctgcctgctccaatcgagaagacaatttctaaagccaagggccagccccgagaacctcaggtgtatacactgcctccatcccgggacgagctgactaaaaaccaggtctctctgctgtgtctggtgaaggggttctacccatctgatattgctgtggagtgggaaagtaatggacagcccgagaacaattatatgacctggccccctgtcctggactccgatggatctttctttctgtacagcaaactgacagtggacaagtccagatggcagcagggcaacgtctttagttgttcagtgatgcacgaggccctgcacaatcattacacccagaaaagcctgtccctgtctcccggcaag

SEQ ID NO: 77 - DNA, 357 bp, Artificial Sequence
  Description of Artificial Sequence: Synthetic polynucleotide
gatattaagctgcagcagagcggagctgagctggcacgaccaggagccagtgtgaaaatgtcatgcaagaccagcggctacacattcactcggtatacaatgcactgggtgaagcagagaccaggacagggactggaatggatcggatatattaacccttcccgaggctacaccaactataatcagaagtttaaagacaaggccactctgaccacagataagagctcctctaccgcttacatgcagctgagttcactgacaagtgaggactcagctgtgtactattgcgcaaggtactatgacgatcattactgtctggattattggggacagggcactaccctgactgtcagctcc

SEQ ID NO: 78 - DNA, 318 bp, Artificial Sequence
  Description of Artificial Sequence: Synthetic polynucleotide
gatatccagctgacccagtccccagcaattatgtccgcctctcccggcgagaaagtcaccatgacatgccgcgcttctagttcagtgagctacatgaactggtatcagcagaaatcaggcactagccccaagagatggatctacgacacctccaaggtcgcatctggggtgccttataggttcagtgggtcaggaagcggcacctcctactctctgacaattagctccatggaggcagaagatgccgctacctactattgtcagcagtggtctagtaatccactgacttttggggccggaaccaaactggagctgaag

SEQ ID NO: 79 - DNA, 330 bp, Artificial Sequence
  Description of Artificial Sequence: Synthetic polynucleotide
gcacccgaactgctggggggacctagcgtgttcctgtttccacccaaaccaaaggatacactgatgatcagccggacacctgaggtcacttgcgtggtcgtggacgtgagccacgaggaccccgaagtcaagttcaactggtacgtggacggcgtcgaagtgcataatgctaaaactaagcctagggaggaacagtacaatagtacatatagagtcgtgtcagtgctgaccgtcctgcatcaggattggctgaacgggaaggagtacaaatgcaaggtgtccaacaaggccctgcctgctccaatcgagaagacaatttctaaagccaag

SEQ ID NO: 80 - DNA, 318 bp, Artificial Sequence
  Description of Artificial Sequence: Synthetic polynucleotide
ggccagccccgagaacctcaggtgtatacactgcctccatcccgggacgagctgactaaaaaccaggtctctctgctgtgtctggtgaaggggttctacccatctgatattgctgtggagtgggaaagtaatggacagcccgagaacaattatatgacctggccccctgtcctggactccgatggatctttctttctgtacagcaaactgacagtggacaagtccagatggcagcagggcaacgtctttagttgttcagtgatgcacgaggccctgcacaatcattacacccagaaaagcctgtccctgtctcccggc

SEQ ID NO: 81 - DNA, 1422 bp, Artificial Sequence
  Description of Artificial Sequence: Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtccagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctccgcagccgaacctaaatctagtgacaagactcatacctgccccccttgtccagcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtgtacactctgcctccatctcgggacgagctgaccaaaaaccaggtcagtctgctgtgtctggtgaagggcttctatccaagcgatattgctgtggagtgggaatccaatgggcagcccgaaaacaattacatgacatggccccctgtcctggactcagatgggagcttctttctgtatagtaaactgactgtggacaagtcacggtggcagcagggaaacgtctttagctgttccgtgatgcatgaggccctgcacaatcattacacccagaaatctctgagtctgtcacccggcaag

SEQ ID NO: 82 - DNA, 318 bp, Artificial Sequence
  Description of Artificial Sequence: Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaat

SEQ ID NO: 83 - DNA, 357 bp, Artificial Sequence
  Description of Artificial Sequence: Synthetic polynucleotide
caggtccagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctcc

SEQ ID NO: 84 - DNA, 330 bp, Artificial Sequence
  Description of Artificial Sequence: Synthetic polynucleotide
gcaccagagctgctgggaggaccttccgtgttcctgtttccacccaaaccaaaggatactctgatgatctcccggacacctgaagtcacttgcgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaag

SEQ ID NO: 85 - DNA, 318 bp, Artificial Sequence
  Description of Artificial Sequence: Synthetic polynucleotide
gggcagccccgagaacctcaggtgtacactctgcctccatctcgggacgagctgaccaaaaaccaggtcagtctgctgtgtctggtgaagggcttctatccaagcgatattgctgtggagtgggaatccaatgggcagcccgaaaacaattacatgacatggccccctgtcctggactcagatgggagcttctttctgtatagtaaactgactgtggacaagtcacggtggcagcagggaaacgtctttagctgttccgtgatgcatgaggccctgcacaatcattacacccagaaatctctgagtctgtcacccggc

SEQ ID NO: 86 - DNA, 1452 bp, Artificial Sequence
  Description of Artificial Sequence: Synthetic polynucleotide
gatattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcttatatgcagctgtctagtctggccagcgaggattccgctgtgtacttttgcgcacggagagaaaccacaactgtgggcaggtactattacgcaatggactactggggccaggggaccacagtcaccgtgtcaagcgcagccgaacccaaatcctctgataagacccacacatgccctccatgtccagcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagacaatttccaaagctaagggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgactgtggataagtcacggtggcagcagggaaacgtctttagttgttcagtgatgcacgaggccctgcacaatcattacacccagaaaagcctgtccctgtctcccggcaag

SEQ ID NO: 87 - DNA, 333 bp, Artificial Sequence
  Description of Artificial Sequence: Synthetic polynucleotide
gatattcagctgacacagagtcctgcttcactggcagtgagcctgggacagcgagcaactatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctgggactgattttaccctgaacattcacccagtcgagaaggtggacgccgctacctaccattgccagcagtctaccgaggacccctggacattcggcgggggaactaaactggaaatcaag

SEQ ID NO: 88 - DNA, 372 bp, Artificial Sequence
  Description of Artificial Sequence: Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacaccaactataatggaaagttcaaaggcaaggctacactgactgcagacgagtcaagctccacagcttatatgcagctgtctagtctggccagcgaggattccgctgtgtacttttgcgcacggagagaaaccacaactgtgggcaggtactattacgcaatggactactggggccaggggaccacagtcaccgtgtcaagc

SEQ ID NO: 89 - DNA, 330 bp, Artificial Sequence
  Description of Artificial Sequence: Synthetic polynucleotide
gcacctgagctgctgggaggaccaagcgtgttcctgtttccacctaaacctaaggacacactgatgatctctcggacacccgaagtcacttgtgtggtcgtggatgtgagccacgaggaccctgaagtcaaattcaactggtacgtggatggcgtcgaggtgcataatgccaaaactaagcctagggaggaacagtataactccacttaccgcgtcgtgtctgtcctgaccgtgctgcatcaggactggctgaacggaaaggagtacaaatgcaaggtgagcaacaaggccctgccagctcccatcgagaagacaatttccaaagctaag

SEQ ID NO: 90 - DNA, 318 bp, Artificial Sequence
  Description of Artificial Sequence: Synthetic polynucleotide
ggccagcctcgagaaccacaggtctatgtgtacccacccagccgggacgagctgaccaaaaaccaggtctccctgacatgtctggtgaagggattttatccttctgatattgccgtggagtgggaaagtaatggccagccagaaaacaattacaagactacccctccagtgctggattctgacgggagtttcgcactggtcagtaaactgactgtggataagtcacggtggcagcagggaaacgtctttagttgttcagtgatgcacgaggccctgcacaatcattacacccagaaaagcctgtccctgtctcccggc

SEQ ID NO: 91 - DNA, 639 bp, Artificial Sequence
  Description of Artificial Sequence: Synthetic polynucleotide
cagattgtcctgtctcagagtcccgctatcctgtcagcaagccctggggagaaggtgaccatgacatgccgagccagctcctctgtcagctacatccactggttccagcagaagccaggcagttcacctaaaccatggatctacgccacatctaacctggctagtggagtgcccgtccggttttccggctctgggagtggaacatcatacagcctgactatttccagagtggaggccgaagacgccgctacctactattgccagcagtggacctctaatccccctacattcggcgggggaactaagctggagatcaaaaggactgtggcagccccttctgtcttcatttttccacccagtgacgaacagctgaaatcaggaaccgcttccgtggtctgtctgctgaacaacttctacccccgcgaggcaaaggtgcagtggaaagtcgataacgccctgcagtccggcaattctcaggagagtgtgaccgaacaggactcaaaggatagcacatattccctgagctccactctgaccctgtccaaagctgattacgaaaagcataaagtgtatgcatgtgaggtcacccaccaggggctgagtagtcccgtcacaaagagtttcaatagaggagagtgt

SEQ ID NO: 92 - DNA, 318 bp, Artificial Sequence
  Description of Artificial Sequence: Synthetic polynucleotide
cagattgtcctgtctcagagtcccgctatcctgtcagcaagccctggggagaaggtgaccatgacatgccgagccagctcctctgtcagctacatccactggttccagcagaagccaggcagttcacctaaaccatggatctacgccacatctaacctggctagtggagtgcccgtccggttttccggctctgggagtggaacatcatacagcctgactatttccagagtggaggccgaagacgccgctacctactattgccagcagtggacctctaatccccctacattcggcgggggaactaagctggagatcaaa

SEQ ID NO: 93 - DNA, 321 bp, Artificial Sequence
  Description of Artificial Sequence: Synthetic polynucleotide
aggactgtggcagccccttctgtcttcatttttccacccagtgacgaacagctgaaatcaggaaccgcttccgtggtctgtctgctgaacaacttctacccccgcgaggcaaaggtgcagtggaaagtcgataacgccctgcagtccggcaattctcaggagagtgtgaccgaacaggactcaaaggatagcacatattccctgagctccactctgaccctgtccaaagctgattacgaaaagcataaagtgtatgcatgtgaggtcacccaccaggggctgagtagtcccgtcacaaagagtttcaatagaggagagtgt

SEQ ID NO: 94 - DNA, 1353 bp, Artificial Sequence
  Description of Artificial Sequence: Synthetic polynucleotide
caggtccagctgcagcagcccggagctgaactggtcaaacctggcgcatccgtgaaaatgtcttgcaaggctagtggctacacattcacttcctataacatgcactgggtgaagcagacaccaggacgaggactggagtggatcggagcaatctaccctggaaacggcgacacttcttataatcagaagtttaaaggcaaggccaccctgacagctgataagagctcctctaccgcctacatgcagctgagttcactgacaagtgaagactcagcagtgtactattgcgccagaagcacctactatggcggggattggtacttcaacgtgtggggggcaggaaccacagtcaccgtgagcgccgcttccacaaaaggaccaagcgtgtttccactggcaccaagctccaagtcaaccagcggaggaacagcagccctgggatgtctggtgaaggactacttcccagagcccgtcaccgtgtcttggaacagtggcgccctgacaagcggggtccatacttttcccgctgtgctgcagtctagtggcctgtacagcctgtcaagcgtggtcaccgtcccttcctctagtctggggactcagacctatatctgcaacgtgaatcacaaaccttctaatacaaaggtcgacaagaaagtggaaccaaaaagttgtgataagacacatacttgcccaccttgtcctgcaccagagctgctgggaggaccatccgtgttcctgtttccacccaaacccaaggacactctgatgattagccggactcctgaagtcacctgcgtggtcgtggacgtgagccacgaggaccccgaagtcaaattcaactggtacgtggatggcgtcgaggtgcataatgccaaaacaaagccccgggaggaacagtacaactcaacatatagagtcgtgagcgtcctgactgtgctgcaccaggactggctgaacggcaaggagtataaatgcaaggtgtccaacaaggccctgcccgcacctatcgagaagactatttctaaagccaagggccagcctagggaaccacaggtgtacgtgctgcctccaagccgcgacgagctgactaaaaaccaggtctccctgctgtgtctggtgaaggggttctatccaagtgatatcgctgtggagtgggaatcaaatggacagcccgagaacaattacctgacttggccccctgtgctggactcagatgggagcttctttctgtattccaaactgaccgtggataagtctcggtggcagcagggaaatgtcttttcctgttctgtgatgcacgaagcactgcacaatcactacacccagaagtccctgagcctgtcacccggcaaa

SEQ ID NO: 95 - DNA, 363 bp, Artificial Sequence
  Description of Artificial Sequence: Synthetic polynucleotide
caggtccagctgcagcagcccggagctgaactggtcaaacctggcgcatccgtgaaaatgtcttgcaaggctagtggctacacattcacttcctataacatgcactgggtgaagcagacaccaggacgaggactggagtggatcggagcaatctaccctggaaacggcgacacttcttataatcagaagtttaaaggcaaggccaccctgacagctgataagagctcctctaccgcctacatgcagctgagttcactgacaagtgaagactcagcagtgtactattgcgccagaagcacctactatggcggggattggtacttcaacgtgtggggggcaggaaccacagtcaccgtgagcgcc

SEQ ID NO: 96 - DNA, 294 bp, Artificial Sequence
  Description of Artificial Sequence: Synthetic polynucleotide
gcttccacaaaaggaccaagcgtgtttccactggcaccaagctccaagtcaaccagcggaggaacagcagccctgggatgtctggtgaaggactacttcccagagcccgtcaccgtgtcttggaacagtggcgccctgacaagcggggtccatacttttcccgctgtgctgcagtctagtggcctgtacagcctgtcaagcgtggtcaccgtcccttcctctagtctggggactcagacctatatctgcaacgtgaatcacaaaccttctaatacaaaggtcgacaagaaagtg

SEQ ID NO: 97 - DNA, 330 bp, Artificial Sequence
  Description of Artificial Sequence: Synthetic polynucleotide
gcaccagagctgctgggaggaccatccgtgttcctgtttccacccaaacccaaggacactctgatgattagccggactcctgaagtcacctgcgtggtcgtggacgtgagccacgaggaccccgaagtcaaattcaactggtacgtggatggcgtcgaggtgcataatgccaaaacaaagccccgggaggaacagtacaactcaacatatagagtcgtgagcgtcctgactgtgctgcaccaggactggctgaacggcaaggagtataaatgcaaggtgtccaacaaggccctgcccgcacctatcgagaagactatttctaaagccaag

SEQ ID NO: 98 - DNA, 318 bp, Artificial Sequence
  Description of Artificial Sequence: Synthetic polynucleotide
ggccagcctagggaaccacaggtgtacgtgctgcctccaagccgcgacgagctgactaaaaaccaggtctccctgctgtgtctggtgaaggggttctatccaagtgatatcgctgtggagtgggaatcaaatggacagcccgagaacaattacctgacttggccccctgtgctggactcagatgggagcttctttctgtattccaaactgaccgtggataagtctcggtggcagcagggaaatgtcttttcctgttctgtgatgcacgaagcactgcacaatcactacacccagaagtccctgagcctgtcacccggc

SEQ ID NO: 99 - DNA, 1344 bp, Artificial Sequence
  Description of Artificial Sequence: Synthetic polynucleotide
caggtccagctggtccagtccggaggaggagtggtccagccaggacggtcactgagactgagctgcaaggcttccgggtacactttcacccgatataccatgcactgggtgcggcaggcaccagggaaaggactggaatggatcgggtacattaaccctagcaggggatacacaaactataatcagaaggtgaaagacaggttcactatctctcgcgataacagtaagaataccgcctttctgcagatggacagcctgcgccccgaggatacaggcgtgtatttctgcgctcgatactatgacgatcactactgtctggactattggggccaggggactccagtcaccgtgagctccgcatcaactaagggacccagcgtgtttccactggccccctctagtaaatccacatctggaggaactgcagctctgggatgcctggtgaaggattacttcccagagcccgtcaccgtgagctggaactccggagccctgacttccggcgtccatacctttcccgctgtgctgcagtcaagcgggctgtactctctgtcctctgtggtcacagtgcctagttcaagcctgggaacacagacttatatctgcaacgtgaatcacaagcctagcaatactaaagtcgacaagaaagtggaaccaaagagctgtgataaaacccatacatgccccccttgtcctgcaccagaggcagcaggaggaccaagcgtgttcctgtttccacccaagcctaaagacaccctgatgattagccggacccctgaagtgacatgtgtggtcgtgagtgtgtcacacgaggacccagaagtcaagttcaactggtacgtggatggcgtcgaggtgcataatgccaagacaaaacctagagaggaacagtacaattccacctatagggtcgtgtctgtcctgacagtgctgcaccaggattggctgaacgggaaagagtataagtgcaaagtgtccaataaggctctgcccgcacctatcgagaaaaccatttctaaggctaaaggccagcctagggaaccacaggtctacgtgtatcctccatctcgcgacgagctgacaaagaaccaggtcagtctgacttgtctggtgaaaggattttacccaagcgatattgccgtggagtgggaatccaatggccagcccgaaaacaattataagaccacaccccctgtgctggactctgatggcagtttcgcactggtcagtaagctgactgtggacaaatcaagatggcagcaggggaacgtctttagctgttccgtgatgcatgaggccctgcacaatcattacacccagaagtctctgagtctgtcacccggc

SEQ ID NO: 100 - DNA, 357 bp, Artificial Sequence
  Description of Artificial Sequence: Synthetic polynucleotide
caggtccagctggtccagtccggaggaggagtggtccagccaggacggtcactgagactgagctgcaaggcttccgggtacactttcacccgatataccatgcactgggtgcggcaggcaccagggaaaggactggaatggatcgggtacattaaccctagcaggggatacacaaactataatcagaaggtgaaagacaggttcactatctctcgcgataacagtaagaataccgcctttctgcagatggacagcctgcgccccgaggatacaggcgtgtatttctgcgctcgatactatgacgatcactactgtctggactattggggccaggggactccagtcaccgtgagctcc

SEQ ID NO: 101 - DNA, 294 bp, Artificial Sequence
  Description of Artificial Sequence: Synthetic polynucleotide
gcatcaactaagggacccagcgtgtttccactggccccctctagtaaatccacatctggaggaactgcagctctgggatgcctggtgaaggattacttcccagagcccgtcaccgtgagctggaactccggagccctgacttccggcgtccatacctttcccgctgtgctgcagtcaagcgggctgtactctctgtcctctgtggtcacagtgcctagttcaagcctgggaacacagacttatatctgcaacgtgaatcacaagcctagcaatactaaagtcgacaagaaagtg

SEQ ID NO: 102 - DNA, 330 bp, Artificial Sequence
  Description of Artificial Sequence: Synthetic polynucleotide
gcaccagaggcagcaggaggaccaagcgtgttcctgtttccacccaagcctaaagacaccctgatgattagccggacccctgaagtgacatgtgtggtcgtgagtgtgtcacacgaggacccagaagtcaagttcaactggtacgtggatggcgtcgaggtgcataatgccaagacaaaacctagagaggaacagtacaattccacctatagggtcgtgtctgtcctgacagtgctgcaccaggattggctgaacgggaaagagtataagtgcaaagtgtccaataaggctctgcccgcacctatcgagaaaaccatttctaaggctaaa

SEQ ID NO: 103 - DNA, 318 bp, Artificial Sequence
  Description of Artificial Sequence: Synthetic polynucleotide
ggccagcctagggaaccacaggtctacgtgtatcctccatctcgcgacgagctgacaaagaaccaggtcagtctgacttgtctggtgaaaggattttacccaagcgatattgccgtggagtgggaatccaatggccagcccgaaaacaattataagaccacaccccctgtgctggactctgatggcagtttcgcactggtcagtaagctgactgtggacaaatcaagatggcagcaggggaacgtctttagctgttccgtgatgcatgaggccctgcacaatcattacacccagaagtctctgagtctgtcacccggc

SEQ ID NO: 104 - DNA, 1434 bp, Artificial Sequence
  Description of Artificial Sequence: Synthetic polynucleotide
gaagtccagctggtcgagagcggaggaggactggtgcagccaggacggtccctgagactgtcttgcgccgctagtgggttcacctttaacgactatgccatgcactgggtccgacaggctccaggaaagggactggaatgggtgtctaccatcagttggaatagtggatcaattggctatgctgactccgtgaaaggcaggttcacaatctcacgcgataacgcaaagaaaagcctgtacctgcagatgaacagcctgcgcgccgaggacacagctctgtactattgcgccaaggatattcagtacgggaactactattacggaatggacgtgtgggggcagggaaccacagtcactgtgagctccggcgggggaggctcaggaggaggagggagcggaggaggaggcagcgaaatcgtgctgactcagagccctgcaaccctgagcctgtccccaggagagcgagctacactgagctgtcgggcatctcagagtgtgtctagttatctggcatggtaccagcagaagccagggcaggcccccagactgctgatctacgatgcatccaacagagccactggcatccccgcaaggttctcaggcagcgggtccggaaccgactttactctgaccatctcaagcctggagcccgaagatttcgctgtgtattactgccagcagaggtctaattggcctatcacatttggccaggggactcgcctggagattaaggcagccgaaccaaagtcctctgacaaaacacacacttgccccccttgtccagcaccagaactgctgggaggaccaagcgtgttcctgtttccacccaagcctaaagataccctgatgattagtaggacccctgaggtcacatgtgtggtcgtggacgtgagccacgaggaccccgaagtcaagtttaactggtacgtggacggcgtcgaagtgcataatgccaagacaaaaccccgcgaggaacagtataattctacctaccgagtcgtgagtgtcctgacagtgctgcatcaggattggctgaacggaaaagagtacaagtgcaaagtgtccaataaggctctgcctgcaccaatcgagaaaactatttctaaggcaaaagggcagccccgggaacctcaggtctatgtgctgcctccatccagagacgagctgaccaagaaccaggtctctctgctgtgtctggtgaaaggattctacccatcagatatcgctgtggagtgggaaagcaatggccagcccgagaacaattatctgacatggccccctgtgctggactcagatggcagcttctttctgtactctaagctgactgtggataaaagtcggtggcagcaggggaacgtcttttcttgtagtgtgatgcatgaggccctgcacaatcattacacccagaagtcactgagcctgtcccctggcaaa

SEQ ID NO: 105 - DNA, 366 bp, Artificial Sequence
  Description of Artificial Sequence: Synthetic polynucleotide
gaagtccagctggtcgagagcggaggaggactggtgcagccaggacggtccctgagactgtcttgcgccgctagtgggttcacctttaacgactatgccatgcactgggtccgacaggctccaggaaagggactggaatgggtgtctaccatcagttggaatagtggatcaattggctatgctgactccgtgaaaggcaggttcacaatctcacgcgataacgcaaagaaaagcctgtacctgcagatgaacagcctgcgcgccgaggacacagctctgtactattgcgccaaggatattcagtacgggaactactattacggaatggacgtgtgggggcagggaaccacagtcactgtgagctcc

SEQ ID NO: 106 - DNA, 321 bp, Artificial Sequence
  Description of Artificial Sequence: Synthetic polynucleotide
gaaatcgtgctgactcagagccctgcaaccctgagcctgtccccaggagagcgagctacactgagctgtcgggcatctcagagtgtgtctagttatctggcatggtaccagcagaagccagggcaggcccccagactgctgatctacgatgcatccaacagagccactggcatccccgcaaggttctcaggcagcgggtccggaaccgactttactctgaccatctcaagcctggagcccgaagatttcgctgtgtattactgccagcagaggtctaattggcctatcacatttggccaggggactcgcctggagattaag

SEQ ID NO: 107 - DNA, 330 bp, Artificial Sequence
  Description of Artificial Sequence: Synthetic polynucleotide
gcaccagaactgctgggaggaccaagcgtgttcctgtttccacccaagcctaaagataccctgatgattagtaggacccctgaggtcacatgtgtggtcgtggacgtgagccacgaggaccccgaagtcaagtttaactggtacgtggacggcgtcgaagtgcataatgccaagacaaaaccccgcgaggaacagtataattctacctaccgagtcgtgagtgtcctgacagtgctgcatcaggattggctgaacggaaaagagtacaagtgcaaagtgtccaataaggctctgcctgcaccaatcgagaaaactatttctaaggcaaaa

SEQ ID NO: 108 - DNA, 318 bp, Artificial Sequence
  Description of Artificial Sequence: Synthetic polynucleotide
gggcagccccgggaacctcaggtctatgtgctgcctccatccagagacgagctgaccaagaaccaggtctctctgctgtgtctggtgaaaggattctacccatcagatatcgctgtggagtgggaaagcaatggccagcccgagaacaattatctgacatggccccctgtgctggactcagatggcagcttctttctgtactctaagctgactgtggataaaagtcggtggcagcaggggaacgtcttttcttgtagtgtgatgcatgaggccctgcacaatcattacacccagaagtcactgagcctgtcccctggc

SEQ ID NO: 109 - DNA, 1452 bp, Artificial Sequence
  Description of Artificial Sequence: Synthetic polynucleotide
gatattcagctgacccagagtcctgcatcactggctgtgagcctgggacagcgagcaacaatctcctgcaaagccagtcagtcagtggactatgatggcgactcctatctgaactggtaccagcagatcccagggcagccccctaagctgctgatctacgacgcttcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacacaaactataatggaaagttcaaaggcaaggccactctgaccgctgacgagtcaagctccactgcttatatgcagctgtctagtctggcaagcgaggattccgccgtctacttttgcgctcggagagaaaccacaactgtgggcaggtactattacgcaatggactactggggccaggggaccacagtcaccgtgtcaagcgcagccgaacccaaatcctctgataagacacacacttgccctccatgtccagcacctgaggctgcaggaggaccaagcgtgttcctgtttccccctaaacctaaggacactctgatgatctctcggactcccgaagtcacctgtgtggtcgtgagcgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggcactgccagcccccatcgagaagaccatttccaaagccaagggccagcctcgagaaccacaggtctatgtgctgccacccagccgggacgagctgacaaaaaaccaggtctccctgctgtgtctggtgaagggattctacccttctgatattgctgtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaag

SEQ ID NO: 110 - DNA, 333 bp, Artificial Sequence
  Description of Artificial Sequence: Synthetic polynucleotide
gatattcagctgacccagagtcctgcatcactggctgtgagcctgggacagcgagcaacaatctcctgcaaagccagtcagtcagtggactatgatggcgactcctatctgaactggtaccagcagatcccagggcagccccctaagctgctgatctacgacgcttcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaag

SEQ ID NO: 111 - DNA, 372 bp, Artificial Sequence
  Description of Artificial Sequence: Synthetic polynucleotide
caggtgcagctgcagcagagcggagcagagctggtcagaccaggaagctccgtgaaaatttcctgtaaggcttctggctatgcattttctagttactggatgaattgggtgaagcagaggccaggacagggcctggaatggatcgggcagatttggcccggggatggagacacaaactataatggaaagttcaaaggcaaggccactctgaccgctgacgagtcaagctccactgcttatatgcagctgtctagtctggcaagcgaggattccgccgtctacttttgcgctcggagagaaaccacaactgtgggcaggtactattacgcaatggactactggggccaggggaccacagtcaccgtgtcaagc

SEQ ID NO: 112 - DNA, 330 bp, Artificial Sequence
  Description of Artificial Sequence: Synthetic polynucleotide
gcacctgaggctgcaggaggaccaagcgtgttcctgtttccccctaaacctaaggacactctgatgatctctcggactcccgaagtcacctgtgtggtcgtgagcgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggcactgccagcccccatcgagaagaccatttccaaagccaag

SEQ ID NO: 113 - DNA, 318 bp, Artificial Sequence
  Description of Artificial Sequence: Synthetic polynucleotide
ggccagcctcgagaaccacaggtctatgtgctgccacccagccgggacgagctgacaaaaaaccaggtctccctgctgtgtctggtgaagggattctacccttctgatattgctgtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggc

SEQ ID NO: 114 - DNA, 1359 bp, Artificial Sequence
  Description of Artificial Sequence: Synthetic polynucleotide
caggtccagctgcagcagtccggagccgaactggtcagacccggcagctccgtgaaaatcagctgcaaggcctccggctatgctttctctagttactggatgaactgggtgaagcagaggcctgggcagggactggaatggatcgggcagatttggccaggcgacggggatacaaactataatgggaagttcaaaggaaaggcaacactgactgccgacgagtcaagctccactgcttatatgcagctgtctagtctggcttcagaggatagcgcagtgtacttttgcgcccggagagaaaccacaactgtgggccgctactattacgcaatggactattggggacagggcaccacagtcacagtgtcaagcgcctctactaaagggcctagtgtgtttccactggctccctcctctaagagcacatccggaggaactgcagctctgggatgtctggtgaaggattacttcccagagcccgtcacagtgtcctggaactctggcgctctgactagcggggtccacacctttcctgcagtgctgcagagttcaggcctgtatagcctgagctccgtggtcaccgtgccatctagttcactggggacccagacatacatctgcaacgtgaatcacaaaccaagcaatacaaaggtcgacaagaaagtggaacccaaaagctgtgataagactcatacctgccccccttgtcctgcaccagaggcagcaggaggaccaagcgtgttcctgtttccacccaaacctaaggacacactgatgatttcccgaaccccagaagtgacatgcgtggtcgtgtctgtgagtcacgaggaccccgaagtcaaattcaactggtacgtggatggggtcgaggtgcataatgccaaaaccaagcccagggaggaacagtataattcaacttaccgcgtcgtgagcgtcctgaccgtgctgcaccaggattggctgaacggaaaggagtacaaatgcaaggtgtccaacaaggctctgcccgcacctatcgagaagaccatttctaaagctaagggccagcctcgagaaccacaggtctatgtgtaccctccatcccgggacgagctgaccaaaaaccaggtctctctgacatgtctggtgaaggggttttatcccagtgatattgccgtggagtgggaaagcaatggacagcctgaaaacaattacaagactaccccccctgtgctggacagtgatggatcattcgcactggtctccaaactgactgtggacaagtctaggtggcagcagggcaacgtcttttcatgtagcgtgatgcatgaggccctgcacaatcattacacccagaagtccctgtctctgagtcccggc

SEQ ID NO: 115 - DNA, 372 bp, Artificial Sequence
  Description of Artificial Sequence: Synthetic polynucleotide
caggtccagctgcagcagtccggagccgaactggtcagacccggcagctccgtgaaaatcagctgcaaggcctccggctatgctttctctagttactggatgaactgggtgaagcagaggcctgggcagggactggaatggatcgggcagatttggccaggcgacggggatacaaactataatgggaagttcaaaggaaaggcaacactgactgccgacgagtcaagctccactgcttatatgcagctgtctagtctggcttcagaggatagcgcagtgtacttttgcgcccggagagaaaccacaactgtgggccgctactattacgcaatggactattggggacagggcaccacagtcacagtgtcaagc

SEQ ID NO: 116 - DNA, 294 bp, Artificial Sequence
  Description of Artificial Sequence: Synthetic polynucleotide
gcctctactaaagggcctagtgtgtttccactggctccctcctctaagagcacatccggaggaactgcagctctgggatgtctggtgaaggattacttcccagagcccgtcacagtgtcctggaactctggcgctctgactagcggggtccacacctttcctgcagtgctgcagagttcaggcctgtatagcctgagctccgtggtcaccgtgccatctagttcactggggacccagacatacatctgcaacgtgaatcacaaaccaagcaatacaaaggtcgacaagaaagtg

SEQ ID NO: 117 - DNA, 330 bp, Artificial Sequence
  Description of Artificial Sequence: Synthetic polynucleotide
gcaccagaggcagcaggaggaccaagcgtgttcctgtttccacccaaacctaaggacacactgatgatttcccgaaccccagaagtgacatgcgtggtcgtgtctgtgagtcacgaggaccccgaagtcaaattcaactggtacgtggatggggtcgaggtgcataatgccaaaaccaagcccagggaggaacagtataattcaacttaccgcgtcgtgagcgtcctgaccgtgctgcaccaggattggctgaacggaaaggagtacaaatgcaaggtgtccaacaaggctctgcccgcacctatcgagaagaccatttctaaagctaag

SEQ ID NO: 118 - DNA, 318 bp, Artificial Sequence
  Description of Artificial Sequence: Synthetic polynucleotide
ggccagcctcgagaaccacaggtctatgtgtaccctccatcccgggacgagctgaccaaaaaccaggtctctctgacatgtctggtgaaggggttttatcccagtgatattgccgtggagtgggaaagcaatggacagcctgaaaacaattacaagactaccccccctgtgctggacagtgatggatcattcgcactggtctccaaactgactgtggacaagtctaggtggcagcagggcaacgtcttttcatgtagcgtgatgcatgaggccctgcacaatcattacacccagaagtccctgtctctgagtcccggc

SEQ ID NO: 119 - DNA, 1422 bp, Artificial Sequence
  Description of Artificial Sequence: Synthetic polynucleotide
cagatcgtcctgacacagagcccagcaatcatgtcagccagccccggcgagaaagtcaccatgacatgctcagccagctcctctgtgagctacatgaactggtatcagcagaaaagcggaacatcccccaagagatggatctacgacacttccaagctggcttctggagtgcctgcacacttcaggggcagcggctctgggactagttattcactgaccatttccggcatggaggccgaagatgccgctacctactattgccagcagtggagttcaaacccattcacatttggatctggcactaagctggaaattaatggcggaggaggctccggaggaggagggtctggaggaggaggaagtcaggtccagctgcagcagagcggagctgagctggcacgaccaggagcaagtgtgaaaatgtcctgtaaggccagcggctacactttcacccggtataccatgcattgggtgaaacagagacccgggcagggactggaatggatcgggtacattaatccttcccgaggatacacaaactacaaccagaagtttaaagacaaggctaccctgaccacagataagagctcctctacagcatatatgcagctgagttcactgacttctgaggacagtgccgtgtactattgcgctaggtactatgacgatcactactccctggattattggggccaggggactaccctgaccgtgagctccgcagccgaacctaaatctagtgacaagacacatacttgcccaccttgtccagcaccagagctgctgggaggacctagcgtgttcctgtttccacccaaaccaaaggatacactgatgatctcccggacccctgaagtcacatgtgtggtcgtggacgtgtctcacgaggaccccgaagtcaagttcaactggtacgtggacggcgtcgaggtgcataatgccaaaactaagcccagggaggaacagtacaactccacttatcgcgtcgtgtctgtcctgaccgtgctgcaccaggattggctgaacggcaaggagtacaaatgcaaggtgagcaacaaggccctgcctgctccaatcgagaagaccattagcaaagcaaaggggcagccccgagaacctcaggtctacgtgtatcctccatctcgggacgagctgaccaaaaaccaggtcagtctgacatgtctggtgaagggcttttacccaagcgatattgctgtggagtgggaatccaatgggcagcccgaaaacaattataagacaactccccctgtgctggactcagatgggagcttcgccctggtcagtaaactgactgtggacaagtcacggtggcagcagggaaacgtctttagctgttccgtgatgcatgaggctctgcacaatcattacacccagaaatctctgagtctgtcacccggcaag

SEQ ID NO: 120 - DNA, 318 bp, Artificial Sequence
  Description of Artificial Sequence: Synthetic polynucleotide
cagatcgtcctgacacagagcccagcaatcatgtcagccagccccggcgagaaagtcaccatgacatgctcagccagctcctctgtgagctacatgaactggtatcagcagaaaagcggaacatcccccaagagatggatctacgacacttccaagctggcttctggagtgcctgcacacttcaggggcagcggctctgggactagttattcactgaccatttccggcatggaggccgaagatgccgctacctactattgccagcagtggagttcaaacccattcacatttggatctggcactaagctggaaattaat

SEQ ID NO: 121 - DNA, 357 bp, Artificial Sequence
  Description of Artificial Sequence: Synthetic polynucleotide
caggtccagctgcagcagagcggagctgagctggcacgaccaggagcaagtgtgaaaatgtcctgtaaggccagcggctacactttcacccggtataccatgcattgggtgaaacagagacccgggcagggactggaatggatcgggtacattaatccttcccgaggatacacaaactacaaccagaagtttaaagacaaggctaccctgaccacagataagagctcctctacagcatatatgcagctgagttcactgacttctgaggacagtgccgtgtactattgcgctaggtactatgacgatcactactccctggattattggggccaggggactaccctgaccgtgagctcc

SEQ ID NO: 122 - DNA, 330 bp, Artificial Sequence
  Description of Artificial Sequence: Synthetic polynucleotide
gcaccagagctgctgggaggacctagcgtgttcctgtttccacccaaaccaaaggatacactgatgatctcccggacccctgaagtcacatgtgtggtcgtggacgtgtctcacgaggaccccgaagtcaagttcaactggtacgtggacggcgtcgaggtgcataatgccaaaactaagcccagggaggaacagtacaactccacttatcgcgtcgtgtctgtcctgaccgtgctgcaccaggattggctgaacggcaaggagtacaaatgcaaggtgagcaacaaggccctgcctgctccaatcgagaagaccattagcaaagcaaag

SEQ ID NO: 123 - DNA, 318 bp, Artificial Sequence
  Description of Artificial Sequence: Synthetic polynucleotide
gggcagccccgagaacctcaggtctacgtgtatcctccatctcgggacgagctgaccaaaaaccaggtcagtctgacatgtctggtgaagggcttttacccaagcgatattgctgtggagtgggaatccaatgggcagcccgaaaacaattataagacaactccccctgtgctggactcagatgggagcttcgccctggtcagtaaactgactgtggacaagtcacggtggcagcagggaaacgtctttagctgttccgtgatgcatgaggctctgcacaatcattacacccagaaatctctgagtctgtcacccggc

SEQ ID NO: 124 - DNA, 1422 bp, Artificial Sequence
  Description of Artificial Sequence: Synthetic polynucleotide
cagatcgtcctgacacagagcccagctatcatgtcagcaagccccggcgagaaagtcacaatgacttgctcagccagctcctctgtgagctacatgaactggtatcagcagaaaagcggaacctcccccaagagatggatctacgacacatccaagctggcctctggagtgcctgctcacttcaggggcagcggctctgggaccagttattcactgacaatttccggcatggaggccgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagcagagctggctcgaccaggagctagtgtgaaaatgtcctgtaaggcaagcggctacaccttcacacggtataccatgcattgggtgaaacagagacccgggcagggactggaatggatcgggtacattaatccttcccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactccctggattattggggccaggggactaccctgacagtgagctccgcagccgaacctaaatctagtgacaagactcatacctgccccccttgtccagcaccagaggctgcaggaggacctagcgtgttcctgtttccacccaaaccaaaggatactctgatgatctcccggacacctgaagtcacttgtgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 125 - DNA, 318 bp, Artificial Sequence; identical to SEQ ID NO: 2
  Description of Artificial Sequence: Synthetic polynucleotide

SEQ ID NO: 126 - DNA, 357 bp, Artificial Sequence
  Description of Artificial Sequence: Synthetic polynucleotide
caggtgcagctgcagcagagcggagcagagctggctcgaccaggagctagtgtgaaaatgtcctgtaaggcaagcggctacaccttcacacggtataccatgcattgggtgaaacagagacccgggcagggactggaatggatcgggtacattaatccttcccgaggatacacaaactacaaccagaagtttaaagacaaggccactctgaccacagataagagctcctctaccgcttatatgcagctgagttcactgacatctgaggacagtgcagtgtactattgcgccaggtactatgacgatcactactccctggattattggggccaggggactaccctgacagtgagctcc

SEQ ID NO: 127 - DNA, 330 bp, Artificial Sequence
  Description of Artificial Sequence: Synthetic polynucleotide
gcaccagaggctgcaggaggacctagcgtgttcctgtttccacccaaaccaaaggatactctgatgatctcccggacacctgaagtcacttgtgtggtcgtgagcgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggcactgcctgccccaatcgagaagacaattagcaaagcaaag

SEQ ID NO: 128 - DNA, 318 bp, Artificial Sequence; identical to SEQ ID NO: 5
  Description of Artificial Sequence: Synthetic polynucleotide

SEQ ID NO: 129 - DNA, 1362 bp, Artificial Sequence
  Description of Artificial Sequence: Synthetic polynucleotide
caggtccagctgcagcagagcggagccgaactggtcagacccggcagctccgtgaaaatcagttgcaaggcttcaggctatgcattctctagttactggatgaactgggtgaagcagaggcctgggcagggactggaatggatcgggcagatttggccaggcgacggggatactaactataatgggaagttcaaaggaaaggccactctgaccgctgacgagtcaagctccaccgcctatatgcagctgtctagtctggcatctgaggatagtgccgtgtacttttgcgctcggagagaaaccacaactgtgggccgctactattacgctatggactattggggacagggcaccacagtcactgtgtcaagcgctagcaccaaagggccttccgtgtttccactggcaccctcctctaagagcacttccggaggaaccgcagctctgggatgtctggtgaaggattacttcccagagcccgtcacagtgtcatggaacagcggagcactgaccagcggagtccacacatttcctgccgtgctgcagagttcaggcctgtattccctgagctccgtggtcacagtgccatctagttcactggggacacagacttacatctgcaacgtgaatcacaaaccatccaatactaaggtcgacaagaaagtggaacccaaatcttgtgataagacccatacatgccccccttgtcctgctccagagctgctgggaggaccaagcgtgttcctgtttccacccaaacctaaggacactctgatgattagccgaacaccagaagtcacttgcgtggtcgtggacgtgagccacgaggaccccgaagtcaagttcaactggtacgtggatggggtcgaggtgcataatgccaaaaccaagcccagggaggaacagtataattctacataccgcgtcgtgagtgtcctgactgtgctgcaccaggactggctgaacggaaaggagtacaaatgcaaggtgtccaacaaggcactgcccgcccctatcgagaagaccatttctaaagcaaagggccagcctcgagaaccacaggtctatgtgctgcctccaagtcgggacgagctgacaaaaaaccaggtcagcctgctgtgtctggtgaaggggttctacccctccgatattgccgtggagtgggaatctaatggacagcctgaaaacaattatctgacctggccccctgtgctggactccgatggatctttctttctgtactcaaaactgacagtggataagagcaggtggcagcagggcaacgtcttttcttgtagtgtgatgcatgaggccctgcacaatcattacacccagaaatcactgagcctgtcccccggcaag

SEQ ID NO: 130 - DNA, 372 bp, Artificial Sequence
  Description of Artificial Sequence: Synthetic polynucleotide
caggtccagctgcagcagagcggagccgaactggtcagacccggcagctccgtgaaaatcagttgcaaggcttcaggctatgcattctctagttactggatgaactgggtgaagcagaggcctgggcagggactggaatggatcgggcagatttggccaggcgacggggatactaactataatgggaagttcaaaggaaaggccactctgaccgctgacgagtcaagctccaccgcctatatgcagctgtctagtctggcatctgaggatagtgccgtgtacttttgcgctcggagagaaaccacaactgtgggccgctactattacgctatggactattggggacagggcaccacagtcactgtgtcaagc

SEQ ID NO: 131 - DNA, 294 bp, Artificial Sequence
  Description of Artificial Sequence: Synthetic polynucleotide
gctagcaccaaagggccttccgtgtttccactggcaccctcctctaagagcacttccggaggaaccgcagctctgggatgtctggtgaaggattacttcccagagcccgtcacagtgtcatggaacagcggagcactgaccagcggagtccacacatttcctgccgtgctgcagagttcaggcctgtattccctgagctccgtggtcacagtgccatctagttcactggggacacagacttacatctgcaacgtgaatcacaaaccatccaatactaaggtcgacaagaaagtg

SEQ ID NO: 132 - DNA, 330 bp, Artificial Sequence
  Description of Artificial Sequence: Synthetic polynucleotide
gctccagagctgctgggaggaccaagcgtgttcctgtttccacccaaacctaaggacactctgatgattagccgaacaccagaagtcacttgcgtggtcgtggacgtgagccacgaggaccccgaagtcaagttcaactggtacgtggatggggtcgaggtgcataatgccaaaaccaagcccagggaggaacagtataattctacataccgcgtcgtgagtgtcctgactgtgctgcaccaggactggctgaacggaaaggagtacaaatgcaaggtgtccaacaaggcactgcccgcccctatcgagaagaccatttctaaagcaaag

SEQ ID NO: 133 - DNA, 318 bp, Artificial Sequence
  Description of Artificial Sequence: Synthetic polynucleotide
ggccagcctcgagaaccacaggtctatgtgctgcctccaagtcgggacgagctgacaaaaaaccaggtcagcctgctgtgtctggtgaaggggttctacccctccgatattgccgtggagtgggaatctaatggacagcctgaaaacaattatctgacctggccccctgtgctggactccgatggatctttctttctgtactcaaaactgacagtggataagagcaggtggcagcagggcaacgtcttttcttgtagtgtgatgcatgaggccctgcacaatcattacacccagaaatcactgagcctgtcccccggc

SEQ ID NO: 134 - DNA, 1419 bp, Artificial Sequence
  Description of Artificial Sequence: Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacatactattgccagcagtggagttcaaacccattcacttttggatctggcaccaagctggaaattaatggcggaggaggctccggaggaggagggtctggaggaggaggaagtcaggtgcagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctccgcagccgaacctaaatctagtgacaagactcatacctgccccccttgtccagcaccagagctgctgggaggaccttccgtgttcctgtttccacccaaaccaaaggatactctgatgatctcccggacacctgaagtcacttgcgtggtcgtggacgtgtctcacgaggaccccgaagtcaagttcaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaagtctctgagtctgtcacccggc

SEQ ID NO: 135 - DNA, 318 bp, Artificial Sequence
  Description of Artificial Sequence: Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacatactattgccagcagtggagttcaaacccattcacttttggatctggcaccaagctggaaattaat

SEQ ID NO: 136 - DNA, 357 bp, Artificial Sequence
  Description of Artificial Sequence: Synthetic polynucleotide
caggtgcagctgcagcagtccggagctgagctggcacgaccaggagcaagtgtgaaaatgtcctgtaaggccagcggctacaccttcacacggtataccatgcattgggtgaaacagagacccgggcagggactggaatggatcgggtacattaatcctagccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactgtctggattattggggccaggggactaccctgaccgtgagctcc

SEQ ID NO: 137 - DNA, 330 bp, Artificial Sequence
  Description of Artificial Sequence: Synthetic polynucleotide
gcaccagagctgctgggaggaccttccgtgttcctgtttccacccaaaccaaaggatactctgatgatctcccggacacctgaagtcacttgcgtggtcgtggacgtgtctcacgaggaccccgaagtcaagttcaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaag

SEQ ID NO: 138 - DNA, 318 bp, Artificial Sequence
  Description of Artificial Sequence: Synthetic polynucleotide
gggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaagtctctgagtctgtcacccggc

SEQ ID NO: 139 - DNA, 1422 bp, Artificial Sequence
  Description of Artificial Sequence: Synthetic polynucleotide
cagatcgtcctgacacagagcccagcaatcatgtcagccagccccggcgagaaagtcacaatgacttgctcagcaagctcctctgtgagctacatgaactggtatcagcagaaaagcggaacctcccccaagagatggatctacgacacatccaagctggcttctggagtgcctgcacacttcaggggcagcggctctgggaccagttattcactgacaatttccggcatggaggctgaagatgccgctacctactattgccagcagtggagttcaaacccattcacttttggatctggcaccaagctggaaattaatggcggaggaggctccggaggaggagggtctggaggaggaggaagtcaggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctccgcagccgaacctaaatctagtgacaagactcatacctgccccccttgtccagcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaaggggcagccccgagaacctcaggtctacgtgctgcctccatctcgggacgagctgactaaaaaccaggtcagtctgctgtgtctggtgaagggcttctatccaagcgatattgctgtggagtgggaatccaatgggcagcccgaaaacaattacctgacttggccccctgtcctggactcagatgggagcttctttctgtatagtaaactgaccgtggacaagtcacggtggcagcagggaaacgtctttagctgttccgtgatgcatgaggccctgcacaatcattacacccagaaatctctgagtctgtcacccggcaag

SEQ ID NO: 140 - DNA, 318 bp, Artificial Sequence; identical to SEQ ID NO: 82
  Description of Artificial Sequence: Synthetic polynucleotide

SEQ ID NO: 141 - DNA, 357 bp, Artificial Sequence
  Description of Artificial Sequence: Synthetic polynucleotide
caggtgcagctgcagcagagcggagctgagctggcacgaccaggagcaagtgtgaaaatgtcctgtaaggccagcggctacaccttcacacggtataccatgcattgggtgaaacagagacccgggcagggactggaatggatcgggtacattaatccttcccgaggatacacaaactacaaccagaagtttaaagacaaggctactctgaccacagataagagctcctctaccgcatatatgcagctgagttcactgacatctgaggacagtgccgtgtactattgcgctaggtactatgacgatcactactccctggattattggggccaggggactaccctgacagtgagctcc

SEQ ID NO: 142 - DNA, 330 bp, Artificial Sequence
  Description of Artificial Sequence: Synthetic polynucleotide
gcaccagagctgctgggaggacctagcgtgttcctgtttccacccaaaccaaaggatactctgatgatctcccggacacctgaagtcacttgtgtggtcgtggacgtgtctcacgaggaccccgaagtcaagtttaactggtacgtggacggcgtcgaggtgcataatgccaaaaccaagcccagggaggaacagtacaactccacatatcgcgtcgtgtctgtcctgactgtgctgcaccaggattggctgaacggcaaggagtacaaatgcaaggtgagcaacaaggccctgcctgctccaatcgagaagacaattagcaaagccaag

SEQ ID NO: 143 - DNA, 318 bp, Artificial Sequence; identical to SEQ ID NO: 5
  Description of Artificial Sequence: Synthetic polynucleotide

SEQ ID NO: 144 - DNA, 1449 bp, Artificial Sequence
  Description of Artificial Sequence: Synthetic polynucleotide
gaagtccagctggtcgagtccggaggaggactggtgcagccaggagggtcactgaaactgagctgcgccgcttccggcttcacttttaacaagtatgcaatgaattgggtgcggcaggcaccagggaagggactggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcatactggggacagggaaccctggtcacagtgagctccggaggaggaggcagcggaggaggaggctctgggggaggcgggagtcagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggaggcgggacaaagctgactgtgctggctgcagagccaaagtcaagcgacaaaactcacacctgcccaccttgtccagctccagaagcagctggaggaccatccgtgttcctgtttccacccaagcccaaagatacactgatgatctctcgcactcccgaggtcacctgtgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacggaaaggagtacaagtgcaaagtgagcaataaggcactgcctgccccaatcgaaaaaaccattagcaaggctaaagggcagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggcagtttcgcactggtctccaagctgaccgtggataaatctaggtggcagcaggggaacgtctttagctgttccgtgatgcatgaagccctgcacaatcattacacacagaagtctctgagtctgtcacccggcaaa

SEQ ID NO: 145 - DNA, 375 bp, Artificial Sequence
  Description of Artificial Sequence: Synthetic polynucleotide
gaagtccagctggtcgagtccggaggaggactggtgcagccaggagggtcactgaaactgagctgcgccgcttccggcttcacttttaacaagtatgcaatgaattgggtgcggcaggcaccagggaagggactggaatgggtggcccggatcagatctaagtacaacaactacgctacctactatgcagacagtgtgaaggataggttcacaatttctcgcgacgatagtaaaaacactgcttacctgcagatgaacaatctgaagacagaggacactgcagtctactattgcgtgagacacggaaactttggcaatagctacatctcctattgggcatactggggacagggaaccctggtcacagtgagctcc

SEQ ID NO: 146 - DNA, 327 bp, Artificial Sequence
  Description of Artificial Sequence: Synthetic polynucleotide
cagactgtggtcacccaggagccctcactgacagtcagccctggaggcactgtgaccctgacatgtgggtctagtaccggagccgtgacatctggcaactatcccaattgggtgcagcagaaacctggacaggctccacgaggactgattggaggaacaaagttcctggcccccggaactcctgctcgattttccggctctctgctgggagggaaagcagcactgaccctgagcggagtgcagcctgaggatgaagccgagtactattgcgtgctgtggtacagcaacagatgggtgttcggaggcgggacaaagctgactgtgctg

SEQ ID NO: 147 - DNA, 330 bp, Artificial Sequence
  Description of Artificial Sequence: Synthetic polynucleotide
gctccagaagcagctggaggaccatccgtgttcctgtttccacccaagcccaaagatacactgatgatctctcgcactcccgaggtcacctgtgtggtcgtgagtgtgtcacacgaagaccctgaggtcaagtttaactggtacgtggatggcgtcgaagtgcataatgccaagaccaaacctcgagaggaacagtataattcaacttaccgggtcgtgagcgtcctgaccgtgctgcatcaggactggctgaacggaaaggagtacaagtgcaaagtgagcaataaggcactgcctgccccaatcgaaaaaaccattagcaaggctaaa

SEQ ID NO: 148 - DNA, 318 bp, Artificial Sequence
  Description of Artificial Sequence: Synthetic polynucleotide
gggcagccaagagagccccaggtctacgtgtatcctccaagcagggacgaactgaccaagaaccaggtctccctgacatgtctggtgaaagggttctatcctagtgatattgcagtggaatgggagtcaaatggacagccagagaacaattacaagaccacaccccctgtgctggactctgatggcagtttcgcactggtctccaagctgaccgtggataaatctaggtggcagcaggggaacgtctttagctgttccgtgatgcatgaagccctgcacaatcattacacacagaagtctctgagtctgtcacccggc

SEQ ID NO: 149 - DNA, 1359 bp, Artificial Sequence
  Description of Artificial Sequence: Synthetic polynucleotide
caggtccagctgcagcagagcggagccgaactggtcagacccggcagctccgtgaaaatcagttgcaaggcttcaggctatgcattctctagttactggatgaactgggtgaagcagaggcctgggcagggactggaatggatcgggcagatttggccaggcgacggggatacaaactataatgggaagttcaaaggaaaggccacactgactgctgacgagtcaagctccactgcatatatgcagctgtctagtctggcatctgaggatagtgccgtgtacttttgcgctcggagagaaaccacaactgtgggccgctactattacgccatggactattggggacagggcaccacagtcacagtgtcaagcgctagcactaaagggccttccgtgtttccactggcaccctcctctaagagcacatccggaggaactgcagctctgggatgtctggtgaaggattacttcccagagcccgtcacagtgtcatggaacagcggcgcactgactagcggggtccacacctttcctgccgtgctgcagagttcaggcctgtattccctgagctccgtggtcaccgtgccatctagttcactggggacccagacatacatctgcaacgtgaatcacaaaccatccaatacaaaggtcgacaagaaagtggaacccaaatcttgtgataagactcatacctgccccccttgtcctgctccagagctgctgggaggaccaagcgtgttcctgtttccacccaaacctaaggacacactgatgattagccgaaccccagaagtgacatgcgtggtcgtggacgtgagccacgaggaccccgaagtcaaattcaactggtacgtggatggggtcgaggtgcataatgccaaaaccaagcccagggaggaacagtataattctacttaccgcgtcgtgagtgtcctgaccgtgctgcaccaggactggctgaacggaaaggagtacaaatgcaaggtgtccaacaaggcactgcccgcccctatcgagaagaccatttctaaagctaagggccagcctcgagaaccacaggtctatgtgtaccctccaagtcgggacgagctgaccaaaaaccaggtcagcctgacatgtctggtgaaggggttttatccctccgatattgcagtggagtgggaatctaatggacagcctgaaaacaattacaagactaccccccctgtgctggactccgatggatctttcgccctggtctcaaaactgactgtggataagagcaggtggcagcagggcaacgtcttttcttgtagtgtgatgcatgaggctctgcacaatcattacacccagaagtcactgagcctgtcccccggc

SEQ ID NO: 150 - DNA, 372 bp, Artificial Sequence
  Description of Artificial Sequence: Synthetic polynucleotide
caggtccagctgcagcagagcggagccgaactggtcagacccggcagctccgtgaaaatcagttgcaaggcttcaggctatgcattctctagttactggatgaactgggtgaagcagaggcctgggcagggactggaatggatcgggcagatttggccaggcgacggggatacaaactataatgggaagttcaaaggaaaggccacactgactgctgacgagtcaagctccactgcatatatgcagctgtctagtctggcatctgaggatagtgccgtgtacttttgcgctcggagagaaaccacaactgtgggccgctactattacgccatggactattggggacagggcaccacagtcacagtgtcaagc

SEQ ID NO: 151 - DNA, 294 bp, Artificial Sequence
  Description of Artificial Sequence: Synthetic polynucleotide
gctagcactaaagggccttccgtgtttccactggcaccctcctctaagagcacatccggaggaactgcagctctgggatgtctggtgaaggattacttcccagagcccgtcacagtgtcatggaacagcggcgcactgactagcggggtccacacctttcctgccgtgctgcagagttcaggcctgtattccctgagctccgtggtcaccgtgccatctagttcactggggacccagacatacatctgcaacgtgaatcacaaaccatccaatacaaaggtcgacaagaaagtg

SEQ ID NO: 152 - DNA, 330 bp, Artificial Sequence
  Description of Artificial Sequence: Synthetic polynucleotide
gctccagagctgctgggaggaccaagcgtgttcctgtttccacccaaacctaaggacacactgatgattagccgaaccccagaagtgacatgcgtggtcgtggacgtgagccacgaggaccccgaagtcaaattcaactggtacgtggatggggtcgaggtgcataatgccaaaaccaagcccagggaggaacagtataattctacttaccgcgtcgtgagtgtcctgaccgtgctgcaccaggactggctgaacggaaaggagtacaaatgcaaggtgtccaacaaggcactgcccgcccctatcgagaagaccatttctaaagctaag

SEQ ID NO: 153 - DNA, 318 bp, Artificial Sequence
  Description of Artificial Sequence: Synthetic polynucleotide
ggccagcctcgagaaccacaggtctatgtgtaccctccaagtcgggacgagctgaccaaaaaccaggtcagcctgacatgtctggtgaaggggttttatccctccgatattgcagtggagtgggaatctaatggacagcctgaaaacaattacaagactaccccccctgtgctggactccgatggatctttcgccctggtctcaaaactgactgtggataagagcaggtggcagcagggcaacgtcttttcttgtagtgtgatgcatgaggctctgcacaatcattacacccagaagtcactgagcctgtcccccggc

SEQ ID NO: 154 - DNA, 1446 bp, Artificial Sequence
  Description of Artificial Sequence: Synthetic polynucleotide
gaagtccagctggtcgagtctggaggaggactggtgaagccaggagggagtctgaaactgtcatgcgccgctagcgggtataccttcacaagctacgtcatgcactgggtgaggcaggcaccagggaagggactggaatggatcggctatattaatccctacaacgacgggactaagtataatgagaaatttcagggcagggtgaccatcagctccgataagtctattagtacagcctacatggagctgtctagtctgcgcagcgaagacacagcaatgtactattgcgccagggggacatactattacggaactcgcgtgttcgattactggggccaggggaccctggtcacagtgtcaagcggaggcgggggaagtggaggaggaggctcaggaggaggagggagcgacatcgtgatgacccagtcccctgctacactgtcactgagcccaggcgagcgggcaactctgtcctgtagatcctctaagtctctgcagaacgtgaatggaaacacctatctgtactggtttcagcagaaaccaggccagagcccccagctgctgatctatagaatgtccaatctgaactctggcgtgcctgataggttctccggatctggcagtgggaccgagttcaccctgaccattagttcactggagccagaagacttcgccgtgtattactgcatgcagcacctggagtaccccatcacttttggagctggcaccaagctggagatcaaggcagccgaaccaaagagctccgataaaacacatacttgcccaccttgtccagcaccagaagctgcaggaggaccaagcgtgttcctgtttccacccaagcctaaagacaccctgatgatctcccggactcccgaggtcacctgtgtggtcgtgtcagtgagccacgaggaccctgaagtcaagttcaattggtacgtggatggcgtcgaagtgcataacgctaagacaaaaccccgagaggaacagtataacagtacataccgggtcgtgtcagtgctgaccgtcctgcaccaggattggctgaatggaaaggagtacaagtgcaaagtgtctaacaaggccctgcctgctccaatcgagaaaaccattagcaaggctaaaggccagccccgcgaacctcaggtctatgtgctgcctccaagccgagatgagctgacaaagaatcaggtctccctgctgtgtctggtgaaagggttctacccttctgacattgcagtggagtgggaaagtaacggacagccagagaacaattatctgacatggccccctgtcctggactccgatggctctttctttctgtacagcaagctgactgtggacaaatccagatggcagcaggggaatgtcttttcctgttctgtgatgcatgaagccctgcacaaccattacacccagaagagtctgtcactgagccctggcaaa

SEQ ID NO: 155 - DNA, 363 bp, Artificial Sequence
  Description of Artificial Sequence: Synthetic polynucleotide
gaagtccagctggtcgagtctggaggaggactggtgaagccaggagggagtctgaaactgtcatgcgccgctagcgggtataccttcacaagctacgtcatgcactgggtgaggcaggcaccagggaagggactggaatggatcggctatattaatccctacaacgacgggactaagtataatgagaaatttcagggcagggtgaccatcagctccgataagtctattagtacagcctacatggagctgtctagtctgcgcagcgaagacacagcaatgtactattgcgccagggggacatactattacggaactcgcgtgttcgattactggggccaggggaccctggtcacagtgtcaagc

SEQ ID NO: 156 - DNA, 336 bp, Artificial Sequence
  Description of Artificial Sequence: Synthetic polynucleotide
gacatcgtgatgacccagtcccctgctacactgtcactgagcccaggcgagcgggcaactctgtcctgtagatcctctaagtctctgcagaacgtgaatggaaacacctatctgtactggtttcagcagaaaccaggccagagcccccagctgctgatctatagaatgtccaatctgaactctggcgtgcctgataggttctccggatctggcagtgggaccgagttcaccctgaccattagttcactggagccagaagacttcgccgtgtattactgcatgcagcacctggagtaccccatcacttttggagctggcaccaagctggagatcaag

SEQ ID NO: 157 - DNA, 330 bp, Artificial Sequence
  Description of Artificial Sequence: Synthetic polynucleotide
gcaccagaagctgcaggaggaccaagcgtgttcctgtttccacccaagcctaaagacaccctgatgatctcccggactcccgaggtcacctgtgtggtcgtgtcagtgagccacgaggaccctgaagtcaagttcaattggtacgtggatggcgtcgaagtgcataacgctaagacaaaaccccgagaggaacagtataacagtacataccgggtcgtgtcagtgctgaccgtcctgcaccaggattggctgaatggaaaggagtacaagtgcaaagtgtctaacaaggccctgcctgctccaatcgagaaaaccattagcaaggctaaa

SEQ ID NO: 158 - DNA, 318 bp, Artificial Sequence
  Description of Artificial Sequence: Synthetic polynucleotide
ggccagccccgcgaacctcaggtctatgtgctgcctccaagccgagatgagctgacaaagaatcaggtctccctgctgtgtctggtgaaagggttctacccttctgacattgcagtggagtgggaaagtaacggacagccagagaacaattatctgacatggccccctgtcctggactccgatggctctttctttctgtacagcaagctgactgtggacaaatccagatggcagcaggggaatgtcttttcctgttctgtgatgcatgaagccctgcacaaccattacacccagaagagtctgtcactgagccctggc

SEQ ID NO: 159 - DNA, 1452 bp, Artificial Sequence
  Description of Artificial Sequence: Synthetic polynucleotide
gacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaagggaggaggaggcagtggcggaggagggtcaggaggaggaggaagccaggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagcgcagccgaacccaaatcctctgataagacacacacttgccctccatgtccagctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggccctgccagctcccatcgagaagaccatttccaaagctaagggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggcaag

SEQ ID NO: 160 - DNA, 333 bp, Artificial Sequence
  Description of Artificial Sequence: Synthetic polynucleotide
gacattcagctgacccagagtcctgcttcactggcagtgagcctgggacagcgagcaacaatctcctgcaaagctagtcagtcagtggactatgatggcgactcctatctgaactggtaccagcagatcccagggcagccccctaagctgctgatctacgacgcctcaaatctggtgagcggcatcccaccacgattcagcggcagcggctctggaaccgattttacactgaacattcacccagtcgagaaggtggacgccgctacctaccattgccagcagtctacagaggacccctggactttcggcgggggaaccaaactggaaatcaag

SEQ ID NO: 161 - DNA, 372 bp, Artificial Sequence
  Description of Artificial Sequence: Synthetic polynucleotide
caggtgcagctgcagcagagcggagcagagctggtcagaccaggaagctccgtgaaaatttcctgtaaggcatctggctatgccttttctagttactggatgaattgggtgaagcagaggccaggacagggcctggaatggatcgggcagatttggcccggggatggagacacaaactataatggaaagttcaaaggcaaggctactctgaccgcagacgagtcaagctccactgcatatatgcagctgtctagtctggccagcgaggattccgctgtctacttttgcgcacggagagaaaccacaactgtgggcaggtactattacgccatggactactggggccaggggaccacagtcaccgtgtcaagc

SEQ ID NO: 162 - DNA, 330 bp, Artificial Sequence
  Description of Artificial Sequence: Synthetic polynucleotide
gctcctgagctgctgggaggaccaagcgtgttcctgtttccacctaaacctaaggacactctgatgatctctcggactcccgaagtcacctgtgtggtcgtggatgtgagccacgaggaccctgaagtcaaattcaactggtacgtggatggcgtcgaggtgcataatgccaaaacaaagcctagggaggaacagtataactccacataccgcgtcgtgtctgtcctgactgtgctgcatcaggactggctgaacggaaaggagtacaaatgcaaggtgagcaacaaggccctgccagctcccatcgagaagaccatttccaaagctaag

SEQ ID NO: 163 - DNA, 318 bp, Artificial Sequence
  Description of Artificial Sequence: Synthetic polynucleotide
ggccagcctcgagaaccacaggtctatgtgctgccacccagccgggacgagctgacaaaaaaccaggtctccctgctgtgtctggtgaagggattctacccttctgatattgcagtggagtgggaaagtaatggccagccagaaaacaattatctgacttggcctccagtgctggattctgacgggagtttctttctgtacagtaaactgaccgtggataagtcacggtggcagcagggaaacgtctttagttgttcagtgatgcacgaggccctgcacaatcattacacccagaaaagcctgtccctgtctcccggc

SEQ ID NO: 164 - DNA, 1347 bp, Artificial Sequence
  Description of Artificial Sequence: Synthetic polynucleotide
caggtccagctggtgcagtccggaggaggagtggtccagccaggacggtccctgagactgtcttgcaaggctagtgggtatactttcacctcttacaccatgcactgggtgcgccaggcaccagggaagggactggaatggatcgggtatattaaccctagctccggatacacaaagtacaaccagaagttcaaagaccggttcaccatctccgctgataagagtaaatcaaccgcattcctgcagatggactctctgcgacccgaggatacaggcgtgtacttctgcgcccggtggcaggactacgatgtgtattttgactactggggccaggggactccagtcaccgtgtctagtgcatcaactaagggacccagcgtgtttccactggccccctcaagcaaaagcacatccggaggaactgcagctctgggatgtctggtgaaggattatttcccagagcccgtcaccgtgtcttggaacagtggagccctgactagcggcgtccatacctttcccgctgtgctgcagtcctctgggctgtatagcctgagttcagtggtcacagtgcctagctcctctctgggaacacagacttacatctgcaacgtgaatcacaagccttcaaatactaaagtcgacaagaaagtggaaccaaagagctgtgataaaacccatacatgcccaccttgtcctgcaccagagctgctgggaggaccaagcgtgttcctgtttccacccaagcctaaagacaccctgatgatttccaggacccctgaagtcacatgcgtggtcgtggacgtgtctcacgaggaccccgaagtcaagttcaactggtacgtggatggcgtcgaggtgcataatgccaagacaaaacctagggaggaacagtataactccacctaccgcgtcgtgtctgtcctgacagtgctgcaccaggactggctgaacgggaaggagtacaagtgcaaagtgagtaataaggcactgcccgcccctatcgagaaaaccattagcaaggcaaaaggccagcctagagaaccacaggtctacgtgtatcctccatctagggacgagctgacaaagaaccaggtcagtctgacttgtctggtgaaaggattttatccaagcgatattgctgtggagtgggaatccaatggccagcccgaaaacaattacaagaccacaccccctgtgctggactcagatggcagcttcgccctggtcagtaagctgactgtggataaatcacggtggcagcaggggaacgtcttttcttgtagtgtgatgcatgaggctctgcacaatcattacacccagaagtcactgagcctgtcccccggcaaa

SEQ ID NO: 165 - DNA, 357 bp, Artificial Sequence
  Description of Artificial Sequence: Synthetic polynucleotide
caggtccagctggtgcagtccggaggaggagtggtccagccaggacggtccctgagactgtcttgcaaggctagtgggtatactttcacctcttacaccatgcactgggtgcgccaggcaccagggaagggactggaatggatcgggtatattaaccctagctccggatacacaaagtacaaccagaagttcaaagaccggttcaccatctccgctgataagagtaaatcaaccgcattcctgcagatggactctctgcgacccgaggatacaggcgtgtacttctgcgcccggtggcaggactacgatgtgtattttgactactggggccaggggactccagtcaccgtgtctagt

SEQ ID NO: 166 - DNA, 294 bp, Artificial Sequence
  Description of Artificial Sequence: Synthetic polynucleotide
gcatcaactaagggacccagcgtgtttccactggccccctcaagcaaaagcacatccggaggaactgcagctctgggatgtctggtgaaggattatttcccagagcccgtcaccgtgtcttggaacagtggagccctgactagcggcgtccatacctttcccgctgtgctgcagtcctctgggctgtatagcctgagttcagtggtcacagtgcctagctcctctctgggaacacagacttacatctgcaacgtgaatcacaagccttcaaatactaaagtcgacaagaaagtg

SEQ ID NO: 167 - DNA, 330 bp, Artificial Sequence
  Description of Artificial Sequence: Synthetic polynucleotide
gcaccagagctgctgggaggaccaagcgtgttcctgtttccacccaagcctaaagacaccctgatgatttccaggacccctgaagtcacatgcgtggtcgtggacgtgtctcacgaggaccccgaagtcaagttcaactggtacgtggatggcgtcgaggtgcataatgccaagacaaaacctagggaggaacagtataactccacctaccgcgtcgtgtctgtcctgacagtgctgcaccaggactggctgaacgggaaggagtacaagtgcaaagtgagtaataaggcactgcccgcccctatcgagaaaaccattagcaaggcaaaa

SEQ ID NO: 168 - DNA, 318 bp, Artificial Sequence
  Description of Artificial Sequence: Synthetic polynucleotide
ggccagcctagagaaccacaggtctacgtgtatcctccatctagggacgagctgacaaagaaccaggtcagtctgacttgtctggtgaaaggattttatccaagcgatattgctgtggagtgggaatccaatggccagcccgaaaacaattacaagaccacaccccctgtgctggactcagatggcagcttcgccctggtcagtaagctgactgtggataaatcacggtggcagcaggggaacgtcttttcttgtagtgtgatgcatgaggctctgcacaatcattacacccagaagtcactgagcctgtcccccggc

SEQ ID NO: 169 - protein, 119 aa, Artificial Sequence
  Description of Artificial Sequence: Synthetic polypeptide
QVQLQQSGAELARPGASVKMSCKASGYTFTRYTMHWVKQRPGQCLEWIGYINPSRGYTNYNQKFKDKATLTTDKSSSTAYMQLSSLTSEDSAVYYCARYYDDHYSLDYWGQGTTLTVSS

SEQ ID NO: 170 - protein, 110 aa, Artificial Sequence
  Description of Artificial Sequence: Synthetic polypeptide
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 171 - protein, 106 aa, Artificial Sequence
  Description of Artificial Sequence: Synthetic polypeptide
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 172 - protein, 483 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIKGGGGSGGGGSGGGGSQVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 173 - protein, 111 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGCGTKLEIK

SEQ ID NO: 174 - protein, 124 aa, Artificial Sequence
  Description of Artificial Sequence: Synthetic polypeptide
QVQLQQSGAELVRPGSSVKISCKASGYAFSSYWMNWVKQRPGQCLEWIGQIWPGDGDTNYNGKFKGKATLTADESSSTAYMQLSSLASEDSAVYFCARRETTTVGRYYYAMDYWGQGTTVTVSS

SEQ ID NO: 175 - protein, 110 aa, Artificial Sequence; identical to SEQ ID NO: 170
  Description of Artificial Sequence: Synthetic polypeptide

SEQ ID NO: 176 - protein, 106 aa, Artificial Sequence; identical to SEQ ID NO: 171
  Description of Artificial Sequence: Synthetic polypeptide

SEQ ID NO: 177 - protein, 484 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 178 - protein, 111 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 179 - protein, 124 aa, Artificial Sequence
  Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 180 - protein, 110 aa, Artificial Sequence
  Description of Artificial Sequence: Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 181 - protein, 106 aa, Artificial Sequence
  Description of Artificial Sequence: Synthetic polypeptide
GQPREPQVYTYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 182 - protein, 477 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 183 - protein, 119 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 184 - protein, 106 aa, Artificial Sequence
  Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 185 - protein, 110 aa, Artificial Sequence; identical to SEQ ID NO: 180
  Description of Artificial Sequence: Synthetic polypeptide

SEQ ID NO: 186 - protein, 106 aa, Artificial Sequence
  Description of Artificial Sequence: Synthetic polypeptide
GQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPG

SEQ ID NO: 187 - protein, 474 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYMTWPPVLDSDGSFFLYSKLTVDKSRWQQGNVFSCSVMHEALHNHYTQKSLSLSPGK

SEQ ID NO: 188 - protein, 106 aa, Artificial Sequence [sequence omitted]

SEQ ID NO: 189 - protein, 119 aa, Artificial Sequence [sequence omitted]

SEQ ID NO: 190 - protein, 110 aa, Artificial Sequence; identical to SEQ ID NO: 180
  Description of Artificial Sequence: Synthetic polypeptide

SEQ ID NO: 191 - protein, 106 aa, Artificial Sequence; identical to SEQ ID NO: 186
  Description of Artificial Sequence: Synthetic polypeptide

SEQ ID NO: 192 - protein, 484 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 193 - protein, 111 aa, Artificial Sequence; identical to SEQ ID NO: 178
  Description of Artificial Sequence: Synthetic polypeptide

SEQ ID NO: 194 - protein, 124 aa, Artificial Sequence; identical to SEQ ID NO: 179
  Description of Artificial Sequence: Synthetic polypeptide

SEQ ID NO: 195 - protein, 110 aa, Artificial Sequence; identical to SEQ ID NO: 180
  Description of Artificial Sequence: Synthetic polypeptide

SEQ ID NO: 196 - protein, 106 aa, Artificial Sequence
  Description of Artificial Sequence: Synthetic polypeptide
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 197 - protein, 213 aa, Artificial Sequence
  Description of Artificial Sequence: Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 106 aa, Artificial Sequence [sequence omitted]

SEQ ID NO: 199 - protein, 107 aa, Artificial Sequence [sequence omitted]

SEQ ID NO: 200 - protein, 451 aa, Artificial Sequence
  Description of Artificial Sequence: Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 201 - protein, 121 aa, Artificial Sequence [sequence omitted]

SEQ ID NO: 202 - protein, 98 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 203 - protein, 110 aa, Artificial Sequence; identical to SEQ ID NO: 180
  Description of Artificial Sequence: Synthetic polypeptide

SEQ ID NO: 204 - protein, 106 aa, Artificial Sequence; identical to SEQ ID NO: 171
  Description of Artificial Sequence: Synthetic polypeptide

SEQ ID NO: 205 - protein, 448 aa, Artificial Sequence
  Description of Artificial Sequence: Synthetic polypeptide
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 206 - protein, 119 aa, Artificial Sequence [sequence omitted]

SEQ ID NO: 207 - protein, 98 aa, Artificial Sequence; identical to SEQ ID NO: 202
  Description of Artificial Sequence: Synthetic polypeptide

SEQ ID NO: 208 - protein, 110 aa, Artificial Sequence; identical to SEQ ID NO: 170
  Description of Artificial Sequence: Synthetic polypeptide

SEQ ID NO: 209 - protein, 106 aa, Artificial Sequence; identical to SEQ ID NO: 196
  Description of Artificial Sequence: Synthetic polypeptide

SEQ ID NO: 210 - protein, 478 aa, Artificial Sequence
  Description of Artificial Sequence: Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 211 - protein, 122 aa, Artificial Sequence
  Description of Artificial Sequence: Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 212 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 213 - protein, 110 aa, Artificial Sequence; identical to SEQ ID NO: 180
  Description of Artificial Sequence: Synthetic polypeptide

SEQ ID NO: 214 - protein, 106 aa, Artificial Sequence; identical to SEQ ID NO: 171
  Description of Artificial Sequence: Synthetic polypeptide

SEQ ID NO: 215 - protein, 484 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 216 - protein, 111 aa, Artificial Sequence; identical to SEQ ID NO: 178
  Description of Artificial Sequence: Synthetic polypeptide

SEQ ID NO: 217 - protein, 124 aa, Artificial Sequence; identical to SEQ ID NO: 179
  Description of Artificial Sequence: Synthetic polypeptide

SEQ ID NO: 218 - protein, 110 aa, Artificial Sequence; identical to SEQ ID NO: 170
  Description of Artificial Sequence: Synthetic polypeptide

SEQ ID NO: 219 - protein, 106 aa, Artificial Sequence; identical to SEQ ID NO: 171
  Description of Artificial Sequence: Synthetic polypeptide

SEQ ID NO: 220 - protein, 453 aa, Artificial Sequence
  Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 221 - protein, 124 aa, Artificial Sequence; identical to SEQ ID NO: 179
  Description of Artificial Sequence: Synthetic polypeptide

SEQ ID NO: 222 - protein, 98 aa, Artificial Sequence; identical to SEQ ID NO: 202
  Description of Artificial Sequence: Synthetic polypeptide

SEQ ID NO: 223 - protein, 110 aa, Artificial Sequence; identical to SEQ ID NO: 170
  Description of Artificial Sequence: Synthetic polypeptide

SEQ ID NO: 224 - protein, 106 aa, Artificial Sequence; identical to SEQ ID NO: 196
  Description of Artificial Sequence: Synthetic polypeptide

SEQ ID NO: 225 - protein, 474 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 226 - protein, 106 aa, Artificial Sequence [sequence omitted]

SEQ ID NO: 227 - protein, 119 aa, Artificial Sequence
  Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 228 - protein, 110 aa, Artificial Sequence; identical to SEQ ID NO: 180
  Description of Artificial Sequence: Synthetic polypeptide

SEQ ID NO: 229 - protein, 106 aa, Artificial Sequence; identical to SEQ ID NO: 196
  Description of Artificial Sequence: Synthetic polypeptide

SEQ ID NO: 230 - protein, 474 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 231 - protein, 106 aa, Artificial Sequence [sequence omitted]

SEQ ID NO: 232 - protein, 119 aa, Artificial Sequence; identical to SEQ ID NO: 227
  Description of Artificial Sequence: Synthetic polypeptide

SEQ ID NO: 233 - protein, 110 aa, Artificial Sequence; identical to SEQ ID NO: 170
  Description of Artificial Sequence: Synthetic polypeptide

SEQ ID NO: 234 - protein, 106 aa, Artificial Sequence; identical to SEQ ID NO: 171
  Description of Artificial Sequence: Synthetic polypeptide

SEQ ID NO: 235 - protein, 454 aa, Artificial Sequence
  Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 236 - protein, 124 aa, Artificial Sequence; identical to SEQ ID NO: 179
  Description of Artificial Sequence: Synthetic polypeptide

SEQ ID NO: 237 - protein, 98 aa, Artificial Sequence; identical to SEQ ID NO: 202
  Description of Artificial Sequence: Synthetic polypeptide

SEQ ID NO: 238 - protein, 110 aa, Artificial Sequence; identical to SEQ ID NO: 180
  Description of Artificial Sequence: Synthetic polypeptide

SEQ ID NO: 239 - protein, 106 aa, Artificial Sequence; identical to SEQ ID NO: 171
  Description of Artificial Sequence: Synthetic polypeptide

SEQ ID NO: 240 - protein, 473 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 241 - protein, 106 aa, Artificial Sequence [sequence omitted]

SEQ ID NO: 242 - protein, 119 aa, Artificial Sequence [sequence omitted]

SEQ ID NO: 243 - protein, 110 aa, Artificial Sequence; identical to SEQ ID NO: 180
  Description of Artificial Sequence: Synthetic polypeptide

SEQ ID NO: 244 - protein, 106 aa, Artificial Sequence; identical to SEQ ID NO: 171
  Description of Artificial Sequence: Synthetic polypeptide

SEQ ID NO: 245 - protein, 474 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 246 - protein, 106 aa, Artificial Sequence [sequence omitted]

SEQ ID NO: 247 - protein, 119 aa, Artificial Sequence; identical to SEQ ID NO: 227
  Description of Artificial Sequence: Synthetic polypeptide

SEQ ID NO: 248 - protein, 110 aa, Artificial Sequence; identical to SEQ ID NO: 180
  Description of Artificial Sequence: Synthetic polypeptide

SEQ ID NO: 249 - protein, 106 aa, Artificial Sequence; identical to SEQ ID NO: 171
  Description of Artificial Sequence: Synthetic polypeptide

SEQ ID NO: 250 - protein, 483 aa, Artificial Sequence
  Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 251 - protein, 125 aa, Artificial Sequence
  Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 252 - protein, 109 aa, Artificial Sequence
  Description of Artificial Sequence: Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 253 - protein, 110 aa, Artificial Sequence; identical to SEQ ID NO: 170
  Description of Artificial Sequence: Synthetic polypeptide

SEQ ID NO: 254 - protein, 106 aa, Artificial Sequence; identical to SEQ ID NO: 196
  Description of Artificial Sequence: Synthetic polypeptide

SEQ ID NO: 255 - protein, 453 aa, Artificial Sequence
  Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 256 - protein, 124 aa, Artificial Sequence; identical to SEQ ID NO: 179
  Description of Artificial Sequence: Synthetic polypeptide

SEQ ID NO: 257 - protein, 98 aa, Artificial Sequence; identical to SEQ ID NO: 202
  Description of Artificial Sequence: Synthetic polypeptide

SEQ ID NO: 258 - protein, 110 aa, Artificial Sequence; identical to SEQ ID NO: 180
  Description of Artificial Sequence: Synthetic polypeptide

SEQ ID NO: 259 - protein, 106 aa, Artificial Sequence; identical to SEQ ID NO: 196
  Description of Artificial Sequence: Synthetic polypeptide

SEQ ID NO: 260 - protein, 482 aa, Artificial Sequence
  Description of Artificial Sequence: Synthetic polypeptide
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 261 - protein, 121 aa, Artificial Sequence
  Description of Artificial Sequence: Synthetic polypeptide
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 262 - protein, 112 aa, Artificial Sequence
  Description of Artificial Sequence: Synthetic polypeptide
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 263 - protein, 110 aa, Artificial Sequence; identical to SEQ ID NO: 170
  Description of Artificial Sequence: Synthetic polypeptide

SEQ ID NO: 264 - protein, 106 aa, Artificial Sequence; identical to SEQ ID NO: 171
  Description of Artificial Sequence: Synthetic polypeptide

SEQ ID NO: 265 - protein, 484 aa, Artificial Sequence
  Description of Artificial Sequence: Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 266 - protein, 111 aa, Artificial Sequence; identical to SEQ ID NO: 178
  Description of Artificial Sequence: Synthetic polypeptide

SEQ ID NO: 267 - protein, 124 aa, Artificial Sequence; identical to SEQ ID NO: 179
  Description of Artificial Sequence: Synthetic polypeptide

SEQ ID NO: 268 - protein, 110 aa, Artificial Sequence; identical to SEQ ID NO: 180
  Description of Artificial Sequence: Synthetic polypeptide

SEQ ID NO: 269 - protein, 106 aa, Artificial Sequence; identical to SEQ ID NO: 171
  Description of Artificial Sequence: Synthetic polypeptide

SEQ ID NO: 270 - protein, 449 aa, Artificial Sequence
  Description of Artificial Sequence: Synthetic polypeptide
QVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 271 - protein, 119 aa, Artificial Sequence
  Description of Artificial Sequence: Synthetic polypeptide
QVQLVQSGGGVVQPGRSLRLSCKASGYTFTSYTMHWVRQAPGKGLEWIGYINPSSGYTKYNQKFKDRFTISADKSKSTAFLQMDSLRPEDTGVYFCARWQDYDVYFDYWGQGTPVTVSS

SEQ ID NO: 272 - protein, 98 aa, Artificial Sequence; identical to SEQ ID NO: 202
  Description of Artificial Sequence: Synthetic polypeptide

SEQ ID NO: 273 - protein, 110 aa, Artificial Sequence; identical to SEQ ID NO: 180
  Description of Artificial Sequence: Synthetic polypeptide

SEQ ID NO: 274 - protein, 106 aa, Artificial Sequence; identical to SEQ ID NO: 196
  Description of Artificial Sequence: Synthetic polypeptide

SEQ ID NO: 275 - protein, 119 aa, Artificial Sequence; identical to SEQ ID NO: 169
  Description of Artificial Sequence: Synthetic polypeptide

SEQ ID NO: 276 - protein, 110 aa, Artificial Sequence; identical to SEQ ID NO: 170
  Description of Artificial Sequence: Synthetic polypeptide

SEQ ID NO: 277 - protein, 106 aa, Artificial Sequence; identical to SEQ ID NO: 171
  Description of Artificial Sequence: Synthetic polypeptide

SEQ ID NO: 278 - protein, 483 aa, Artificial Sequence; identical to SEQ ID NO: 172
  Description of Artificial Sequence: Synthetic polypeptide

SEQ ID NO: 279 - protein, 111 aa, Artificial Sequence; identical to SEQ ID NO: 173
  Description of Artificial Sequence: Synthetic polypeptide

SEQ ID NO: 280 - protein, 124 aa, Artificial Sequence; identical to SEQ ID NO: 174
  Description of Artificial Sequence: Synthetic polypeptide

SEQ ID NO: 281 - protein, 110 aa, Artificial Sequence; identical to SEQ ID NO: 170
  Description of Artificial Sequence: Synthetic polypeptide

SEQ ID NO: 282 - protein, 106 aa, Artificial Sequence; identical to SEQ ID NO: 171
  Description of Artificial Sequence: Synthetic polypeptide

SEQ ID NO: 283 - protein, 484 aa, Artificial Sequence; identical to SEQ ID NO: 177
  Description of Artificial Sequence: Synthetic polypeptide

SEQ ID NO: 284 - protein, 111 aa, Artificial Sequence; identical to SEQ ID NO: 178
  Description of Artificial Sequence: Synthetic polypeptide

SEQ ID NO: 285 - protein, 124 aa, Artificial Sequence; identical to SEQ ID NO: 179
  Description of Artificial Sequence: Synthetic polypeptide

SEQ ID NO: 286 - protein, 110 aa, Artificial Sequence; identical to SEQ ID NO: 180
  Description of Artificial Sequence: Synthetic polypeptide

SEQ ID NO: 287 - protein, 106 aa, Artificial Sequence; identical to SEQ ID NO: 181
  Description of Artificial Sequence: Synthetic polypeptide

SEQ ID NO: 288 - protein, 477 aa, Artificial Sequence; identical to SEQ ID NO: 182
  Description of Artificial Sequence: Synthetic polypeptide

SEQ ID NO: 289 - protein, 119 aa, Artificial Sequence; identical to SEQ ID NO: 183
  Description of Artificial Sequence: Synthetic polypeptide

SEQ ID NO: 290 - protein, 106 aa, Artificial Sequence; identical to SEQ ID NO: 184
  Description of Artificial Sequence: Synthetic polypeptide

SEQ ID NO: 291 - protein, 110 aa, Artificial Sequence; identical to SEQ ID NO: 180
  Description of Artificial Sequence: Synthetic polypeptide

SEQ ID NO: 292 - protein, 106 aa, Artificial Sequence; identical to SEQ ID NO: 186
  Description of Artificial Sequence: Synthetic polypeptide

SEQ ID NO: 293 - protein, 474 aa, Artificial Sequence; identical to SEQ ID NO: 187
  Description of Artificial Sequence: Synthetic polypeptide

SEQ ID NO: 294 - protein, 106 aa, Artificial Sequence [sequence omitted]

SEQ ID NO: 295 - protein, 119 aa, Artificial Sequence [sequence omitted]

SEQ ID NO: 296 - protein, 110 aa, Artificial Sequence; identical to SEQ ID NO: 180
  Description of Artificial Sequence: Synthetic polypeptide

SEQ ID NO: 297 - protein, 106 aa, Artificial Sequence; identical to SEQ ID NO: 186
  Description of Artificial Sequence: Synthetic polypeptide

SEQ ID NO: 298 - protein, 484 aa, Artificial Sequence; identical to SEQ ID NO: 192
  Description of Artificial Sequence: Synthetic polypeptide

SEQ ID NO: 299 - protein, 111 aa, Artificial Sequence; identical to SEQ ID NO: 178
  Description of Artificial Sequence: Synthetic polypeptide

SEQ ID NO: 300 - protein, 124 aa, Artificial Sequence; identical to SEQ ID NO: 179
  Description of Artificial Sequence: Synthetic polypeptide

SEQ ID NO: 301 - protein, 110 aa, Artificial Sequence; identical to SEQ ID NO: 180
  Description of Artificial Sequence: Synthetic polypeptide

SEQ ID NO: 302 - protein, 106 aa, Artificial Sequence; identical to SEQ ID NO: 196
  Description of Artificial Sequence: Synthetic polypeptide

SEQ ID NO: 303 - protein, 213 aa, Artificial Sequence; identical to SEQ ID NO: 197
  Description of Artificial Sequence: Synthetic polypeptide

SEQ ID NO: 304 - protein, 106 aa, Artificial Sequence [sequence omitted]

SEQ ID NO: 305 - protein, 107 aa, Artificial Sequence [sequence omitted]

SEQ ID NO: 306 - protein, 451 aa, Artificial Sequence; identical to SEQ ID NO: 200
  Description of Artificial Sequence: Synthetic polypeptide

SEQ ID NO: 307 - protein, 121 aa, Artificial Sequence [sequence omitted]

SEQ ID NO: 308 - protein, 98 aa, Artificial Sequence; identical to SEQ ID NO: 202
  Description of Artificial Sequence: Synthetic polypeptide

SEQ ID NO: 309 - protein, 110 aa, Artificial Sequence; identical to SEQ ID NO: 180
  Description of Artificial Sequence: Synthetic polypeptide

SEQ ID NO: 310 - protein, 106 aa, Artificial Sequence; identical to SEQ ID NO: 171
  Description of Artificial Sequence: Synthetic polypeptide

SEQ ID NO: 311 - protein, 448 aa, Artificial Sequence; identical to SEQ ID NO: 205
  Description of Artificial Sequence: Synthetic polypeptide

SEQ ID NO: 312 - protein, 119 aa, Artificial Sequence [sequence omitted]

SEQ ID NO: 313 - protein, 98 aa, Artificial Sequence; identical to SEQ ID NO: 202
  Description of Artificial Sequence: Synthetic polypeptide

SEQ ID NO: 314 - protein, 110 aa, Artificial Sequence; identical to SEQ ID NO: 170
  Description of Artificial Sequence: Synthetic polypeptide

SEQ ID NO: 315 - protein, 106 aa, Artificial Sequence; identical to SEQ ID NO: 196
  Description of Artificial Sequence: Synthetic polypeptide

SEQ ID NO: 316 - protein, 478 aa, Artificial Sequence; identical to SEQ ID NO: 210
  Description of Artificial Sequence: Synthetic polypeptide

SEQ ID NO: 317 - protein, 122 aa, Artificial Sequence; identical to SEQ ID NO: 211
  Description of Artificial Sequence: Synthetic polypeptide

SEQ ID NO: 318 - protein, 107 aa, Artificial Sequence; identical to SEQ ID NO: 212
  Description of Artificial Sequence: Synthetic polypeptide

SEQ ID NO: 319 - protein, 110 aa, Artificial Sequence; identical to SEQ ID NO: 180
  Description of Artificial Sequence: Synthetic polypeptide

SEQ ID NO: 320 - protein, 106 aa, Artificial Sequence; identical to SEQ ID NO: 171
  Description of Artificial Sequence: Synthetic polypeptide

SEQ ID NO: 321 - protein, 484 aa, Artificial Sequence; identical to SEQ ID NO: 215
  Description of Artificial Sequence: Synthetic polypeptide

SEQ ID NO: 322 - protein, 111 aa, Artificial Sequence; identical to SEQ ID NO: 178
  Description of Artificial Sequence: Synthetic polypeptide

SEQ ID NO: 323 - protein, 124 aa, Artificial Sequence; identical to SEQ ID NO: 179
  Description of Artificial Sequence: Synthetic polypeptide

SEQ ID NO: 324 - protein, 110 aa, Artificial Sequence; identical to SEQ ID NO: 170
  Description of Artificial Sequence: Synthetic polypeptide

SEQ ID NO: 325 - protein, 106 aa, Artificial Sequence; identical to SEQ ID NO: 171
  Description of Artificial Sequence: Synthetic polypeptide

SEQ ID NO: 326 - protein, 453 aa, Artificial Sequence; identical to SEQ ID NO: 220
  Description of Artificial Sequence: Synthetic polypeptide

SEQ ID NO: 327 - protein, 124 aa, Artificial Sequence; identical to SEQ ID NO: 179
  Description of Artificial Sequence: Synthetic polypeptide

SEQ ID NO: 328 - protein, 98 aa, Artificial Sequence; identical to SEQ ID NO: 202
  Description of Artificial Sequence: Synthetic polypeptide

SEQ ID NO: 329 - protein, 110 aa, Artificial Sequence; identical to SEQ ID NO: 170
  Description of Artificial Sequence: Synthetic polypeptide

SEQ ID NO: 330 - protein, 106 aa, Artificial Sequence; identical to SEQ ID NO: 196
  Description of Artificial Sequence: Synthetic polypeptide

SEQ ID NO: 331 - protein, 474 aa, Artificial Sequence; identical to SEQ ID NO: 225
  Description of Artificial Sequence: Synthetic polypeptide

SEQ ID NO: 332 - protein, 106 aa, Artificial Sequence [sequence omitted]

SEQ ID NO: 333 - protein, 119 aa, Artificial Sequence; identical to SEQ ID NO: 227
  Description of Artificial Sequence: Synthetic polypeptide

SEQ ID NO: 334 - protein, 110 aa, Artificial Sequence; identical to SEQ ID NO: 180
  Description of Artificial Sequence: Synthetic polypeptide

SEQ ID NO: 335 - protein, 106 aa, Artificial Sequence; identical to SEQ ID NO: 196
  Description of Artificial Sequence: Synthetic polypeptide

SEQ ID NO: 336 - protein, 474 aa, Artificial Sequence; identical to SEQ ID NO: 230
  Description of Artificial Sequence: Synthetic polypeptide

SEQ ID NO: 337 - protein, 5 aa, Artificial Sequence
  Description of Artificial Sequence: Synthetic peptide
SSVSY

SEQ ID NO: 338 - protein, 3 aa, Artificial Sequence
  Description of Artificial Sequence: Synthetic peptide
DTS

SEQ ID NO: 339 - protein, 7 aa, Artificial Sequence
  Description of Artificial Sequence: Synthetic peptide
QQWSSNP

SEQ ID NO: 340 - protein, 8 aa, Artificial Sequence
  Description of Artificial Sequence: Synthetic peptide
GYTFTRYT

SEQ ID NO: 341 - protein, 8 aa, Artificial Sequence
  Description of Artificial Sequence: Synthetic peptide
INPSRGYT

SEQ ID NO: 342 - protein, 12 aa, Artificial Sequence
  Description of Artificial Sequence: Synthetic peptide
ARYYDDHYCLDY

SEQ ID NO: 343 - protein, 5 aa, Artificial Sequence
  Description of Artificial Sequence: Synthetic peptide
SSVSY

SEQ ID NO: 344 - protein, 3 aa, Artificial Sequence
  Description of Artificial Sequence: Synthetic peptide
DTS

SEQ ID NO: 345 - protein, 7 aa, Artificial Sequence
  Description of Artificial Sequence: Synthetic peptide
QQWSSNP

SEQ ID NO: 346 - protein, 8 aa, Artificial Sequence
  Description of Artificial Sequence: Synthetic peptide
GYTFTRYT

SEQ ID NO: 347 - protein, 8 aa, Artificial Sequence
  Description of Artificial Sequence: Synthetic peptide
INPSRGYT

SEQ ID NO: 348 - protein, 12 aa, Artificial Sequence
  Description of Artificial Sequence: Synthetic peptide
ARYYDDHYSLDY

SEQ ID NO: 349 - protein, 3 aa, Artificial Sequence
  Description of Artificial Sequence: Synthetic peptide
DAS

SEQ ID NO: 350 - protein, 9 aa, Artificial Sequence
  Description of Artificial Sequence: Synthetic peptide
QQSTEDPWT

SEQ ID NO: 351 - protein, 8 aa, Artificial Sequence
  Description of Artificial Sequence: Synthetic peptide
GYAFSSYW

SEQ ID NO: 352 - protein, 8 aa, Artificial Sequence
  Description of Artificial Sequence: Synthetic peptide
IWPGDGDT

SEQ ID NO: 353 - protein, 17 aa, Artificial Sequence
  Description of Artificial Sequence: Synthetic peptide
ARRETTTVGRYYYAMDY

SEQ ID NO: 354 - protein, 11 aa, Artificial Sequence
  Description of Artificial Sequence: Synthetic peptide
QSVDYDGDSYL

SEQ ID NO: 355 - protein, 17 aa, Unknown
  Description of Unknown: Stanniocalcin signal peptide
MLQNSAVLLLLVISASA

SEQ ID NO: 356 - protein, 22 aa, Artificial Sequence
  Description of Artificial Sequence: Synthetic peptide
MPTWAWWLFLVLLLALWAPARG

SEQ ID NO: 357 - protein, 50 aa, Artificial Sequence
  Description of Artificial Sequence: Synthetic polypeptide
  misc_feature (1)..(50): This sequence may encompass 1-10 “Gly Gly Gly Gly Ser” repeating units wherein some residues may be absent
  See specification as filed for detailed description of substitutions and preferred embodiments
GGGGSGGGGSGGGGSGGGGSGGGGSGGGGSGGGGSGGGGSGGGGSGGGGS

SEQ ID NO: 358 - protein, 50 aa, Artificial Sequence
  Description of Artificial Sequence: Synthetic polypeptide
  misc_feature (1)..(50): This sequence may encompass 1-10 “Ser Gly Gly Gly Gly” repeating units wherein some residues may be absent
  See specification as filed for detailed description of substitutions and preferred embodiments
SGGGGSGGGGSGGGGSGGGGSGGGGSGGGGSGGGGSGGGGSGGGGSGGGG

SEQ ID NO: 359 - protein, 54 aa, Artificial Sequence
  Description of Artificial Sequence: Synthetic polypeptide
  misc_feature (5)..(54): This region may encompass 1-10 “Ser Gly Gly Gly Gly” repeating units wherein some residues may be absent
  See specification as filed for detailed description of substitutions and preferred embodiments
GGGGSGGGGSGGGGSGGGGSGGGGSGGGGSGGGGSGGGGSGGGGSGGGGSGGGG

SEQ ID NO: 360 - protein, 422 aa, Artificial Sequence
  Description of Artificial Sequence: Synthetic polypeptide
  misc_feature (1)..(420): This region may encompass 0-20 “Gly Gly Gly Gly Gly Gly Gly Gly Gly Gly Gly Gly Gly Gly Gly Gly Gly Gly Gly Gly Ser” repeating units or embodiments thereof, wherein some residues may be absent
  misc_feature (1)..(20): This region may encompass 0-20 residues wherein some residues may be absent
  misc_feature (22)..(41): This region may encompass 0-20 residues wherein some residues may be absent
  misc_feature (43)..(62): This region may encompass 0-20 residues wherein some residues may be absent
  misc_feature (64)..(83): This region may encompass 0-20 residues wherein some residues may be absent
  misc_feature (85)..(104): This region may encompass 0-20 residues wherein some residues may be absent
  misc_feature (106)..(125): This region may encompass 0-20 residues wherein some residues may be absent
  misc_feature (127)..(146): This region may encompass 0-20 residues wherein some residues may be absent
  misc_feature (148)..(167): This region may encompass 0-20 residues wherein some residues may be absent
  misc_feature (169)..(188): This region may encompass 0-20 residues wherein some residues may be absent
  misc_feature (190)..(209): This region may encompass 0-20 residues wherein some residues may be absent
  misc_feature (211)..(230): This region may encompass 0-20 residues wherein some residues may be absent
  misc_feature (232)..(251): This region may encompass 0-20 residues wherein some residues may be absent
  misc_feature (253)..(272): This region may encompass 0-20 residues wherein some residues may be absent
  misc_feature (274)..(293): This region may encompass 0-20 residues wherein some residues may be absent
  misc_feature (295)..(314): This region may encompass 0-20 residues wherein some residues may be absent
  misc_feature (316)..(335): This region may encompass 0-20 residues wherein some residues may be absent
  misc_feature (337)..(356): This region may encompass 0-20 residues wherein some residues may be absent
  misc_feature (358)..(377): This region may encompass 0-20 residues wherein some residues may be absent
  misc_feature (379)..(398): This region may encompass 0-20 residues wherein some residues may be absent
  misc_feature (400)..(419): This region may encompass 0-20 residues wherein some residues may be absent
  See specification as filed for detailed description of substitutions and preferred embodiments
GGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGG

SEQ ID NO: 361 - protein, 420 aa, Artificial Sequence
  Description of Artificial Sequence: Synthetic polypeptide
  misc_feature (1)..(420): This sequence may encompass 0-20 “Ser Gly Gly Gly Gly Gly Gly Gly Gly Gly Gly Gly Gly Gly Gly Gly Gly Gly Gly Gly Gly” repeating units or embodiments thereof, wherein some residues may be absent
  misc_feature (2)..(21): This region may encompass 0-20 residues wherein some residues may be absent
  misc_feature (23)..(42): This region may encompass 0-20 residues wherein some residues may be absent
  misc_feature (44)..(63): This region may encompass 0-20 residues wherein some residues may be absent
  misc_feature (65)..(84): This region may encompass 0-20 residues wherein some residues may be absent
  misc_feature (86)..(105): This region may encompass 0-20 residues wherein some residues may be absent
  misc_feature (107)..(126): This region may encompass 0-20 residues wherein some residues may be absent
  misc_feature (128)..(147): This region may encompass 0-20 residues wherein some residues may be absent
  misc_feature (149)..(168): This region may encompass 0-20 residues wherein some residues may be absent
  misc_feature (170)..(189): This region may encompass 0-20 residues wherein some residues may be absent
  misc_feature (191)..(210): This region may encompass 0-20 residues wherein some residues may be absent
  misc_feature (212)..(231): This region may encompass 0-20 residues wherein some residues may be absent
  misc_feature (233)..(252): This region may encompass 0-20 residues wherein some residues may be absent
  misc_feature (254)..(273): This region may encompass 0-20 residues wherein some residues may be absent
  misc_feature (275)..(294): This region may encompass 0-20 residues wherein some residues may be absent
  misc_feature (296)..(315): This region may encompass 0-20 residues wherein some residues may be absent
  misc_feature (317)..(336): This region may encompass 0-20 residues wherein some residues may be absent
  misc_feature (338)..(357): This region may encompass 0-20 residues wherein some residues may be absent
  misc_feature (359)..(378): This region may encompass 0-20 residues wherein some residues may be absent
  misc_feature (380)..(399): This region may encompass 0-20 residues wherein some residues may be absent
  misc_feature (401)..(420): This region may encompass 0-20 residues wherein some residues may be absent
  See specification as filed for detailed description of substitutions and preferred embodiments
SGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGG

SEQ ID NO: 362 - protein, 440 aa, Artificial Sequence
  Description of Artificial Sequence: Synthetic polypeptide
  misc_feature (1)..(440): This sequence may encompass 0-20 “Ser Glu Gly Gly Gly Gly Gly Gly Gly Gly Gly Gly Gly Gly Gly Gly Gly Gly Gly Gly Gly Gly” repeating units or embodiments thereof, wherein some residues may be absent
  misc_feature (3)..(22): This region may encompass 0-20 residues wherein some residues may be absent
  misc_feature (25)..(44): This region may encompass 0-20 residues wherein some residues may be absent
  misc_feature (47)..(66): This region may encompass 0-20 residues wherein some residues may be absent
  misc_feature (69)..(88): This region may encompass 0-20 residues wherein some residues may be absent
  misc_feature (91)..(110): This region may encompass 0-20 residues wherein some residues may be absent
  misc_feature (113)..(132): This region may encompass 0-20 residues wherein some residues may be absent
  misc_feature (135)..(154): This region may encompass 0-20 residues wherein some residues may be absent
  misc_feature (157)..(176): This region may encompass 0-20 residues wherein some residues may be absent
  misc_feature (179)..(198): This region may encompass 0-20 residues wherein some residues may be absent
  misc_feature (201)..(220): This region may encompass 0-20 residues wherein some residues may be absent
  misc_feature (223)..(242): This region may encompass 0-20 residues wherein some residues may be absent
  misc_feature (245)..(264): This region may encompass 0-20 residues wherein some residues may be absent
  misc_feature (267)..(286): This region may encompass 0-20 residues wherein some residues may be absent
  misc_feature (289)..(308): This region may encompass 0-20 residues wherein some residues may be absent
  misc_feature (311)..(330): This region may encompass 0-20 residues wherein some residues may be absent
  misc_feature (333)..(352): This region may encompass 0-20 residues wherein some residues may be absent
  misc_feature (355)..(374): This region may encompass 0-20 residues wherein some residues may be absent
  misc_feature (377)..(396): This region may encompass 0-20 residues wherein some residues may be absent
  misc_feature (399)..(418): This region may encompass 0-20 residues wherein some residues may be absent
  misc_feature (421)..(440): This region may encompass 0-20 residues wherein some residues may be absent
  See specification as filed for detailed description of substitutions and preferred embodiments
SEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGG

SEQ ID NO: 363 - protein, 422 aa, Artificial Sequence; identical to SEQ ID NO: 360
  Description of Artificial Sequence: Synthetic polypeptide
  misc_feature (1)..(420): This region may encompass 0-20 “Gly Gly Gly Gly Gly Gly Gly Gly Gly Gly Gly Gly Gly Gly Gly Gly Gly Gly Gly Gly Ser” repeating units or embodiments thereof, wherein some residues may be absent
  misc_feature (1)..(20): This region may encompass 0-20 residues wherein some residues may be absent
  misc_feature (22)..(41): This region may encompass 0-20 residues wherein some residues may be absent
  misc_feature (43)..(62): This region may encompass 0-20 residues wherein some residues may be absent
  misc_feature (64)..(83): This region may encompass 0-20 residues wherein some residues may be absent
  misc_feature (85)..(104): This region may encompass 0-20 residues wherein some residues may be absent
  misc_feature (106)..(125): This region may encompass 0-20 residues wherein some residues may be absent
  misc_feature (127)..(146): This region may encompass 0-20 residues wherein some residues may be absent
  misc_feature (148)..(167): This region may encompass 0-20 residues wherein some residues may be absent
  misc_feature (169)..(188): This region may encompass 0-20 residues wherein some residues may be absent
  misc_feature (190)..(209): This region may encompass 0-20 residues wherein some residues may be absent
  misc_feature (211)..(230): This region may encompass 0-20 residues wherein some residues may be absent
  misc_feature (232)..(251): This region may encompass 0-20 residues wherein some residues may be absent
  misc_feature (253)..(272): This region may encompass 0-20 residues wherein some residues may be absent
  misc_feature (274)..(293): This region may encompass 0-20 residues wherein some residues may be absent
  misc_feature (295)..(314): This region may encompass 0-20 residues wherein some residues may be absent
  misc_feature (316)..(335): This region may encompass 0-20 residues wherein some residues may be absent
  misc_feature (337)..(356): This region may encompass 0-20 residues wherein some residues may be absent
  misc_feature (358)..(377): This region may encompass 0-20 residues wherein some residues may be absent
  misc_feature (379)..(398): This region may encompass 0-20 residues wherein some residues may be absent
  misc_feature (400)..(419): This region may encompass 0-20 residues wherein some residues may be absent
  See specification as filed for detailed description of substitutions and preferred embodiments

SEQ ID NO: 364 - protein, 420 aa, Artificial Sequence; identical to SEQ ID NO: 361
  Description of Artificial Sequence: Synthetic polypeptide
  misc_feature (1)..(420): This sequence may encompass 0-20 “Ser Gly Gly Gly Gly Gly Gly Gly Gly Gly Gly Gly Gly Gly Gly Gly Gly Gly Gly Gly Gly” repeating units or embodiments thereof, wherein some residues may be absent
  misc_feature (2)..(21): This region may encompass 0-20 residues wherein some residues may be absent
  misc_feature (23)..(42): This region may encompass 0-20 residues wherein some residues may be absent
  misc_feature (44)..(63): This region may encompass 0-20 residues wherein some residues may be absent
  misc_feature (65)..(84): This region may encompass 0-20 residues wherein some residues may be absent
  misc_feature (86)..(105): This region may encompass 0-20 residues wherein some residues may be absent
  misc_feature (107)..(126): This region may encompass 0-20 residues wherein some residues may be absent
  misc_feature (128)..(147): This region may encompass 0-20 residues wherein some residues may be absent
  misc_feature (149)..(168): This region may encompass 0-20 residues wherein some residues may be absent
  misc_feature (170)..(189): This region may encompass 0-20 residues wherein some residues may be absent
  misc_feature (191)..(210): This region may encompass 0-20 residues wherein some residues may be absent
  misc_feature (212)..(231): This region may encompass 0-20 residues wherein some residues may be absent
  misc_feature (233)..(252): This region may encompass 0-20 residues wherein some residues may be absent
  misc_feature (254)..(273): This region may encompass 0-20 residues wherein some residues may be absent
  misc_feature (275)..(294): This region may encompass 0-20 residues wherein some residues may be absent
  misc_feature (296)..(315): This region may encompass 0-20 residues wherein some residues may be absent
  misc_feature (317)..(336): This region may encompass 0-20 residues wherein some residues may be absent
  misc_feature (338)..(357): This region may encompass 0-20 residues wherein some residues may be absent
  misc_feature (359)..(378): This region may encompass 0-20 residues wherein some residues may be absent
  misc_feature (380)..(399): This region may encompass 0-20 residues wherein some residues may be absent
  misc_feature (401)..(420): This region may encompass 0-20 residues wherein some residues may be absent
  See specification as filed for detailed description of substitutions and preferred embodiments

SEQ ID NO: 365 - protein, 440 aa, Artificial Sequence; identical to SEQ ID NO: 362
  Description of Artificial Sequence: Synthetic polypeptide
  misc_feature (1)..(440): This sequence may encompass 0-20 “Ser Glu Gly Gly Gly Gly Gly Gly Gly Gly Gly Gly Gly Gly Gly Gly Gly Gly Gly Gly Gly Gly” repeating units or embodiments thereof, wherein some residues may be absent
  misc_feature (3)..(22): This region may encompass 0-20 residues wherein some residues may be absent
  misc_feature (25)..(44): This region may encompass 0-20 residues wherein some residues may be absent
  misc_feature (47)..(66): This region may encompass 0-20 residues wherein some residues may be absent
  misc_feature (69)..(88): This region may encompass 0-20 residues wherein some residues may be absent
  misc_feature (91)..(110): This region may encompass 0-20 residues wherein some residues may be absent
  misc_feature (113)..(132): This region may encompass 0-20 residues wherein some residues may be absent
  misc_feature (135)..(154): This region may encompass 0-20 residues wherein some residues may be absent
  misc_feature (157)..(176): This region may encompass 0-20 residues wherein some residues may be absent
  misc_feature (179)..(198): This region may encompass 0-20 residues wherein some residues may be absent
  misc_feature (201)..(220): This region may encompass 0-20 residues wherein some residues may be absent
  misc_feature (223)..(242): This region may encompass 0-20 residues wherein some residues may be absent
  misc_feature (245)..(264): This region may encompass 0-20 residues wherein some residues may be absent
  misc_feature (267)..(286): This region may encompass 0-20 residues wherein some residues may be absent
  misc_feature (289)..(308): This region may encompass 0-20 residues wherein some residues may be absent
  misc_feature (311)..(330): This region may encompass 0-20 residues wherein some residues may be absent
  misc_feature (333)..(352): This region may encompass 0-20 residues wherein some residues may be absent
  misc_feature (355)..(374): This region may encompass 0-20 residues wherein some residues may be absent
  misc_feature (377)..(396): This region may encompass 0-20 residues wherein some residues may be absent
  misc_feature (399)..(418): This region may encompass 0-20 residues wherein some residues may be absent
  misc_feature (421)..(440): This region may encompass 0-20 residues wherein some residues may be absent
  See specification as filed for detailed description of substitutions and preferred embodiments

SEQ ID NO: 366 - protein, 32 aa, Artificial Sequence
  Description of Artificial Sequence: Synthetic polypeptide
  misc_feature (3)..(32): This sequence may encompass 1-10 “Ser Gly Gly” repeating units wherein some residues may be absent
  See specification as filed for detailed description of substitutions and preferred embodiments
GGSGGSGGSGGSGGSGGSGGSGGSGGSGGSGG

SEQ ID NO: 367-369 - skipped (3 entries)

SEQ ID NO: 370 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371-379 - skipped (9 entries)

SEQ ID NO: 380 - protein, 15 aa, Artificial Sequence
  Description of Artificial Sequence: Synthetic peptide
GGGGSGGGGSGGGGS

(19) United States
(12) Patent Application Publication
(10) Pub. No.: US 2016/0355588 A1
(43) Pub. Date: Dec. 8, 2016

(54) Bispecific CD3 and CD19 Antigen Binding Constructs

(71) Applicant: ZYMEWORKS INC., Vancouver (CA)

(72) Inventors: Gordon Yiu Kon Ng, Vancouver (CA); Surjit Bhimarao Dixit, Richmond (CA); Thomas Spreter von Kreudenstein, Vancouver (CA)

(21) Appl. No.: 14/903,184

(22) PCT Filed: Jul. 11, 2014
(86) PCT No.: PCT/US14/46436

§ 371 Date: Jan. 6, 2016

Related U.S. Application Data
(60) Provisional application No. 61/845,948, filed on Jul. 12, 2013.
(60) Provisional application No. 61/927,877, filed on Jan. 15, 2014.
(60) Provisional application No. 61/978,719, filed on Apr. 11, 2014.

(51) Int. Cl. C07K 16/28; C07K 16/30; G01N 33/68; A61K 47/48

(52) U.S. Cl. CPC C07K 16/2809; C07K 16/2803; A61K 47/48561; C07K 16/30; G01N 33/6872; C07K 2317/31; C07K 2317/55; C07K 2317/622; C07K 2317/526; C07K 2317/24; C07K 2317/21; A61K 2039/505

(57) ABSTRACT

Bispecific antigen binding constructs are described that bind to CD3 and CD19 or CD20 antigens.

### CROSS-REFERENCE TO RELATED APPLICATIONS

This application claims the benefit of U.S. provisional application No. 61/845,948, filed on Jul. 12, 2013 and U.S. provisional application No. 61/927,877, filed on Jan. 15, 2014 and U.S. provisional application No. 61/978,719, filed Apr. 11, 2014. These applications are hereby incorporated in their entirety by reference.

### SEQUENCE LISTING

The instant application contains a Sequence Listing which has been submitted via EFS-Web and is hereby incorporated by reference in its entirety. Said ASCII copy, created on Month XX, 2014, is named XXXXX_CRF_sequencelisting.txt, and is XXX,XXX bytes in size.

### FIELD OF THE INVENTION

The field of the invention is the rational design of multispecific scaffolds, e.g., antigen binding constructs, comprising a CD3 binding domain for custom development of biotherapeutics.

### BACKGROUND OF THE INVENTION

In the realm of therapeutic proteins, antibodies with their multivalent target binding features are excellent scaffolds for the design of drug candidates. Advancing these features further, designed bispecific antibodies and other fused multispecific therapeutics exhibit dual or multiple target specificities and an opportunity to create drugs with novel modes of action. The development of such multivalent and multispecific therapeutic proteins with favorable manufacturability, pharmacokinetics and functional activity has been a challenge.

Bi-specific antibodies capable of targeting T cells to tumor cells have been identified and tested for their efficacy in the treatment of cancers. Blinatumomab is an example of a bi-specific anti-CD3-CD19 antibody in a format called BiTE™ (Bi-specific T-cell Engager) that has been identified for the treatment of B-cell diseases such as relapsed B-cell non-Hodgkin lymphoma and chronic lymphocytic leukemia (Baeuerle et al (2009)12:4941-4944). The BiTE™ format is a bi-specific single chain antibody construct that links variable domains derived from two different antibodies. Blinatumomab, however, possesses poor half-life in vivo, and is difficult to manufacture in terms of production and stability. Thus, there is a need for improved bi-specific antibodies, capable of targeting T-cells to tumor cells and having improved manufacturability.

### SUMMARY OF THE INVENTION

Disclosed herein are isolated bispecific antigen binding constructs comprising a first antigen-binding polypeptide construct which monovalently and specifically binds a CD19 or CD20 antigen; a second antigen-binding polypeptide construct which monovalently and specifically binds a CD3 antigen; a heterodimeric Fc comprising first and second Fc polypeptides each comprising a modified CH3 domain, wherein each modified CH3 domain comprises asymmetric amino acid modifications that promote the formation of a heterodimeric Fc and the dimerized CH3 domains having a melting temperature (Tm) of about 68° C. or higher, wherein the first Fc polypeptide is linked to the first antigen-binding polypeptide construct, with or without a first linker, and the second monomeric Fc polypeptide is linked to the second antigen-binding polypeptide construct with or without a second linker; and wherein the first antigen binding polypeptide construct is a Fab and the second antigen binding polypeptide construct is an scFv or the first antigen binding polypeptide construct is an scFv and the second antigen binding polypeptide construct is a Fab.

### BRIEF DESCRIPTION OF THE FIGURES

The patent application file contains at least one drawing executed in color. If publicly available, copies of this patent application with color drawings will be provided by the U.S. Patent and Trademark Office upon request and payment of the necessary fee.

FIG. 1 depicts exemplary schematic representations of bi-specific antigen-binding constructs described herein. FIG. 1A represents a dual scFv heterodimer Fc format; FIG. 1B represents a hybrid heterodimer Fc format in an embodiment where the CD3-binding polypeptide is in scFv format and the CD19-binding polypeptide is in Fab format; FIG. 1C represents a hybrid heterodimer Fc format in an embodiment where the CD19-binding polypeptide is in scFv format and the CD3-binding polypeptide is in Fab format; FIG. 1D represents a full-size antibody format.

FIG. 2 provides a summary of exemplary CD3/CD19 bi-specific variants in dual scFv-Fc (also referred to herein as dual scFv format), hybrid or full size monoclonal antibody formats. The bi-specific variants shown in this Figure comprise antigen binding domains based on the mono-specific anti-CD3 antibody OKT3, and the mono-specific anti-CD19 antibody HD37. Potential modifications to the antigen binding domains that improve the biophysical and functional characteristics of the bi-specific variants are identified here, including cysteine to serine mutations in the CDR (CDR C→S), modifications to the scFv linker sequence (VHVL linker), and disulphide stabilizing modifications (VHVL SS). In addition, modification to the Fc region to knock-out FcγR binding activity is also identified as a means to modify functional characteristics of the variants.

FIG. 3 provides a summary of variant optimization for improved biophysical properties for selected bi-specific variants. This Figure indicates the optimization strategy that was used to improve the biophysical and functional characteristics, as well as manufacturability of the variants, and summarizes the expression yield after the final purification step, and heterodimer purity for each.

FIG. 4 provides a summary of the selected variants with respect to certain physical properties, protein yield from transient expression, binding properties and stage of validation (i.e. whether tested in in vivo or ex vivo models).

FIG. 5 demonstrates that selected variants are able to bridge CD19+ Raji B cells and Jurkat T cells. The left panel shows FACS bridging data for variants 875 and 891 compared to the control IgG. The right panel provides a summary of the T:B bridging analysis for variants 875, 1853, and 6476.

FIG. 6 depicts the ability of selected variants to bridge B and T cells with the formation of pseudopodia. The table on the left provides a summary of B:T cell bridging analysis for variants 875, 1661, 1853, 6476, and 6518; the photo on the right shows the formation of pseudopodia for variants 875, 1853, and 6518 as measured by bridging microscopy.

FIG. 7 depicts the ability of selected variants to mediate autologous B cell depletion in a human whole blood assay. The presence of CD20+ B cells was determined following 48 h IL-2 incubation in human whole blood (Average of 2 donors, n=4). FIG. 7A depicts the results for variants having the dual scFv heterodimer Fc format or hybrid heterodimer Fc format. FIG. 7B shows the results for a variant in the full-size antibody format.

FIG. 8 depicts the ability of selected variants to bind to the human G2 ALL tumor cell line.

FIG. 9 depicts the efficacy of variant 1661 (an FcγR knockout variant) compared to controls in an in vivo mouse B-ALL leukemia model. Panel A shows the amount of bioluminescence in the whole body in the prone position; Panel B shows the amount of bioluminescence in the whole body in the supine position; Panel C is an image of whole body bioluminescence; and Panel D shows the amount of bioluminescence detected in the spleen.

FIG. 10 depicts the efficacy of the hybrid variant 1853 and the dual scFv-Fc variant 875 compared to controls in an in vivo mouse B-ALL leukemia model. Panel A shows the amount of bioluminescence in the whole body in the prone position; Panel B shows the amount of bioluminescence in the whole body in the supine position; Panel C is an image of whole body bioluminescence; and Panel D shows the amount of bioluminescence detected in the spleen.

FIG. 11 depicts the pharmacokinetic analysis of exemplary CD3-CD19 heterodimer variants. The figure shows the PK profile of v875 at 0.8 mg/kg single IV dose in NSG (NOD SCID GAMMA) mice in comparison to a control antibody at 1.2 mg/kg. The control antibody is a mono-specific antibody that binds to HER2.

FIG. 12 depicts target B-cell dependence of T-cell activation by an exemplary bi-specific anti-CD3-CD19 antigen-binding construct.

FIG. 13 depicts the effect of an exemplary bi-specific anti-CD3-CD19 antigen-binding construct on T-cell proliferation in human PBMCs.

FIG. 14 depicts the effect of an exemplary bi-specific anti-CD3-CD19 antigen-binding construct on the release of IFNγ, TNFα, IL-2, IL-6 and IL-10 cytokines in human PBMCs.

FIG. 15 (A and B) demonstrates that a single IV dose of an exemplary bi-specific anti-CD3-CD19 antigen-binding construct 1853 at 3 mg/kg in NSG (NOD scid gamma, NOD.Cg-Prkdc^{scid}Il2rg^{tm1Wj1}/SzJ) mice has typical human IgG-like pharmacokinetics with respect to half-life, distribution and clearance in mice. FIG. 15C shows the analysis of the serum concentration of bi-specific CD3/CD19 variants at 24 h following 3 mg/kg IV injection. The analysis was done as part of the in vivo efficacy study (see Example 10 and FIGS. 9,10).

FIG. 16 depicts the ability of an exemplary bi-specific anti-CD3-CD19 antigen-binding construct to deplete autologous B-cells in an in vivo human B-ALL xenograft model in humanized NSG mice.

FIG. 17 depicts the activation and redistribution kinetics of autologous T-cells in response to treatment with an exemplary bi-specific anti-CD3-CD19 antigen-binding construct in an in vivo human B-ALL xenograft model in humanized NSG mice.

FIG. 18 depicts the effect of an exemplary bi-specific anti-CD3-CD19 antigen-binding construct on release of human cytokines IFNγ, TNFα, IL2, IL6, and IL10 in an in vivo human B-ALL xenograft model in humanized NSG mice.

FIG. 19 depicts the ability of a cross-species reactive variant 5851 to mediate autologous B cell depletion in a whole blood assay. The presence of CD20+ B cells was determined following 48 h IL-2 incubation in human whole blood (Average of 2 donors, n=4).

### DETAILED DESCRIPTION OF THE INVENTION

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

Terms understood by those in the art of antibody technology are each given the meaning acquired in the art, unless expressly defined differently herein.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or antigen binding constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All documents, or portions of documents, cited in the application including, but not limited to, patents, patent applications, articles, books, manuals, and treatises are hereby expressly incorporated by reference in their entirety for any purpose. All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985).

### Antigen Binding Constructs

Antigen binding construct refers to any agent, e.g., polypeptide or polypeptide complex capable of binding to an antigen. An antigen binding construct can be a monomer, dimer, multimer, a protein, a peptide, or a protein or peptide complex; an antibody or an antibody fragment; an scFv and the like.

The term “bispecific” is intended to include any agent, e.g., antigen binding construct, which has two different binding specificities. For example, in some embodiments, the agent may bind to, or interact with, (a) a cell surface target molecule and (b) an Fc receptor on the surface of an effector cell. In another embodiment, the agent may bind to, or interact with (a) a first cell surface target molecule and (b) a second cell surface target molecule that is different from the first cells surface target molecule. In another embodiment, the agent may bind to and bridge two cells, i.e. interact with (a) a first cell surface target molecule on a first call and (b) a second cell surface target molecule on a second cell that is different from the first cell's surface target molecule.

The term “multispecific” or “heterospecific” is intended to include any agent, e.g., antigen binding construct, which has more than two different binding specificities. For example, the agent may bind to, or interact with, (a) a cell surface target molecule such as but not limited to cell surface antigens, (b) an Fc receptor on the surface of an effector cell, and optionally (c) at least one other component. In another embodiment, the agent may bind to, or interact with two or more of (a) cell surface target molecule such as but not limited to cell surface antigens, (b) target molecules on the surface of an effector cell, and/or (c) other biologically relevant molecular component. Accordingly, embodiments of the antigen-binding constructs described herein, are inclusive of, but not limited to, bispecific, trispecific, tetraspecific, and other multispecific molecules. In certain embodiments, these molecules are directed to, e.g., CD3 antigens and/or CD19 antigens, CD20 antigens, and to other targets, such as Fc receptors on effector cells.

As used herein, “isolated” means an agent that has been identified and separated and/or recovered from a component of its natural cell culture environment. Contaminant components of its natural environment are materials that would interfere with diagnostic or therapeutic uses for the antigen-binding construct, and may include enzymes, hormones, and other proteinaceous or non-proteinaceous solutes.

### Antibodies

An antigen binding construct can be an antibody. As used herein, an “antibody” or “immunoglobulin” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG₂, IgG₃, IgG₄, IgAi, and IgA₂. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antigen binding construct provided herein, is from or derived from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

Fused or linked means that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. In certain other embodiments, one of the antigen binding moieties is a single-chain Fv molecule (scFv). As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the promote association of the two Fc domain subunits and the formation of heterodimers. For example in certain embodiments, a modification promoting association may alter the structure or charge of one or both of the Fc domain subunits so as to make their association favorable.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (AD CP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD 16a), FcγRI (CD64), and FcγRIIa (CD32).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain.

### Fc

The antigen-binding constructs according to the invention comprise a dimeric Fc. In some aspects, the Fc comprises at least one or two C_{H3} sequences. In some aspects, the Fc is coupled, with or without one or more linkers, to a first heterodimer and/or a second heterodimer. In some aspects, the Fc is a human Fc. In some aspects, the Fc is a human IgG or IgG1 Fc. In some aspects, the Fc is a heterodimeric Fc. In some aspects, the Fc comprises at least one or two C_{H2} sequences.

In some aspects, the Fc comprises one or more modifications in at least one of the C_{H3} sequences. In some aspects, the Fc comprises one or more modifications in at least one of the C_{H2} sequences. In some aspects, an Fc is a single polypeptide. In some aspects, an Fc is multiple peptides, e.g., two polypeptides.

In some aspects, Fc is an Fc described in patent applications PCT/CA2011/001238, filed Nov. 4, 2011 or PCT/CA2012/050780, filed Nov. 2, 2012, the entire disclosure of each of which is hereby incorporated by reference in its entirety for all purposes.

### Modified CH3

In some aspects, a construct described herein comprises a heterodimeric Fc comprising a modified CH3 domain that has been asymmetrically modified. The heterodimeric Fc can comprise two heavy chain constant domain polypeptides: a first heavy chain polypeptide and a second heavy chain polypeptide, which can be used interchangeably provided that Fc comprises one first heavy chain polypeptide and one second heavy chain polypeptide. Generally, the first heavy chain polypeptide comprises a first CH3 sequence and the second heavy chain polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table A provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, 5400, and/or N390, using EU numbering. In some aspects, an Fc includes a mutant sequence shown in Table X. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A-B. In some aspects, an Fc includes the mutations of Variant 5 A-B.

TABLE A
IgG1 Fc sequences

| | |
|---|---|
| Human IgG1 Fc | APELLGGPSVFLFPPKPKDTLMISRTPEVTCVVV |
| sequence 231-447 | DVSHEDPEVKFNWYVDGVEVHNAKTKPREEQYNS |
| (EU-numbering) | TYRVVSVLTVLHQDWLNGKEYKCKVSNKALPAPI |
| | EKTISKAKGQPREPQVYTLPPSRDELTKNQVSLT |
| | CLVKGFYPSDIAVEWESNGQPENNYKTTPPVLDS |
| | DGSFFLYSKLTVDKSRWQQGNVFSCSVMHEALHN |
| | HYTQKSLSLSPGK (SEQ ID NO: 370) |

| Variant IgG1 | | |
|---|---|---|
| Fc sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A:L351Y_F405A_Y407V. B:T366L_K392M_T394W, A:L351Y_F405A_Y407V, B:T366L_K392L_T394W, A:T350V_L351Y_F405A_Y407V. B:T350V_T366L_K392L_T394W, A:T350V_L351Y_F405A_Y407V, B:T350V_T366L_K392M_T394W, A:T350V_L351Y_S400E_F405A_Y407V, and/or B:T350V_T366L_N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the C_{H3} sequences that promote the formation of a heterodimeric Fc with stability comparable to a wild-type homodimeric Fc.

In one embodiment, the stability of the CH3 domain can be assessed by measuring the melting temperature of the CH3 domain, for example by differential scanning calorimetry (DSC). Thus, in a further embodiment, the CH3 domain has a melting temperature of about 68° C. or higher. In another embodiment, the CH3 domain has a melting temperature of about 70° C. or higher. In another embodiment, the CH3 domain has a melting temperature of about 72° C. or higher. In another embodiment, the CH3 domain has a melting temperature of about 73° C. or higher. In another embodiment, the CH3 domain has a melting temperature of about 75° C. or higher. In another embodiment, the CH3 domain has a melting temperature of about 78° C. or higher. In some aspects, the dimerized C_{H3} sequences have a melting temperature (Tm) of about 68, 69, 70, 71, 72, 73, 74, 75, 76, 77, 77.5, 78, 79, 80, 81, 82, 83, 84, or 85° C. or higher.

In some embodiments, a heterodimeric Fc comprising modified CH3 sequences can be formed with a purity of at least about 75% as compared to homodimeric Fc in the expressed product. In another embodiment, the heterodimeric Fc is formed with a purity greater than about 80%. In another embodiment, the heterodimeric Fc is formed with a purity greater than about 85%. In another embodiment, the heterodimeric Fc is formed with a purity greater than about 90%. In another embodiment, the heterodimeric Fc is formed with a purity greater than about 95%. In another embodiment, the heterodimeric Fc is formed with a purity greater than about 97%. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed. In some aspects, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed via a single cell.

Additional methods for modifying monomeric Fc polypeptides to promote heterodimeric Fc formation are described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bispecific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

In some embodiments an isolated construct described herein comprises an antibody construct which binds an antigen; and a dimeric Fc polypeptide construct that has superior biophysical properties like stability and ease of manufacture relative to an antibody construct which does not include the same Fc polypeptide. A number of mutations in the heavy chain sequence of the Fc are known in the art for selectively altering the affinity of the antibody Fc for the different Fcgamma receptors. In some aspects, the Fc comprises one or more modifications to promote selective binding of Fc-gamma receptors.

### CH2 Domain

The CH2 domain of an Fc is amino acid 231-340 of the sequence shown in Table a. Exemplary mutations are listed below:
S298A/E333A/K334A, S298A/E333A/K334A/K326A (Lu Y, Vernes J M, Chiang N, et al. J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13 (6):R123);
F243L (Stewart R, Thom G, Levens M, et al. Protein Eng Des Sel. 2011 September; 24(9):671-8.), S298A/E333A/K334A (Shields R L, Namenuk A K, Hong K, et al. J Biol Chem. 2001 Mar. 2; 276(9):6591-604);
S239D/I332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S, et al. Mol Immunol. 2008 September; 45(15):3926-33);
S239D/D265S/S298A/1332E, S239E/S298A/K326A/A327H, G237F/S298A/A330L/I 332E, S239D/I332E/S298A, S239D/K326E/A330L/1332E/S298A, G236A/S239D/D270L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments a CH2 domain comprises one or more asymmetric amino acid modifications. In some embodiments a CH2 domain comprises one or more asymmetric amino acid modifications to promote selective binding of a FcγR. In some embodiments the CH2 domain allows for separation and purification of an isolated construct described herein.

### Additional Modifications to Improve Effector Function.

In some embodiments a construct described herein can be modified to improve its effector function. Such modifications are known in the art and include afucosylation, or engineering of the affinity of the Fc portion of antibodies towards an activating receptor, mainly FCGR3a for ADCC, and towards C1q for CDC. The following Table B summarizes various designs reported in the literature for effector function engineering.

Thus, in one embodiment, a construct described herein can include a dimeric Fc that comprises one or more amino acid modifications as noted in Table B that confer improved effector function. In another embodiment, the construct can be afucosylated to improve effector function.

TABLE B
CH2 and effector function engineering.

| Reference | Mutations | Effect |
|---|---|---|
| Lu, 2011, | Afucosylated | Increased |
| Ferrara 2011, | | ADCC |
| Mizushima 2011 | | |
| Lu, 2011 | S298A/E333A/K334A | Increased |
| | | ADCC |
| Lu, 2011 | S298A/E333A/K334A/K326A | Increased |
| | | ADCC |
| Stavenhagen, | F243L/R292P/Y300L/V305I/P396L | Increased |
| 2007 | | ADCC |
| Nordstrom, | F243L/R292P/Y300L/L235V/P396L | Increased |
| 2011 | | ADCC |
| Stewart, | F243L | Increased |
| 2011 | | ADCC |
| Shields, | S298A/E333A/K334A | Increased |
| 2001 | | ADCC |
| Lazar, | S239D/I332E/A330L | Increased |
| 2006 | | ADCC |
| Lazar, | S239D/I332E | Increased |
| 2006 | | ADCC |
| Bowles, | AME-D, not specified mutations | Increased |
| 2006 | | ADCC |
| Heider, | 37.1, mutations not disclosed | Increased |
| 2011 | | ADCC |
| Moore, | S267E/H268F/S324T | Increased |
| 2010 | | CDC |

### FcRn Binding and PK Parameters

As is known in the art, binding to FcRn recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full-length molecule, results in favorable antibody serum half-lives ranging from one to three weeks. Binding of Fc to FcRn also plays a key role in antibody transport. Thus, in one embodiment, the constructs of the invention are able to bind FcRn.

Fc modifications reducing FcγR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc region of the antibody. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcγR or complement binding to the Fc.

Specific, non-limiting examples of known amino acid modifications include those identified in the following table:

TABLE C
modifications to reduce FcγR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

In one embodiment, the Fc comprises at least one amino acid modification identified in the above table. In another embodiment the Fc comprises amino acid modification of at least one of L234, L235, or D265. In another embodiment, the Fc comprises amino acid modification at L234, L235 and D265. In another embodiment, the Fc comprises the amino acid modification L234A, L235A and D265S.

### Linkers

The constructs described herein can include one or more heterodimers described herein operatively coupled to an Fc described herein. In some aspects, Fc is coupled to the one or more heterodimers with or without one or more linkers. In some aspects, Fc is directly coupled to the one or more heterodimers. In some aspects, Fc is coupled to the one or more heterodimers by one or more linkers. In some aspects, Fc is coupled to the heavy chain of each heterodimer by a linker.

In some aspects, the one or more linkers are one or more polypeptide linkers. In some aspects, the one or more linkers comprise one or more IgG1 hinge regions.

Format scFv

The antigen binding constructs described herein are bi-specific, e.g., they comprise at least two antigen binding polypeptide constructs each capable of specific binding to two distinct antigens. One antigen binding polypeptide construct is in an scFv format. (i.e. antigen binding domains composed of a heavy chain variable domain and a light chain variable domain). In one embodiment said scFv molecules are human. In another embodiment said scFv molecules are humanized.

In the scFv molecule the C-terminus of the light chain variable region may be connected to the N-terminus of the heavy chain variable region, or the C-terminus of the heavy chain variable region may be connected to the N-terminus of the light chain variable region.

The variable regions may be connected directly or, typically, via a linker peptide that allows the formation of a functional antigen binding moiety. Typical peptide linkers comprise about 2-20 amino acids, and are described herein or known in the art. Suitable, non-immunogenic linker peptides include, for example, (G4S)n, (SG4)n, (G4S)n, G4(SG4)n or G2(SG2)n linker peptides, wherein n is generally a number between 1 and 10, typically between 2 and 4.

The scFv molecule may be further stabilized by disulfide bridges between the heavy and light chain variable domains, for example as described in Reiter et al. (Nat Biotechnol 14, 1239-1245 (1996)). Hence, in one embodiment the T cell activating bi-specific antigen binding molecule of the invention comprises a scFv molecule wherein an amino acid in the heavy chain variable domain and an amino acid in the light chain variable domain have been replaced by cysteine so that a disulfide bridge can be formed between the heavy and light chain variable domain. In a specific embodiment the amino acid at position 44 of the light chain variable domain and the amino acid at position 100 of the heavy chain variable domain have been replaced by cysteine (Kabat numbering).

As is known in the art, scFvs can also be stabilized by mutation of CDR sequences, as described in [Miller et al., Protein Eng Des Sel. 2010 July; 23(7):549-57; Igawa et al., MAbs. 2011 May-June; 3(3):243-5; Perchiacca & Tessier, Annu Rev Chem Biomol Eng. 2012; 3:263-86.].

### HVR and CDR

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

### Antigens

The antigen binding construct specifically binds at least one antigen, e.g., a CD3 antigen and/or a CD19 antigen. As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Examples include CD3 antigens, CD19 antigens, and CD20 antigens.

Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g., CD3, CD19, and C20) can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants. Other human proteins useful as antigens include, but are not limited to: Melanoma-associated Chondroitin Sulfate Proteoglycan (MCSP), also known as Chondroitin Sulfate Proteoglycan 4 (UniProt no. Q6UVK1 (version 70), NCBI RefSeq no. NP 001888.2); Fibroblast Activation Protein (FAP), also known as Seprase (Uni Prot nos. Q12884, Q86Z29, Q99998, NCBI Accession no. NP 004451); Carcinoembroynic antigen (CEA), also known as Carcinoembryonic antigen-related cell adhesion molecule 5 (UniProt no. P06731 (version 119), NCBI RefSeq no. NP 004354.2); CD33, also known as gp67 or Siglec-3 (UniProt no. P20138, NCBI Accession nos. NP 001076087, NP 001171079); Epidermal Growth Factor Receptor (EGFR), also known as ErbB-1 or Her1 (UniProt no. P0053, NCBI Accession nos. NP 958439, NP 958440), and CD3, particularly the epsilon subunit of CD3 (see UniProt no. P07766 (version 130), NCBI RefSeq no. NP 000724.1, for the human sequence; or UniProt no. Q95LI5 (version 49), NCBI GenBank no. BAB71849.1, for the cynomolgus [Macaca fascicularis] sequence).

In certain embodiments the T cell activating bispecific antigen binding molecule of the invention binds to an epitope of an activating T cell antigen or a target cell antigen that is conserved among the activating T cell antigen or target antigen from different species.

By “specific binding” or “selective binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al, Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of <1 μM, <100 nM, <10 nM, <1 nM, <0.1 nM, <0.01 nM, or <0.001 nM (e.g. 10⁻⁸ M or less, e.g. from 10⁻⁸ M to 10¹³ M, e.g., from 10″⁹ M to 10″¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

A “target cell antigen” as used herein refers to an antigenic determinant presented on the surface of a target cell, for example a B cell in a tumor such as a cancer cell or a cell of the tumor stroma. As used herein, the terms “first” and “second” with respect to antigen binding moieties etc., are used for convenience of distinguishing when there is more than one of each type of moiety. Use of these terms is not intended to confer a specific order or orientation of the T cell activating bispecific antigen binding molecule unless explicitly so stated.

The term “cross-species binding” or “interspecies binding” as used herein means binding of a binding domain described herein to the same target molecule in humans and other organisms for instance, but not restricted to non-chimpanzee primates. Thus, “cross-species binding” or “interspecies binding” is to be understood as an interspecies reactivity to the same molecule “X” (i.e. the homolog) expressed in different species, but not to a molecule other than “X”. Cross-species specificity of a monoclonal antibody recognizing e.g. human CD3 epsilon, to a non-chimpanzee primate CD3 epsilon, e.g. macaque CD3 epsilon, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate CD3 epsilon antigens, respectively. Additional assays are well known to one of skill in the art. The above-mentioned subject matter applies mutatis mutandis for the PSCA, CD19, C-MET, Endosialin, EpCAM, IGF-1R and FAPα antigen: Cross-species specificity of a monoclonal antibody recognizing e.g. human PSCA, CD19, C-MET, Endosialin, EpCAM, IGF-1R or FAPα, to a non-chimpanzee primate PSCA, CD19, C-MET, Endosialin, EpCAM, IGF-1R or FAPα, e.g. macaque PSCA, CD19, C-MET, Endosialin, EpCAM, IGF-1R or FAPα, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate PSCA, CD19, C-MET, Endosialin, EpCAM, IGF-1R or FAPα antigens, respectively.

### CD3, CD19, and CD20

The antigen binding constructs of the invention include antigen binding polypeptide constructs that monovalently and specifically bind a CD3 antigen and/or a CD19 antigen and/or a CD20 antigen.

“CD3” or “CD3 complex” as described herein is a complex of at least five membrane-bound polypeptides in mature T-lymphocytes that are non-covalently associated with one another and with the T-cell receptor. The CD3 complex includes the gamma, delta, epsilon, zeta, and eta chains (also referred to as subunits). Non-human monoclonal antibodies have been developed against some of these chains, as exemplified by the murine antibodies OKT3, SP34, UCHT1 or 64.1. (See e.g., June, et al., J. Immunol. 136:3945-3952 (1986); Yang, et al., J. Immunol. 137:1097-1100 (1986); and Hayward, et al., Immunol. 64:87-92 (1988)). Clustering of CD3 on T cells, e.g., by immobilized anti-CD3-antibodies, leads to T cell activation similar to the engagement of the T cell receptor but independent from its clone typical specificity. Most anti-CD3-antibodies recognize the CD3ε-chain.

In one embodiment, the bi-specific antigen-binding construct comprises a CD3 antigen binding polypeptide which monovalently and specifically binds a CD3 antigen derived from OKT3 (ORTHOCLONE-OKT3™ (muromonab-CD3); Teplizumab™ (MGA031, Eli Lilly); Species cross reactive anti-CD3 (Micromet, US2011/0275787); Blinatumomab™; UCHT1 (Pollard et al. 1987 J Histochem Cytochem. 35(11):1329-38); NI0401 (WO2007/033230); visilizumab (US25834597). In one embodiment the bi-specific antigen-binding construct comprises a CD3 antigen binding polypeptide which monovalently and specifically binds a CD3 antigen, the VH and VL regions of said CD3 antigen-binding polypeptide derived from a CD3 specific antibody selected from the group consisting of X35-3, VIT3, BMA030 (BW264/56), CLB-T3/3, CRIS7, YTH12.5, F111-409, CLB-T3.4.2, WT31, WT32, SPv-T3b, 11D8, XIII-141, XIII-46, XIII-87, 12F6, T3/RW2-8C8, T3/RW2-4B6, OKT3D, M-T301, SMC2 and F101.01.

In accordance with this invention, said VH and VL regions are derived from antibodies/antibody derivatives and the like which are capable of specifically recognizing human CD3 epsilon in the context of other TCR subunits.

Antibodies/antibody molecules/antibody derivatives directed against human CD19 which provide for variable regions (VH and VL) to be employed in the bispecific antigen binding construct(s) comprised in the inventive pharmaceutical composition are also well known in the art. In one embodiment, the CD19-binding antigen-binding polypeptide is derived from antibodies directed to human CD19 such as, for example: 4G7 (Meecker (1984) Hybridoma 3, 305-20); B4 (Freedman (1987) Blood 70, 418-27; B43 (Bejcek (1995) Cancer Res. 55, 2346-51); BU12 (Callard et al., J. Immunology, 148(10):2983-7 (1992), Flavell (1995) Br. J. Cancer 72, 1373-9); CLB-CD19 (De Rie (1989) Cell. Immunol. 118, 368-81); Leu-12 (MacKenzie (1987), J. Immunol. 139, 24-8); SJ25-C1 (GenTrak, Plymouth Meeting, Pa.), J4.119 (Beckman Coulter, Krefeld, Germany), B43 (PharMingen, San Diego, Calif.), SJ25C1 (BD PharMingen, San Diego, Calif.), FMC63 (IgG2a) (Zola et al., Immunol. Cell. Biol. 69(PT6): 411-22 (1991); Nicholson et al., Mol. Immunol., 34:1157-1165 (1997); Pietersz et al., Cancer Immunol. Immunotherapy, 41:53-60 (1995)), and/or HD237 (IgG2b) (Fourth International Workshop on Human Leukocyte Differentiation Antigens, Vienna, Austria, 1989; and Pezzutto et al., J. Immunol., 138(9):2793-2799 (1987)). The CD19 antigen-binding polypeptide can also be derived from an antibody such as Mor-208, MEDI-551, MDX-1342, or other anti-CD19 antibodies as described in Hammer (2012) Mabs 4:5, 571-577. In yet another embodiment said VH(CD19) and VL(CD19) regions (or parts, like CDRs, thereof) are derived from the antibody provided by the HD37 hybridoma (Pezzutto (1997), J. Immunol. 138, 2793-9).

CD20 is a non-glycosylated phosphoprotein expressed on the cell membranes of mature B cells. CD20 is considered a B cell tumor-associated antigen because it is expressed by more than 95% of B-cell non-Hodgkin lymphomas (NHLs) and other B-cell malignancies, but it is absent on precursor B-cells, dendritic cells and plasma cells. Anti-CD20 antibodies are believed to kill CD20-expressing tumor cells by complement dependent cytotoxicity (CDC), antibody-dependent cell mediated cytotoxicity (ADCC) and/or induction of apoptosis and sensitization to chemotherapy. Bi-specific antigen-binding constructs can be derived from the anti-CD20 antibodies rituximab, ofatumumab, or tositumumab. The rituximab (RITUXAN®) antibody is a genetically engineered chimeric murine/human monoclonal antibody directed against CD20. Rituximab is the antibody called “C2B8” in U.S. Pat. No. 5,736,137 (Anderson et al.). CD20 antigen-binding polypeptides can also be derived from additional anti-CD20 antibodies as described in Lim et al., Haematologica 2010; 95(1): 135-143.

The expression of certain CD antigens is highly restricted to specific lineages of lymphohematopoietic cells and over the past several years, antibodies directed against lymphoid-specific antigens have been used to develop treatments that were effective either in vitro or in animal models. In this respect CD19 has proved to be a very useful target. CD19 is expressed in the whole B lineage from the pro B cell to the mature B cell, it is not shed, is uniformly expressed on all lymphoma cells, and is absent from stem cells.

### CD3 Complex Binding Polypeptide Constructs:

In certain embodiments of the antigen-binding constructs provided herein, said antigen-binding construct comprises at least one CD3 binding polypeptide construct that binds to a CD3 complex on at least one CD3 expressing cell. In some embodiments, the at least one CD3 binding polypeptide construct comprises at least one CD3 binding domain from a CD3 specific antibody, a nanobody, fibronectin, affibody, anticalin, cysteine knot protein, DARPin, avimer, Kunitz domain or variant or derivative thereof. In some embodiments, the at least one CD3 binding domain comprises at least one amino acid modification that reduces immunogenicity as compared to a corresponding CD3 binding domain not comprising said modification. In an embodiment, the at least one CD3 binding domain comprises at least one amino acid modification that increases its stability as measured by Tₘ, as compared to a corresponding CD3 binding domain not comprising said modification. In some embodiments, there is about a 3 degree increase in the Tₘ as compared to the native CD3 binding domain not comprising said at least one modification. In some embodiments, there is about a 5 degree increase in the Tₘ as compared to the native CD3 binding domain not comprising said at least one modification. In some embodiments, there is about a 8 degree increase in the Tₘ as compared to the native CD3 binding domain not comprising said at least one modification. In some embodiments, there is about a 10 degree increase in the Tₘ as compared to the native CD3 binding domain not comprising said at least one modification.

In some embodiments, the at least one CD3 binding polypeptide construct described herein comprises at least one CD3 binding domain from a CD3 specific antibody wherein said CD3 specific antibody is a heavy chain antibody devoid of light chains.

In certain other embodiments, the at least one CD3 binding polypeptide construct described herein comprises at least one CD3 binding domain derived from a non-antibody protein scaffold domain.

In certain embodiments, the CD3 binding polypeptide constructs are CD3 binding Fab constructs (i.e. antigen binding constructs comprising a heavy and a light chain, each comprising a variable and a constant region). In some embodiment said Fab construct is mammalian. In one embodiment said Fab construct is human. In another embodiment said Fab construct is humanized. In yet another embodiment said Fab construct comprises at least one of human heavy and light chain constant regions. In a further embodiment said Fab construct is a single chain Fab (scFab).

In certain embodiments the CD3 binding polypeptide constructs comprise CD3 binding scFab constructs wherein the C-terminus of the Fab light chain is connected to the N-terminus of the Fab heavy chain by a peptide linker. The peptide linker allows arrangement of the Fab heavy and light chain to form a functional CD3 binding moiety. In certain embodiments, the peptide linkers suitable for connecting the Fab heavy and light chain include sequences comprising glycine-serine linkers for instance, but not limited to (GₘS)ₙ-GG (SEQ ID NO: 360), (SGₙ)ₘ, (SEQ ID NO: 361), (SEGₙ)ₘ (SEQ ID NO: 362), wherein m and n are between 0-20. In certain embodiments, the scFab construct is a cross-over construct wherein the constant regions of the Fab light chain and the Fab heavy chain are exchanged. In another embodiment of a cross-over Fab, the variable regions of the Fab light chain and the Fab heavy chain are exchanged.

In certain embodiments, the CD3 binding polypeptide constructs comprise CD3 binding Fv constructs (i.e. antigen binding constructs comprising a heavy and a light chain, each comprising a variable region). In some embodiment said Fv construct is mammalian. In one embodiment said Fv construct is human. In another embodiment said Fv construct is humanized. In yet another embodiment said Fv construct comprises at least one of human heavy and light chain variable regions. In a further embodiment said Fv construct is a single chain Fv (scFv).

In some embodiments, the CD3 binding polypeptide construct of an antigen-binding construct described herein bind to at least one component of the CD3 complex. In a specific embodiment, the CD3 binding polypeptide construct binds to at least one of CD3 epsilon, CD3 gamma, CD3 delta or CD3 zeta of the CD3 complex. In certain embodiments, the CD3 binding polypeptide construct binds the CD3 epsilon domain. In certain embodiments, binding polypeptide construct binds a human CD3 complex. In certain embodiments, the CD3 binding polypeptide construct exhibits cross-species binding to a least one member of the CD3 complex.

Provided herein are antigen-binding constructs comprising at least one CD3 binding polypeptide construct that binds to a CD3 complex on at least one CD3 expressing cell, where in the CD3 expressing cell is a T-cell. In certain embodiments, the CD3 expressing cell is a human cell. In some embodiments, the CD3 expressing cell is a non-human, mammalian cell. In some embodiments, the T cell is a cytotoxic T cell. In some embodiments the T cell is a CD4⁺ or a CD8⁺ T cell.

In certain embodiments of the antigen-binding constructs provided herein, the construct is capable of activating and redirecting cytotoxic activity of a T cell to a target cell such as a B cell. In a particular embodiment, said redirection is independent of MHC-mediated peptide antigen presentation by the target cell and and/or specificity of the T cell.

Provided herein are antigen-binding constructs that are capable of simultaneous binding to a B cell antigen for instance a tumor cell antigen, and an activating T cell antigen. In one embodiment, the antigen-binding construct is capable of crosslinking a T cell and a target B cell by simultaneous binding to a B cell antigen for instance CD19 or CD20 and an activating T cell antigen for instance CD3. In one embodiment, the simultaneous binding results in lysis of a target B cell, for instance a tumor cell. In one embodiment, such simultaneous binding results in activation of the T cell. In other embodiments, such simultaneous binding results in a cellular response of a T lymphocyte, for instance a cytotoxic T lymphocyte, selected from the group of: proliferation, differentiation, cytokine secretion, cytotoxic effector molecule release, cytotoxic activity, and expression of activation markers. In one embodiment, binding of the T cell activating bispecific antigen binding molecule to the activating T cell antigen without simultaneous binding to the target cell antigen does not result in T cell activation.

CD19 and/or CD20 B Cell Binding Polypeptide Constructs:

Provided herein are isolated antigen-binding constructs comprising at least one antigen binding polypeptide construct that binds to a target antigen on at least one B cell. In certain embodiments, the antigen binding polypeptide construct binds at least one member of a B cell CD21-CD19-CD81 complex. In some embodiments, the antigen binding polypeptide construct comprises at least one CD19 binding domain or fragment thereof. In an embodiment, the antigen binding polypeptide construct comprises at least one CD20 binding domain.

In some embodiments, the at least one antigen binding domain is a CD19 or CD20 binding domain which is obtained from a CD19 or CD20 specific antibody, a nanobody, fibronectin, affibody, anticalin, cysteine knot protein, DARPin, avimer, Kunitz domain or variant or derivative thereof. In some embodiments, the at least one antigen binding polypeptide construct described herein comprises at least one antigen binding domain which is a CD19 or CD20 binding domain from an antibody which is a heavy chain antibody devoid of light chains.

In some embodiments, the at least one antigen binding domain is a CD19 or CD20 binding domain that comprises at least one amino acid modification that reduces immunogenicity as compared to a corresponding antigen binding domain not comprising said modification. In an embodiment, the at least one antigen binding domain is a CD19 or CD20 binding domain comprising at least one amino acid modification that increases its stability as measured by Tₘ, as compared to a corresponding domain not comprising said modification.

In certain embodiments, the at least one antigen binding polypeptide construct is a Fab construct that binds at least one of CD19 and CD20 on a B cell. In some embodiment said Fab construct is mammalian. In one embodiment said Fab construct is human. In another embodiment said Fab construct is humanized. In yet another embodiment said Fab construct comprises at least one of human heavy and light chain constant regions. In a further embodiment said Fab construct is a single chain Fab (scFab).

In certain embodiments the CD19 and/or CD20 binding polypeptide construct comprises a scFab construct wherein the C-terminus of the Fab light chain is connected to the N-terminus of the Fab heavy chain by a peptide linker. The peptide linker allows arrangement of the Fab heavy and light chain to form a functional CD19 and/or CD20 binding moiety. In certain embodiments, the peptide linkers suitable for connecting the Fab heavy and light chain include sequences comprising glycine-serine linkers for instance, but not limited to (GₘS)ₙ-GG (SEQ ID NO: 363), (SGₙ)ₘ (SEQ ID NO: 364) (SEGₙ)ₘ (SEQ ID NO: 365), wherein m and n are between 0-20. In certain embodiments, the scFab construct is a cross-over construct wherein the constant regions of the Fab light chain and the Fab heavy chain are exchanged. In another embodiment of a cross-over Fab, the variable regions of the Fab light chain and the Fab heavy chain are exchanged.

In certain embodiments, the at least one antigen binding polypeptide construct is a Fv construct that binds at least one of CD19 and CD20 on a B cell. In some embodiment said Fv construct is mammalian. In one embodiment said Fv construct is human. In another embodiment said Fv construct is humanized. In yet another embodiment said Fv construct comprises at least one of human heavy and light chain variable regions. In a further embodiment said Fv construct is a single chain Fv (scFv).

In certain embodiments, the antigen binding polypeptide construct exhibits cross-species binding to a least one antigen expressed on the surface of a B cell. In some embodiments, the antigen binding polypeptide construct of an antigen-binding construct described herein bind to at least one of mammalian CD19 and CD20. In certain embodiments, binding polypeptide construct binds a human CD19 or CD20.

Provided herein are constructs that are capable of simultaneous binding to a B cell antigen for instance a tumor cell antigen, and an activating T cell antigen. In one embodiment, the antigen-binding construct is capable of crosslinking a T cell and a target B cell by simultaneous binding to a B cell antigen for instance CD19 or CD20 and an activating T cell antigen for instance CD3.

In certain embodiments, an antigen-binding construct described herein comprises at least one antigen binding polypeptide construct that binds to a target antigen such as a CD19 or CD20 on at least one B cell associated with a disease. In some embodiments, the disease is a cancer selected from a carcinoma, a sarcoma, leukemia, lymphoma and glioma. In an embodiment, the cancer is at least one of squamous cell carcinoma, adenocarcinoma, transition cell carcinoma, osteosarcoma and soft tissue sarcoma. In certain embodiments, the at least one B cell is an autoimmune reactive cell that is a lymphoid or myeloid cell.

### Additional Antigen Binding Constructs:

In certain embodiments, an antigen-binding construct described herein further comprises at least one binding domain that binds at least one of: GPA133, EpCAM, EGFR, IGFR, HER-2 neu, HER-3, HER-4, PSMA, CEA, MUC-1 (mucin), MUC2, MUC3, MUC4, MUC5, MUC7, CCR4, CCR5, CD19, CD20, CD33, CD30, ganglioside GD3, 9-O-Acetyl-GD3, GM2, Poly SA, GD2, Carboanhydrase IX (MN/CA IX), CD44v6, Sonic Hedgehog (Shh), Wue-1, Plasma Cell Antigen, (membrane-bound), Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, LG, SAS, EPHA4 CD63, CD3 BsAb immunocytokines TNF which comprise a CD3 antibody attached to the cytokine, IFN•, IL-2, and TRAIL.

### Polypeptides and Polynucleotides

The antigen binding constructs comprise at least one polypeptide. The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

Also included in the invention are polynucleotides encoding polypeptides of the antigen binding constructs. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

By “isolated nucleic acid molecule or polynucleotide” is intended a nucleic acid molecule, DNA or RNA, which has been removed from its native environment. For example, a recombinant polynucleotide encoding a polypeptide contained in a vector is considered isolated. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids described herein, further include such molecules produced synthetically, e.g., via PCR or chemical synthesis. In addition, a polynucleotide or a nucleic acid, in certain embodiments, include a regulatory element such as a promoter, ribosome binding site, or a transcription terminator.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridizing preferentially to a template nucleic acid.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles of the invention.

Conservative substitution tables providing functionally similar amino acids are known to those of ordinary skill in the art. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins: Structures and Molecular Properties (W H Freeman & Co.; 2nd edition (December 1993)

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 50% identity, about 55% identity, 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide of the present invention, including homologs from species other than human, may be obtained by a process comprising the steps of screening a library under stringent hybridization conditions with a labeled probe having a polynucleotide sequence of the invention or a fragment thereof, and isolating full-length cDNA and genomic clones containing said polynucleotide sequence. Such hybridization techniques are well known to the skilled artisan.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

### Methods of Recombinant and Synthetic Production of Antigen-Binding Constructs:

Also described herein are methods of producing the antigen binding constructs via expression of the polypeptide(s) in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. In certain embodiments, progeny are not completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

Provided are methods of producing an expression product containing an antigen-binding construct as described herein, in stable mammalian cells, the method comprising: transfecting at least one mammalian cell with: at least a first DNA sequence encoding said first polypeptide construct and at least a second DNA sequence encoding said second polypeptide construct, such that said at least one first DNA sequence, said at least one second DNA sequence are transfected in said at least one mammalian cell in a pre-determined ratio to generate stable mammalian cells; culturing said stable mammalian cells to produce said expression product comprising said antigen-binding construct. In certain embodiments, said predetermined ratio of the at least one first DNA sequence: at least one second DNA sequence is about 1:1. In certain other embodiments, said predetermined ratio of the at least one first DNA sequence: at least one second DNA sequence is skewed towards a larger amount of the one first DNA sequence such as about 2:1. In yet other embodiments, said predetermined ratio of the at least one first DNA sequence: at least one second DNA sequence is skewed towards a larger amount of the one first DNA sequence such as about 1:2. In select embodiments, the mammalian cell is selected from the group consisting of a VERO, HeLa, HEK, NSO, Chinese Hamster Ovary (CHO), W138, BHK, COS-7, Caco-2 and MDCK cell, and subclasses and variants thereof.

In certain embodiments are antigen-binding constructs produced as recombinant molecules by secretion from yeast, a microorganism such as a bacterium, or a human or animal cell line. In embodiments, the polypeptides are secreted from the host cells.

Embodiments include a cell, such as a yeast cell transformed to express an antigen-binding construct protein described herein. In addition to the transformed host cells themselves, are provided culture of those cells, preferably a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells and insect cells.

An antigen-binding construct described herein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well-known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary photopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase 1, enzymes that remove protruding, _single-stranded termini with their 3′ 5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc., New Haven, Conn., USA.

Exemplary genera of yeast contemplated to be useful in the practice of the present invention as hosts for expressing the proteins are Pichua (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii.

Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063, all of which are incorporated herein by reference.

Exemplary species of Saccharomyces useful for the synthesis of antigen-binding constructs described herein include S. cerevisiae, S. italicus, S. diastaticus, and Zygosaccharomyces rouxii. Preferred exemplary species of Kluyveromyces include K. fragilis and K. lactis. Preferred exemplary species of Hansenula include H. polymorpha (now Pichia angusta), H. anomala (now Pichia anomala), and Pichia capsulata. Additional preferred exemplary species of Pichia include P. pastoris. Preferred exemplary species of Aspergillusinclude A. niger and A. nidulans. Preferred exemplary species of Yarrowia include Y. lipolytica. Many preferred yeast species are available from the ATCC. For example, the following preferred yeast species are available from the ATCC and are useful in the expression of proteins: Saccharomyces cerevisiae, Hansen, teleomorph strain BY4743 yap3 mutant (ATCC Accession No. 4022731); Saccharomyces cerevisiae Hansen, teleomorph strain BY4743 hsp150 mutant (ATCC Accession No. 4021266); Saccharomyces cerevisiae Hansen, teleomorph strain BY4743 pmt1 mutant (ATCC Accession No. 4023792); Saccharomyces cerevisiae Hansen, teleomorph (ATCC Accession Nos. 20626; 44773; 44774; and 62995); Saccharomyces diastaticus Andrews et Gilliland ex van der Walt, teleomorph (ATCC Accession No. 62987); Kluyveromyces lactis (Dombrowski) van der Walt, teleomorph (ATCC Accession No. 76492); Pichia angusta (Teunisson et al.) Kurtzman, teleomorph deposited as Hansenula polymorpha de Morais et Maia, teleomorph (ATCC Accession No. 26012); Aspergillus niger van Tieghem, anamorph (ATCC Accession No. 9029); Aspergillus niger van Tieghem, anamorph (ATCC Accession No. 16404); Aspergillus nidulans (Eidam) Winter, anamorph (ATCC Accession No. 48756); and Yarrowia lipolytica (Wickerham et al.) van der Walt et von Arx, teleomorph (ATCC Accession No. 201847).

Suitable promoters for S. cerevisiae include those associated with the PGKI gene, GAL1 or GAL10 genes, CYCI, PH05, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOX1 and AOX2. Gleeson et al. (1986) J. Gen. Microbiol. 132, 3459-3465 include information on Hansenula vectors and transformation, suitable promoters being MOX1 and FMD1; whilst EP 361 991, Fleer et al. (1991) and other publications from Rhone-Poulenc Rorer teach how to express foreign proteins in Kluyveromyces spp., a suitable promoter being PGKI.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is preferred.

In certain embodiments, the desired antigen-binding construct protein is initially expressed with a secretion leader sequence, which may be any leader effective in the yeast chosen. Leaders useful in S. cerevisiae include that from the mating factor alpha polypeptide (MFα-1) and the hybrid leaders of EP-A-387 319. Such leaders (or signals) are cleaved by the yeast before the mature protein is released into the surrounding medium. Further such leaders include those of S. cerevisiae invertase (SUC2) disclosed in JP 62-096086 (granted as 911036516), acid phosphatase (PHOS), the pre-sequence of MFα-1, 0 glucanase (BGL2) and killer toxin; S. diastaticus glucoarnylase Il; S. carlsbergensis α-galactosidase (MEL1); K. lactis killer toxin; and Candida glucoamylase.

Provided are vectors containing polynucleotides encoding an antigen-binding construct described herein, host cells, and the production of the antigen-binding construct proteins by synthetic and recombinant techniques. The vector may be, for example, a phage, plasmid, viral, or retroviral vector. Retroviral vectors may be replication competent or replication defective. In the latter case, viral propagation generally will occur only in complementing host cells.

In certain embodiments, the polynucleotides encoding antigen-binding construct proteins described herein are joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In certain embodiments, the polynucleotide insert is operatively linked to an appropriate promoter, such as the phage lambda PL promoter, the E. coli lac, trp, phoA and rac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A; pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlsbad, Calif.). Other suitable vectors will be readily apparent to the skilled artisan.

In one embodiment, polynucleotides encoding an antigen-binding construct described herein are fused to signal sequences that will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the antigen-binding construct proteins are fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-.rho. series) available from New England Biolabs. In a specific embodiment, polynucleotides encoding proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that are fused to an antigen-binding construct protein in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA) (SEQ ID NO: 276), and a consensus signal sequence (MPTWAWWLFLVLLLALWAPARG) (SEQ ID NO: 277). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/10036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169(1992) and in Biblia and Robinson Biotechnol. Prog. 11:1(1995) which are herein incorporated by reference.

Also provided are host cells containing vector constructs described herein, and additionally host cells containing nucleotide sequences that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Cargo polypeptide is replaced with an antigen-binding construct protein corresponding to the Cargo polypeptide), and/or to include genetic material. The genetic material operably associated with the endogenous polynucleotide may activate, alter, and/or amplify endogenous polynucleotides.

In addition, techniques known in the art may be used to operably associate heterologous polynucleotides (e.g., polynucleotides encoding a protein, or a fragment or variant thereof) and/or heterologous control regions (e.g., promoter and/or enhancer) with endogenous polynucleotide sequences encoding a Therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Antigen-binding construct proteins described herein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography such as with protein A, hydroxylapatite chromatography, hydrophobic charge interaction chromatography and lectin chromatography. Most preferably, high performance liquid chromatography (“HPLC”) is employed for purification.

In certain embodiments the antigen-binding construct proteins of the invention are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antigen-binding construct proteins described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

### Post Translational Modifications:

In certain embodiments are antigen-binding constructs described herein, which are differentially modified during or after translation. In some embodiments, the modification is at least one of: glycosylation, acetylation, phosphorylation, amidation, derivatization by known protecting/blocking groups, proteolytic cleavage and linkage to an antibody molecule or other cellular ligand. In some embodiments, the antigen-binding construct is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional post-translational modifications of antigen-binding constructs described herein include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antigen-binding constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In specific embodiments, antigen-binding constructs described herein are attached to macrocyclic chelators that associate with radiometal ions.

In some embodiments, the antigen-binding constructs described herein are modified by either natural processes, such as post-translational processing, or by chemical modification techniques which are well known in the art. In certain embodiments, the same type of modification may be present in the same or varying degrees at several sites in a given polypeptide. In certain embodiments, polypeptides from antigen-binding constructs described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, antigen-binding constructs described herein are attached to solid supports, which are particularly useful for immunoassays or purification of polypeptides that are bound by, that bind to, or associate with proteins of the invention. Such solid supports include, but are not limited to, glass, cellulose, polyacrylamide, nylon, polystyrene, polyvinyl chloride or polypropylene.

### Assays:

The antigen-binding constructs described herein can be assayed for functional activity (e.g., biological activity) using or routinely modifying assays known in the art, as well as assays described herein.

For example, in one embodiment where one is assaying for the ability of an antigen-binding construct described herein to bind an antigen or to compete with another polypeptide for binding to an antigen, or bind to an Fc receptor and/or antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In certain embodiments, where a binding partner (e.g., a receptor or a ligand) is identified for an antigen binding domain comprised by an antigen-binding construct described herein, binding to that binding partner by an antigen-binding construct described herein is assayed, e.g., by means well-known in the art, such as, for example, reducing and non-reducing gel chromatography, protein affinity chromatography, and affinity blotting. See generally, Phizicky et al., Microbiol. Rev. 59:94-123 (1995). In another embodiment, the ability of physiological correlates of an antigen-binding constructs to bind to a substrate(s) of antigen binding polypeptide constructs of the antigen-binding constructs described herein can be routinely assayed using techniques known in the art.

### Pharmaceutical Compositions

Also and as described in more detail herein, included are compositions comprising the antigen binding construct and a carrier.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding constructs described herein are used to delay development of a disease or to slow the progression of a disease. The term “instructions” is used to refer to instructions customarily included in commercial packages of therapeutic products that contain information about the indications, usage, dosage, administration, combination therapy, contraindications and/or warnings concerning the use of such therapeutic products.

An “effective amount” of an agent such as an antigen-binding construct described herein, refers to the amount that is necessary to result in a physiological change in the cell or tissue to which it is administered.

A “therapeutically effective amount” of an agent, e.g. a pharmaceutical composition comprising an antigen-binding construct described herein, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an antigen-binding construct contained therein to be effective, and which contains no additional components which are unacceptably toxic to a subject to which the formulation would be administered.

### Therapeutic Uses:

In an aspect, antigen-binding constructs described herein are directed to antibody-based therapies which involve administering antigen-binding constructs described comprising cargo polypeptide(s) which is an antibody, a fragment or variant of an antibody, to a patient for treating one or more of the disclosed diseases, disorders, or conditions. Therapeutic compounds described herein include, but are not limited to, antigen-binding constructs described herein, nucleic acids encoding antigen-binding constructs described herein.

In certain embodiments is provided a method for the prevention, treatment or amelioration of at least one of: a proliferative disease, a minimal residual cancer, a tumorous disease, an inflammatory disease, an immunological disorder, an autoimmune disease, an infectious disease, viral disease, allergic reactions, parasitic reactions, graft-versus-host diseases or host-versus-graft diseases or cell malignancies, said method comprising administering to a subject in need of such a prevention, treatment or amelioration a pharmaceutical composition comprising an antigen-binding construct described herein.

In certain embodiments is a method of treating cancer in a mammal in need thereof, comprising administering to the mammal a composition comprising an effective amount of the pharmaceutical composition described herein, optionally in combination with other pharmaceutically active molecules. In certain embodiments, the cancer is a solid tumor. In some embodiments, the solid tumor is one or more of sarcoma, carcinoma, and lymphoma. In certain other embodiments, the cancer is a hematological cancer. In some embodiments, the cancer is one or more of B-cell lymphoma, non-Hodgkin's lymphoma, and leukemia.

Provided is a method of treating cancer cells comprising providing to said cell a composition comprising an antigen-binding construct described herein. In some embodiments, the method further comprising providing said antigen-binding construct in conjugation with another therapeutic agent.

Provided is a method of treating a cancer non-responsive to blinatumomab in a mammal in need thereof, comprising administering to the mammal a composition comprising an effective amount of the pharmaceutical composition comprising an antigen-binding construct described herein.

In some embodiments is a method of treating a cancer cell regressive after treatment with blinatumomab, comprising providing to said cancer cell a composition comprising an effective amount of the pharmaceutical composition comprising an antigen-binding construct described herein.

In some embodiments is a method of treating an individual suffering from a disease characterized by expression of B cells, said method comprising providing to said individual an effective amount of a composition comprising an effective amount of the pharmaceutical composition comprising an antigen-binding construct described herein. In some embodiments the disease is not responsive to treatment with at least one of an anti-CD19 antibody and an anti-CD20 antibody. In certain embodiments the disease is a cancer or autoimmune condition resistant to CD19 or CD20 lytic antibodies

Provided is a method of treating an autoimmune condition in a mammal in need thereof, comprising administering to said mammal a composition comprising an effective amount of the pharmaceutical composition described herein. In certain embodiments, the autoimmune condition is one or more of multiple sclerosis, rheumatoid arthritis, lupus erytematosus, psoriatic arthritis, psoriasis, vasculitis, uveitis, Crohn's disease, and type 1 diabetes.

Provided is a method of treating an inflammatory condition in a mammal in need thereof, comprising administering to said mammal a composition comprising an effective amount of the pharmaceutical composition comprising an antigen-binding construct described herein.

Armed with the teachings provided herein, one of ordinary skill in the art will know how to use the antigen-binding constructs described herein for diagnostic, monitoring or therapeutic purposes without undue experimentation.

The antigen-binding constructs described herein, comprising at least a fragment or variant of an antibody may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in an embodiment, human antibodies, fragments derivatives, analogs, or nucleic acids, are administered to a human patient for therapy or prophylaxis.

### Gene Therapy:

In a specific embodiment, nucleic acids comprising sequences encoding antigen-binding construct proteins described herein are administered to treat, inhibit or prevent a disease or disorder associated with aberrant expression and/or activity of a protein, by way of gene therapy. Gene therapy refers to therapy performed by the administration to a subject of an expressed or expressible nucleic acid. In this embodiment of the invention, the nucleic acids produce their encoded protein that mediates a therapeutic effect. Any of the methods for gene therapy available in the art can be used.

### Demonstration of Therapeutic or Prophylactic Activity:

The antigen-binding constructs or pharmaceutical compositions described herein are tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered an antigen-binding construct, and the effect of such antigen-binding construct upon the tissue sample is observed.

### Therapeutic/Prophylactic Administration and Composition:

Provided are methods of treatment, inhibition and prophylaxis by administration to a subject of an effective amount of an antigen-binding construct or pharmaceutical composition described herein. In an embodiment, the antigen-binding construct is substantially purified (e.g., substantially free from substances that limit its effect or produce undesired side-effects). In certain embodiments, the subject is an animal, including but not limited to animals such as cows, pigs, horses, chickens, cats, dogs, etc., and in certain embodiments, a mammal, and most preferably human.

Various delivery systems are known and can be used to administer an antigen-binding construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the antigen-binding construct compositions described herein into the central nervous system by any suitable route, including intraventricular and intrathecal injection; intraventricular injection may be facilitated by an intraventricular catheter, for example, attached to a reservoir, such as an Ommaya reservoir Pulmonary administration can also be employed, e.g., by use of an inhaler or nebulizer, and formulation with an aerosolizing agent.

In a specific embodiment, it is desirable to administer the antigen-binding constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the antigen-binding constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antigen-binding constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment comprising a nucleic acid encoding an antigen-binding construct described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

Also provided herein are pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In certain embodiments, the composition comprising the antigen-binding construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

The amount of the composition described herein which will be effective in the treatment, inhibition and prevention of a disease or disorder associated with aberrant expression and/or activity of a Therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

In certain embodiments, an antigen binding construct described herein is suitably administered to the patient at one time or over a series of treatments. Depending on the type and severity of the disease, about 1 μg/kg to 15 mg/kg (e.g. 0.1 mg/kg-10 mg/kg) of T cell activating bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen binding construct described herein would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram kg body weight to about 500 milligram kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the T cell activating bispecific antigen binding molecule). An initial higher loading dose, followed by one or more lower doses may be administered. However, other dosage regimens may be useful. The progress of this therapy is easily monitored by conventional techniques and assays.

The antigen-binding constructs described herein are generally used in an amount effective to achieve the intended purpose. For use to treat or prevent a disease condition, an antigen-binding construct described herein, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the antigen-binding construct described herein which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the antigen-binding construct described herein may not be related to plasma concentration. One having skill in the art will be able to optimize therapeutically effective local dosages without undue experimentation.

A therapeutically effective dose of the antigen-binding constructs described herein will generally provide therapeutic benefit without causing substantial toxicity. Toxicity and therapeutic efficacy of an antigen-binding construct described herein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. T cell activating bispecific antigen binding molecules that exhibit large therapeutic indices are preferred. In one embodiment, the antigen-binding construct described herein according to the present invention exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al, 1975, in: The Pharmacological Basis of Therapeutics, Ch. 1, p. 1, incorporated herein by reference in its entirety).

The attending physician for patients treated with antigen-binding constructs described herein would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Also provided is a process for the production of a pharmaceutical composition comprising an antigen binding construct described herein, said process comprising: culturing a host cell under conditions allowing the expression of an antigen-binding construct; recovering the produced antigen-binding construct from the culture; and producing the pharmaceutical composition.

### Other Agents and Treatments:

In certain embodiments, the antigen-binding constructs described herein are administered in combination with one or more other agents in therapy. For instance, in one embodiment, an antigen-binding construct described herein is co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangio genie agent.

Such other agents are suitably present in combination in amounts that are effective for the purpose intended. The effective amount of such other agents depends on the amount of T cell activating bispecific antigen binding molecule used, the type of disorder or treatment, and other factors discussed above. The antigen-binding constructs described herein are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the antigen-binding construct described herein can occur prior to, simultaneously, and/or following, administration of the additional therapeutic agent and/or adjuvant. Antigen-binding constructs described herein can also be used in combination with radiation therapy.

### Articles of Manufacture:

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating bispecific antigen binding molecule of the invention. The label or package insert indicates that the composition is used for treating the condition of choice. Moreover, the article of manufacture may comprise (a) a first container with a composition contained therein, wherein the composition comprises an antigen-binding construct described herein; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Description of Bi-Specific Anti-CD19-CD3 Antigen-Binding Constructs

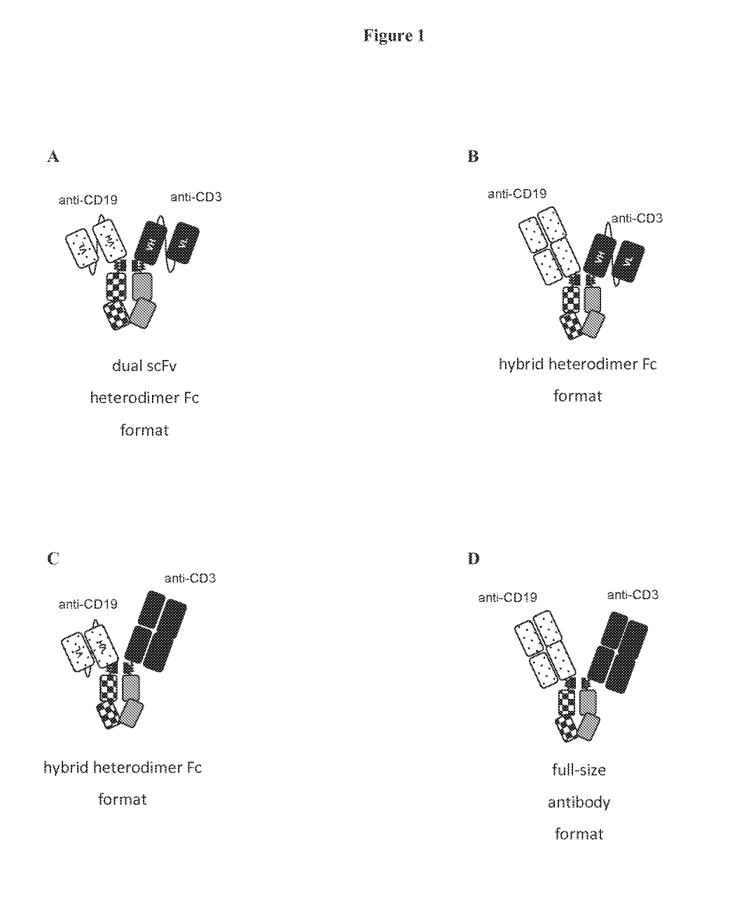

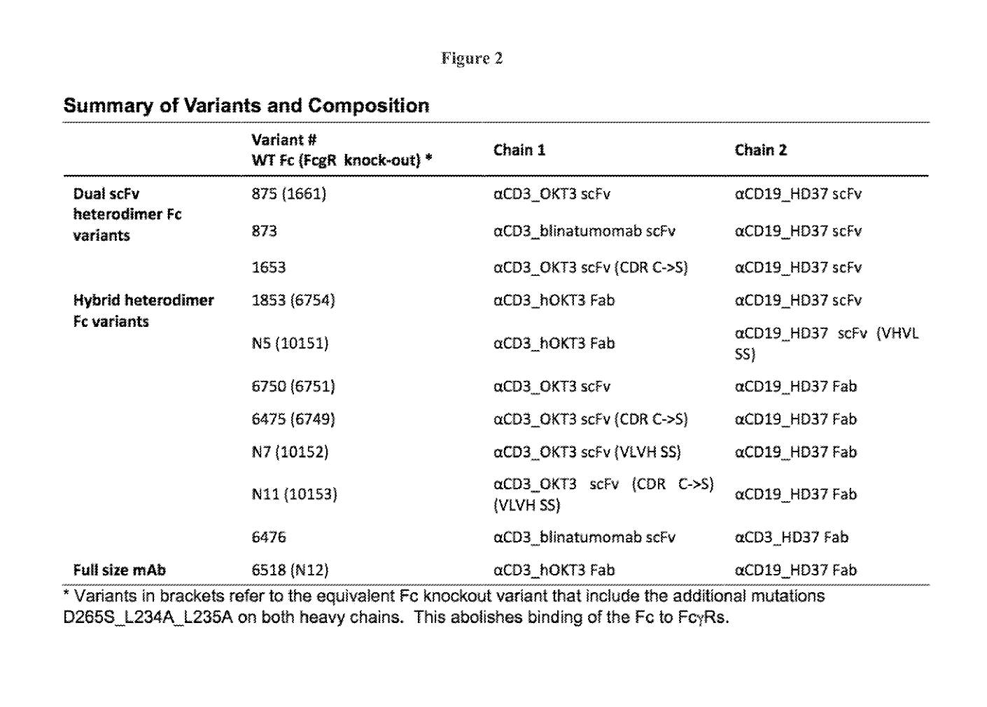

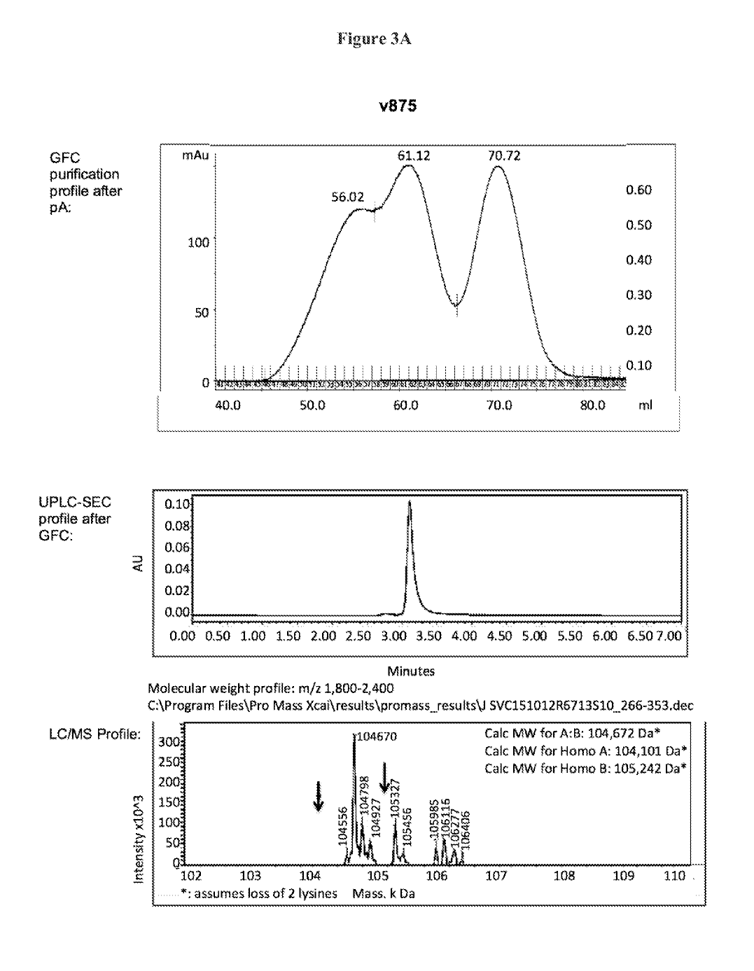

A number of exemplary bi-specific anti-CD3-CD19 antigen-binding constructs were designed as described below. An exemplary schematic representation of this type of constructs is shown in FIGS. 1A-C. A summary of these variants is shown in FIG. 2. All formats are based on the heterodimeric Fc constructed by known mutations in the CH3 domain (Von Kreudenstein et al., MAbs. 2013 5(5):646-54):
Dual scFv heterodimer Fc molecules contain the heterodimeric Fc with an anti-CD19 scFv and anti-CD3 scFv
Hybrid heterodimer Fc molecules contain the heterodimeric Fc with an anti-CD19 scFv and an anti-CD3 Fab or the heterodimeric Fc with an anti-CD19 Fab and an anti-CD3 scFv
Full size heterodimer Fc molecules contain the heterodimeric Fc with an anti-CD19 Fab and anti-CD3 Fab; the full size molecule can be constructed by a common light chain or and anti-CD19 light chain and anti-CD3 light chain.
   Dual scFv Heterodimer Fc Constructs:

v873 and v875 exemplify dual scFv heterodimer Fc bi-specific anti-CD3-CD19 antigen-binding constructs.

The anti-CD19 scFv (HD37 scFv) sequence of variants v873 and v875 was generated from the known anti-CD19 scFv (VL-VH) HD37 (Kipriyanov et. al., 1998, Int. J Cancer: 77, 763-772). The anti-CD3 scFv (OKT3 scFv) of variant v875 was generated by fusing the published OKT3 (Orthoclone OKT3, muronomab) variable light chain sequence to the variable heavy chain sequences with a (GGGGS)3 linker between the light and heavy chain. The anti-CD3 scFv (blinatumomab scFv) of variant v873 was generated from the known blinatumomab (Amgen) anti-CD3 scFv (VH-VL) sequence.

v873 has the anti-CD19-(HD37) scFv on chain A and the anti-CD3 (blinatumomab) scFv on chain B of the heterodimer Fc with the following mutations L351Y_F405A_Y407V on chain A and T366L_K392M_T394W on chain B.

V875 has the anti-CD19 (HD37) scFv on chain A and the anti-CD3 (OKT3) scFv on chain B of the heterodimer Fc with the following mutations L351Y_F405A_Y407V on chain A and T366L_K392M_T394W on chain B.

The following variant is an Fc knockout variant that includes the mutations D265S_L234A_L235A on both heavy chains. This set of mutations abolishes binding of the Fc to FcγRs. v1661 has the anti-CD19 BiTE™ (HD37) scFv on chain A and the anti-CD3 (OKT3) scFv on chain B of the heterodimer Fc with the following mutations D265S_L234A_L235A_T350V_L351Y_F405A_Y407V on chain A and D265S_L234A_L235A_T350V_T366L_K392L_T394W on chain B.

### Hybrid Heterodimer Fc and Engineered Constructs for Improved Biophysical Properties:

Additional bi-specific anti-CD3-CD19 antigen-binding constructs 1853, 6754, 10151, 6750, 6751, 6475, 6749, 10152, 10153, and 6518 were prepared. These constructs are based on the same antigen-binding domains as variant 875 but have been engineered for improved yield and biophysical properties. The modifications include changing one or both scFvs to the equivalent Fab format and/or stabilization of the scFv by VL-VH disulfide engineering and stabilizing CDR mutations.

The anti-CD19 scFv and anti-CD3 scFv sequences were generated as described above. The anti-CD19 Fab (HD37 Fab) is a chimeric Fab using the HD37 VH and VL sequences fused to human IgG1 CH and CL sequences respectively. The scFv or VH-CH domains are fused to one chain of the heterodimeric Fc. The anti-CD3 Fab (hOKT3 Fab) was generated from the known sequence of humanized OKT3 antibody teplizumab (Eli Lilly). The VH-CH domain was fused to one chain of the heterodimeric Fc.

The scFv disulfide engineering strategy (VHVL SS) for both the anti-CD3 and anti-CD19 scFvs utilized the published positions VH 44 and VL 100, according to the Kabat numbering system, to introduce a disulphide link between the VH and VL of the scFv [Reiter et al., Nat. Biotechnol. 14:1239-1245 (1996)].

The following variants contain a mutation to the anti-CD3 scFv to improve stability and yield, as reported previously [Kipriyanov et al., Prot. Eng. 10(4):445-453 (1997)]. v1653, v6475 and v10153 have an anti-CD3 (OKT3) with Cysteine to Serine mutation at position 100A of the VH CDR3.

Additional bi-specific anti-CD3-CD19 antigen-binding constructs were designed as described in Example 7. The clones that correspond to each bi-specific anti-CD3-CD19 antigen-binding construct are shown in Table XX, and the corresponding sequence composition of each clone is shown in Table YY.

### Benchmark Control

v891 has a polypeptide sequence that is identical to blinatumomab (BiTE™) and includes an anti-CD3 scFv and anti-CD19 scFv (50 kDa).

### Example 2

### Cloning, Expression and Purification of Exemplary Antigen-Binding Constructs

The variants (antigen-binding constructs) and controls described in Example 1 were cloned and expressed as follows. The genes encoding the antibody heavy and light chains were constructed via gene synthesis using codons optimized for human/mammalian expression. The scFv and Fab sequences were generated from known anti-CD19 antibody HD37 (HD37, Kipriyanov et. al., 1998, Int. J Cancer: 77, 763-772), and known anti-CD3 monoclonal antibodies OKT3 (ORTHOCLONE OKT3, Drug Bank reference: DB00075), Teplizumab (MGA031, Eli Lilly), blinatumomab (Amgen, US2011/0275787) sequences, and constructed as described in Example 1.

The final gene products were sub-cloned into the mammalian expression vector pTTS (NRC-BRI, Canada) and expressed in CHO cells (Durocher, Y., Perret, S. & Kamen, A. High-level and high-throughput recombinant protein production by transient transfection of suspension-growing CHO cells. Nucleic acids research 30, E9 (2002)).

The CHO cells were transfected in exponential growth phase (1.5 to 2 million cells/mL) with aqueous 1 mg/mL 25 kDa polyethylenimine (PEI, Polysciences) at a PEI:DNA ratio of 2.5:1. (Raymond C. et al. A simplified polyethylenimine-mediated transfection process for large-scale and high-throughput applications. Methods. 55(1):44-51 (2011)). In order to determine the optimal concentration range for forming heterodimers, the DNA was transfected in optimal DNA ratios of the heavy chain A (HC-A), light chain (LC), and heavy chain B that allow for heterodimer formation (e.g. HC-A/HC-B/ratios=50:50% (OAAs; HC/Fc), 50:50%. Transfected cells were harvested after 5-6 days with the culture medium collected after centrifugation at 4000 rpm and clarified using a 0.45 mm filter.

The clarified culture medium was loaded onto a MabSelect SuRe (GE Healthcare) protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. The protein was finally desalted using an Econo-Pac 10DG column (Bio-Rad).

In some cases, the protein was further purified by protein L chromatography by the method as follows. Capto L resin PBS was equilibrated with PBS and protein A purified v875, neutralized with 1 M Tris, was added to resin and incubated at RT for 30 min. Resin washed with PBS and flow through collected, bound protein was eluted with 0.5 ml 0.1 M Glycine, pH 3.

In some cases, the protein was further purified by gel filtration, 3.5 mg of the antibody mixture was concentrated to 1.5 mL and loaded onto a Superdex 200 HiLoad 16/600 200 pg column (GE Healthcare) via an AKTA Express FPLC at a flow-rate of 1 mL/min. PBS buffer at pH 7.4 was used at a flow-rate of 1 mL/min. Fractions corresponding to the purified antibody were collected, concentrated to −1 mg/mL and stored at −80° C.

All exemplary antigen-binding constructs were expressed transiently in CHO3E7 cells with a cell viability of >80%.

### Example 3

### Description, Expression and Purification of Exemplary Bi-Specific Antigen-Binding Constructs (Anti-CD3-CD19 or Anti-CD3-CD20) in a Hybrid Heterodimer Fc Format or in Full-Size Antibody Format

V5850, v5851, v5852, v6325, v1813, v1821, and v1823 exemplify bi-specific CD3/CD19 or CD3/CD20 hybrid antigen-binding constructs. These bi-specific hybrid variants are composed of a Fab on either chain A or B paired with an scFv-Fc on the alternate polypeptide chain. Chain A of the heterodimer Fc is comprised of the following mutations: T350V_L351Y_F405A_Y407V and Chain B of the heterodimer Fc is comprised of the following mutations: T350V_T366L_K392L_T394W. V1813, v1821, and v1823 exemplify CD3/CD20 common light chain antigen-binding constructs. Common light chain variants are composed of two different Fab)s, each on complimentary heterodimer Fc, which share a single light chain. The specific variant composition is indicated in Table 1.

With respect to the common light chain variants, combinations other than those shown in Table 1 were also prepared and tested.

TABLE 1
Composition of CD3/CD19 or CD20 hybrid variants

| | v5850 | V5851 | V5852 | V6325 |
|---|---|---|---|---|
| Format | Hybrid | Hybrid | Hybrid | Hybrid |
| Chain A | aCD3- | aCD3- | aCD3- | aCD3- |
| | BiTEx_I2C_scFvFc | BiTEx_I2C_scFvFc | Teplizumab- | Teplizumab- |
| | (VHVL) | (VHVL) | hOKT3_Fab | hOKT3_Fab |
| Chain B | aCD20- | aCD19- | aCD19- | aCD20- |
| | Ofatumumab_Fab | MOR208_Fab | MOR208_scFvFc_(VHVL) | Ofatumumab_scFvFc |
| | | | | VHVL) |
| Light | aCD20- | aCD19- | aCD3- | aCD3- |
| Chain | Ofatumumab_Fab | MOR208_Fab | Teplizumab- | Teplizumab- |
| | | | hOKT3 | hOKT3 |
| Reference | Chain A- | Chain A- | Chain A- | Chain A- |
| | US2011/0275787 | US2011/0275787 | US20070077246 | US20070077246 |
| | Chain B- | Chain B- | Chain B- | Chain B- |
| | WO2004035607 | WO2008022152 | Light | Light Chain- |
| | Light | Light Chain- | Chain- | US20070077246 |
| | Chain- | WO2008022152 | US20070077246 | |
| | WO2004035607 | | | |

| | v1813 | V1821 | V1823 |
|---|---|---|---|
| Format | Common | Common | Common |
| | light chain | light chain | light chain |
| Chain A | aCD3- | aCD3- | aCD3- |
| | foralumab_Fab | 12F6_Fab | 12F6_Fab |
| Chain B | aCD20- | aCD20- | aCD20- |
| | Ofatumumab_Fab | Rituximab_Fab | Tositumumab_Fab |
| Light | aCD20- | aCD20- | aCD20- |
| Chain | Ofatumumab_Fab | Rituximab_Fab | Tositumumab_Fab |
| Reference | Chain A- | Chain A- | Chain A- |
| | WHO | Pubmed ID: | Pubmed |
| | drug | 16313362 | ID: |
| | information | Chain B- | 16313362 |
| | Vol. 24, | Drug bank | Chain B- |
| | no2, 2010 | accession | Drug bank |
| | Chain B- | number: | accession |
| | WO2004035607 | DB00073 | number: |
| | Light | Light Chain- | DB00081 |
| | Chain- | Drug bank | Light Chain- |
| | WO2004035607 | accession | Drug bank |
| | | number: | accession |
| | | DB00073 | number: |
| | | | DB00081 |

The anti-CD19 MOR208_scFv-Fc(VHVL) used in v5852 was generated by fusing the published variable heavy chain sequence to the variable light chain sequences indicated in Table 1 with a (GGGGS)3 (SEQ ID NO: 380) linker between the heavy and light chain. The variable domains were fused to Chain B of the heterodimer Fc.

The anti-CD20 Ofatumumab_scFv-Fc(VHVL) used in v6325 was generated by fusing the published variable heavy chain sequence to the variable light chain sequences indicated in Table 1 with a (GGGGS)3 (SEQ ID NO: 380) linker between the heavy and light chain. The variable domains were fused to Chain B of the heterodimer Fc.

Cloning, expression and purification was performed as indicated in Example 2.

Yield and purity of the variants is indicated in Table 2 below. Heterodimer purity was determined by LCMS analysis as described below. The purity of exemplary antigen-binding constructs was tested by LC-MS. The antigen-binding constructs were first purified by protein A, protein L and SEC purification as described in Example 2. LC-MS analysis for heterodimer purity was performed as described below.

The purified samples were de-glycosylated with PNGase F for 6 hr at 370 C. Prior to MS analysis the samples were injected onto a Poros R2 column and eluted in a gradient with 20-90% ACN, 0.1% FA in 3 minutes, resulting in one single peak.

The peak of the LC column was analyzed with a LTQ-Orbitrap XL mass spectrometer using the following setup: Cone Voltage: 50 V′ Tube lens: 215 V; FT Resolution: 7,500. The mass spectrum was integrated with the software Promass or Max Ent. to generate molecular weight profiles

Hybrid heterodimer Fc constructs and full size mAb variants show comparable expression and purification yield. All variants demonstrated heterodimer purity in excess of 73.8% with an average purity of 89.6% for all variants tested. The samples had low amounts of incorrectly paired homodimers ranging from 0 to 5.3% of the total product. Reported values represent the sum of all observed homodimer species. The presence of half-antibodies was more commonly observed than homodimers and ranged from 0 to 20.7% of the total product. Reported values represent the sum of all observed half-antibody species.

TABLE 2
Variant expression and purity

| | Format | | | | | | |
|---|---|---|---|---|---|---|---|
| | | | | | full size mAb | | |
| | Hybrid | | | | (common light chain) | | |
| | Target | | | | | | |
| | CD20/ | CD19/ | CD19/ | CD20/ | CD20/ | CD20/ | CD20/ |
| | CD3 | CD3 | CD3 | CD3 | CD3 | CD3 | CD3 |
| | V5850 | V5851 | V5852 | V6325 | v1813 | V1821 | V1823 |
| Expression | 50 | 50 | 50 | 50 | 500 | 500 | 500 |
| scale (ml) | | | | | | | |
| Amount | 1.25 | 0.72 | 0.57 | 0.42 | 17.4 | 2.16 | 8.8 |
| after SEC | | | | | | | |
| (mg) | | | | | | | |
| % | 95.6 | 100 | 95.1 | 97.5 | 78.4 | 91.4 | 73.8 |
| Heterodimer | | | | | | | |
| (AB) | | | | | | | |
| % | 0 | 0 | 4.9 | 0 | 1.36 | 3.7 | 5.3 |
| Homodimer | | | | | | | |
| (AA + BB) | | | | | | | |
| % half- | 4.4 | 0 | 0 | 2.5 | 20.2 | 4.8 | 20.7 |
| antibody | | | | | | | |
| (A + B) | | | | | | | |

### Example 4

### Bi-Specific Antigen-Binding Constructs Bind to T Cells and B Cells

The ability of the exemplary CD3/CD20 bi-specific antigen-binding constructs v5850, v6325, v1813, v1821, v1823 to bind to CD3- and CD20-expressing cells were assessed via FACS analysis as described below. Additionally, the ability of exemplary bi-specific anti-CD3-CD19 antigen-binding constructs v5851 and v5852 to bind to CD3- and CD19-expressing cells were similarly assessed. The variant v875, an anti-CD3-CD19 BiTE Fc antibody construct in the dual scFv format, was also tested as a benchmark. In variants belonging to both bispecific families, binding affinity to the target B cell is higher than the effector T cell as designed.

### Whole Cell Binding by FACS Protocol:

2×10⁶ cells/ml cells (>80% viability) were resuspended in L10+GS1 media, mixed with antibody dilutions, and incubated on ice for 1 h.

Cells were washed by adding 10 ml of cold R-2 buffer, and centrifuging at 233 g for 10 min at 4° C. The cell pellet was resuspended with 100 μl (1/100 dilution in L10+GS1 media) of fluorescently labeled anti-mouse or anti-human IgG and incubated for 1 hour at RT.

Cell treatments were washed by adding 10 ml of cold R-2 as previously described, and the cell pellet resuspended with 400 μl of cold L-2 and the sample was filtered through Nitex and added to a tube containing 4 μl of propidium iodide.

Samples were analyzed by flow cytometry.

The binding results for each variant expressed in kinetic constants Bmax and Kd are listed below in Tables 4 and 5. Table 4 describes the binding to the CD19- and CD20-expressing Raji B cells, while Table 5 describes binding to the CD3-expressing Jurkat T cells. In Raji binding studies (Table 4) CD19-CD3 bispecific dual scFv heterodimer Fc and hybrid heterodimer Fc variants bound target B cells with low nM apparent affinity and comparable Bmax. Anti CD20-CD3 bispecific hybrid heterodimer Fc and full size common light chain variants bound target B cells with comparable Bmax and 2 out of the 3 common light chain variants showed low nM binding affinity to target B cells.

In Jurkat binding studies (Table 5) CD19-CD3 bispecific dual scFv heterodimer Fc and hybrid heterodimer Fc variants bound T cells with nM affinity and comparable Bmax. Anti CD20-CD3 bispecific hybrid heterodimer Fc and full size common light chain variants bound T cells with comparable Bmax and 1 out of the 3 common light chain variants showed nM binding affinity to T cells.

All bispecific anti-CD19-CD3 constructs bind to CD19 B cells with high affinity and with lower affinity to CD3 T cells, as anticipated. Dual scFv heterodimer Fc constructs and hybrid heterodimer Fc constructs showed comparable binding affinities.

Although several other the common light chain anti-CD20-CD3 full size constructs were tested (data not shown), only variants 1813, 1821, and 1823 showed good binding to both the target CD20 B cells and the CD3 T cells.

TABLE 4
(Raji)

| | Format | | | | | | | | |
|---|---|---|---|---|---|---|---|---|---|
| | | | | | | | Full size mAb | | |
| | Dual scFv | | hybrid | | | | (common light chain) | | |
| | Target | | | | | | | | |
| | CD19/ | CD19/ | CD20/ | CD19/ | CD19/ | CD20/ | CD20/ | CD20/ | CD20/ |
| | CD3 | CD3 | CD3 | CD3 | CD3 | CD3 | CD3 | CD3 | CD3 |
| | Variant | | | | | | | | |
| | v875 | v4542 | v5850 | v5851 | v5852 | v6325 | v1813 | v1821 | v1823 |
| Bmax | 2.78 | 2.96 | 4.24 | 3.88 | na | 6.44 | 6.40 | 4.71 | 4.14 |
| (OD450) | | | | | | | | | |
| KD | 0.36 | 0.70 | 3.60 | 1.38 | na | 11.87 | 4.04 | 122.5 | 21.05 |
| (nM) | | | | | | | | | |

TABLE 5
(Jurkat)

| | | | | | | | Full size mAb | | |
|---|---|---|---|---|---|---|---|---|---|
| | Dual scFv | | | Hybrid | | | (common light chain) | | |
| | v875 | v4542 | v5850 | v5851 | v5852 | v6325 | v1813 | v1821 | v1823 |
| | Target | | | | | | | | |
| | CD19/ | CD19/ | CD20/ | CD19/ | CD19/ | CD20/ | CD20/ | CD20/ | CD20/ |
| | CD3 | CD3 | CD3 | CD3 | CD3 | CD3 | CD3 | CD3 | CD3 |
| Bmax | 1.59 | 2.27 | 2.06 | 2.51 | 2.21 | 2.51 | 2.54 | 2.11 | 0.88 |
| (OD450) | | | | | | | | | |
| KD (nM) | 21.36 | 6.66 | 4.04 | 4.24 | 25.24 | 1.58 | 691.4 | 181.5 | 68.77 |

### Example 5

### Bi-Specific Anti-CD3-CD19 Antigen-Binding Constructs and Bi-Specific Anti-CD3-CD20 Antigen-Binding Constructs Bridge T Cells and B Cells

The ability of five exemplary anti-CD3-CD20 antigen-binding constructs—namely v5850, v6325, v1813, v1821 and v1823—and two exemplary anti-CD3-CD19 antigen-binding constructs—namely v5851 and v5852—to bridge T cells and B cells were tested via FACS analysis as per procedures described below. Additional constructs, namely v792 and v875, were also tested as controls. V792 is a bivalent anti HER2 antibody with identical anti-Her2 F(ab′) based on trastuzumab on chain A and chain B of the heterodimer Fc with the following mutations T350V_L351Y_F405A_Y407V on chain A and T350V_T366L_K392L_T394W on chain B (drug bank accession number—DB00072)

### Whole Cell Bridging by FACS

1×10⁶ cells/ml suspended in RPMI were labeled with 0.3 μM of the appropriate CellTrace label and mixed and incubated at 37° C. in a water bath for 25 minutes

Pellets were resuspended in 2 ml of L10+GS1+NaN3 to a final concentration 5×106 cells/ml.

Cell suspensions were analyzed (1/5 dilution) by flow cytometry to verify the appropriate cell labeling and laser settings. Flow-check and flow-set Fluorospheres were used to verify instrument standardization, optical alignment and fluidics.

After flow cytometry verification, and prior to bridging, each cell line was mixed together at the desired ratio, at a final concentration of 1×10⁶ cells/ml.

T:T bridging was assessed with Jurkat-violet+Jurkat-FarRed, B:B was assessed with RAJI-violet+ RAJI-FarRed and T:B bridging was assessed with Jurkat-violet+RAJI-FarRed.

Antibodies were diluted to 2× in L10+GS1+NaN3 at room temperature then added to cells followed by gentle mixing and a 30 min incubation.

Following the 30 min incubation 2 μl of propidium iodide was added and slowly mixed and immediately analyze by flow cytometry.

Bridging % was calculated as the percentage of events that are simultaneously labeled violet and Far-red.

Tables 6 and 7 provides the percentage bridging between Jurkat-Jurkat, Raji-Raji, and Jurkat-Raji for each variant, each table represents an individual experiment. All variants, belonging to dual scFv, hybrid and full size (common light chain) heterodimer Fc format with a T and B cell binding paratope were effective at bridging Jurkat and Raji cells. Furthermore, none of the variants bridged two Jurkat cells and some Raji-Raji cell bridging was observed to different extents. The negative control v792 showed no specific (background) T-B, B:B, T:T bridging.

Analysis shows that despite the difference in geometry and spatial distance of the binding domains, all formats, dual scFv heterodimer Fc, hybrid heterodimer Fc and also full size antibody format are able to effectively bridge T and B cells. Further, both CD19 and CD20 can be targeted to induce T:B cell bridging.

TABLE 6
Whole cell FACS B:T cell bridging analysis

| Format | | |
|---|---|---|
| Dual | | Full size mAb |
| scFv | Hybrid | (common light chain) |

| | Variant | | | | | | |
|---|---|---|---|---|---|---|---|
| | v792 | v875 | v5850 | v5851 | v1813 | v1821 | v1823 |
| | Target | | | | | | |
| | neg. | CD19/ | CD20/ | CD19/ | CD20/ | CD20/ | CD20/ |
| % Bridging | control | CD3 | CD3 | CD3 | CD3 | CD3 | CD3 |
| Jurkat/Jurkat | 0.5 | 1.6 | 0.8 | 1.0 | 0.6 | 0.5 | 0.7 |
| Raji/Raji | 2.6 | 10.2 | 2.1 | 1.6 | 7.0 | 2.4 | 2.1 |
| Jurkat/Raji | 2.6 | 17.0 | 11.6 | 23.2 | 16.2 | 7.3 | 8.1 |

TABLE 7
Whole cell FACS B: T cell bridging analysis

| | | Dual scFv | Hybrid | |
|---|---|---|---|---|
| Variant | v792 | v875 | v5852 | v6325 |
| Target | neg. | CD19/ | CD19/ | CD20/ |
| | control | CD3 | CD3 | CD3 |
| % Bridging | | | | |
| Jurkat/Jurkat | 0.7 | 0.5 | 0.9 | 1.1 |
| Raji/Raji | 0.7 | 8.6 | 7.2 | 0.7 |
| Jurkat/Raji | 1.9 | 15.7 | 30.4 | 15.7 |

### Example 6

### Expression, Purification and Biophysical Characterization of Bi-Specific Anti-CD3-CD19 Antigen-Binding Constructs for Improved Biophysical Properties

The antigen-binding constructs described in Example 1 were cloned, expressed and purified as described in Example 2 and the purity and yield of the final product was estimated by LC/MS and UPLC-SEC as described in Example 3. Whole cell saturable binding to CD19+ target Raji B cells and to CD3+ Jurkat T cells was measured as described in Example 4.

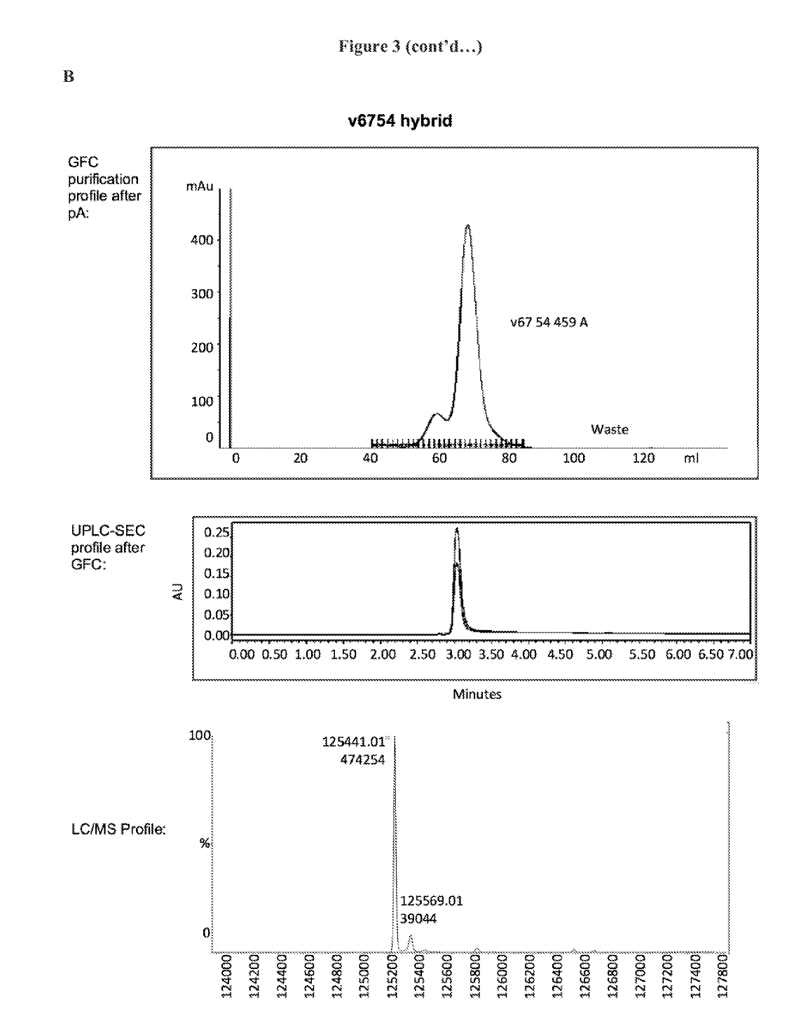

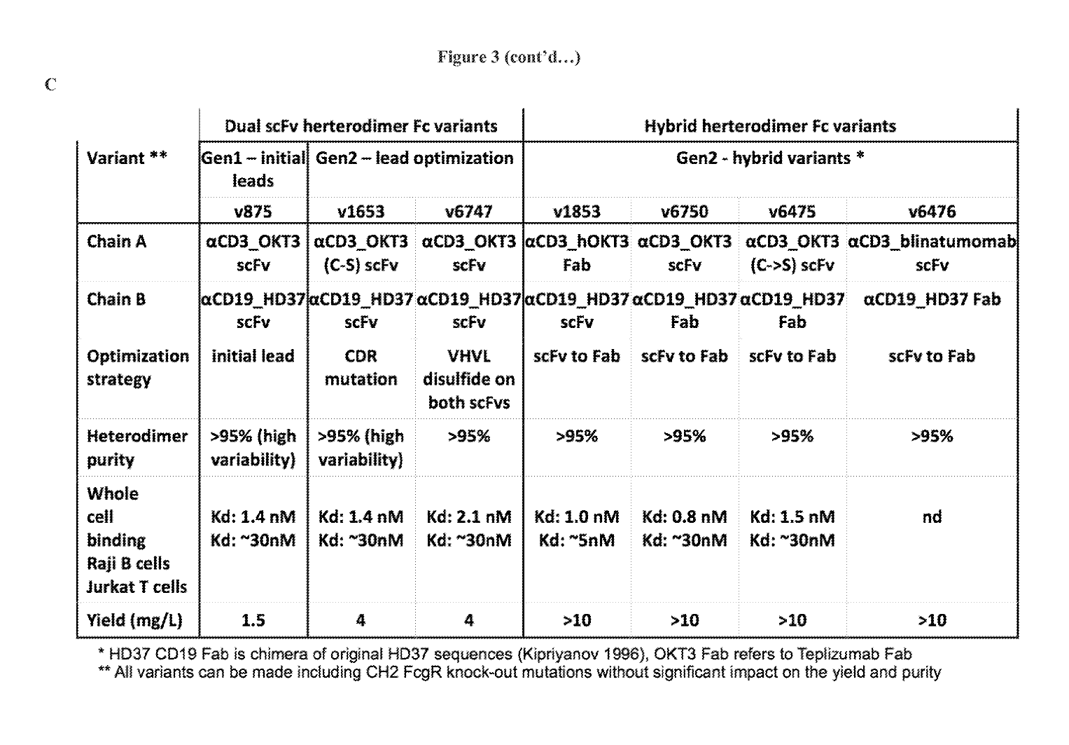

The results for purification of v875 and v6754 are shown in FIGS. 3A and 3B. The dual scFv heterodimer Fc variant v875 shows significant amounts of high molecular weight aggregates after protein A purification, whereas the hybrid heterodimer Fc variant v6754 shows one main peak similar to what is observed for standard therapeutic monoclonal antibodies. Both the dual scFv heterodimer Fc variant and the hybrid heterodimer Fc variant were purified to >98% homogeneity, as confirmed by LC/MS and HPLC-SEC.

FIG. 3C illustrates the improved yield of the optimized variants and the corresponding optimization strategy. Specifically, hybrid variants showed overall improvement in yield and heterodimer purity compared to v875.

It is contemplated that variants can be further improved for manufacturability by VHVL disulfide stabilization and adding stabilizing CDR mutations to the scFv as described in Example 1. Variable domain disulfide engineering is known to be highly dependent on the specific variable and light chain and the VH-VL interface. It is not applicable to all scFv and can lead to significantly reduced yields and/or loss of antigen binding [Miller et al., Protein Eng Des Sel. 2010 July; 23(7):549-57; Igawa et al., MAbs. 2011 May-June; 3(3):243-5; Perchiacca & Tessier, Annu Rev Chem Biomol Eng. 2012; 3:263-86.]. Variant v6747 is the equivalent variant to v875, with both scFvs stabilized by VL-VH disulfide as described in Example 1. FIG. 3C shows higher yield for the disulfide stabilized variant v6747 compared to v875 and no loss in apparent binding affinity. These experiments demonstrate that both the anti-CD19 and the anti-CD3 scFv can be stabilized by disulfide engineering with increase in yield and no loss in binding affinity.

### Example 7

### Binding of Bi-Specific Antigen-Binding Constructs to Raji and Jurkat Cells

The ability of the bi-specific antigen-binding constructs 1853, 6754, 6750, and 6751, described in FIG. 2 to bind to CD19- and CD3-expressing cells was assessed by FACS as described in Example 4. The binding properties of v875 and v1661, variants described in Example 1, were used as comparators.

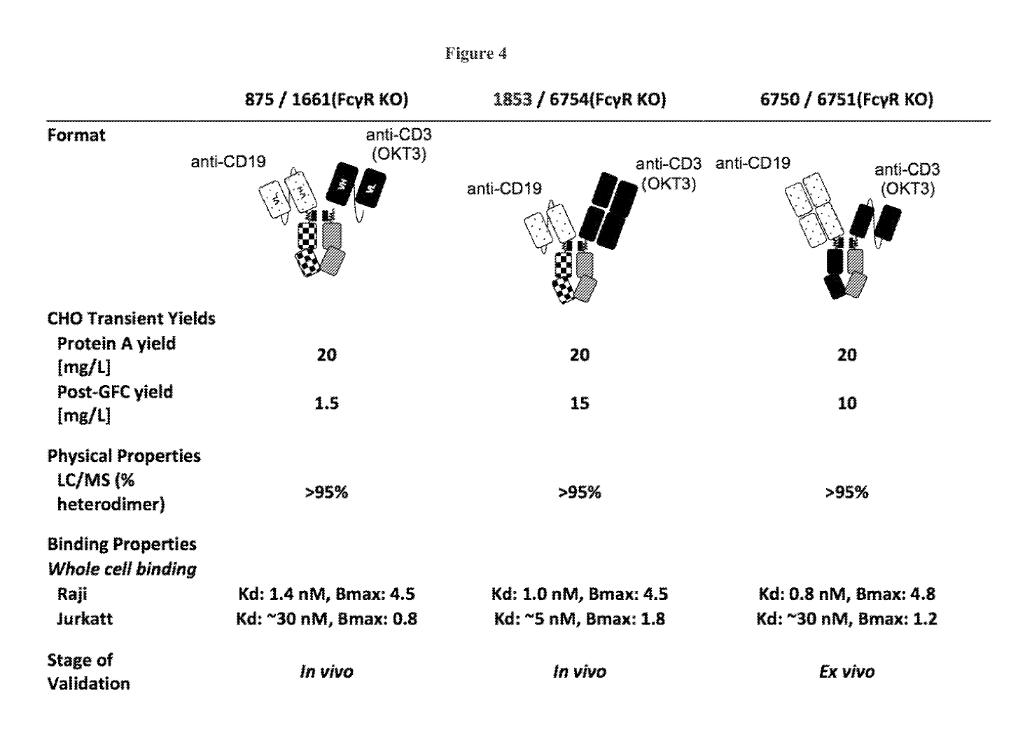

FIG. 4 provides a summary of the results. All variants, including dual scFv heterodimer Fc and hybrid heterodimer Fc variants bind CD19 Raji B cells with low nM affinity and CD3 T cells with lower apparent affinity of 5-30 nM. Example 9: Analysis of T:B-cell bridging of bi-specific antigen-binding constructs by FACS.

The ability of the improved bi-specific anti-CD3-CD19 antigen-binding constructs to bridge and cluster T cells and B cells was tested by FACS analysis as follows.

Briefly, 1×10⁶ cells/ml suspended in RPMI were labeled with 0.3 μM of the appropriate CellTrace label and mixed and incubated at 37° C. in a water bath for 25 minutes.

The Jurkat or RAJI cells were prepared as follows. Cell cultures were grown to exponential phase and then centrifuged. Cell pellets were resuspended in 2 ml of L10+GS1+NaN3 to a final concentration 5×10⁶ cells/mL. Cell suspensions were analyzed (1/5 dilution) by flow cytometry to verify the appropriate cell labeling and laser settings. Flow-check and flow-set Fluorospheres were used to verify instrument standardization, optical alignment and fluidics. After flow cytometry verification, and prior to bridging, each cell line was mixed together at the desired ratio, at a final concentration of 1×10⁶ cells/ml.

T:T bridging was assessed with Jurkat-violet+Jurkat-FarRed, B:B was assessed with RAJI-violet+ RAJI-FarRed and T:B bridging was assessed with Jurkat-violet+RAJI-FarRed. Test antibodies were diluted to 2× in L10+GS1+NaN3 at room temperature then added to cells followed by gentle mixing and a 30 min incubation. Following the 30 min incubation 2 μl of propidium iodide was added and slowly mixed and immediately analyze by flow cytometry. Bridging % was calculated as the percentage of events that are simultaneously labeled violet and Far-red.

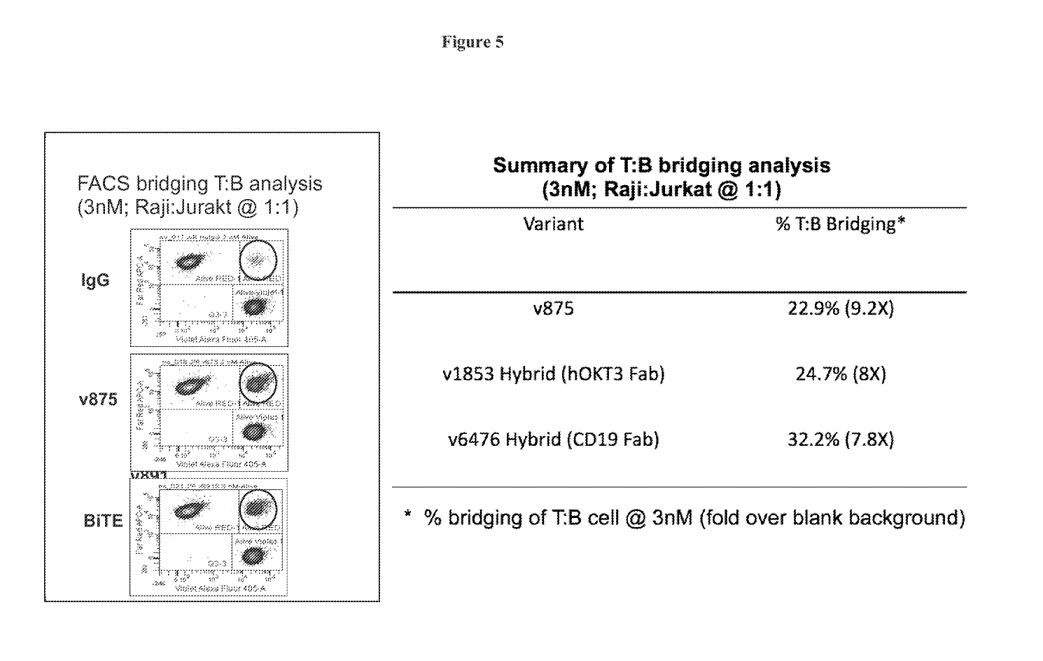

FIG. 5 summarized the % T:B bridging for the hybrid variants tested. These results indicate that both hybrid heterodimer Fc variants 1853 and v6476 were able to bridge CD19+ RAJI cells and CD3+Jurkat cells (Table on right) comparable to the dual scFv heterodimer Fc variant v875. The panel on the left in FIG. 5 shows the bridging results for the variants 875 (dual scFv) and 891 (scFv) for reference, in CD19+ RAJI cells and CD3+ Jurkat cells.

### Example 8

### Analysis of T:B Cell Synapse (T Cell Pseudopodia) Formation by Microscopy

The ability of exemplary variants to mediate the formation of T cell synapses and pseudopodia was assessed as follows. The variants tested in this assay included 875, 1661, 1853, and 6476. The variant 6518, which is a full-size CD3/CD19 bi-specific antibody (both the CD3 and CD19 antigen binding domains are in the Fab format) was also tested.

Labeled Raji B cells (red) and labeled Jurkat T cells (blue) were incubated for 30 min at room temperature with 3 nM of human IgG or v875. The cell suspension was concentrated by centrifugation, followed by removal of 180 μl of supernatant. Cell were resuspended in the remaining volume and imaged at 200× and 400×.

Microscopy images (200×) were acquired, pseudo colored, overlaid and converted to TIFF using Openlab software. The cells were then counted using the cell counter in Image J software and binned into 5 different populations:
1. T alone (also include T:T)
2. T associated with B (no pseudopodia)
3. T associated with B (with pseudopodia, i.e. T-cells that showed a crescent-like structure)
4. B alone (also include B:B)
5. B associated with T

For some cells, a review of original and phase images in Openlab software was necessary for proper binning. Then, % of total T-cell associated with B-cells, % of total T-cell associated with B-cells that have filopodia, % of T-cell associated with B-cells that have filopodia, % of B-cells associated with T-cells and overall B:T (%) could be determined.

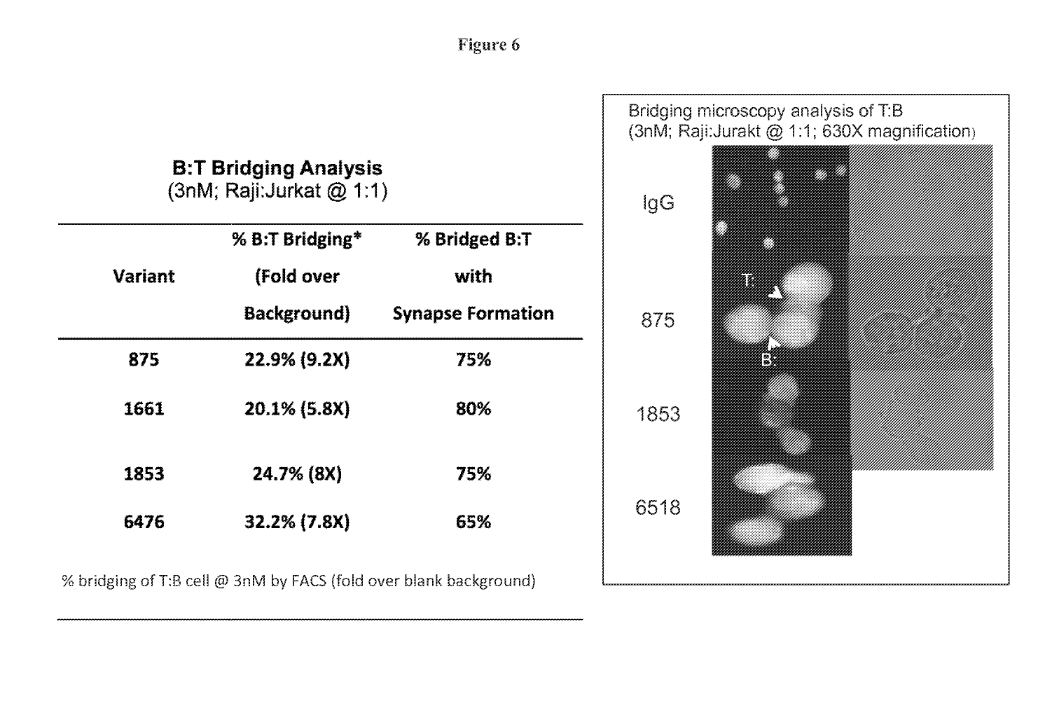

The results are shown in FIG. 6 and demonstrate that hybrid heterodimer Fc variants (1853 and 6476), full size bi-specific (6518), and dual scFv heterodimer Fc (875 and 1661) formats can also bridge CD19⁺ Raji B cells and Jurkat T cells with the formation of T:B cell synapses (T cell pseudopodia), as quantified by whole cell FACS analysis of synapses to be 5-8 fold over background and as shown by phase contrast microscopy and specific synapse formation between T:B and not B:B cells.

The analysis shows that despite the difference in geometry and spatial distance of the binding domains, dual scFv heterodimer Fc and hybrid heterodimer Fc and also full size antibody format are able to effectively bridge T and B cells and mediate T cell synapse and pseudopodia formation, as indication of T cell mediated target cell lysis.

### Example 9

### Autologous B Cell Depletion in Human Whole Blood

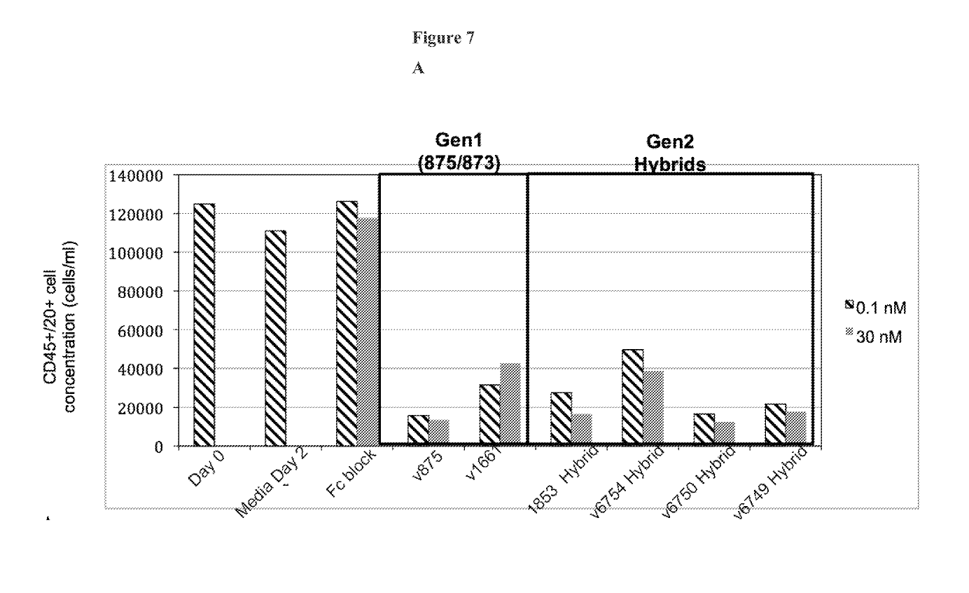

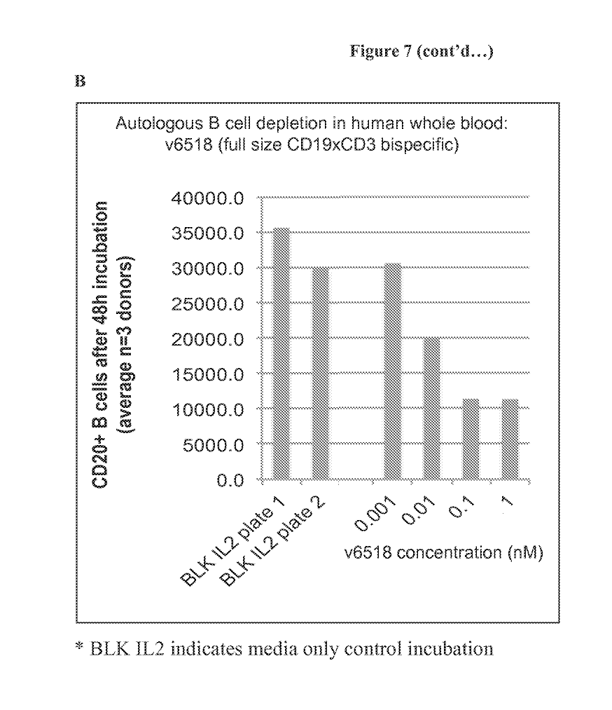

Bi-specific CD19-CD3 variants were analyzed for their ability to deplete autologous B cells in human whole blood primary cell culture under IL2 activation. The variants tested in this assay were the dual scFv heterodimer Fc variants 875 and 1661, as well as the hybrid heterodimer Fc variants 1853, 6754, 6750, and 6749 (FIG. 7A). The full size bispecific antibody v6518 was also tested in this assay in a separate experiment (FIG. 7B). As a nonspecific control, termed Fc block in FIG. 7A, a homodimeric Fc without Fab binding arms was used.

Briefly, variants were incubated in heparinized human whole blood in the presence of IL2 for 2 days. Quadruplicate wells were plated for each control and experimental condition and cultures are incubated in 5% CO2, 37° C. and stopped at 48 hours. The red blood cells were lysed after harvesting of the cultures and the collected primary cells were stained for CD45, CD20 and 7-AAD FACS detection. FACS analysis of the CD45+, CD45+/CD20+ and CD45+/CD20+/7AAD+/− populations was carried out by InCyte/FlowJo as follows: Between 5,000 event for FSC/SSC and compensation wells, and 30,000 events for experimental wells were analyzed by cytometry. A threshold was set to skip debris and RBCs. Gating was performed on lymphocytes, CD45+, CD20+, and 7AAD+ cells.

FIGS. 7A and 7B show the cytotoxic effect of the bi-specific anti-CD19-CD3 antigen-binding constructs on the autologous B cell concentration in human whole blood following IL2 incubation All of the variants were able to maximally deplete CD20+ B cells in this assay at the 0.1 nM

The analysis shows that despite the difference in geometry and spatial distance of the binding domains, dual scFv, heterodimer Fc, and hybrid heterodimer Fc variant and also full size antibody format can efficaciously deplete B cells in human primary blood culture.

### Example 10

### In Vivo Efficacy of CD19-CD3 Heterodimer Variants in NSG Mice Engrafted with IL2 Activated Human PBMC and G2 Leukemia Cells

The efficacy of selected variants in an in vivo mouse leukemia model was determined. In this model, NSG (NOD scid gamma) mice were engrafted with IL2 activated human PBMC and G2 leukemia cells.

As a preliminary experiment the ability of selected variants to bind to the G2 leukemia cell line was tested.

### In Vitro FACS Binding to Human G2 ALL Tumor Cell Line:

Pre-chilled G2 cells (1×10⁶ viable cells/tube) were incubated in triplicate on ice for 2 h in the absence of CO₂ with ice cold bi-specific reagent huCD3×huCD19 at concentrations of 0, 0.1, 0.3, 1, 3, 10, 30, and 100 nM in Leibovitz L15 buffer containing 10% heat inactivated fetal bovine serum and 1% goat serum (L-10+GS1) in a final volume of 200 ul/tube. After the incubation, cells were washed in 4 ml ice cold Leibovitz L15, and the pellet resuspended in 100 ul ice cold Alexa fluor 488-tagged anti-human antibody (Jackson Immunoresearch) diluted 1/100 in L-10+GS1. After >15 min in the dark, 4 ml Leibovitz L15 was added, cells were pelleted, and then resuspended in 200 ul ice cold flow cytometry running buffer containing 2 ug/ml 7AAD before analysis by flow cytometry. Mean fluorescence intensity was used to establish binding curves from which the Kd was determined for each bi-specific reagent for each cell line.

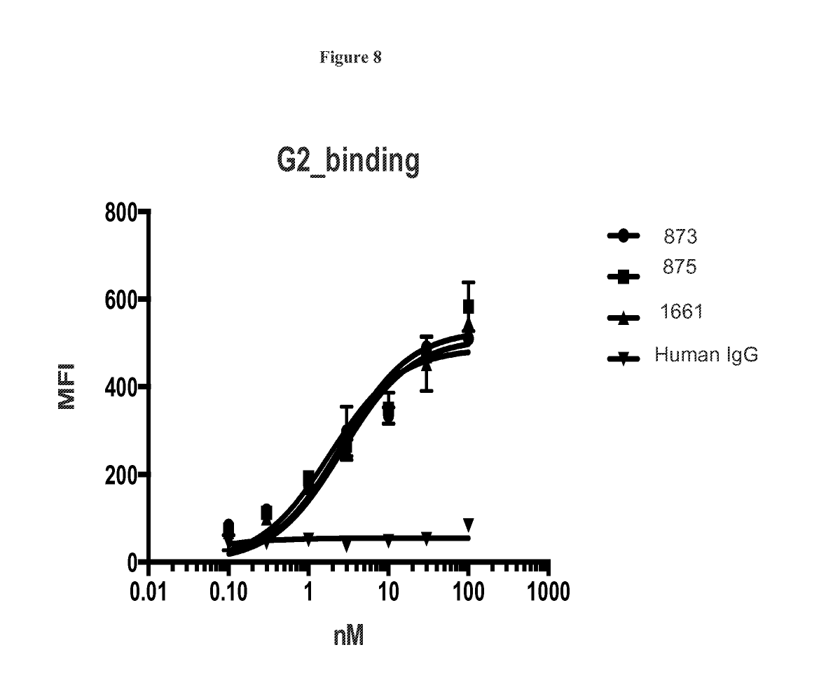

FIG. 8 shows that the exemplary variants 873, 875, and 1661 were able to bind to G2 ALL cells.

In vivo efficacy in NSG mice engrafted with IL2 activated human PBMC and G2 leukemia cells:

NOD/SCID/_{c}^{null} (NSG) mice (n=5/group) were implanted intravenously with 1×10⁵ G2-CBRluc/eGFP cells mixed with 3×10⁶ activated (anti-CD3/antiCD28 s [1 bead/CD3+ cell]+50 U IL2/ml for 5 d) human PBMC using a single donor as the source of cells for all groups of mice. Flow cytometry was used to assess the activation state (CD3, CD4, CD8, CD25, CD69, CD45RO, CD62L, and CCR7) and viability (7AAD) of the T cells.

1 h after PBMC and G2 engraftment the mice received the first dose (n-5/group) of the bi-specific variants with dosing at 3 mg/kg on day 0, 2, and 4, ending at Day 5. Tumor progression was followed by injecting mice with D-luciferin (150 micrograms/g body weight) followed by whole body bioluminescence imaging (BLI) 10 min later at baseline and on days 9, 14 and 18 post-implant. On day 18 animals were terminated and the spleen harvested for ex vivo BLI (bioluminescence imaging).

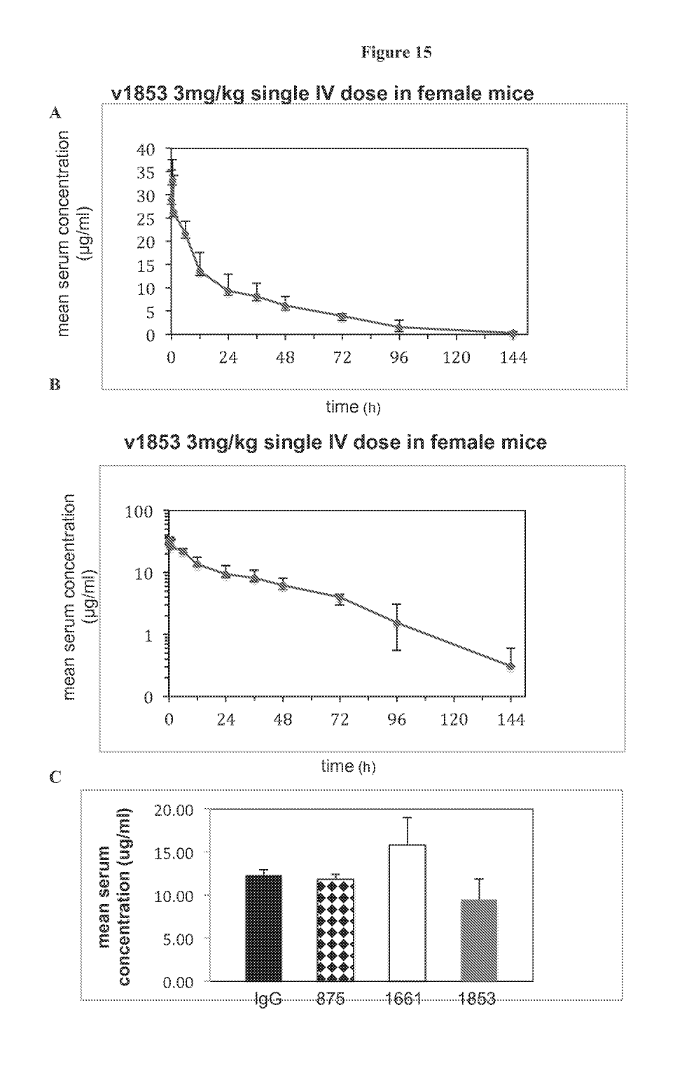

In addition serum samples were collected for 2 animals per cohort at 24 h after the first 3 mg/kg IV dose. The serum samples were analysed as described in Example 17 and the 24 h serum concentrations are shown in FIG. 15. The results are shown in FIG. 15C, confirming the IgG1-like PK of the CD3-CD19 bi-specific variants tested.

FIG. 9 shows the effect of the dual scFv heterodimer Fc FcgR knock-out variant 1661 on the G2 leukemia cell engraftment in whole body and the isolated spleen. The V1661 shows complete depletion of the G2 ALL cells and no significant G2 engraftment in major organs and tissue affected in ALL.

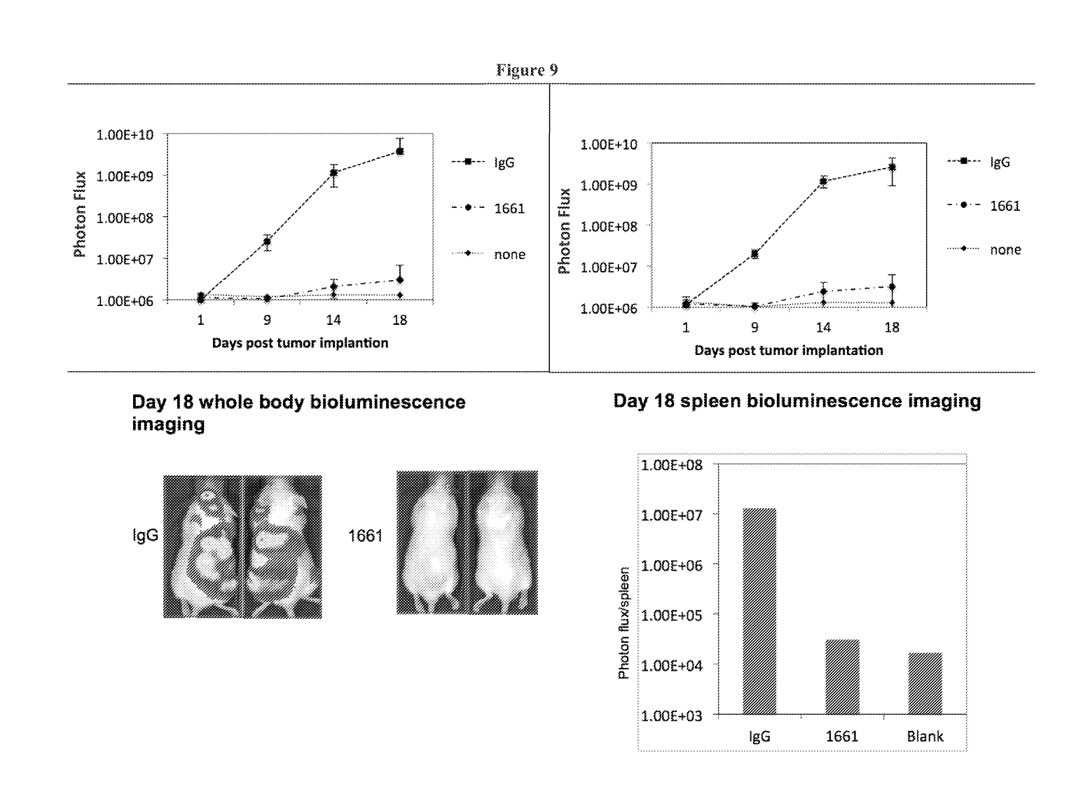

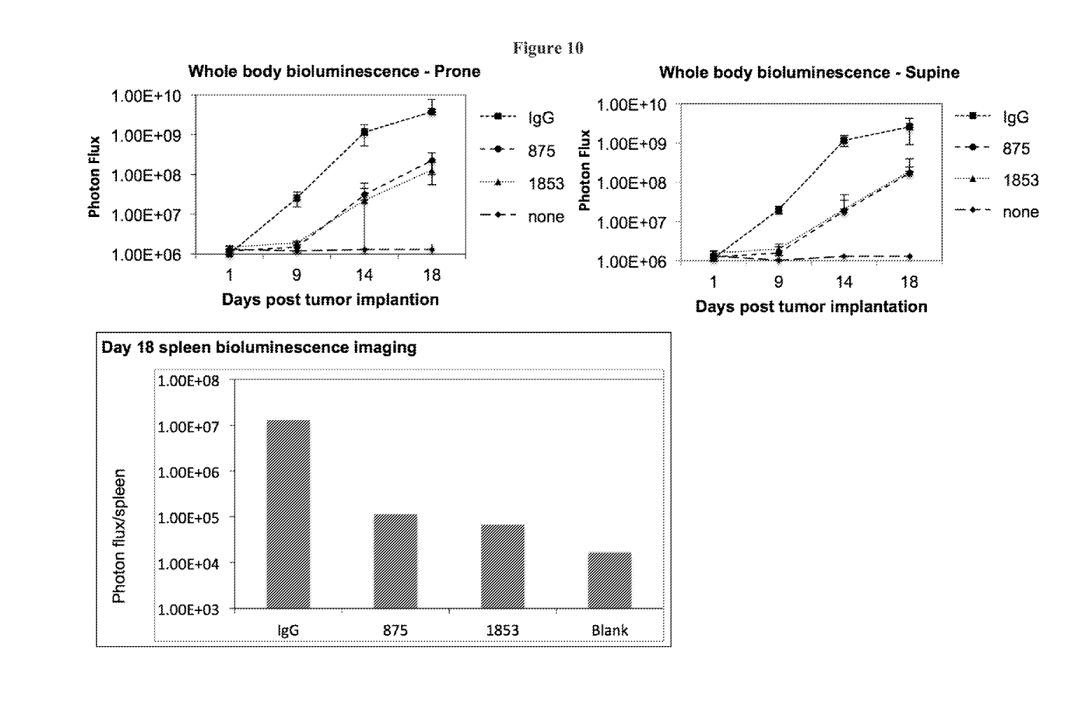

FIG. 10 shows the effect of the dual scFv heterodimer Fc variant v875 and the hybrid heterodimer Fc variant v1853, both with wild-type IgG1 Fc (no FcgR KO mutations). Under these conditions the variants 875 and the hybrid 1853, which both contain a wild-type Fc, show a reduced level of G2 depletion in whole body imaging compared to the equivalent dual scFv heterodimer Fc variant 1661 with Fc knock-out. Both the dual scFv heterodimer and the hybrid heterodimer Fc construct show despite the difference in format comparable level of G2 depletion in whole body bioluminescent imaging.

### Example 11

### Pharmacokinetics of Bi-Specific Anti-CD3-CD19 Antigen-Binding Constructs in NSG Mice

The pharmacokinetics (PK) of v875 at one dose level following a single 0.8 mg/kg IV administration in female NSG mice (NOD.Cg-Prkdc^{scid}Il2rg^{tm1Wjl}/SzJ) was determined. The PK of a control monospecific antibody that binds to Her2 was also determined.

Briefly, purified v875 was administered on Day 1 by an IV injection into the tail vein at a dose of 1 mg/kg. Blood samples, approximately 0.050 mL, were collected from the submandibular or saphenous vein at selected time points (3 animals per time points) up to 72 h post injection. Pre-treatment serum samples were obtained from a naïve animal. Blood samples were allowed to clot at room temperature for 15 to 30 minutes. Blood samples were centrifuged to obtain serum at 2700 rpm for 10 min at room temperature. Serum samples were split into 3 tubes and kept at −80° C. pending analysis.

The serum concentrations were determined by standard anti-human-Fc alphaLISA. A separately measured standard curve of purified v875 was used to determine the serum concentration of v875. The serum concentrations were analyzed using the WinnonLin software version 5.3.

FIG. 11 shows the PK profile of the dual scFv heterodimer Fc variant v875 in NSG mice for the first 12 h and the first 72 h post injection, with a PK profile comparable to IgG control antibody v506 (v506 is the therapeutic antibody TRASTUZUMAB (Herceptin (Genentech)), used as control).

### Example 12

### Target B Cell-Dependence of T Cell Activation by Bi-Specific Heterodimer Variants in Human PBMC

The dependence of T-cell activation by the exemplary bi-specific heterodimer variant 6754 on target B cells was determined in human PBMCs. The experiment was carried out as described below.

Human blood (120-140 mL) was collected from donors and PBMC were freshly isolated from donors. PBMCs were further processed to derive the subpopulations i) PBMC and ii) PBMC without B cells (PBMC-B). Autologous B cells and T cells, at day 0, were determined by FACS. Quadruplicate wells were plated for each control and experimental condition and PBMC cultures were incubated in 5% CO2, 37° C. and stopped at 72 hours. Autologous T and B cells were assessed for their respective proportions in the culture and their 7AAD+ cell contents. The cell pellets were resuspended in various antibody cocktails for flow cytometry analysis. A Guava 8HT flow cytometer was used for analysis of cell subpopulations.

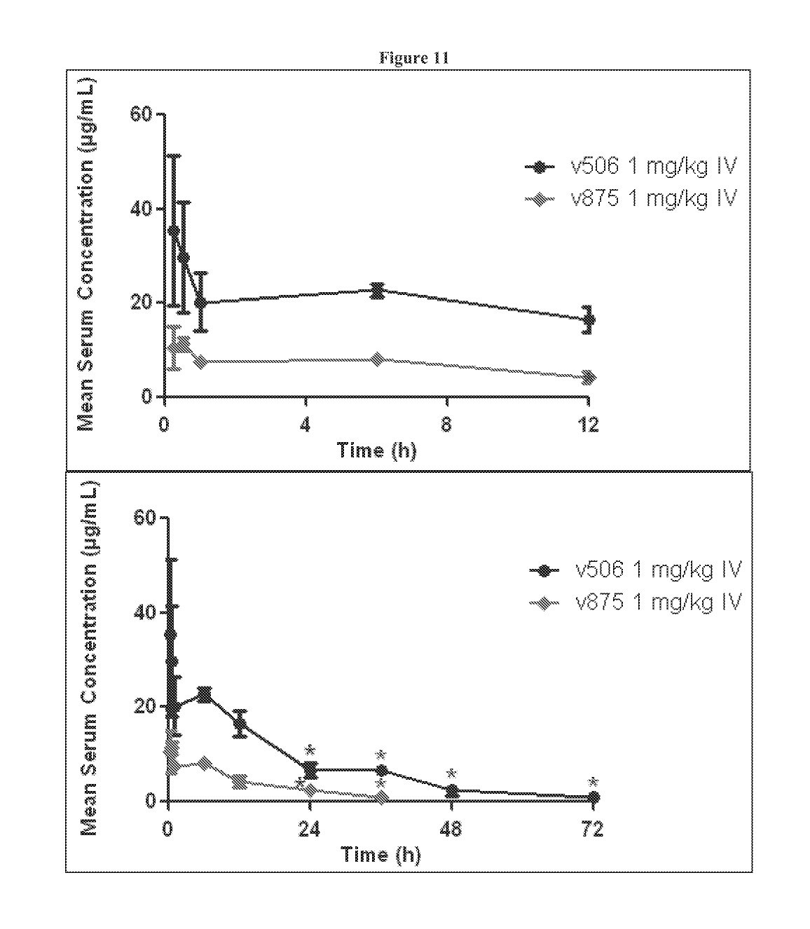

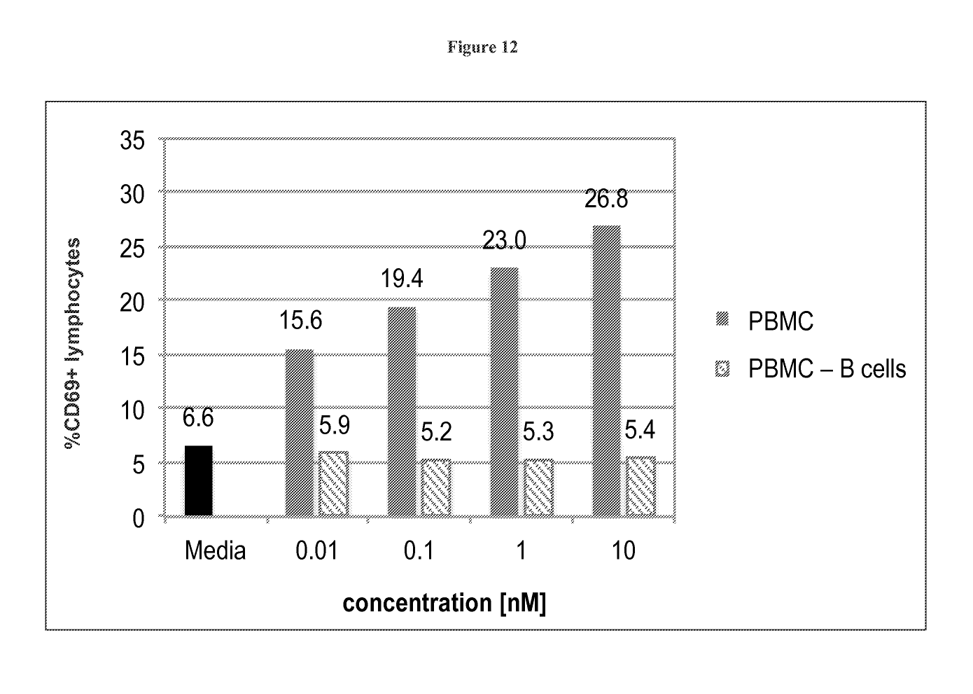

The results are shown in Table 8 and FIG. 12. Table 13 provides the donor PBMC profile. Average E:T ratio in human PBMC collected from healthy donors was ˜10:1 CD3+ T cells to CD19+ B cells.

TABLE 8

| | % CD3+ | % CD4+ | % CD8+ | % CD19+ |
|---|---|---|---|---|
| | T cells/ | T cells/ | T cells/ | B cells/ |
| | CD45+ | CD3+ | CD3+ | CD45+ |
| | lymphocytes | fraction | fraction | lymphocytes |
| Donor 1 | 74.1 | 60.5 | 39.2 | 9.4 |
| Donor 2 | 69.1 | 69.1 | 39.2 | 11.2 |
| Donor 3 | 77.1 | 72.8 | 32.2 | 7.5 |

FIG. 12 indicates that v6754 does not activate T cells in cultures of PBMC lacking B cells up to 10 nM, but activates T cells in presence of b cells in whole PBMC at a concentration as low as 0.01 nM. v6754 shows strictly target dependent T cell activation at concentrations mediating maximal ex vivo B cell depletion (FIG. 7)).

### Example 13

### Bi-Specific Heterodimer Variants Stimulate Less Human T Cell Proliferation than Controls in Human Primary Blood Culture

The ability of the exemplary hybrid heterodimer Fc variant 6754 to induce autologous T cell proliferation in human PBMC was assessed as described below.

Cell Proliferation Assay:

On Day 1, blood was collected from each of 4 donors and PBMCs were freshly isolated. The test items were prepared for a final concentration of 0.3 and 100 nM and combined with the PBMCs, plated at 250,000 cells/well. The mixtures were incubated for 3 days, after which tritiated thymidine was added to the cell containing wells for a final of 0.5 μCi thymidine/well; the plates were incubated for an additional 18 hours, after which the plates were frozen. Total incubation time was 4 days. The plates were filtered and counted (CPMs) using a β-counter. From the averages, a Stimulation Index (SI) was calculated as follows and the data was tabulated: average CPM of test item/average CPM of media only.

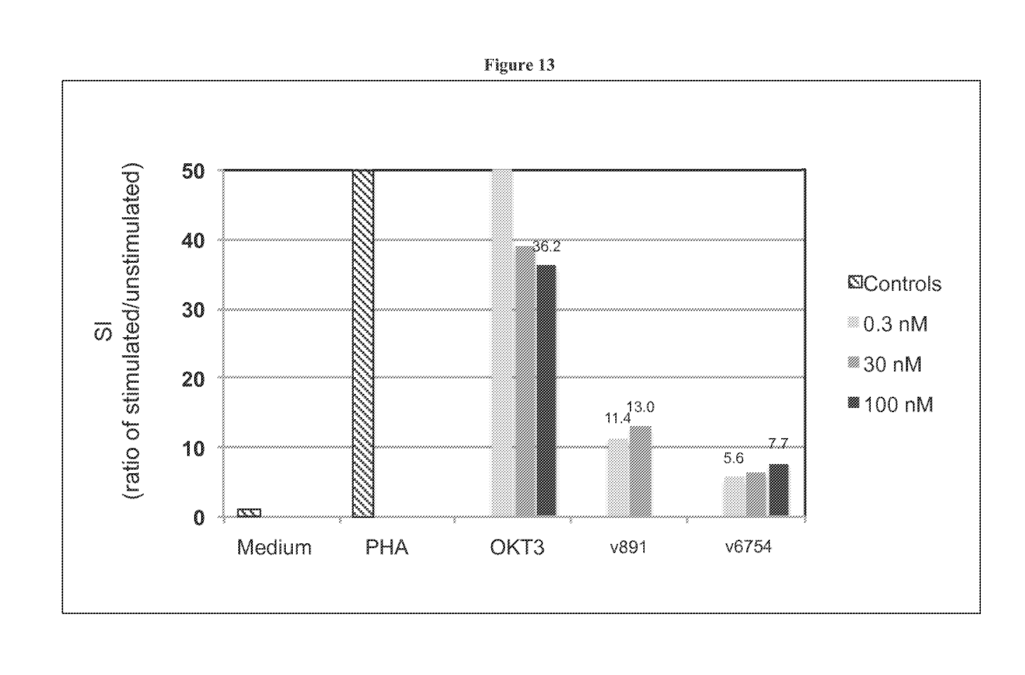

The results are shown in Table 9 and FIG. 13. The average E:T ratio in human PBMC collected from healthy donors was ˜10:1 CD3+ T cells to CD19+ B cells.

TABLE 9

| | % CD3+ | % CD4+ | % CD8+ | % CD19+ |
|---|---|---|---|---|
| | T cells of | T cells of | T cells of | B cells of |
| | CD45+ | CD3+ | CD3+ | CD45+ |
| | lymphocytes | fraction | fraction | lymphocytes |
| Donor 1 | 61.7 | 60.1 | 32.8 | 7.9 |
| Donor 2 | 66.7 | 75.1 | 26.3 | 7.8 |
| Donor 3 | 72.8 | 72.2 | 30.0 | 8.4 |

As shown in FIG. 13, the commercial therapeutic antibody muronomab-OKT3 mediates maximum T cell proliferation at 0.3 nM followed in descending rank order: 891 (BiTE)>6754: At this serum concentration, OKT3 and BiTE are associated with adverse effects (see for example, Chatenoud et al., J Immunol 137(3):830-8 (1986); Abramowicz et al., Transplantation 47(4):606-8 (1989); Goebeler et al. Annals Oncology 22, Supl 4: abstract 068 (2011); Bargou et al. Science 321(5891):974-7 (2008); Topp et al., J. Clin. Oncol. 29(18):2493-8 (2011); Klinger et al. Blood 119(28): 6226-33 (2010); and International Patent Publication No. WO2011051307A1). T cell proliferation induced by 6754 is significantly below the T cell proliferation levels induced by OKT3 and BiTE at 0.3 nM and up to 100 nM. v6754 induces sufficient T cell proliferation (but at much lower levels than benchmarks) for maximal B cell depletion (FIG. 7).

### Example 14

### Bi-Specific Heterodimer Variants Exhibit Low Levels of Cytokine Release in Human Primary Blood Culture Compared to Controls

The degree of cytokine release induced by the exemplary variant 6754 in resting human PBMC was determined.

Cytokine release assay: On Day 1, blood was collected from each of 4 donors and PBMCs were freshly isolated. The test items were prepared for a final concentration of 0.3 and 100 nM and combined with the PBMCs, plated at 250,000 cells/well. The mixtures were incubated for 4 days. After incubation the supernatants from the replicates were pooled and used for cytokine measurements, in duplicates, using the CBA Human Th1/Th2 Cytokine Kit II from BD Biosciences. This kit measures IL-2, IL-4, IL-6, IL-10, TNF and IFNγ.

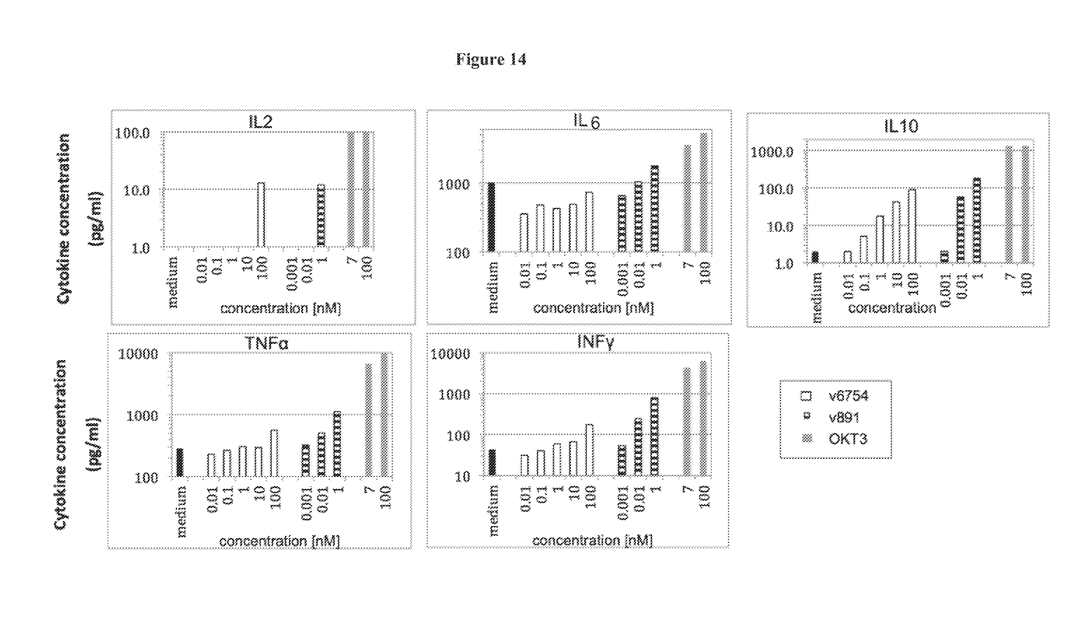

The results are shown in Table 10 and FIG. 14. The average E:T ratio in human PBMC collected from healthy donors was ˜10:1 CD3+ T cells to CD19+ B cells.

TABLE 10

| | % CD3+ | % CD4+ | % CD8+ | % CD19+ |
|---|---|---|---|---|
| | T cells/ | T cells/ | T cells/ | B cells/ |
| | CD45+ | CD3+ | CD3+ | CD45+ |
| | lymphocytes | fraction | fraction | lymphocytes |
| Donor 1 | 74.1 | 60.5 | 39.2 | 9.4 |
| Donor 2 | 69.1 | 69.1 | 39.2 | 11.2 |
| Donor 3 | 77.1 | 72.8 | 32.2 | 7.5 |

FIG. 14 shows that at a concentration of 100 nM, v6754 induced IFNγ, TNFα, IL-2, IL-6 and IL-10 cytokine levels to a significantly lower level than the commercial therapeutic antibody muronomab-OKT3 at a 7 nM concentration. At a 7 nM serum concentration, OKT3 is associated with adverse effects (see for example Chatenoud et al., J Immunol 137(3):830-8 (1986), and

Abramowicz et al., Transplantation 47(4):606-8 (1989)). BiTE induces similar and higher levels of IFNγ, TNFα, IL-2, IL-6 and IL-10 cytokines at comparable concentration s of v6754. v6754 induces low levels of cytokines at concentrations mediating maximal ex vivo B cell depletion (FIG. 7).

### Example 15

### In Vivo Mouse Pharmacokinetics of an Exemplary Bi-Specific Hybrid Heterodimer Variant

The pharmacokinetics (PK) of an exemplary bi-specific heterodimer variant, 1853, was determined in mice. Variant 1853 is identical to variant 6754, except that variant 1853 does not include the CH2 mutations that knockout binding of the Fc to FcγR. The experiment was carried out as described below.

Pharmacokinetics in NSG mice: The pharmacokinetics of 1853 at one dose level following a single 3 mg/kg IV administrations in female NSG mice (NOD.Cg-Prkdc^{scid}Il2rg^{tm1Wjl}/SzJ) was determined 1853 was administered on Day 1 by an IV injection into the tail vein at a dose of 3 mg/kg. Blood samples, approximately 0.050 mL, were collected from the submandibular or saphenous vein at selected time points (3 animals per time points). Pre-treatment serum samples were obtained from a naïve animal. Blood samples were allowed to clot at room temperature for 15 to 30 minutes. Blood samples were centrifuged to obtain serum at 2700 rpm for 10 min at room temperature. Serum samples were split into 3 tubes and kept at −80° C. pending analysis. The serum concentrations were determined by standard anti-human-Fc Luminex A separately measured standard curve of purified 1853 was used to determine the serum concentration of 1853. PK parameters were calculated with WinNonLin using non-compartmental model analysis.

The results are shown in Table 11 and FIGS. 15A and B.

TABLE 11
PK parameters of v1853 in NSG mice
PK parameters

| | |
|---|---|
| Cₘₐₓ [μg/mL] | 33.1 |
| AUC [h*μg/mL] | 811.8 |
| Vₛₛ [mL/kg] | 131.8 |
| CI [mL/h/kg] | 3.6 |
| MRT [h] | 36.7 |
| t_{1/2} [h] | 25.7 |

Table 11 shows the PK parameters measured for 1853. FIG. 15 demonstrates that a single IV dose of 6754 at 3 mg/kg in NSG (NOD scid gamma, NOD.Cg-Prkdc^{scid}Il2rg^{tm1Wjl}/SzJ) mice shows IgG1-like clearance and a half-life in mice of >24 h (FIG. 15B shows the data of FIG. 15A plotted using a logarithmic scale). v6754 shows typical human IgG-like pharmacokinetics: half-life, distribution and clearance in mice.

In addition, as part of the in vivo efficacy study as described in detail in Example 12, serum samples were collected for 2 animals at 24 h after the first 3 mg/kg IV dose (Example 12). The serum samples were analysed as described above and the 24 h serum concentrations are shown in FIG. 15C. The exposure at 24 h after IV injection (FIG. 15C) is equivalent to the exposure observed in the PK study (FIG. 15A,B), confirming the IgG1-like PK of the CD3-CD19 bi-specific variants tested.

### Example 16

### Effect of Bi-Specific Heterodimer Variants in an In Vivo Human B-ALL Xenograft Model in Humanized NSG Mice

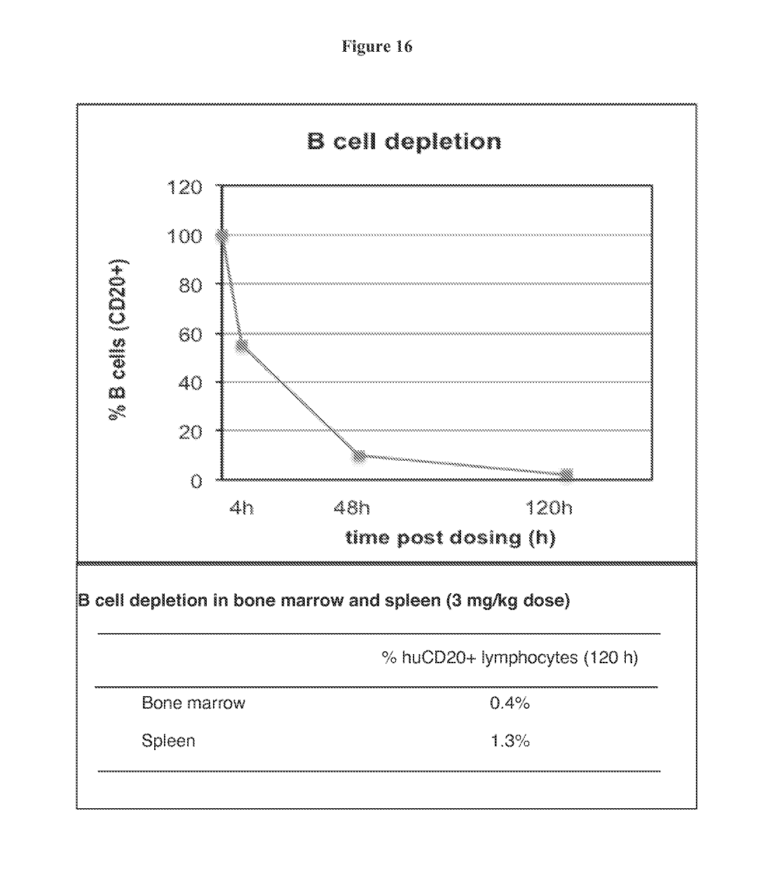

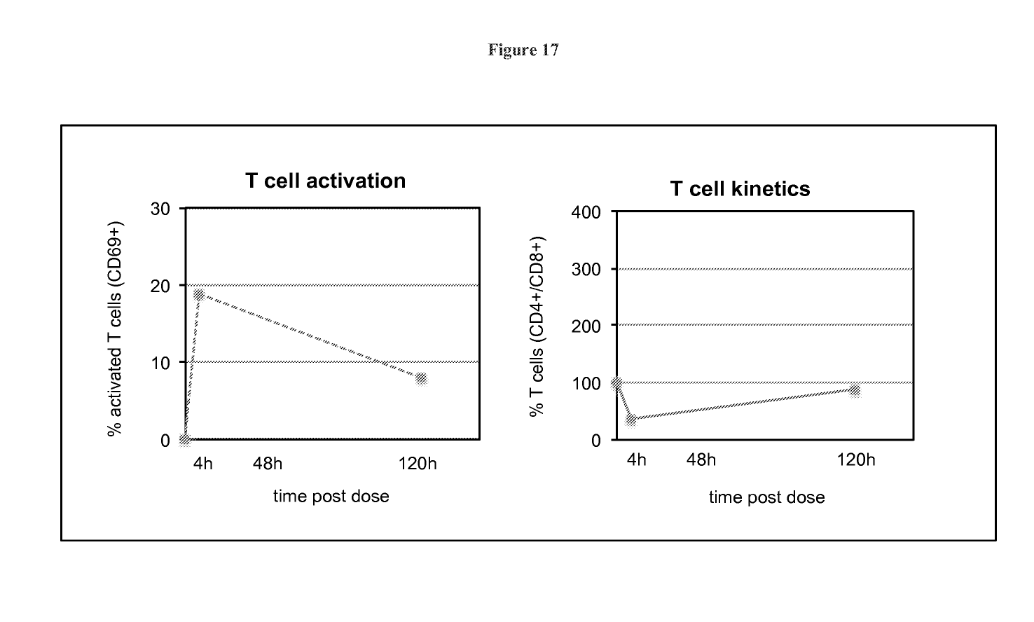

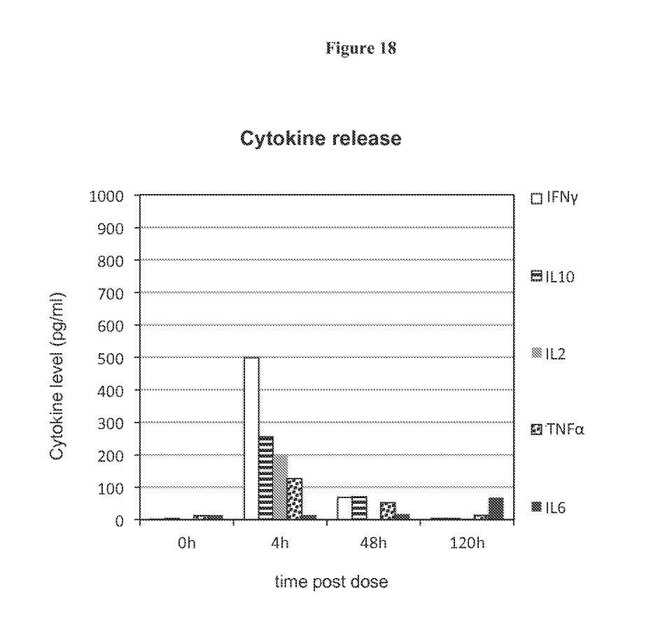

The effect of an exemplary variant, v6754, in an in vivo human B-ALL xenograft model in humanized (CD34+) NSG mice (E:T˜1:5) was evaluated. The ability of v6754 to deplete autologous B-cells (FIG. 16), activate and redistribute T-cells (FIG. 17), and modulate cytokine release (FIG. 18) in this model was determined as described below.

Humanized (CD34+) NSG mice were purchased from Jackson laboratory. 2 week old NSG (NOD scid gamma, NOD.Cg-Prkdc^{scid}Il2rg^{tm1Wjl}/SzJ) mice were injected with human (CD34+) hematopoietic stem cells HSC from human fetal liver. Humanized (CD34+) NSG mice develop human T cell and B cell linages within 12 weeks. Average T cell to B cell ratio in humanized (CD34+) NSG mice is −1:5. v6754 was dosed as single 3 mg/kg IV injection.

In Vivo Efficacy in Humanized NSG Mice:

The in vivo cytotoxicity of the bi-specific antigen-binding constructs was tested as follows. Briefly, humanized (hCD34+) NSG mice were injected with 1 IV bolus of v6754 at 3 mg/kg on Day 1 and autologous circulating B and T cell numbers and human cytokine levels in peripheral blood, bone marrow and spleen was measured at 4-6 h past injection and at days 2 and 5. The T cell and B cell populations were analyzed by FACS after labeling for human CD45, CD20, CD4, CD8 and CD69. Human cytokines IFNγ, TNFα, IL2, IL6, IL10 were measured. Autologous B cell depletion in peripheral blood, bone marrow and spleen was monitored by FACS. The B and T cell populations in peripheral blood were normalized to the levels analyzed 2 weeks prior to the Day 1 injections.

Autologous B Cell Depletion:

The results depicting the effect of v6754 on depletion of autologous B cells are shown in FIG. 16. Table 12 shows the average lymphocyte populations in the humanized NSG mice before treatment. FIG. 16 depicts the in vivo efficacy of 6754 in humanized NSG mice.

TABLE 12

| | % huCD19+ | % huCD3+ | % huCD4+ | % huCD8+ |
|---|---|---|---|---|
| | B cells in | T cells in | T cells in | T cells in |
| huCD45+ in | huCD45+ | huCD45+ | huCD45+ | huCD45+ |
| lymphocytes | fraction | fraction | fraction | fraction |
| 30-50% | 60-80% | 20-30% | 15-20% | 5-10% |

As shown in FIG. 16, after single IV dose (3 mg/kg) with v6754, no B cells were observed 5 days post-dosing in the peripheral blood, bone marrow and spleen in humanized NSG mice with low E:T ratio of 1:5.

In vivo activation and redistribution kinetics of autologous T cells: Evaluation of v6754-mediated in vivo activation and redistribution kinetics of autologous T cells in humanized (CD34+) NSG mice (E:T˜1:5) was carried out as described above.

The results are shown in FIG. 17. At a dose of v6754 that completely depletes autologous B cells in vivo (FIG. 16), autologous T cells were transiently activated as measured by CD69+ staining after 4 h. Peripheral T cells counts decreased reaching a nadir several hours after injection of v6754, and recovered to baseline after <5 days. T cell activation and reduced serum counts profile were similar to published findings with blinatumomab (see Klinger et al. Blood 119(28): 6226-33 (2010)), but the effects are more modest suggesting bi-specific antigen-binding constructs can mediate maximal B cell depletion with ‘appropriate’ levels of T cell activation vs. blinatumomab. The CD3-CD19 hybrid and dual scFv heterodimer Fc formats permits a more controlled T cell activation by virtue of their specific geometry and the resulting nature of T cell engagement, synapse formation and kinetics.

In Vivo Cytokine Release in Humanized NSG Mice:

As indicated above, human cytokines IFNγ, TNFα, IL2, IL6, IL10 were measured. The results are shown in FIG. 18 and indicate that v6754 induced cytokine release in humanized NSG mice after a single 3 mg/kg IV injection. Cytokine release was transient and peaked in the first hours. The peak levels at a 3 mg/kg dose were below published clinical cytokine levels. v6754 induced modest and transient cytokine release following single 3 mg/kg IV injection. Cytokine release patterns were similar to published findings with blinatumomab (see Klinger (2010), supra), but the effects are more modest, again suggesting bi-specific antigen-binding constructs can activate T cells at an ‘appropriate’ level for maximal B cell depletion.

### Example 17

### In Vitro and Ex Vivo Characterization of a Bispecific CD3-CD19 Binding Construct with Cross Species Binding Activity to Human and Cynomolgus Monkey

The CD19-CD3 hybrid heterodimer Fc variant 5851 (cloning and construction described in Examples 2 and 3) is constructed from known variable domains, known to bind to human and cynomolgus monkey CD19 and CD3. V5851 was expressed, purified and characterized by LC/MS and whole cell FACS binding as described in Examples 3-5. The purified v5851 was further analyzed for ex vivo activity in human primary blood cultures as described Example 11.

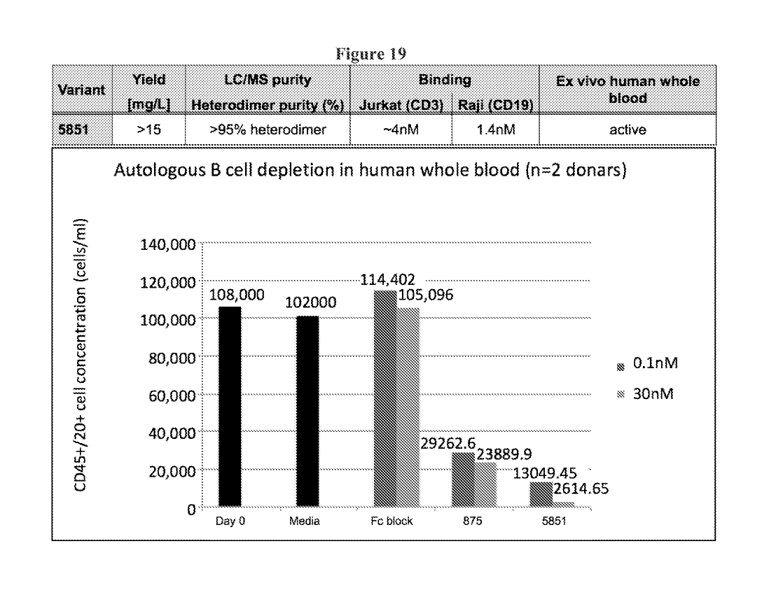

FIG. 19 show the cytotoxic effect of the species cross reactive v5851 constructs on the autologous B cell concentration in human whole blood following IL2 incubation in comparison to the dual scFv heterodimer Fc variant v875. Both variants were able to maximally deplete CD20+ B cells in this assay at the 0.1 nM.

The analysis shows that despite the difference in both the anti-CD3 and the anti-CD19 variable domains and the difference in geometry of the binding domains between the dual scFv heterodimer Fc vs. hybrid heterodimer Fc variant, dual scFv heterodimer Fc variant v875 and the species cross reactive hybrid heterodimer Fc variant v5851 show comparable ex vivo efficacy in human primary blood culture at the minimal measure concentration of 0.1 nM.

### Additional Tables

TABLE XX
Variant numbers of exemplary anti-CD3-CD19 or anti CD3-CD20
antigen-binding constructs and clone name of heavy chains (H1
and H2) and, if applicable, light chains (L1 and L2). See Table
YY for nucleic acid and polypeptide sequences of clones.

| Variant | | | | |
|---|---|---|---|---|
| Number | H1 (Clone) | H2 (Clone) | L1 (Clone) | L2 (Clone) |
| 873 | 1064 | 1065 | n/a | n/a |
| 875 | 1064 | 1067 | n/a | n/a |
| 1661 | 2183 | 2176 | n/a | n/a |
| 1653 | 1842 | 2167 | n/a | n/a |
| 5850 | 3320 | 2317 | 2325 | n/a |
| 5851 | 3320 | 2307 | 2312 | n/a |
| 5852 | 2304 | 3322 | 2309 | n/a |
| 6325 | 2304 | 3916 | 2309 | n/a |
| 1813 | 2313 | 2317 | 2325 | 2325 |
| 1821 | 2303 | 1342 | 1335 | 1335 |
| 1823 | 2303 | 2316 | 2323 | 2323 |
| 1853 | 2304 | 2175 | 2309 | n/a |
| 6754 | 5239 | 2185 | 2309 | n/a |
| 10151 | 5239 | 6691 | 2309 | n/a |
| 6750 | 5241 | 5238 | 2310 | n/a |
| 6751 | 5242 | 2176 | 2310 | n/a |
| 6475 | 2305 | 2171 | 2310 | n/a |
| 6749 | 5242 | 2177 | 2310 | n/a |
| 10152 | 5242 | 6689 | 2310 | n/a |
| 10153 | 5242 | 6690 | 2310 | n/a |
| 6518 | 2304 | 2305 | 2309 | 2310 |
| 6476 | 2305 | 2170 | 2310 | n/a |

TABLE YY1
Nucleic acid sequences of clones described in Table XX.

| SEQ | | | |
|---|---|---|---|
| ID | | | |
| NO: | Clone | Desc | Nucleic acid (coding) sequence |
| 1. | 2176 | Full | CAGATCGTCCTGACACAGAGCCCAGCTATCATGTCAGCAAGCCCCGGCGAGAAAGTCACAATGACTTGCTCAG |
| | | | CCAGCTCCTCTGTGAGCTACATGAACTGGTATCAGCAGAAAAGCGGAACCTCCCCCAAGAGATGGATCTACGA |
| | | | CACATCCAAGCTGGCCTCTGGAGTGCCTGCTCACTTCAGGGGCAGCGGCTCTGGGACCAGTTATTCACTGACA |
| | | | ATTTCCGGCATGGAGGCCGAAGATGCCGCTACCTACTATTGCCAGCAGTGGAGTTCAAACCCATTCACTTTTG |
| | | | GATCTGGCACCAAGCTGGAAATTAATGGCGGAGGAGGCTCCGGAGGAGGAGGGTCTGGAGGAGGAGGAAGTCA |
| | | | GGTGCAGCTGCAGCAGTCCGGAGCAGAGCTGGCTCGACCAGGAGCTAGTGTGAAAATGTCCTGTAAGGCAAGC |
| | | | GGCTACACCTTCACACGGTATACCATGCATTGGGTGAAACAGAGACCCGGGCAGGGACTGGAATGGATCGGGT |
| | | | ACATTAATCCTAGCCGAGGATACACAAACTACAACCAGAAGTTTAAAGACAAGGCCACTCTGACCACAGATAA |
| | | | GAGCTCCTCTACCGCTTATATGCAGCTGAGTTCACTGACATCTGAGGACAGTGCAGTGTACTATTGCGCCAGG |
| | | | TACTATGACGATCACTACTGTCTGGATTATTGGGGCCAGGGGACTACCCTGACAGTGAGCTCCGCAGCCGAAC |
| | | | CTAAATCTAGTGACAAGACTCATACCTGCCCCCCTTGTCCAGCACCAGAGGCTGCAGGAGGACCTTCCGTGTT |
| | | | CCTGTTTCCACCCAAACCAAAGGATACTCTGATGATCTCCCGGACACCTGAAGTCACTTGCGTGGTCGTGAGC |
| | | | GTGTCTCACGAGGACCCCGAAGTCAAGTTTAACTGGTACGTGGACGGCGTCGAGGTGCATAATGCCAAAACCA |
| | | | AGCCCAGGGAGGAACAGTACAACTCCACATATCGCGTCGTGTCTGTCCTGACTGTGCTGCACCAGGATTGGCT |
| | | | GAACGGCAAGGAGTACAAATGCAAGGTGAGCAACAAGGCACTGCCTGCCCCAATCGAGAAGACAATTAGCAAA |
| | | | GCAAAGGGGCAGCCCCGAGAACCTCAGGTCTACGTGCTGCCTCCATCTCGGGACGAGCTGACTAAAAACCAGG |
| | | | TCAGTCTGCTGTGTCTGGTGAAGGGCTTCTATCCAAGCGATATTGCTGTGGAGTGGGAATCCAATGGGCAGCC |
| | | | CGAAAACAATTACCTGACTTGGCCCCCTGTCCTGGACTCAGATGGGAGCTTCTTTCTGTATAGTAAACTGACC |
| | | | GTGGACAAGTCACGGTGGCAGCAGGGAAACGTCTTTAGCTGTTCCGTGATGCATGAGGCCCTGCACAATCATT |
| | | | ACACCCAGAAATCTCTGAGTCTGTCACCCGGCAAG |
| 2. | 2176 | VL | CAGATCGTCCTGACACAGAGCCCAGCTATCATGTCAGCAAGCCCCGGCGAGAAAGTCACAATGACTTGCTCAG |
| | | | CCAGCTCCTCTGTGAGCTACATGAACTGGTATCAGCAGAAAAGCGGAACCTCCCCCAAGAGATGGATCTACGA |
| | | | CACATCCAAGCTGGCCTCTGGAGTGCCTGCTCACTTCAGGGGCAGCGGCTCTGGGACCAGTTATTCACTGACA |
| | | | ATTTCCGGCATGGAGGCCGAAGATGCCGCTACCTACTATTGCCAGCAGTGGAGTTCAAACCCATTCACTTTTG |
| | | | GATCTGGCACCAAGCTGGAAATTAAT |
| 3. | 2176 | VH | CAGGTGCAGCTGCAGCAGTCCGGAGCAGAGCTGGCTCGACCAGGAGCTAGTGTGAAAATGTCCTGTAAGGCAA |
| | | | GCGGCTACACCTTCACACGGTATACCATGCATTGGGTGAAACAGAGACCCGGGCAGGGACTGGAATGGATCGG |
| | | | GTACATTAATCCTAGCCGAGGATACACAAACTACAACCAGAAGTTTAAAGACAAGGCCACTCTGACCACAGAT |
| | | | AAGAGCTCCTCTACCGCTTATATGCAGCTGAGTTCACTGACATCTGAGGACAGTGCAGTGTACTATTGCGCCA |
| | | | GGTACTATGACGATCACTACTGTCTGGATTATTGGGGCCAGGGGACTACCCTGACAGTGAGCTCC |
| 4. | 2176 | CH2 | GCACCAGAGGCTGCAGGAGGACCTTCCGTGTTCCTGTTTCCACCCAAACCAAAGGATACTCTGATGATCTCCC |
| | | | GGACACCTGAAGTCACTTGCGTGGTCGTGAGCGTGTCTCACGAGGACCCCGAAGTCAAGTTTAACTGGTACGT |
| | | | GGACGGCGTCGAGGTGCATAATGCCAAAACCAAGCCCAGGGAGGAACAGTACAACTCCACATATCGCGTCGTG |
| | | | TCTGTCCTGACTGTGCTGCACCAGGATTGGCTGAACGGCAAGGAGTACAAATGCAAGGTGAGCAACAAGGCAC |
| | | | TGCCTGCCCCAATCGAGAAGACAATTAGCAAAGCAAAG |
| 5. | 2176 | CH3 | GGGCAGCCCCGAGAACCTCAGGTCTACGTGCTGCCTCCATCTCGGGACGAGCTGACTAAAAACCAGGTCAGTC |
| | | | TGCTGTGTCTGGTGAAGGGCTTCTATCCAAGCGATATTGCTGTGGAGTGGGAATCCAATGGGCAGCCCGAAAA |
| | | | CAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCC |
| | | | AGAAATCTCTGAGTCTGTCACCCGGC |
| 6. | 2304 | Full | CAGGTCCAGCTGGTGCAGAGCGGAGGAGGAGTGGTCCAGCCAGGACGGTCTCTGAGACTGAGTTGCAAGGCAT |
| | | | CAGGGTACACTTTCACCCGATATACCATGCACTGGGTGCGGCAGGCACCAGGGAAAGGACTGGAATGGATCGG |
| | | | GTACATTAACCCTTCCAGGGGATACACAAACTATAATCAGAAGGTGAAAGACAGGTTCACTATCAGCCGCGAT |
| | | | AACTCCAAGAATACCGCTTTTCTGCAGATGGACTCTCTGCGCCCCGAGGATACAGGCGTGTATTTCTGCGCAC |
| | | | GATACTATGACGATCACTACTGTCTGGACTATTGGGGCCAGGGGACTCCAGTCACCGTGAGCTCCGCCTCTAC |
| | | | TAAGGGACCCAGTGTGTTTCCACTGGCTCCCTCTAGTAAATCCACATCTGGAGGAACTGCAGCTCTGGGATGC |
| | | | CTGGTGAAGGATTACTTCCCAGAGCCCGTCACCGTGAGTTGGAACTCAGGAGCTCTGACTAGCGGCGTCCATA |
| | | | CCTTTCCCGCAGTGCTGCAGTCAAGCGGGCTGTACAGCCTGTCCTCTGTGGTCACAGTGCCTAGTTCAAGCCT |
| | | | GGGAACACAGACTTATATCTGCAACGTGAATCACAAGCCTTCTAATACTAAAGTCGACAAGAAAGTGGAACCA |
| | | | AAGAGTTGTGATAAAACCCATACATGCCCACCTTGTCCTGCACCAGAGCTGCTGGGAGGACCAAGCGTGTTCC |
| | | | TGTTTCCACCCAAGCCTAAAGACACCCTGATGATTAGCCGGACCCCTGAAGTCACATGTGTGGTCGTGGACGT |
| | | | GAGCCACGAGGACCCCGAAGTCAAGTTCAACTGGTACGTGGATGGCGTCGAGGTGCATAATGCCAAGACAAAA |
| | | | CCTAGAGAGGAACAGTACAATTCAACCTATAGGGTCGTGAGCGTCCTGACAGTGCTGCACCAGGACTGGCTGA |
| | | | ACGGGAAGGAGTATAAGTGCAAAGTGTCCAATAAGGCACTGCCCGCCCCTATCGAGAAAACCATTTCTAAGGC |
| | | | AAAAGGCCAGCCTAGGGAACCACAGGTCTACGTGTATCCTCCAAGCCGCGACGAGCTGACAAAGAACCAGGTC |
| | | | TCCCTGACTTGTCTGGTGAAAGGATTTTACCCAAGTGATATTGCTGTGGAGTGGGAATCAAATGGCCAGCCCG |
| | | | AAAACAATTATAAGACCACACCCCCTGTGCTGGACAGCGATGGCTCCTTCGCCCTGGTCTCCAAGCTGACTGT |
| | | | GGATAAATCTAGATGGCAGCAGGGGAACGTCTTTAGTTGTTCAGTGATGCATGAGGCTCTGCACAATCATTAC |
| | | | ACCCAGAAGAGCCTGTCCCTGTCTCCCGGCAAA |
| 7. | 2304 | VH | CAGGTCCAGCTGGTGCAGAGCGGAGGAGGAGTGGTCCAGCCAGGACGGTCTCTGAGACTGAGTTGCAAGGCAT |
| | | | CAGGGTACACTTTCACCCGATATACCATGCACTGGGTGCGGCAGGCACCAGGGAAAGGACTGGAATGGATCGG |
| | | | GTACATTAACCCTTCCAGGGGATACACAAACTATAATCAGAAGGTGAAAGACAGGTTCACTATCAGCCGCGAT |
| | | | AACTCCAAGAATACCGCTTTTCTGCAGATGGACTCTCTGCGCCCCGAGGATACAGGCGTGTATTTCTGCGCAC |
| | | | GATACTATGACGATCACTACTGTCTGGACTATTGGGGCCAGGGGACTCCAGTCACCGTGAGCTCC |
| 8. | 2304 | CH1 | GCCTCTACTAAGGGACCCAGTGTGTTTCCACTGGCTCCCTCTAGTAAATCCACATCTGGAGGAACTGCAGCTC |
| | | | TGGGATGCCTGGTGAAGGATTACTTCCCAGAGCCCGTCACCGTGAGTTGGAACTCAGGAGCTCTGACTAGCGG |
| | | | CGTCCATACCTTTCCCGCAGTGCTGCAGTCAAGCGGGCTGTACAGCCTGTCCTCTGTGGTCACAGTGCCTAGT |
| | | | TCAAGCCTGGGAACACAGACTTATATCTGCAACGTGAATCACAAGCCTTCTAATACTAAAGTCGACAAGAAAG |
| | | | TG |
| 9. | 2304 | CH2 | GCACCAGAGCTGCTGGGAGGACCAAGCGTGTTCCTGTTTCCACCCAAGCCTAAAGACACCCTGATGATTAGCC |
| | | | GGACCCCTGAAGTCACATGTGTGGTCGTGGACGTGAGCCACGAGGACCCCGAAGTCAAGTTCAACTGGTACGT |
| | | | GGATGGCGTCGAGGTGCATAATGCCAAGACAAAACCTAGAGAGGAACAGTACAATTCAACCTATAGGGTCGTG |
| | | | AGCGTCCTGACAGTGCTGCACCAGGACTGGCTGAACGGGAAGGAGTATAAGTGCAAAGTGTCCAATAAGGCAC |
| | | | TGCCCGCCCCTATCGAGAAAACCATTTCTAAGGCAAAA |
| 10. | 2304 | CH3 | GGCCAGCCTAGGGAACCACAGGTCTACGTGTATCCTCCAAGCCGCGACGAGCTGACAAAGAACCAGGTCTCCC |
| | | | TGACTTGTCTGGTGAAAGGATTTTACCCAAGTGATATTGCTGTGGAGTGGGAATCAAATGGCCAGCCCGAAAA |
| | | | CAATTATAAGACCACACCCCCTGTGCTGGACAGCGATGGCTCCTTCGCCCTGGTCTCCAAGCTGACTGTGGAT |
| | | | AAATCTAGATGGCAGCAGGGGAACGTCTTTAGTTGTTCAGTGATGCATGAGGCTCTGCACAATCATTACACCC |
| | | | AGAAGAGCCTGTCCCTGTCTCCCGGC |
| 11. | 2307 | Full | GAGGTCCAGCTGGTCGAATCCGGAGGAGGACTGGTGAAGCCAGGAGGGAGTCTGAAACTGTCATGCGCCGCTA |
| | | | GCGGCTATACCTTCACATCTTACGTCATGCACTGGGTGAGGCAGGCACCTGGCAAGGGACTGGAATGGATCGG |
| | | | ATATATTAACCCATACAATGACGGCACTAAGTATAACGAGAAATTTCAGGGCAGAGTGACCATCAGCTCCGAT |
| | | | AAGAGCATTTCCACAGCTTACATGGAGCTGTCTAGTCTGAGGAGCGAAGACACCGCCATGTACTATTGCGCTC |
| | | | GGGGGACCTACTATTACGGAACAAGAGTGTTCGATTATTGGGGACAGGGCACCCTGGTCACAGTGTCAAGCGC |
| | | | TTCCACAAAGGGGCCTTCTGTGTTTCCACTGGCACCCTCCTCTAAATCTACTAGTGGAGGCACCGCAGCCCTG |
| | | | GGATGTCTGGTGAAGGACTACTTCCCAGAGCCCGTCACAGTGTCATGGAACAGCGGCGCACTGACTAGCGGGG |
| | | | TCCATACCTTTCCTGCCGTGCTGCAGAGTTCAGGCCTGTATAGCCTGAGCTCCGTGGTCACAGTGCCATCTAG |
| | | | TTCACTGGGGACTCAGACCTACATCTGCAACGTGAATCACAAGCCATCCAATACTAAAGTCGACAAGAAAGTG |
| | | | GAACCCAAGTCTTGTGATAAAACACATACTTGCCCACCTTGTCCTGCACCAGAGCTGCTGGGAGGACCATCCG |
| | | | TGTTCCTGTTTCCACCCAAGCCTAAAGATACTCTGATGATTAGTCGCACACCAGAAGTGACTTGCGTGGTCGT |
| | | | GGACGTGAGCCACGAGGACCCCGAAGTCAAGTTCAACTGGTACGTGGACGGCGTCGAGGTGCATAATGCCAAG |
| | | | ACCAAACCCAGGGAGGAACAGTATAATAGTACATACAGAGTCGTGTCAGTGCTGACCGTCCTGCACCAGGATT |
| | | | GGCTGAACGGCAAGGAGTACAAGTGCAAAGTGTCCAATAAGGCTCTGCCCGCACCTATCGAGAAAACCATTTC |
| | | | TAAGGCAAAAGGGCAGCCTCGAGAACCACAGGTCTATGTGCTGCCTCCATCACGGGATGAGCTGACAAAGAAC |
| | | | CAGGTCAGCCTGCTGTGTCTGGTGAAAGGGTTCTACCCCTCTGACATCGCTGTGGAGTGGGAAAGTAATGGAC |
| | | | AGCCTGAAAACAATTATCTGACTTGGCCCCCTGTGCTGGACTCCGATGGATCTTTCTTTCTGTACAGCAAGCT |
| | | | GACCGTGGACAAATCCCGATGGCAGCAGGGCAACGTCTTTTCATGTAGCGTGATGCATGAGGCCCTGCACAAT |
| | | | CATTACACCCAGAAGTCCCTGTCTCTGAGTCCCGGCAAA |
| 12. | 2307 | VH | GAGGTCCAGCTGGTCGAATCCGGAGGAGGACTGGTGAAGCCAGGAGGGAGTCTGAAACTGTCATGCGCCGCTA |
| | | | GCGGCTATACCTTCACATCTTACGTCATGCACTGGGTGAGGCAGGCACCTGGCAAGGGACTGGAATGGATCGG |
| | | | ATATATTAACCCATACAATGACGGCACTAAGTATAACGAGAAATTTCAGGGCAGAGTGACCATCAGCTCCGAT |
| | | | AAGAGCATTTCCACAGCTTACATGGAGCTGTCTAGTCTGAGGAGCGAAGACACCGCCATGTACTATTGCGCTC |
| | | | GGGGGACCTACTATTACGGAACAAGAGTGTTCGATTATTGGGGACAGGGCACCCTGGTCACAGTGTCAAGC |
| 13. | 2307 | CH1 | GCTTCCACAAAGGGGCCTTCTGTGTTTCCACTGGCACCCTCCTCTAAATCTACTAGTGGAGGCACCGCAGCCC |
| | | | TGGGATGTCTGGTGAAGGACTACTTCCCAGAGCCCGTCACAGTGTCATGGAACAGCGGCGCACTGACTAGCGG |
| | | | GGTCCATACCTTTCCTGCCGTGCTGCAGAGTTCAGGCCTGTATAGCCTGAGCTCCGTGGTCACAGTGCCATCT |
| | | | AGTTCACTGGGGACTCAGACCTACATCTGCAACGTGAATCACAAGCCATCCAATACTAAAGTCGACAAGAAAG |
| | | | TG |
| 14. | 2307 | CH2 | GCACCAGAGCTGCTGGGAGGACCATCCGTGTTCCTGTTTCCACCCAAGCCTAAAGATACTCTGATGATTAGTC |
| | | | GCACACCAGAAGTGACTTGCGTGGTCGTGGACGTGAGCCACGAGGACCCCGAAGTCAAGTTCAACTGGTACGT |
| | | | GGACGGCGTCGAGGTGCATAATGCCAAGACCAAACCCAGGGAGGAACAGTATAATAGTACATACAGAGTCGTG |
| | | | TCAGTGCTGACCGTCCTGCACCAGGATTGGCTGAACGGCAAGGAGTACAAGTGCAAAGTGTCCAATAAGGCTC |
| | | | TGCCCGCACCTATCGAGAAAACCATTTCTAAGGCAAAA |
| 15. | 2307 | CH3 | GGGCAGCCTCGAGAACCACAGGTCTATGTGCTGCCTCCATCACGGGATGAGCTGACAAAGAACCAGGTCAGCC |
| | | | TGCTGTGTCTGGTGAAAGGGTTCTACCCCTCTGACATCGCTGTGGAGTGGGAAAGTAATGGACAGCCTGAAAA |
| | | | CAATTATCTGACTTGGCCCCCTGTGCTGGACTCCGATGGATCTTTCTTTCTGTACAGCAAGCTGACCGTGGAC |
| | | | AAATCCCGATGGCAGCAGGGCAACGTCTTTTCATGTAGCGTGATGCATGAGGCCCTGCACAATCATTACACCC |
| | | | AGAAGTCCCTGTCTCTGAGTCCCGGC |
| 16. | 2309 | Full | GATATTCAGATGACCCAGAGCCCAAGCTCCCTGAGTGCCTCAGTGGGCGACCGAGTCACCATCACATGCTCCG |
| | | | CTTCTAGTTCAGTGTCTTACATGAACTGGTATCAGCAGACTCCAGGGAAGGCACCCAAACGGTGGATCTACGA |
| | | | TACCTCAAAGCTGGCCAGCGGAGTGCCCTCCAGATTCAGCGGCTCCGGGTCTGGAACAGACTATACTTTTACC |
| | | | ATCAGCTCCCTGCAGCCTGAGGATATTGCTACTTACTATTGCCAGCAGTGGTCTAGTAATCCATTCACTTTTG |
| | | | GCCAGGGGACCAAGCTGCAGATCACAAGGACTGTGGCCGCTCCCAGCGTCTTCATTTTTCCCCCTAGCGACGA |
| | | | GCAGCTGAAATCTGGCACAGCCAGTGTGGTCTGTCTGCTGAACAATTTCTACCCTCGCGAAGCAAAGGTGCAG |
| | | | TGGAAAGTCGATAACGCCCTGCAGAGTGGCAACAGCCAGGAGAGCGTGACAGAACAGGACTCCAAGGATTCTA |
| | | | CTTATAGTCTGTCAAGCACCCTGACACTGTCCAAAGCTGACTACGAGAAGCACAAAGTGTATGCATGCGAAGT |
| | | | CACCCATCAGGGACTGTCCTCTCCTGTGACAAAATCTTTTAACAGAGGCGAATGT |
| 17. | 2309 | VL | GATATTCAGATGACCCAGAGCCCAAGCTCCCTGAGTGCCTCAGTGGGCGACCGAGTCACCATCACATGCTCCG |
| | | | CTTCTAGTTCAGTGTCTTACATGAACTGGTATCAGCAGACTCCAGGGAAGGCACCCAAACGGTGGATCTACGA |
| | | | TACCTCAAAGCTGGCCAGCGGAGTGCCCTCCAGATTCAGCGGCTCCGGGTCTGGAACAGACTATACTTTTACC |
| | | | ATCAGCTCCCTGCAGCCTGAGGATATTGCTACTTACTATTGCCAGCAGTGGTCTAGTAATCCATTCACTTTTG |
| | | | GCCAGGGGACCAAGCTGCAGATCACA |
| 18. | 2309 | CL | AGGACTGTGGCCGCTCCCAGCGTCTTCATTTTTCCCCCTAGCGACGAGCAGCTGAAATCTGGCACAGCCAGTG |
| | | | TGGTCTGTCTGCTGAACAATTTCTACCCTCGCGAAGCAAAGGTGCAGTGGAAAGTCGATAACGCCCTGCAGAG |
| | | | TGGCAACAGCCAGGAGAGCGTGACAGAACAGGACTCCAAGGATTCTACTTATAGTCTGTCAAGCACCCTGACA |
| | | | CTGTCCAAAGCTGACTACGAGAAGCACAAAGTGTATGCATGCGAAGTCACCCATCAGGGACTGTCCTCTCCTG |
| | | | TGACAAAATCTTTTAACAGAGGCGAATGT |
| 19. | 2310 | Full | GATATTCAGCTGACTCAGTCACCCGCTAGCCTGGCAGTGAGTCTGGGCCAGAGGGCCACCATCAGCTGCAAGG |
| | | | CTTCACAGAGCGTCGACTACGATGGCGACAGCTACCTGAACTGGTATCAGCAGATCCCTGGGCAGCCCCCTAA |
| | | | ACTGCTGATCTACGACGCCTCTAATCTGGTGAGTGGCATCCCCCCACGCTTCTCCGGCTCTGGGAGTGGAACT |
| | | | GATTTTACCCTGAACATTCACCCCGTGGAGAAGGTCGACGCCGCTACATACCATTGCCAGCAGTCCACAGAGG |
| | | | ACCCCTGGACTTTCGGCGGGGGAACCAAGCTGGAAATCAAACGGACAGTGGCAGCCCCATCCGTCTTCATTTT |
| | | | TCCTCCATCTGACGAGCAGCTGAAATCAGGGACTGCTAGCGTGGTCTGTCTGCTGAACAATTTTTACCCAAGA |
| | | | GAAGCAAAGGTGCAGTGGAAAGTCGATAACGCCCTGCAGTCCGGAAATTCTCAGGAGAGTGTGACAGAACAGG |
| | | | ATTCAAAGGACAGCACTTATTCCCTGAGCTCCACCCTGACACTGTCCAAAGCTGATTACGAGAAGCACAAAGT |
| | | | GTATGCATGCGAAGTCACCCATCAGGGACTGTCTAGTCCCGTGACAAAGTCTTTCAATCGAGGCGAATGT |
| 20. | 2310 | VL | GATATTCAGCTGACTCAGTCACCCGCTAGCCTGGCAGTGAGTCTGGGCCAGAGGGCCACCATCAGCTGCAAGG |
| | | | CTTCACAGAGCGTCGACTACGATGGCGACAGCTACCTGAACTGGTATCAGCAGATCCCTGGGCAGCCCCCTAA |
| | | | ACTGCTGATCTACGACGCCTCTAATCTGGTGAGTGGCATCCCCCCACGCTTCTCCGGCTCTGGGAGTGGAACT |
| | | | GATTTTACCCTGAACATTCACCCCGTGGAGAAGGTCGACGCCGCTACATACCATTGCCAGCAGTCCACAGAGG |
| | | | ACCCCTGGACTTTCGGCGGGGGAACCAAGCTGGAAATCAAA |
| 21. | 2310 | CL | CGGACAGTGGCAGCCCCATCCGTCTTCATTTTTCCTCCATCTGACGAGCAGCTGAAATCAGGGACTGCTAGCG |
| | | | TGGTCTGTCTGCTGAACAATTTTTACCCAAGAGAAGCAAAGGTGCAGTGGAAAGTCGATAACGCCCTGCAGTC |
| | | | CGGAAATTCTCAGGAGAGTGTGACAGAACAGGATTCAAAGGACAGCACTTATTCCCTGAGCTCCACCCTGACA |
| | | | CTGTCCAAAGCTGATTACGAGAAGCACAAAGTGTATGCATGCGAAGTCACCCATCAGGGACTGTCTAGTCCCG |
| | | | TGACAAAGTCTTTCAATCGAGGCGAATGT |
| 22. | 2183 | Full | GATATTCAGCTGACACAGAGTCCTGCATCACTGGCTGTGAGCCTGGGACAGCGAGCAACTATCTCCTGCAAAG |
| | | | CCAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAGGGAGGAGGAGGCAGTGGCGGAGGAGGGTCAGG |
| | | | AGGAGGAGGAAGCCAGGTGCAGCTGCAGCAGAGCGGAGCAGAGCTGGTCAGACCAGGAAGCTCCGTGAAAATT |
| | | | TCCTGTAAGGCTTCTGGCTATGCATTTTCTAGTTACTGGATGAATTGGGTGAAGCAGAGGCCAGGACAGGGCC |
| | | | TGGAATGGATCGGGCAGATTTGGCCCGGGGATGGAGACACCAACTATAATGGAAAGTTCAAAGGCAAGGCCAC |
| | | | ACTGACTGCTGACGAGTCAAGCTCCACAGCCTATATGCAGCTGTCTAGTCTGGCAAGCGAGGATTCCGCCGTG |
| | | | TACTTTTGCGCTCGGAGAGAAACCACAACTGTGGGCAGGTACTATTACGCTATGGACTACTGGGGCCAGGGGA |
| | | | CCACAGTCACCGTGTCAAGCGCAGCCGAACCCAAATCCTCTGATAAGACCCACACATGCCCTCCATGTCCAGC |
| | | | TCCTGAGGCTGCAGGAGGACCAAGCGTGTTCCTGTTTCCCCCTAAACCTAAGGACACACTGATGATCTCTCGG |
| | | | ACACCCGAAGTCACTTGTGTGGTCGTGAGCGTGAGCCACGAGGACCCTGAAGTCAAATTCAACTGGTACGTGG |
| | | | ATGGCGTCGAGGTGCATAATGCCAAAACTAAGCCTAGGGAGGAACAGTATAACTCCACTTACCGCGTCGTGTC |
| | | | TGTCCTGACCGTGCTGCATCAGGACTGGCTGAACGGAAAGGAGTACAAATGCAAGGTGAGCAACAAGGCACTG |
| | | | CCAGCCCCCATCGAGAAGACAATTTCCAAAGCAAAGGGCCAGCCTCGAGAACCACAGGTCTATGTGTACCCAC |
| | | | CCAGCCGGGACGAGCTGACCAAAAACCAGGTCTCCCTGACATGTCTGGTGAAGGGATTTTATCCTTCTGATAT |
| | | | TGCCGTGGAGTGGGAAAGTAATGGCCAGCCAGAAAACAATTACAAGACTACCCCTCCAGTGCTGGATTCTGAC |
| | | | GGGAGTTTCGCTCTGGTCAGTAAACTGACTGTGGATAAGTCACGGTGGCAGCAGGGAAACGTCTTTAGTTGTT |
| | | | CAGTGATGCACGAGGCACTGCACAATCATTACACCCAGAAAAGCCTGTCCCTGTCTCCCGGCAAG |
| 23. | 2183 | VL | GATATTCAGCTGACACAGAGTCCTGCATCACTGGCTGTGAGCCTGGGACAGCGAGCAACTATCTCCTGCAAAG |
| | | | CCAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAG |
| 24. | 2183 | VH | CAGGTGCAGCTGCAGCAGAGCGGAGCAGAGCTGGTCAGACCAGGAAGCTCCGTGAAAATTTCCTGTAAGGCTT |
| | | | CTGGCTATGCATTTTCTAGTTACTGGATGAATTGGGTGAAGCAGAGGCCAGGACAGGGCCTGGAATGGATCGG |
| | | | GCAGATTTGGCCCGGGGATGGAGACACCAACTATAATGGAAAGTTCAAAGGCAAGGCCACACTGACTGCTGAC |
| | | | GAGTCAAGCTCCACAGCCTATATGCAGCTGTCTAGTCTGGCAAGCGAGGATTCCGCCGTGTACTTTTGCGCTC |
| | | | GGAGAGAAACCACAACTGTGGGCAGGTACTATTACGCTATGGACTACTGGGGCCAGGGGACCACAGTCACCGT |
| | | | GTCAAGC |
| 25. | 2183 | CH2 | GCTCCTGAGGCTGCAGGAGGACCAAGCGTGTTCCTGTTTCCCCCTAAACCTAAGGACACACTGATGATCTCTC |
| | | | GGACACCCGAAGTCACTTGTGTGGTCGTGAGCGTGAGCCACGAGGACCCTGAAGTCAAATTCAACTGGTACGT |
| | | | GGATGGCGTCGAGGTGCATAATGCCAAAACTAAGCCTAGGGAGGAACAGTATAACTCCACTTACCGCGTCGTG |
| | | | TCTGTCCTGACCGTGCTGCATCAGGACTGGCTGAACGGAAAGGAGTACAAATGCAAGGTGAGCAACAAGGCAC |
| | | | TGCCAGCCCCCATCGAGAAGACAATTTCCAAAGCAAAG |
| 26. | 2183 | CH3 | GGCCAGCCTCGAGAACCACAGGTCTATGTGTACCCACCCAGCCGGGACGAGCTGACCAAAAACCAGGTCTCCC |
| | | | TGACATGTCTGGTGAAGGGATTTTATCCTTCTGATATTGCCGTGGAGTGGGAAAGTAATGGCCAGCCAGAAAA |
| | | | CAATTACAAGACTACCCCTCCAGTGCTGGATTCTGACGGGAGTTTCGCTCTGGTCAGTAAACTGACTGTGGAT |
| | | | AAGTCACGGTGGCAGCAGGGAAACGTCTTTAGTTGTTCAGTGATGCACGAGGCACTGCACAATCATTACACCC |
| | | | AGAAAAGCCTGTCCCTGTCTCCCGGC |
| 27. | 2312 | Full | GACATTGTGATGACACAGTCCCCTGCCACTCTGAGTCTGTCACCAGGCGAGCGGGCTACCCTGAGTTGCAGAA |
| | | | GCTCCAAGAGCCTGCAGAACGTGAATGGAAACACATACCTGTATTGGTTCCAGCAGAAACCAGGCCAGTCTCC |
| | | | CCAGCTGCTGATCTACAGGATGTCAAATCTGAACAGCGGAGTGCCTGACCGCTTCAGCGGCTCCGGGTCTGGA |
| | | | ACCGAGTTCACCCTGACAATTTCTAGTCTGGAGCCCGAAGATTTCGCAGTCTACTATTGCATGCAGCACCTGG |
| | | | AGTATCCTATCACCTTTGGCGCTGGGACAAAGCTGGAGATCAAGCGAACTGTGGCCGCTCCATCCGTCTTCAT |
| | | | CTTTCCCCCTTCTGACGAGCAGCTGAAGTCCGGCACAGCCTCTGTGGTCTGTCTGCTGAACAATTTCTACCCC |
| | | | AGAGAAGCAAAGGTGCAGTGGAAAGTCGATAATGCCCTGCAGAGTGGGAACTCACAGGAGAGCGTGACTGAAC |
| | | | AGGACTCCAAGGATTCTACCTATAGTCTGTCAAGCACTCTGACCCTGAGCAAAGCTGACTACGAGAAGCACAA |
| | | | AGTGTATGCATGCGAAGTCACACATCAGGGGCTGTCCTCTCCCGTGACTAAAAGCTTTAATCGGGGAGAGTGT |
| 28. | 2312 | VL | GACATTGTGATGACACAGTCCCCTGCCACTCTGAGTCTGTCACCAGGCGAGCGGGCTACCCTGAGTTGCAGAA |
| | | | GCTCCAAGAGCCTGCAGAACGTGAATGGAAACACATACCTGTATTGGTTCCAGCAGAAACCAGGCCAGTCTCC |
| | | | CCAGCTGCTGATCTACAGGATGTCAAATCTGAACAGCGGAGTGCCTGACCGCTTCAGCGGCTCCGGGTCTGGA |
| | | | ACCGAGTTCACCCTGACAATTTCTAGTCTGGAGCCCGAAGATTTCGCAGTCTACTATTGCATGCAGCACCTGG |
| | | | AGTATCCTATCACCTTTGGCGCTGGGACAAAGCTGGAGATCAAG |
| 29. | 2312 | CL | CGAACTGTGGCCGCTCCATCCGTCTTCATCTTTCCCCCTTCTGACGAGCAGCTGAAGTCCGGCACAGCCTCTG |
| | | | TGGTCTGTCTGCTGAACAATTTCTACCCCAGAGAAGCAAAGGTGCAGTGGAAAGTCGATAATGCCCTGCAGAG |
| | | | TGGGAACTCACAGGAGAGCGTGACTGAACAGGACTCCAAGGATTCTACCTATAGTCTGTCAAGCACTCTGACC |
| | | | CTGAGCAAAGCTGACTACGAGAAGCACAAAGTGTATGCATGCGAAGTCACACATCAGGGGCTGTCCTCTCCCG |
| | | | TGACTAAAAGCTTTAATCGGGGAGAGTGT |
| 30. | 2313 | Full | CAGGTCCAGCTGGTGGAATCCGGAGGAGGAGTGGTCCAGCCTGGACGATCTCTGAGACTGAGTTGCGCCGCTT |
| | | | CAGGGTTCAAGTTTAGCGGGTACGGAATGCACTGGGTGAGGCAGGCACCAGGCAAAGGGCTGGAGTGGGTCGC |
| | | | CGTGATCTGGTATGACGGCAGCAAGAAGTACTATGTCGATTCTGTGAAGGGCAGGTTCACCATTAGCCGCGAC |
| | | | AACTCCAAAAATACACTGTACCTGCAGATGAACTCCCTGAGAGCCGAAGACACCGCTGTGTACTATTGCGCCA |
| | | | GGCAGATGGGCTATTGGCACTTCGATCTGTGGGGACGAGGAACCCTGGTCACAGTGAGCTCCGCATCTACAAA |
| | | | GGGGCCCAGTGTGTTTCCACTGGCTCCCTCTAGTAAATCCACTTCTGGAGGAACCGCAGCACTGGGATGTCTG |
| | | | GTGAAGGATTACTTCCCAGAGCCCGTCACCGTGAGTTGGAACTCAGGGGCTCTGACCTCCGGAGTCCATACAT |
| | | | TTCCAGCAGTGCTGCAGTCAAGCGGCCTGTACAGCCTGTCCTCTGTGGTCACTGTGCCCAGTTCAAGCCTGGG |
| | | | GACTCAGACCTATATCTGCAACGTGAATCACAAGCCATCAAATACCAAAGTCGACAAGAAAGTGGAACCCAAG |
| | | | AGCTGTGATAAAACACATACTTGCCCACCTTGTCCTGCACCAGAGCTGCTGGGAGGACCAAGCGTGTTCCTGT |
| | | | TTCCACCCAAGCCTAAAGACACTCTGATGATTTCCCGGACACCCGAAGTGACTTGCGTGGTCGTGGACGTGTC |
| | | | TCACGAGGACCCCGAAGTCAAGTTCAACTGGTACGTGGATGGCGTCGAGGTGCATAATGCTAAGACAAAACCC |
| | | | CGAGAGGAACAGTACAATTCAACATATCGGGTCGTGAGCGTCCTGACTGTGCTGCACCAGGACTGGCTGAACG |
| | | | GCAAGGAGTATAAGTGCAAAGTGAGTAATAAGGCTCTGCCCGCACCTATCGAGAAAACCATTTCTAAGGCTAA |
| | | | AGGGCAGCCTCGCGAACCACAGGTCTACGTGTATCCTCCATCTCGAGACGAGCTGACTAAGAACCAGGTCAGT |
| | | | CTGACCTGTCTGGTGAAAGGGTTTTACCCTAGCGATATCGCAGTGGAGTGGGAATCCAATGGACAGCCAGAAA |
| | | | ACAATTATAAGACCACACCCCCTGTGCTGGACAGCGATGGCAGCTTCGCACTGGTCAGTAAGCTGACAGTGGA |
| | | | TAAATCAAGATGGCAGCAGGGCAACGTCTTTAGTTGTTCAGTGATGCATGAGGCCCTGCACAATCATTACACT |
| | | | CAGAAGAGCCTGTCCCTGTCTCCTGGCAAA |
| 31. | 2313 | VH | CAGGTCCAGCTGGTGGAATCCGGAGGAGGAGTGGTCCAGCCTGGACGATCTCTGAGACTGAGTTGCGCCGCTT |
| | | | CAGGGTTCAAGTTTAGCGGGTACGGAATGCACTGGGTGAGGCAGGCACCAGGCAAAGGGCTGGAGTGGGTCGC |
| | | | CGTGATCTGGTATGACGGCAGCAAGAAGTACTATGTCGATTCTGTGAAGGGCAGGTTCACCATTAGCCGCGAC |
| | | | AACTCCAAAAATACACTGTACCTGCAGATGAACTCCCTGAGAGCCGAAGACACCGCTGTGTACTATTGCGCCA |
| | | | GGCAGATGGGCTATTGGCACTTCGATCTGTGGGGACGAGGAACCCTGGTCACAGTGAGCTCC |
| 32. | 2313 | CH1 | GCATCTACAAAGGGGCCCAGTGTGTTTCCACTGGCTCCCTCTAGTAAATCCACTTCTGGAGGAACCGCAGCAC |
| | | | TGGGATGTCTGGTGAAGGATTACTTCCCAGAGCCCGTCACCGTGAGTTGGAACTCAGGGGCTCTGACCTCCGG |
| | | | AGTCCATACATTTCCAGCAGTGCTGCAGTCAAGCGGCCTGTACAGCCTGTCCTCTGTGGTCACTGTGCCCAGT |
| | | | TCAAGCCTGGGGACTCAGACCTATATCTGCAACGTGAATCACAAGCCATCAAATACCAAAGTCGACAAGAAAG |
| | | | TG |
| 33. | 2313 | CH2 | GCACCAGAGCTGCTGGGAGGACCAAGCGTGTTCCTGTTTCCACCCAAGCCTAAAGACACTCTGATGATTTCCC |
| | | | GGACACCCGAAGTGACTTGCGTGGTCGTGGACGTGTCTCACGAGGACCCCGAAGTCAAGTTCAACTGGTACGT |
| | | | GGATGGCGTCGAGGTGCATAATGCTAAGACAAAACCCCGAGAGGAACAGTACAATTCAACATATCGGGTCGTG |
| | | | AGCGTCCTGACTGTGCTGCACCAGGACTGGCTGAACGGCAAGGAGTATAAGTGCAAAGTGAGTAATAAGGCTC |
| | | | TGCCCGCACCTATCGAGAAAACCATTTCTAAGGCTAAA |
| 34. | 2313 | CH3 | GGGCAGCCTCGCGAACCACAGGTCTACGTGTATCCTCCATCTCGAGACGAGCTGACTAAGAACCAGGTCAGTC |
| | | | TGACCTGTCTGGTGAAAGGGTTTTACCCTAGCGATATCGCAGTGGAGTGGGAATCCAATGGACAGCCAGAAAA |
| | | | CAATTATAAGACCACACCCCCTGTGCTGGACAGCGATGGCAGCTTCGCACTGGTCAGTAAGCTGACAGTGGAT |
| | | | AAATCAAGATGGCAGCAGGGCAACGTCTTTAGTTGTTCAGTGATGCATGAGGCCCTGCACAATCATTACACTC |
| | | | AGAAGAGCCTGTCCCTGTCTCCTGGC |
| 35. | 2316 | Full | CAGGCTTACCTGCAGCAGTCCGGAGCAGAACTGGTCCGACCAGGAGCTTCCGTGAAAATGTCTTGCAAAGCAA |
| | | | GTGGCTACACTTTCACCAGCTATAACATGCACTGGGTGAAACAGACACCTCGACAGGGACTGGAGTGGATCGG |
| | | | AGCAATCTACCCAGGGAACGGAGACACTAGCTATAATCAGAAGTTTAAAGGGAAGGCTACACTGACTGTGGAT |
| | | | AAGAGCTCCTCTACTGCATACATGCAGCTGAGTTCACTGACCAGCGAAGACTCCGCTGTGTATTTCTGCGCAA |
| | | | GGGTGGTCTACTACTCCAATTCTTACTGGTACTTCGATGTGTGGGGCACTGGGACCACAGTCACCGTGAGCTC |
| | | | CGCCTCAACCAAAGGACCTAGCGTGTTCCCACTGGCTCCCTCTAGTAAGAGTACATCAGGAGGAACTGCAGCT |
| | | | CTGGGATGTCTGGTGAAGGACTACTTCCCAGAGCCCGTCACAGTGTCTTGGAACAGTGGGGCACTGACATCTG |
| | | | GAGTCCATACTTTTCCTGCCGTGCTGCAGTCAAGCGGGCTGTACAGCCTGTCCTCTGTGGTCACTGTGCCAAG |
| | | | TTCAAGCCTGGGAACCCAGACATATATCTGCAACGTGAATCACAAACCAAGCAATACCAAGGTCGACAAGAAA |
| | | | GTGGAACCCAAATCCTGTGATAAGACTCATACCTGCCCACCTTGTCCTGCACCAGAGCTGCTGGGAGGACCAT |
| | | | CCGTGTTCCTGTTTCCACCCAAACCTAAGGACACCCTGATGATTTCTAGAACCCCAGAAGTCACATGCGTGGT |
| | | | CGTGGACGTGAGCCACGAGGACCCCGAAGTCAAGTTTAACTGGTACGTGGATGGCGTCGAGGTGCATAATGCT |
| | | | AAAACAAAGCCCCGGGAGGAACAGTACAACTCCACCTATAGAGTCGTGTCTGTCCTGACAGTGCTGCACCAGG |
| | | | ACTGGCTGAACGGGAAGGAGTATAAATGCAAGGTGAGCAACAAGGCACTGCCCGCCCCTATCGAGAAGACAAT |
| | | | TTCCAAAGCTAAGGGACAGCCTAGGGAACCACAGGTCTACGTGCTGCCTCCATCTCGCGACGAGCTGACTAAA |
| | | | AACCAGGTCAGTCTGCTGTGTCTGGTGAAGGGATTCTATCCCAGCGATATCGCAGTGGAGTGGGAATCCAATG |
| | | | GCCAGCCTGAAAACAATTACCTGACCTGGCCCCCTGTGCTGGACTCAGATGGCAGCTTCTTTCTGTATAGTAA |
| | | | ACTGACAGTGGATAAGTCACGCTGGCAGCAGGGGAACGTCTTTAGCTGTTCCGTGATGCATGAGGCCCTGCAC |
| | | | AATCATTACACCCAGAAATCTCTGAGTCTGTCACCCGGCAAG |
| 36. | 2316 | VH | CAGGCTTACCTGCAGCAGTCCGGAGCAGAACTGGTCCGACCAGGAGCTTCCGTGAAAATGTCTTGCAAAGCAA |
| | | | GTGGCTACACTTTCACCAGCTATAACATGCACTGGGTGAAACAGACACCTCGACAGGGACTGGAGTGGATCGG |
| | | | AGCAATCTACCCAGGGAACGGAGACACTAGCTATAATCAGAAGTTTAAAGGGAAGGCTACACTGACTGTGGAT |
| | | | AAGAGCTCCTCTACTGCATACATGCAGCTGAGTTCACTGACCAGCGAAGACTCCGCTGTGTATTTCTGCGCAA |
| | | | GGGTGGTCTACTACTCCAATTCTTACTGGTACTTCGATGTGTGGGGCACTGGGACCACAGTCACCGTGAGCTC |
| | | | C |
| 37. | 2316 | CH1 | GCCTCAACCAAAGGACCTAGCGTGTTCCCACTGGCTCCCTCTAGTAAGAGTACATCAGGAGGAACTGCAGCTC |
| | | | TGGGATGTCTGGTGAAGGACTACTTCCCAGAGCCCGTCACAGTGTCTTGGAACAGTGGGGCACTGACATCTGG |
| | | | AGTCCATACTTTTCCTGCCGTGCTGCAGTCAAGCGGGCTGTACAGCCTGTCCTCTGTGGTCACTGTGCCAAGT |
| | | | TCAAGCCTGGGAACCCAGACATATATCTGCAACGTGAATCACAAACCAAGCAATACCAAGGTCGACAAGAAAG |
| | | | TG |
| 38. | 2316 | CH2 | GCACCAGAGCTGCTGGGAGGACCATCCGTGTTCCTGTTTCCACCCAAACCTAAGGACACCCTGATGATTTCTA |
| | | | GAACCCCAGAAGTCACATGCGTGGTCGTGGACGTGAGCCACGAGGACCCCGAAGTCAAGTTTAACTGGTACGT |
| | | | GGATGGCGTCGAGGTGCATAATGCTAAAACAAAGCCCCGGGAGGAACAGTACAACTCCACCTATAGAGTCGTG |
| | | | TCTGTCCTGACAGTGCTGCACCAGGACTGGCTGAACGGGAAGGAGTATAAATGCAAGGTGAGCAACAAGGCAC |
| | | | TGCCCGCCCCTATCGAGAAGACAATTTCCAAAGCTAAG |
| 39. | 2316 | CH3 | GGACAGCCTAGGGAACCACAGGTCTACGTGCTGCCTCCATCTCGCGACGAGCTGACTAAAAACCAGGTCAGTC |
| | | | TGCTGTGTCTGGTGAAGGGATTCTATCCCAGCGATATCGCAGTGGAGTGGGAATCCAATGGCCAGCCTGAAAA |
| | | | CAATTACCTGACCTGGCCCCCTGTGCTGGACTCAGATGGCAGCTTCTTTCTGTATAGTAAACTGACAGTGGAT |
| | | | AAGTCACGCTGGCAGCAGGGGAACGTCTTTAGCTGTTCCGTGATGCATGAGGCCCTGCACAATCATTACACCC |
| | | | AGAAATCTCTGAGTCTGTCACCCGGC |
| 40. | 2317 | Full | GAAGTCCAGCTGGTCGAATCTGGAGGAGGACTGGTGCAGCCTGGACGATCCCTGAGACTGTCTTGCGCCGCTA |
| | | | GTGGCTTCACTTTTAACGACTATGCAATGCACTGGGTGCGCCAGGCACCAGGGAAGGGACTGGAGTGGGTGAG |
| | | | CACCATCTCCTGGAACAGCGGATCTATTGGCTATGCAGACAGCGTGAAAGGCAGGTTCACAATCAGTCGCGAT |
| | | | AACGCCAAGAAATCACTGTACCTGCAGATGAATAGCCTGCGAGCCGAAGACACAGCTCTGTACTATTGCGCCA |
| | | | AGGATATTCAGTATGGGAACTACTATTACGGAATGGACGTGTGGGGCCAGGGGACCACAGTCACCGTGAGCTC |
| | | | CGCCTCAACAAAGGGGCCCAGCGTGTTTCCACTGGCTCCCTCTAGTAAAAGTACCTCAGGCGGGACAGCAGCC |
| | | | CTGGGATGTCTGGTGAAGGATTACTTCCCAGAGCCCGTCACCGTGTCTTGGAACAGTGGCGCTCTGACAAGCG |
| | | | GGGTCCATACTTTTCCAGCAGTGCTGCAGTCAAGCGGCCTGTATTCCCTGTCCTCTGTGGTCACTGTGCCCAG |
| | | | TTCAAGCCTGGGGACTCAGACCTACATCTGCAACGTGAATCACAAGCCATCTAATACCAAAGTCGACAAGAAA |
| | | | GTGGAACCCAAGAGTTGTGATAAAACACATACTTGCCCACCTTGTCCTGCACCAGAGCTGCTGGGAGGACCAT |
| | | | CCGTGTTCCTGTTTCCACCCAAGCCTAAAGACACCCTGATGATTAGCAGGACTCCCGAAGTCACCTGCGTGGT |
| | | | CGTGGACGTGTCCCACGAGGACCCCGAAGTCAAGTTCAACTGGTACGTGGATGGCGTCGAGGTGCATAATGCT |
| | | | AAGACAAAACCCCGAGAGGAACAGTATAATTCCACTTACCGGGTCGTGTCTGTCCTGACCGTGCTGCACCAGG |
| | | | ACTGGCTGAACGGCAAGGAGTACAAGTGCAAAGTGTCTAATAAGGCTCTGCCCGCACCTATCGAGAAAACAAT |
| | | | TAGCAAGGCTAAAGGGCAGCCTAGAGAACCACAGGTCTATGTGCTGCCTCCAAGCAGGGACGAGCTGACTAAG |
| | | | AACCAGGTCTCCCTGCTGTGTCTGGTGAAAGGGTTCTACCCTAGTGATATCGCAGTGGAGTGGGAATCAAATG |
| | | | GACAGCCAGAAAACAATTATCTGACATGGCCCCCTGTGCTGGACTCAGATGGAAGCTTCTTTCTGTACTCCAA |
| | | | GCTGACTGTGGATAAATCTCGGTGGCAGCAGGGCAACGTCTTTAGCTGTTCCGTGATGCATGAGGCCCTGCAC |
| | | | AATCATTACACCCAGAAGTCTCTGAGTCTGTCACCTGGCAAA |
| 41. | 2317 | VH | GAAGTCCAGCTGGTCGAATCTGGAGGAGGACTGGTGCAGCCTGGACGATCCCTGAGACTGTCTTGCGCCGCTA |
| | | | GTGGCTTCACTTTTAACGACTATGCAATGCACTGGGTGCGCCAGGCACCAGGGAAGGGACTGGAGTGGGTGAG |
| | | | CACCATCTCCTGGAACAGCGGATCTATTGGCTATGCAGACAGCGTGAAAGGCAGGTTCACAATCAGTCGCGAT |
| | | | AACGCCAAGAAATCACTGTACCTGCAGATGAATAGCCTGCGAGCCGAAGACACAGCTCTGTACTATTGCGCCA |
| | | | AGGATATTCAGTATGGGAACTACTATTACGGAATGGACGTGTGGGGCCAGGGGACCACAGTCACCGTGAGCTC |
| | | | C |
| 42. | 2317 | CH1 | GCCTCAACAAAGGGGCCCAGCGTGTTTCCACTGGCTCCCTCTAGTAAAAGTACCTCAGGCGGGACAGCAGCCC |
| | | | TGGGATGTCTGGTGAAGGATTACTTCCCAGAGCCCGTCACCGTGTCTTGGAACAGTGGCGCTCTGACAAGCGG |
| | | | GGTCCATACTTTTCCAGCAGTGCTGCAGTCAAGCGGCCTGTATTCCCTGTCCTCTGTGGTCACTGTGCCCAGT |
| | | | TCAAGCCTGGGGACTCAGACCTACATCTGCAACGTGAATCACAAGCCATCTAATACCAAAGTCGACAAGAAAG |
| | | | TG |
| 43. | 2317 | CH2 | GCACCAGAGCTGCTGGGAGGACCATCCGTGTTCCTGTTTCCACCCAAGCCTAAAGACACCCTGATGATTAGCA |
| | | | GGACTCCCGAAGTCACCTGCGTGGTCGTGGACGTGTCCCACGAGGACCCCGAAGTCAAGTTCAACTGGTACGT |
| | | | GGATGGCGTCGAGGTGCATAATGCTAAGACAAAACCCCGAGAGGAACAGTATAATTCCACTTACCGGGTCGTG |
| | | | TCTGTCCTGACCGTGCTGCACCAGGACTGGCTGAACGGCAAGGAGTACAAGTGCAAAGTGTCTAATAAGGCTC |
| | | | TGCCCGCACCTATCGAGAAAACAATTAGCAAGGCTAAA |
| 44. | 2317 | CH3 | GGGCAGCCTAGAGAACCACAGGTCTATGTGCTGCCTCCAAGCAGGGACGAGCTGACTAAGAACCAGGTCTCCC |
| | | | TGCTGTGTCTGGTGAAAGGGTTCTACCCTAGTGATATCGCAGTGGAGTGGGAATCAAATGGACAGCCAGAAAA |
| | | | CAATTATCTGACATGGCCCCCTGTGCTGGACTCAGATGGAAGCTTCTTTCTGTACTCCAAGCTGACTGTGGAT |
| | | | AAATCTCGGTGGCAGCAGGGCAACGTCTTTAGCTGTTCCGTGATGCATGAGGCCCTGCACAATCATTACACCC |
| | | | AGAAGTCTCTGAGTCTGTCACCTGGC |
| 45. | 2323 | Full | CAGATCGTCCTGTCACAGAGCCCCGCTATCCTGTCCGCATCTCCTGGCGAGAAGGTGACCATGACATGCCGAG |
| | | | CTAGCTCCTCTGTCTCCTACATGCACTGGTATCAGCAGAAGCCCGGGAGTTCACCTAAACCATGGATCTACGC |
| | | | CCCATCAAACCTGGCTAGCGGAGTGCCAGCACGGTTCAGTGGCTCAGGGAGCGGAACATCCTATTCTCTGACT |
| | | | ATTTCTAGAGTGGAGGCTGAAGACGCCGCTACCTACTATTGCCAGCAGTGGTCCTTCAATCCCCCTACCTTTG |
| | | | GCGCAGGGACAAAGCTGGAGCTGAAAAGGACCGTGGCAGCCCCTAGTGTCTTCATTTTTCCACCCTCCGACGA |
| | | | ACAGCTGAAGTCCGGCACAGCCTCTGTGGTCTGTCTGCTGAACAATTTCTACCCACGCGAGGCCAAGGTGCAG |
| | | | TGGAAAGTCGATAACGCTCTGCAGAGTGGCAACAGCCAGGAGAGCGTGACTGAACAGGACTCCAAGGATTCTA |
| | | | CCTATAGTCTGAGCTCCACTCTGACCCTGAGCAAAGCAGATTACGAGAAGCACAAAGTGTATGCCTGCGAAGT |
| | | | CACACATCAGGGACTGTCTAGTCCTGTGACTAAAAGCTTTAACAGAGGCGAATGT |
| 46. | 2323 | VL | CAGATCGTCCTGTCACAGAGCCCCGCTATCCTGTCCGCATCTCCTGGCGAGAAGGTGACCATGACATGCCGAG |
| | | | CTAGCTCCTCTGTCTCCTACATGCACTGGTATCAGCAGAAGCCCGGGAGTTCACCTAAACCATGGATCTACGC |
| | | | CCCATCAAACCTGGCTAGCGGAGTGCCAGCACGGTTCAGTGGCTCAGGGAGCGGAACATCCTATTCTCTGACT |
| | | | ATTTCTAGAGTGGAGGCTGAAGACGCCGCTACCTACTATTGCCAGCAGTGGTCCTTCAATCCCCCTACCTTTG |
| | | | GCGCAGGGACAAAGCTGGAGCTGAAA |
| 47. | 2323 | CL | AGGACCGTGGCAGCCCCTAGTGTCTTCATTTTTCCACCCTCCGACGAACAGCTGAAGTCCGGCACAGCCTCTG |
| | | | TGGTCTGTCTGCTGAACAATTTCTACCCACGCGAGGCCAAGGTGCAGTGGAAAGTCGATAACGCTCTGCAGAG |
| | | | TGGCAACAGCCAGGAGAGCGTGACTGAACAGGACTCCAAGGATTCTACCTATAGTCTGAGCTCCACTCTGACC |
| | | | CTGAGCAAAGCAGATTACGAGAAGCACAAAGTGTATGCCTGCGAAGTCACACATCAGGGACTGTCTAGTCCTG |
| | | | TGACTAAAAGCTTTAACAGAGGCGAATGT |
| 48. | 2325 | Full | GAAATCGTCCTGACACAGTCCCCTGCCACTCTGAGTCTGTCACCAGGCGAGAGGGCTACCCTGTCTTGCCGCG |
| | | | CAAGCCAGTCCGTGAGCTCCTACCTGGCCTGGTATCAGCAGAAGCCAGGGCAGGCTCCCAGACTGCTGATCTA |
| | | | CGACGCATCCAACCGAGCAACCGGCATCCCCGCACGGTTCTCTGGCAGTGGGTCAGGAACAGACTTTACCCTG |
| | | | ACAATCTCTAGTCTGGAGCCCGAAGATTTCGCTGTGTACTATTGCCAGCAGAGGTCTAATTGGCCTATCACCT |
| | | | TTGGCCAGGGGACACGGCTGGAGATTAAGAGAACTGTGGCCGCTCCAAGTGTCTTCATTTTTCCCCCTAGCGA |
| | | | CGAACAGCTGAAATCCGGCACAGCCTCTGTGGTCTGTCTGCTGAACAATTTCTACCCCCGCGAGGCAAAGGTG |
| | | | CAGTGGAAAGTCGATAACGCCCTGCAGAGCGGCAACAGCCAGGAGTCTGTGACTGAACAGGACAGTAAGGATT |
| | | | CAACCTATAGCCTGTCAAGCACTCTGACCCTGAGCAAAGCTGATTACGAGAAGCACAAAGTGTATGCATGCGA |
| | | | AGTCACACATCAGGGACTGTCCTCTCCCGTCACTAAAAGCTTTAACCGAGGCGAATGT |
| 49. | 2325 | VL | GAAATCGTCCTGACACAGTCCCCTGCCACTCTGAGTCTGTCACCAGGCGAGAGGGCTACCCTGTCTTGCCGCG |
| | | | CAAGCCAGTCCGTGAGCTCCTACCTGGCCTGGTATCAGCAGAAGCCAGGGCAGGCTCCCAGACTGCTGATCTA |
| | | | CGACGCATCCAACCGAGCAACCGGCATCCCCGCACGGTTCTCTGGCAGTGGGTCAGGAACAGACTTTACCCTG |
| | | | ACAATCTCTAGTCTGGAGCCCGAAGATTTCGCTGTGTACTATTGCCAGCAGAGGTCTAATTGGCCTATCACCT |
| | | | TTGGCCAGGGGACACGGCTGGAGATTAAG |
| 50. | 2325 | CL | AGAACTGTGGCCGCTCCAAGTGTCTTCATTTTTCCCCCTAGCGACGAACAGCTGAAATCCGGCACAGCCTCTG |
| | | | TGGTCTGTCTGCTGAACAATTTCTACCCCCGCGAGGCAAAGGTGCAGTGGAAAGTCGATAACGCCCTGCAGAG |
| | | | CGGCAACAGCCAGGAGTCTGTGACTGAACAGGACAGTAAGGATTCAACCTATAGCCTGTCAAGCACTCTGACC |
| | | | CTGAGCAAAGCTGATTACGAGAAGCACAAAGTGTATGCATGCGAAGTCACACATCAGGGACTGTCCTCTCCCG |
| | | | TCACTAAAAGCTTTAACCGAGGCGAATGT |
| 51. | 2170 | Full | GACATCAAACTGCAGCAGAGCGGAGCTGAGCTGGCACGACCAGGAGCCAGTGTGAAAATGTCATGCAAGACAA |
| | | | GCGGCTACACCTTCACACGGTATACTATGCACTGGGTGAAACAGAGACCCGGCCAGGGGCTGGAATGGATCGG |
| | | | ATATATTAACCCTTCCCGAGGCTACACCAACTATAATCAGAAGTTTAAAGACAAGGCCACCCTGACCACAGAT |
| | | | AAGAGCTCCTCTACAGCTTACATGCAGCTGAGTTCACTGACTAGTGAGGACTCAGCTGTGTACTATTGCGCAA |
| | | | GGTACTATGACGATCATTACTGTCTGGATTATTGGGGACAGGGCACTACCCTGACTGTCAGCTCCGTGGAAGG |
| | | | AGGGAGCGGAGGCTCCGGAGGATCTGGCGGGAGTGGAGGCGTGGACGATATCCAGCTGACCCAGTCCCCAGCA |
| | | | ATTATGTCCGCCTCTCCCGGCGAGAAAGTGACTATGACCTGCCGCGCCTCTAGTTCAGTGAGCTACATGAACT |
| | | | GGTATCAGCAGAAATCAGGCACCAGCCCCAAGAGATGGATCTACGACACATCCAAGGTCGCTTCTGGGGTGCC |
| | | | TTATAGGTTCAGTGGGTCAGGAAGCGGCACTTCCTACTCTCTGACCATTAGCTCCATGGAGGCAGAAGATGCC |
| | | | GCTACATACTATTGTCAGCAGTGGTCTAGTAATCCACTGACATTTGGGGCCGGAACTAAACTGGAGCTGAAGG |
| | | | CAGCCGAACCCAAATCAAGCGACAAGACACACACTTGCCCACCTTGTCCAGCACCAGAACTGCTGGGAGGACC |
| | | | TAGCGTGTTCCTGTTTCCACCCAAACCAAAGGATACACTGATGATCAGCCGGACCCCTGAGGTCACATGCGTG |
| | | | GTCGTGGACGTGAGCCACGAGGACCCCGAAGTCAAGTTCAACTGGTACGTGGACGGCGTCGAAGTGCATAATG |
| | | | CCAAAACCAAGCCTAGGGAGGAACAGTACAATAGTACTTATCGCGTCGTGTCAGTCCTGACCGTGCTGCATCA |
| | | | GGATTGGCTGAACGGGAAGGAGTACAAATGCAAGGTGTCCAACAAGGCCCTGCCTGCTCCAATCGAGAAGACC |
| | | | ATTTCTAAAGCAAAGGGCCAGCCCCGAGAACCTCAGGTCTACGTGTATCCTCCATCCCGGGACGAGCTGACCA |
| | | | AAAACCAGGTCTCTCTGACATGTCTGGTGAAGGGGTTTTATCCATCTGATATTGCTGTGGAGTGGGAAAGTAA |
| | | | TGGACAGCCCGAGAACAATTACAAGACAACTCCCCCTGTGCTGGACTCCGATGGATCTTTCGCTCTGGTCAGC |
| | | | AAACTGACAGTGGACAAGTCCAGATGGCAGCAGGGCAACGTCTTTAGTTGTTCAGTGATGCACGAGGCACTGC |
| | | | ACAATCATTACACTCAGAAAAGCCTGTCCCTGTCTCCCGGCAAG |
| 52. | 2170 | VH | GACATCAAACTGCAGCAGAGCGGAGCTGAGCTGGCACGACCAGGAGCCAGTGTGAAAATGTCATGCAAGACAA |
| | | | GCGGCTACACCTTCACACGGTATACTATGCACTGGGTGAAACAGAGACCCGGCCAGGGGCTGGAATGGATCGG |
| | | | ATATATTAACCCTTCCCGAGGCTACACCAACTATAATCAGAAGTTTAAAGACAAGGCCACCCTGACCACAGAT |
| | | | AAGAGCTCCTCTACAGCTTACATGCAGCTGAGTTCACTGACTAGTGAGGACTCAGCTGTGTACTATTGCGCAA |
| | | | GGTACTATGACGATCATTACTGTCTGGATTATTGGGGACAGGGCACTACCCTGACTGTCAGCTCC |
| 53. | 2170 | VL | GATATCCAGCTGACCCAGTCCCCAGCAATTATGTCCGCCTCTCCCGGCGAGAAAGTGACTATGACCTGCCGCG |
| | | | CCTCTAGTTCAGTGAGCTACATGAACTGGTATCAGCAGAAATCAGGCACCAGCCCCAAGAGATGGATCTACGA |
| | | | CACATCCAAGGTCGCTTCTGGGGTGCCTTATAGGTTCAGTGGGTCAGGAAGCGGCACTTCCTACTCTCTGACC |
| | | | ATTAGCTCCATGGAGGCAGAAGATGCCGCTACATACTATTGTCAGCAGTGGTCTAGTAATCCACTGACATTTG |
| | | | GGGCCGGAACTAAACTGGAGCTGAAG |
| 54. | 2170 | CH2 | GCACCAGAACTGCTGGGAGGACCTAGCGTGTTCCTGTTTCCACCCAAACCAAAGGATACACTGATGATCAGCC |
| | | | GGACCCCTGAGGTCACATGCGTGGTCGTGGACGTGAGCCACGAGGACCCCGAAGTCAAGTTCAACTGGTACGT |
| | | | GGACGGCGTCGAAGTGCATAATGCCAAAACCAAGCCTAGGGAGGAACAGTACAATAGTACTTATCGCGTCGTG |
| | | | TCAGTCCTGACCGTGCTGCATCAGGATTGGCTGAACGGGAAGGAGTACAAATGCAAGGTGTCCAACAAGGCCC |
| | | | TGCCTGCTCCAATCGAGAAGACCATTTCTAAAGCAAAG |
| 55. | 2170 | CH3 | GGCCAGCCCCGAGAACCTCAGGTCTACGTGTATCCTCCATCCCGGGACGAGCTGACCAAAAACCAGGTCTCTC |
| | | | TGACATGTCTGGTGAAGGGGTTTTATCCATCTGATATTGCTGTGGAGTGGGAAAGTAATGGACAGCCCGAGAA |
| | | | CAATTACAAGACAACTCCCCCTGTGCTGGACTCCGATGGATCTTTCGCTCTGGTCAGCAAACTGACAGTGGAC |
| | | | AAGTCCAGATGGCAGCAGGGCAACGTCTTTAGTTGTTCAGTGATGCACGAGGCACTGCACAATCATTACACTC |
| | | | AGAAAAGCCTGTCCCTGTCTCCCGGC |
| 56. | 6689 | Full | CAGATCGTCCTGACTCAGAGCCCCGCTATTATGTCCGCTTCCCCTGGAGAAAAGGTCACTATGACTTGTTCCG |
| | | | CCTCTAGTTCCGTCTCCTACATGAACTGGTATCAGCAGAAATCTGGAACAAGTCCCAAGCGATGGATCTACGA |
| | | | CACTTCCAAGCTGGCATCTGGAGTGCCTGCCCACTTCCGAGGCAGCGGCTCTGGGACAAGTTATTCACTGACT |
| | | | ATTTCTGGCATGGAGGCCGAAGATGCCGCTACATACTATTGCCAGCAGTGGAGCTCCAACCCATTCACCTTTG |
| | | | GATGTGGCACAAAGCTGGAGATCAATGGCGGAGGAGGCTCCGGAGGAGGAGGGTCTGGAGGAGGAGGAAGTCA |
| | | | GGTCCAGCTGCAGCAGAGCGGAGCAGAACTGGCTAGACCAGGAGCCAGTGTGAAAATGTCATGCAAGGCCAGC |
| | | | GGCTACACATTCACTCGGTATACCATGCATTGGGTGAAACAGAGACCAGGACAGTGTCTGGAGTGGATCGGCT |
| | | | ACATTAATCCCAGCAGGGGGTACACAAACTACAACCAGAAGTTTAAAGACAAGGCAACCCTGACCACCGATAA |
| | | | GTCTAGTTCAACAGCTTATATGCAGCTGAGCTCCCTGACTTCAGAAGACAGCGCTGTGTACTATTGCGCACGC |
| | | | TACTATGACGATCACTACTGTCTGGATTATTGGGGGCAGGGAACTACCCTGACCGTGTCTAGTGCAGCCGAGC |
| | | | CTAAATCAAGCGACAAGACCCATACATGCCCCCCTTGTCCGGCGCCAGAAGCTGCAGGCGGACCAAGCGTGTT |
| | | | CCTGTTTCCACCCAAACCTAAGGATACTCTGATGATTAGCCGAACTCCTGAGGTCACCTGCGTGGTCGTGAGC |
| | | | GTGTCCCACGAGGACCCAGAAGTCAAGTTCAACTGGTACGTGGATGGGGTCGAAGTGCATAATGCCAAAACCA |
| | | | AGCCCAGGGAGGAACAGTACAACTCCACTTATCGCGTCGTGTCTGTCCTGACCGTGCTGCACCAGGACTGGCT |
| | | | GAATGGCAAGGAGTACAAATGTAAGGTCTCAAATAAGGCTCTGCCCGCCCCTATCGAAAAAACTATCTCAAAG |
| | | | GCAAAAGGCCAGCCTCGCGAACCACAGGTCTACGTGCTGCCCCCTAGCCGCGACGAACTGACTAAAAATCAGG |
| | | | TCTCTCTGCTGTGTCTGGTCAAAGGATTCTACCCTTCCGACATCGCCGTGGAGTGGGAAAGTAACGGCCAGCC |
| | | | CGAGAACAATTACCTGACCTGGCCCCCTGTGCTGGACTCTGATGGGAGTTTCTTTCTGTATTCAAAGCTGACA |
| | | | GTCGATAAAAGCCGGTGGCAGCAGGGCAATGTGTTCAGCTGCTCCGTCATGCACGAAGCACTGCACAACCATT |
| | | | ACACTCAGAAGTCCCTGTCCCTGTCACCTGGC |
| 57. | 6689 | VL | CAGATCGTCCTGACTCAGAGCCCCGCTATTATGTCCGCTTCCCCTGGAGAAAAGGTCACTATGACTTGTTCCG |
| | | | CCTCTAGTTCCGTCTCCTACATGAACTGGTATCAGCAGAAATCTGGAACAAGTCCCAAGCGATGGATCTACGA |
| | | | CACTTCCAAGCTGGCATCTGGAGTGCCTGCCCACTTCCGAGGCAGCGGCTCTGGGACAAGTTATTCACTGACT |
| | | | ATTTCTGGCATGGAGGCCGAAGATGCCGCTACATACTATTGCCAGCAGTGGAGCTCCAACCCATTCACCTTTG |
| | | | GATGTGGCACAAAGCTGGAGATCAAT |
| 58. | 6689 | VH | CAGGTCCAGCTGCAGCAGAGCGGAGCAGAACTGGCTAGACCAGGAGCCAGTGTGAAAATGTCATGCAAGGCCA |
| | | | GCGGCTACACATTCACTCGGTATACCATGCATTGGGTGAAACAGAGACCAGGACAGTGTCTGGAGTGGATCGG |
| | | | CTACATTAATCCCAGCAGGGGGTACACAAACTACAACCAGAAGTTTAAAGACAAGGCAACCCTGACCACCGAT |
| | | | AAGTCTAGTTCAACAGCTTATATGCAGCTGAGCTCCCTGACTTCAGAAGACAGCGCTGTGTACTATTGCGCAC |
| | | | GCTACTATGACGATCACTACTGTCTGGATTATTGGGGGCAGGGAACTACCCTGACCGTGTCTAGT |
| 59. | 6689 | CH2 | GCGCCAGAAGCTGCAGGCGGACCAAGCGTGTTCCTGTTTCCACCCAAACCTAAGGATACTCTGATGATTAGCC |
| | | | GAACTCCTGAGGTCACCTGCGTGGTCGTGAGCGTGTCCCACGAGGACCCAGAAGTCAAGTTCAACTGGTACGT |
| | | | GGATGGGGTCGAAGTGCATAATGCCAAAACCAAGCCCAGGGAGGAACAGTACAACTCCACTTATCGCGTCGTG |
| | | | TCTGTCCTGACCGTGCTGCACCAGGACTGGCTGAATGGCAAGGAGTACAAATGTAAGGTCTCAAATAAGGCTC |
| | | | TGCCCGCCCCTATCGAAAAAACTATCTCAAAGGCAAAA |
| 60. | 6689 | CH3 | GGCCAGCCTCGCGAACCACAGGTCTACGTGCTGCCCCCTAGCCGCGACGAACTGACTAAAAATCAGGTCTCTC |
| | | | TGCTGTGTCTGGTCAAAGGATTCTACCCTTCCGACATCGCCGTGGAGTGGGAAAGTAACGGCCAGCCCGAGAA |
| | | | CAATTACCTGACCTGGCCCCCTGTGCTGGACTCTGATGGGAGTTTCTTTCTGTATTCAAAGCTGACAGTCGAT |
| | | | AAAAGCCGGTGGCAGCAGGGCAATGTGTTCAGCTGCTCCGTCATGCACGAAGCACTGCACAACCATTACACTC |
| | | | AGAAGTCCCTGTCCCTGTCACCTGGC |
| 61. | 6690 | Full | CAGATCGTCCTGACTCAGAGCCCCGCTATTATGTCCGCAAGCCCTGGAGAGAAAGTGACTATGACCTGTTCCG |
| | | | CATCTAGTTCCGTGTCCTACATGAACTGGTATCAGCAGAAATCTGGAACAAGTCCCAAGCGATGGATCTACGA |
| | | | CACTTCCAAGCTGGCATCTGGAGTGCCTGCCCACTTCCGAGGCAGCGGCTCTGGGACAAGTTATTCACTGACT |
| | | | ATTAGCGGCATGGAGGCCGAAGATGCCGCTACATACTATTGCCAGCAGTGGAGCTCCAACCCATTCACCTTTG |
| | | | GATGTGGCACAAAGCTGGAGATCAATGGCGGAGGAGGCTCCGGAGGAGGAGGGTCTGGAGGAGGAGGAAGTCA |
| | | | GGTCCAGCTGCAGCAGTCCGGAGCAGAACTGGCTAGACCAGGAGCCAGTGTGAAAATGTCATGCAAGGCCAGC |
| | | | GGCTACACATTCACTCGGTATACCATGCATTGGGTGAAACAGAGACCAGGACAGTGTCTGGAGTGGATCGGCT |
| | | | ACATTAATCCCAGCAGGGGGTACACAAACTACAACCAGAAGTTTAAAGACAAGGCAACCCTGACCACCGATAA |
| | | | GTCTAGTTCAACAGCTTATATGCAGCTGAGCTCCCTGACTTCAGAAGACAGCGCTGTGTACTATTGCGCACGC |
| | | | TACTATGACGATCACTACTCCCTGGATTATTGGGGGCAGGGAACTACCCTGACCGTGTCTAGTGCAGCCGAGC |
| | | | CTAAATCAAGCGACAAGACCCATACATGCCCCCCTTGTCCGGCGCCAGAAGCTGCAGGCGGACCAAGTGTGTT |
| | | | CCTGTTTCCACCCAAACCTAAGGATACTCTGATGATTTCTCGAACTCCTGAGGTCACCTGCGTGGTCGTGAGC |
| | | | GTGTCCCACGAGGACCCAGAAGTCAAGTTCAACTGGTACGTGGATGGGGTCGAAGTGCATAATGCCAAAACCA |
| | | | AGCCCAGGGAGGAACAGTACAACTCAACTTATCGCGTCGTGTCTGTCCTGACCGTGCTGCACCAGGACTGGCT |
| | | | GAATGGCAAGGAGTACAAATGTAAGGTCTCAAATAAGGCTCTGCCCGCCCCTATCGAAAAAACTATCTCTAAG |
| | | | GCAAAAGGACAGCCTCGCGAACCACAGGTCTACGTGCTGCCCCCTAGCCGCGACGAACTGACTAAAAATCAGG |
| | | | TCTCTCTGCTGTGTCTGGTCAAAGGATTCTACCCTTCCGACATCGCCGTGGAGTGGGAAAGTAACGGCCAGCC |
| | | | CGAGAACAATTACCTGACCTGGCCCCCTGTGCTGGACTCTGATGGGAGTTTCTTTCTGTATTCAAAGCTGACA |
| | | | GTCGATAAAAGCCGGTGGCAGCAGGGCAATGTGTTCAGCTGCTCCGTCATGCACGAAGCACTGCACAACCATT |
| | | | ACACTCAGAAGTCCCTGTCCCTGTCACCTGGC |
| 62. | 6690 | VL | CAGATCGTCCTGACTCAGAGCCCCGCTATTATGTCCGCAAGCCCTGGAGAGAAAGTGACTATGACCTGTTCCG |
| | | | CATCTAGTTCCGTGTCCTACATGAACTGGTATCAGCAGAAATCTGGAACAAGTCCCAAGCGATGGATCTACGA |
| | | | CACTTCCAAGCTGGCATCTGGAGTGCCTGCCCACTTCCGAGGCAGCGGCTCTGGGACAAGTTATTCACTGACT |
| | | | ATTAGCGGCATGGAGGCCGAAGATGCCGCTACATACTATTGCCAGCAGTGGAGCTCCAACCCATTCACCTTTG |
| | | | GATGTGGCACAAAGCTGGAGATCAAT |
| 63. | 6690 | VH | CAGGTCCAGCTGCAGCAGTCCGGAGCAGAACTGGCTAGACCAGGAGCCAGTGTGAAAATGTCATGCAAGGCCA |
| | | | GCGGCTACACATTCACTCGGTATACCATGCATTGGGTGAAACAGAGACCAGGACAGTGTCTGGAGTGGATCGG |
| | | | CTACATTAATCCCAGCAGGGGGTACACAAACTACAACCAGAAGTTTAAAGACAAGGCAACCCTGACCACCGAT |
| | | | AAGTCTAGTTCAACAGCTTATATGCAGCTGAGCTCCCTGACTTCAGAAGACAGCGCTGTGTACTATTGCGCAC |
| | | | GCTACTATGACGATCACTACTCCCTGGATTATTGGGGGCAGGGAACTACCCTGACCGTGTCTAGT |
| 64. | 6690 | CH2 | GCGCCAGAAGCTGCAGGCGGACCAAGTGTGTTCCTGTTTCCACCCAAACCTAAGGATACTCTGATGATTTCTC |
| | | | GAACTCCTGAGGTCACCTGCGTGGTCGTGAGCGTGTCCCACGAGGACCCAGAAGTCAAGTTCAACTGGTACGT |
| | | | GGATGGGGTCGAAGTGCATAATGCCAAAACCAAGCCCAGGGAGGAACAGTACAACTCAACTTATCGCGTCGTG |
| | | | TCTGTCCTGACCGTGCTGCACCAGGACTGGCTGAATGGCAAGGAGTACAAATGTAAGGTCTCAAATAAGGCTC |
| | | | TGCCCGCCCCTATCGAAAAAACTATCTCTAAGGCAAAA |
| 65. | 6690 | CH3 | GGACAGCCTCGCGAACCACAGGTCTACGTGCTGCCCCCTAGCCGCGACGAACTGACTAAAAATCAGGTCTCTC |
| | | | TGCTGTGTCTGGTCAAAGGATTCTACCCTTCCGACATCGCCGTGGAGTGGGAAAGTAACGGCCAGCCCGAGAA |
| | | | CAATTACCTGACCTGGCCCCCTGTGCTGGACTCTGATGGGAGTTTCTTTCTGTATTCAAAGCTGACAGTCGAT |
| | | | AAAAGCCGGTGGCAGCAGGGCAATGTGTTCAGCTGCTCCGTCATGCACGAAGCACTGCACAACCATTACACTC |
| | | | AGAAGTCCCTGTCCCTGTCACCTGGC |
| 66. | 6691 | Full | GATATTCAGCTGACACAGAGCCCCGCATCCCTGGCCGTGAGCCTGGGACAGAGAGCAACTATTTCCTGCAAAG |
| | | | CCTCACAGAGCGTGGACTATGATGGAGACAGCTATCTGAACTGGTACCAGCAGATCCCAGGCCAGCCCCCTAA |
| | | | ACTGCTGATCTACGACGCCAGCAATCTGGTGTCCGGCATCCCACCCAGGTTCAGTGGATCAGGCAGCGGGACC |
| | | | GATTTTACACTGAACATTCACCCTGTCGAGAAGGTGGACGCCGCTACCTACCATTGCCAGCAGTCCACAGAGG |
| | | | ACCCCTGGACTTTCGGATGTGGCACCAAACTGGAAATCAAGGGCGGGGGAGGCTCAGGAGGAGGAGGGAGCGG |
| | | | AGGAGGAGGCAGCCAGGTGCAGCTGCAGCAGAGCGGAGCAGAACTGGTCCGACCTGGAAGCTCCGTGAAAATT |
| | | | TCTTGCAAGGCCAGTGGCTATGCTTTTTCTAGTTACTGGATGAATTGGGTGAAGCAGCGACCAGGACAGTGTC |
| | | | TGGAGTGGATCGGGCAGATTTGGCCTGGGGATGGAGACACCAACTATAATGGAAAGTTCAAAGGCAAGGCAAC |
| | | | TCTGACCGCCGACGAATCAAGCTCCACAGCTTATATGCAGCTGTCTAGTCTGGCTAGTGAGGATTCAGCAGTG |
| | | | TACTTTTGCGCCCGGAGAGAAACCACAACTGTGGGCAGATACTATTACGCAATGGACTACTGGGGCCAGGGGA |
| | | | CCACAGTCACCGTGTCAAGCGCAGCCGAGCCCAAATCCTCTGATAAGACACACACTTGCCCTCCATGTCCGGC |
| | | | GCCAGAAGCTGCAGGCGGACCTTCCGTGTTCCTGTTTCCCCCTAAACCAAAGGACACTCTGATGATCTCTCGC |
| | | | ACTCCAGAGGTCACCTGCGTGGTCGTGTCCGTGTCTCACGAGGACCCCGAAGTCAAATTCAACTGGTATGTGG |
| | | | ACGGGGTCGAAGTGCATAATGCCAAAACAAAGCCTAGGGAGGAACAGTATAACTCTACATACCGCGTCGTGAG |
| | | | TGTCCTGACTGTGCTGCATCAGGATTGGCTGAATGGCAAGGAGTACAAATGTAAGGTCTCAAATAAGGCTCTG |
| | | | CCCGCCCCTATCGAAAAAACTATCTCTAAAGCTAAAGGCCAGCCTCGCGAACCACAGGTCTACGTGCTGCCCC |
| | | | CTAGCCGCGACGAACTGACTAAAAATCAGGTCTCTCTGCTGTGTCTGGTCAAAGGATTCTACCCTTCCGACAT |
| | | | CGCCGTGGAGTGGGAAAGTAACGGCCAGCCCGAGAACAATTACCTGACCTGGCCCCCTGTGCTGGACTCTGAT |
| | | | GGGAGTTTCTTTCTGTATTCAAAGCTGACAGTCGATAAAAGCCGGTGGCAGCAGGGCAATGTGTTCAGCTGCT |
| | | | CCGTCATGCACGAAGCACTGCACAACCATTACACTCAGAAGTCCCTGTCCCTGTCACCTGGC |
| 67. | 6691 | VL | GATATTCAGCTGACACAGAGCCCCGCATCCCTGGCCGTGAGCCTGGGACAGAGAGCAACTATTTCCTGCAAAG |
| | | | CCTCACAGAGCGTGGACTATGATGGAGACAGCTATCTGAACTGGTACCAGCAGATCCCAGGCCAGCCCCCTAA |
| | | | ACTGCTGATCTACGACGCCAGCAATCTGGTGTCCGGCATCCCACCCAGGTTCAGTGGATCAGGCAGCGGGACC |
| | | | GATTTTACACTGAACATTCACCCTGTCGAGAAGGTGGACGCCGCTACCTACCATTGCCAGCAGTCCACAGAGG |
| | | | ACCCCTGGACTTTCGGATGTGGCACCAAACTGGAAATCAAG |
| 68. | 6691 | VH | CAGGTGCAGCTGCAGCAGAGCGGAGCAGAACTGGTCCGACCTGGAAGCTCCGTGAAAATTTCTTGCAAGGCCA |
| | | | GTGGCTATGCTTTTTCTAGTTACTGGATGAATTGGGTGAAGCAGCGACCAGGACAGTGTCTGGAGTGGATCGG |
| | | | GCAGATTTGGCCTGGGGATGGAGACACCAACTATAATGGAAAGTTCAAAGGCAAGGCAACTCTGACCGCCGAC |
| | | | GAATCAAGCTCCACAGCTTATATGCAGCTGTCTAGTCTGGCTAGTGAGGATTCAGCAGTGTACTTTTGCGCCC |
| | | | GGAGAGAAACCACAACTGTGGGCAGATACTATTACGCAATGGACTACTGGGGCCAGGGGACCACAGTCACCGT |
| | | | GTCAAGC |
| 69. | 6691 | CH2 | GCGCCAGAAGCTGCAGGCGGACCTTCCGTGTTCCTGTTTCCCCCTAAACCAAAGGACACTCTGATGATCTCTC |
| | | | GCACTCCAGAGGTCACCTGCGTGGTCGTGTCCGTGTCTCACGAGGACCCCGAAGTCAAATTCAACTGGTATGT |
| | | | GGACGGGGTCGAAGTGCATAATGCCAAAACAAAGCCTAGGGAGGAACAGTATAACTCTACATACCGCGTCGTG |
| | | | AGTGTCCTGACTGTGCTGCATCAGGATTGGCTGAATGGCAAGGAGTACAAATGTAAGGTCTCAAATAAGGCTC |
| | | | TGCCCGCCCCTATCGAAAAAACTATCTCTAAAGCTAAA |
| 70. | 6691 | CH3 | GGCCAGCCTCGCGAACCACAGGTCTACGTGCTGCCCCCTAGCCGCGACGAACTGACTAAAAATCAGGTCTCTC |
| | | | TGCTGTGTCTGGTCAAAGGATTCTACCCTTCCGACATCGCCGTGGAGTGGGAAAGTAACGGCCAGCCCGAGAA |
| | | | CAATTACCTGACCTGGCCCCCTGTGCTGGACTCTGATGGGAGTTTCTTTCTGTATTCAAAGCTGACAGTCGAT |
| | | | AAAAGCCGGTGGCAGCAGGGCAATGTGTTCAGCTGCTCCGTCATGCACGAAGCACTGCACAACCATTACACTC |
| | | | AGAAGTCCCTGTCCCTGTCACCTGGC |
| 71. | 1064 | Full | GACATTCAGCTGACACAGAGTCCTGCTTCACTGGCAGTGAGCCTGGGACAGCGAGCAACTATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAGGGAGGAGGAGGCAGTGGCGGAGGAGGGTCAGG |
| | | | AGGAGGAGGAAGCCAGGTGCAGCTGCAGCAGAGCGGAGCAGAGCTGGTCAGACCAGGAAGCTCCGTGAAAATT |
| | | | TCCTGTAAGGCATCTGGCTATGCCTTTTCTAGTTACTGGATGAATTGGGTGAAGCAGAGGCCAGGACAGGGCC |
| | | | TGGAATGGATCGGGCAGATTTGGCCCGGGGATGGAGACACTAACTATAATGGAAAGTTCAAAGGCAAGGCTAC |
| | | | ACTGACTGCAGACGAGTCAAGCTCCACCGCTTATATGCAGCTGTCTAGTCTGGCCAGCGAGGATTCCGCTGTC |
| | | | TACTTTTGCGCACGGAGAGAAACCACAACTGTGGGCAGGTACTATTACGCAATGGACTACTGGGGCCAGGGGA |
| | | | CCACAGTCACCGTGTCAAGCGCAGCCGAACCCAAATCCTCTGATAAGACCCACACATGCCCTCCATGTCCAGC |
| | | | ACCTGAGCTGCTGGGAGGACCAAGCGTGTTCCTGTTTCCACCTAAACCTAAGGACACCCTGATGATCTCTCGG |
| | | | ACACCCGAAGTCACTTGTGTGGTCGTGGATGTGAGCCACGAGGACCCTGAAGTCAAATTCAACTGGTACGTGG |
| | | | ATGGCGTCGAGGTGCATAATGCCAAAACAAAGCCTAGGGAGGAACAGTATAACTCCACTTACCGCGTCGTGTC |
| | | | TGTCCTGACCGTGCTGCATCAGGACTGGCTGAACGGAAAGGAGTACAAATGCAAGGTGAGCAACAAGGCCCTG |
| | | | CCAGCTCCCATCGAGAAGACCATTTCCAAAGCTAAGGGCCAGCCTCGAGAACCACAGGTGTATACATACCCAC |
| | | | CCAGCCGGGACGAGCTGACCAAAAACCAGGTCTCCCTGACATGTCTGGTGAAGGGATTTTATCCTTCTGATAT |
| | | | TGCCGTGGAGTGGGAAAGTAATGGCCAGCCAGAAAACAATTACAAGACTACCCCTCCAGTGCTGGATTCTGAC |
| | | | GGGAGTTTCGCACTGGTCAGTAAACTGACAGTGGATAAGTCACGGTGGCAGCAGGGAAACGTCTTTAGTTGTT |
| | | | CAGTGATGCACGAGGCCCTGCACAATCATTACACTCAGAAAAGCCTGTCCCTGTCTCCCGGCAAG |
| 72. | 1064 | VL | GACATTCAGCTGACACAGAGTCCTGCTTCACTGGCAGTGAGCCTGGGACAGCGAGCAACTATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAG |
| 73. | 1064 | VH | CAGGTGCAGCTGCAGCAGAGCGGAGCAGAGCTGGTCAGACCAGGAAGCTCCGTGAAAATTTCCTGTAAGGCAT |
| | | | CTGGCTATGCCTTTTCTAGTTACTGGATGAATTGGGTGAAGCAGAGGCCAGGACAGGGCCTGGAATGGATCGG |
| | | | GCAGATTTGGCCCGGGGATGGAGACACTAACTATAATGGAAAGTTCAAAGGCAAGGCTACACTGACTGCAGAC |
| | | | GAGTCAAGCTCCACCGCTTATATGCAGCTGTCTAGTCTGGCCAGCGAGGATTCCGCTGTCTACTTTTGCGCAC |
| | | | GGAGAGAAACCACAACTGTGGGCAGGTACTATTACGCAATGGACTACTGGGGCCAGGGGACCACAGTCACCGT |
| | | | GTCAAGC |
| 74. | 1064 | CH2 | GCACCTGAGCTGCTGGGAGGACCAAGCGTGTTCCTGTTTCCACCTAAACCTAAGGACACCCTGATGATCTCTC |
| | | | GGACACCCGAAGTCACTTGTGTGGTCGTGGATGTGAGCCACGAGGACCCTGAAGTCAAATTCAACTGGTACGT |
| | | | GGATGGCGTCGAGGTGCATAATGCCAAAACAAAGCCTAGGGAGGAACAGTATAACTCCACTTACCGCGTCGTG |
| | | | TCTGTCCTGACCGTGCTGCATCAGGACTGGCTGAACGGAAAGGAGTACAAATGCAAGGTGAGCAACAAGGCCC |
| | | | TGCCAGCTCCCATCGAGAAGACCATTTCCAAAGCTAAG |
| 75. | 1064 | CH3 | GGCCAGCCTCGAGAACCACAGGTGTATACATACCCACCCAGCCGGGACGAGCTGACCAAAAACCAGGTCTCCC |
| | | | TGACATGTCTGGTGAAGGGATTTTATCCTTCTGATATTGCCGTGGAGTGGGAAAGTAATGGCCAGCCAGAAAA |
| | | | CAATTACAAGACTACCCCTCCAGTGCTGGATTCTGACGGGAGTTTCGCACTGGTCAGTAAACTGACAGTGGAT |
| | | | AAGTCACGGTGGCAGCAGGGAAACGTCTTTAGTTGTTCAGTGATGCACGAGGCCCTGCACAATCATTACACTC |
| | | | AGAAAAGCCTGTCCCTGTCTCCCGGC |
| 76. | 1065 | Full | GATATTAAGCTGCAGCAGAGCGGAGCTGAGCTGGCACGACCAGGAGCCAGTGTGAAAATGTCATGCAAGACCA |
| | | | GCGGCTACACATTCACTCGGTATACAATGCACTGGGTGAAGCAGAGACCAGGACAGGGACTGGAATGGATCGG |
| | | | ATATATTAACCCTTCCCGAGGCTACACCAACTATAATCAGAAGTTTAAAGACAAGGCCACTCTGACCACAGAT |
| | | | AAGAGCTCCTCTACCGCTTACATGCAGCTGAGTTCACTGACAAGTGAGGACTCAGCTGTGTACTATTGCGCAA |
| | | | GGTACTATGACGATCATTACTGTCTGGATTATTGGGGACAGGGCACTACCCTGACTGTCAGCTCCGTGGAAGG |
| | | | AGGGAGCGGAGGCTCCGGAGGATCTGGCGGGAGTGGAGGCGTGGACGATATCCAGCTGACCCAGTCCCCAGCA |
| | | | ATTATGTCCGCCTCTCCCGGCGAGAAAGTCACCATGACATGCCGCGCTTCTAGTTCAGTGAGCTACATGAACT |
| | | | GGTATCAGCAGAAATCAGGCACTAGCCCCAAGAGATGGATCTACGACACCTCCAAGGTCGCATCTGGGGTGCC |
| | | | TTATAGGTTCAGTGGGTCAGGAAGCGGCACCTCCTACTCTCTGACAATTAGCTCCATGGAGGCAGAAGATGCC |
| | | | GCTACCTACTATTGTCAGCAGTGGTCTAGTAATCCACTGACTTTTGGGGCCGGAACCAAACTGGAGCTGAAGG |
| | | | CAGCCGAACCCAAATCAAGCGACAAGACTCACACCTGCCCCCCTTGTCCAGCACCCGAACTGCTGGGGGGACC |
| | | | TAGCGTGTTCCTGTTTCCACCCAAACCAAAGGATACACTGATGATCAGCCGGACACCTGAGGTCACTTGCGTG |
| | | | GTCGTGGACGTGAGCCACGAGGACCCCGAAGTCAAGTTCAACTGGTACGTGGACGGCGTCGAAGTGCATAATG |
| | | | CTAAAACTAAGCCTAGGGAGGAACAGTACAATAGTACATATAGAGTCGTGTCAGTGCTGACCGTCCTGCATCA |
| | | | GGATTGGCTGAACGGGAAGGAGTACAAATGCAAGGTGTCCAACAAGGCCCTGCCTGCTCCAATCGAGAAGACA |
| | | | ATTTCTAAAGCCAAGGGCCAGCCCCGAGAACCTCAGGTGTATACACTGCCTCCATCCCGGGACGAGCTGACTA |
| | | | AAAACCAGGTCTCTCTGCTGTGTCTGGTGAAGGGGTTCTACCCATCTGATATTGCTGTGGAGTGGGAAAGTAA |
| | | | TGGACAGCCCGAGAACAATTATATGACCTGGCCCCCTGTCCTGGACTCCGATGGATCTTTCTTTCTGTACAGC |
| | | | AAACTGACAGTGGACAAGTCCAGATGGCAGCAGGGCAACGTCTTTAGTTGTTCAGTGATGCACGAGGCCCTGC |
| | | | ACAATCATTACACCCAGAAAAGCCTGTCCCTGTCTCCCGGCAAG |
| 77. | 1065 | VH | GATATTAAGCTGCAGCAGAGCGGAGCTGAGCTGGCACGACCAGGAGCCAGTGTGAAAATGTCATGCAAGACCA |
| | | | GCGGCTACACATTCACTCGGTATACAATGCACTGGGTGAAGCAGAGACCAGGACAGGGACTGGAATGGATCGG |
| | | | ATATATTAACCCTTCCCGAGGCTACACCAACTATAATCAGAAGTTTAAAGACAAGGCCACTCTGACCACAGAT |
| | | | AAGAGCTCCTCTACCGCTTACATGCAGCTGAGTTCACTGACAAGTGAGGACTCAGCTGTGTACTATTGCGCAA |
| | | | GGTACTATGACGATCATTACTGTCTGGATTATTGGGGACAGGGCACTACCCTGACTGTCAGCTCC |
| 78. | 1065 | VL | GATATCCAGCTGACCCAGTCCCCAGCAATTATGTCCGCCTCTCCCGGCGAGAAAGTCACCATGACATGCCGCG |
| | | | CTTCTAGTTCAGTGAGCTACATGAACTGGTATCAGCAGAAATCAGGCACTAGCCCCAAGAGATGGATCTACGA |
| | | | CACCTCCAAGGTCGCATCTGGGGTGCCTTATAGGTTCAGTGGGTCAGGAAGCGGCACCTCCTACTCTCTGACA |
| | | | ATTAGCTCCATGGAGGCAGAAGATGCCGCTACCTACTATTGTCAGCAGTGGTCTAGTAATCCACTGACTTTTG |
| | | | GGGCCGGAACCAAACTGGAGCTGAAG |
| 79. | 1065 | CH2 | GCACCCGAACTGCTGGGGGGACCTAGCGTGTTCCTGTTTCCACCCAAACCAAAGGATACACTGATGATCAGCC |
| | | | GGACACCTGAGGTCACTTGCGTGGTCGTGGACGTGAGCCACGAGGACCCCGAAGTCAAGTTCAACTGGTACGT |
| | | | GGACGGCGTCGAAGTGCATAATGCTAAAACTAAGCCTAGGGAGGAACAGTACAATAGTACATATAGAGTCGTG |
| | | | TCAGTGCTGACCGTCCTGCATCAGGATTGGCTGAACGGGAAGGAGTACAAATGCAAGGTGTCCAACAAGGCCC |
| | | | TGCCTGCTCCAATCGAGAAGACAATTTCTAAAGCCAAG |
| 80. | 1065 | CH3 | GGCCAGCCCCGAGAACCTCAGGTGTATACACTGCCTCCATCCCGGGACGAGCTGACTAAAAACCAGGTCTCTC |
| | | | TGCTGTGTCTGGTGAAGGGGTTCTACCCATCTGATATTGCTGTGGAGTGGGAAAGTAATGGACAGCCCGAGAA |
| | | | CAATTATATGACCTGGCCCCCTGTCCTGGACTCCGATGGATCTTTCTTTCTGTACAGCAAACTGACAGTGGAC |
| | | | AAGTCCAGATGGCAGCAGGGCAACGTCTTTAGTTGTTCAGTGATGCACGAGGCCCTGCACAATCATTACACCC |
| | | | AGAAAAGCCTGTCCCTGTCTCCCGGC |
| 81. | 1067 | Full | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACCTACTATTGCCAGCAGTGGAGTTCAAACCCATTCACTTTTG |
| | | | GATCTGGCACCAAGCTGGAAATTAATGGCGGAGGAGGCTCCGGAGGAGGAGGGTCTGGAGGAGGAGGAAGTCA |
| | | | GGTCCAGCTGCAGCAGTCCGGAGCTGAGCTGGCACGACCAGGAGCAAGTGTGAAAATGTCCTGTAAGGCCAGC |
| | | | GGCTACACCTTCACACGGTATACCATGCATTGGGTGAAACAGAGACCCGGGCAGGGACTGGAATGGATCGGGT |
| | | | ACATTAATCCTAGCCGAGGATACACAAACTACAACCAGAAGTTTAAAGACAAGGCTACTCTGACCACAGATAA |
| | | | GAGCTCCTCTACCGCATATATGCAGCTGAGTTCACTGACATCTGAGGACAGTGCCGTGTACTATTGCGCTAGG |
| | | | TACTATGACGATCACTACTGTCTGGATTATTGGGGCCAGGGGACTACCCTGACCGTGAGCTCCGCAGCCGAAC |
| | | | CTAAATCTAGTGACAAGACTCATACCTGCCCCCCTTGTCCAGCACCAGAGCTGCTGGGAGGACCTTCCGTGTT |
| | | | CCTGTTTCCACCCAAACCAAAGGATACTCTGATGATCTCCCGGACACCTGAAGTCACTTGCGTGGTCGTGGAC |
| | | | GTGTCTCACGAGGACCCCGAAGTCAAGTTTAACTGGTACGTGGACGGCGTCGAGGTGCATAATGCCAAAACCA |
| | | | AGCCCAGGGAGGAACAGTACAACTCCACATATCGCGTCGTGTCTGTCCTGACTGTGCTGCACCAGGATTGGCT |
| | | | GAACGGCAAGGAGTACAAATGCAAGGTGAGCAACAAGGCCCTGCCTGCTCCAATCGAGAAGACAATTAGCAAA |
| | | | GCCAAGGGGCAGCCCCGAGAACCTCAGGTGTACACTCTGCCTCCATCTCGGGACGAGCTGACCAAAAACCAGG |
| | | | TCAGTCTGCTGTGTCTGGTGAAGGGCTTCTATCCAAGCGATATTGCTGTGGAGTGGGAATCCAATGGGCAGCC |
| | | | CGAAAACAATTACATGACATGGCCCCCTGTCCTGGACTCAGATGGGAGCTTCTTTCTGTATAGTAAACTGACT |
| | | | GTGGACAAGTCACGGTGGCAGCAGGGAAACGTCTTTAGCTGTTCCGTGATGCATGAGGCCCTGCACAATCATT |
| | | | ACACCCAGAAATCTCTGAGTCTGTCACCCGGCAAG |
| 82. | 1067 | VL | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACCTACTATTGCCAGCAGTGGAGTTCAAACCCATTCACTTTTG |
| | | | GATCTGGCACCAAGCTGGAAATTAAT |
| 83. | 1067 | VH | CAGGTCCAGCTGCAGCAGTCCGGAGCTGAGCTGGCACGACCAGGAGCAAGTGTGAAAATGTCCTGTAAGGCCA |
| | | | GCGGCTACACCTTCACACGGTATACCATGCATTGGGTGAAACAGAGACCCGGGCAGGGACTGGAATGGATCGG |
| | | | GTACATTAATCCTAGCCGAGGATACACAAACTACAACCAGAAGTTTAAAGACAAGGCTACTCTGACCACAGAT |
| | | | AAGAGCTCCTCTACCGCATATATGCAGCTGAGTTCACTGACATCTGAGGACAGTGCCGTGTACTATTGCGCTA |
| | | | GGTACTATGACGATCACTACTGTCTGGATTATTGGGGCCAGGGGACTACCCTGACCGTGAGCTCC |
| 84. | 1067 | CH2 | GCACCAGAGCTGCTGGGAGGACCTTCCGTGTTCCTGTTTCCACCCAAACCAAAGGATACTCTGATGATCTCCC |
| | | | GGACACCTGAAGTCACTTGCGTGGTCGTGGACGTGTCTCACGAGGACCCCGAAGTCAAGTTTAACTGGTACGT |
| | | | GGACGGCGTCGAGGTGCATAATGCCAAAACCAAGCCCAGGGAGGAACAGTACAACTCCACATATCGCGTCGTG |
| | | | TCTGTCCTGACTGTGCTGCACCAGGATTGGCTGAACGGCAAGGAGTACAAATGCAAGGTGAGCAACAAGGCCC |
| | | | TGCCTGCTCCAATCGAGAAGACAATTAGCAAAGCCAAG |
| 85. | 1067 | CH3 | GGGCAGCCCCGAGAACCTCAGGTGTACACTCTGCCTCCATCTCGGGACGAGCTGACCAAAAACCAGGTCAGTC |
| | | | TGCTGTGTCTGGTGAAGGGCTTCTATCCAAGCGATATTGCTGTGGAGTGGGAATCCAATGGGCAGCCCGAAAA |
| | | | CAATTACATGACATGGCCCCCTGTCCTGGACTCAGATGGGAGCTTCTTTCTGTATAGTAAACTGACTGTGGAC |
| | | | AAGTCACGGTGGCAGCAGGGAAACGTCTTTAGCTGTTCCGTGATGCATGAGGCCCTGCACAATCATTACACCC |
| | | | AGAAATCTCTGAGTCTGTCACCCGGC |
| 86. | 1842 | Full | GATATTCAGCTGACACAGAGTCCTGCTTCACTGGCAGTGAGCCTGGGACAGCGAGCAACTATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAGGGAGGAGGAGGCAGTGGCGGAGGAGGGTCAGG |
| | | | AGGAGGAGGAAGCCAGGTGCAGCTGCAGCAGAGCGGAGCAGAGCTGGTCAGACCAGGAAGCTCCGTGAAAATT |
| | | | TCCTGTAAGGCATCTGGCTATGCCTTTTCTAGTTACTGGATGAATTGGGTGAAGCAGAGGCCAGGACAGGGCC |
| | | | TGGAATGGATCGGGCAGATTTGGCCCGGGGATGGAGACACCAACTATAATGGAAAGTTCAAAGGCAAGGCTAC |
| | | | ACTGACTGCAGACGAGTCAAGCTCCACAGCTTATATGCAGCTGTCTAGTCTGGCCAGCGAGGATTCCGCTGTG |
| | | | TACTTTTGCGCACGGAGAGAAACCACAACTGTGGGCAGGTACTATTACGCAATGGACTACTGGGGCCAGGGGA |
| | | | CCACAGTCACCGTGTCAAGCGCAGCCGAACCCAAATCCTCTGATAAGACCCACACATGCCCTCCATGTCCAGC |
| | | | ACCTGAGCTGCTGGGAGGACCAAGCGTGTTCCTGTTTCCACCTAAACCTAAGGACACACTGATGATCTCTCGG |
| | | | ACACCCGAAGTCACTTGTGTGGTCGTGGATGTGAGCCACGAGGACCCTGAAGTCAAATTCAACTGGTACGTGG |
| | | | ATGGCGTCGAGGTGCATAATGCCAAAACTAAGCCTAGGGAGGAACAGTATAACTCCACTTACCGCGTCGTGTC |
| | | | TGTCCTGACCGTGCTGCATCAGGACTGGCTGAACGGAAAGGAGTACAAATGCAAGGTGAGCAACAAGGCCCTG |
| | | | CCAGCTCCCATCGAGAAGACAATTTCCAAAGCTAAGGGCCAGCCTCGAGAACCACAGGTCTATGTGTACCCAC |
| | | | CCAGCCGGGACGAGCTGACCAAAAACCAGGTCTCCCTGACATGTCTGGTGAAGGGATTTTATCCTTCTGATAT |
| | | | TGCCGTGGAGTGGGAAAGTAATGGCCAGCCAGAAAACAATTACAAGACTACCCCTCCAGTGCTGGATTCTGAC |
| | | | GGGAGTTTCGCACTGGTCAGTAAACTGACTGTGGATAAGTCACGGTGGCAGCAGGGAAACGTCTTTAGTTGTT |
| | | | CAGTGATGCACGAGGCCCTGCACAATCATTACACCCAGAAAAGCCTGTCCCTGTCTCCCGGCAAG |
| 87. | 1842 | VL | GATATTCAGCTGACACAGAGTCCTGCTTCACTGGCAGTGAGCCTGGGACAGCGAGCAACTATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGGACT |
| | | | GATTTTACCCTGAACATTCACCCAGTCGAGAAGGTGGACGCCGCTACCTACCATTGCCAGCAGTCTACCGAGG |
| | | | ACCCCTGGACATTCGGCGGGGGAACTAAACTGGAAATCAAG |
| 88. | 1842 | VH | CAGGTGCAGCTGCAGCAGAGCGGAGCAGAGCTGGTCAGACCAGGAAGCTCCGTGAAAATTTCCTGTAAGGCAT |
| | | | CTGGCTATGCCTTTTCTAGTTACTGGATGAATTGGGTGAAGCAGAGGCCAGGACAGGGCCTGGAATGGATCGG |
| | | | GCAGATTTGGCCCGGGGATGGAGACACCAACTATAATGGAAAGTTCAAAGGCAAGGCTACACTGACTGCAGAC |
| | | | GAGTCAAGCTCCACAGCTTATATGCAGCTGTCTAGTCTGGCCAGCGAGGATTCCGCTGTGTACTTTTGCGCAC |
| | | | GGAGAGAAACCACAACTGTGGGCAGGTACTATTACGCAATGGACTACTGGGGCCAGGGGACCACAGTCACCGT |
| | | | GTCAAGC |
| 89. | 1842 | CH2 | GCACCTGAGCTGCTGGGAGGACCAAGCGTGTTCCTGTTTCCACCTAAACCTAAGGACACACTGATGATCTCTC |
| | | | GGACACCCGAAGTCACTTGTGTGGTCGTGGATGTGAGCCACGAGGACCCTGAAGTCAAATTCAACTGGTACGT |
| | | | GGATGGCGTCGAGGTGCATAATGCCAAAACTAAGCCTAGGGAGGAACAGTATAACTCCACTTACCGCGTCGTG |
| | | | TCTGTCCTGACCGTGCTGCATCAGGACTGGCTGAACGGAAAGGAGTACAAATGCAAGGTGAGCAACAAGGCCC |
| | | | TGCCAGCTCCCATCGAGAAGACAATTTCCAAAGCTAAG |
| 90. | 1842 | CH3 | GGCCAGCCTCGAGAACCACAGGTCTATGTGTACCCACCCAGCCGGGACGAGCTGACCAAAAACCAGGTCTCCC |
| | | | TGACATGTCTGGTGAAGGGATTTTATCCTTCTGATATTGCCGTGGAGTGGGAAAGTAATGGCCAGCCAGAAAA |
| | | | CAATTACAAGACTACCCCTCCAGTGCTGGATTCTGACGGGAGTTTCGCACTGGTCAGTAAACTGACTGTGGAT |
| | | | AAGTCACGGTGGCAGCAGGGAAACGTCTTTAGTTGTTCAGTGATGCACGAGGCCCTGCACAATCATTACACCC |
| | | | AGAAAAGCCTGTCCCTGTCTCCCGGC |
| 91. | 1335 | Full | CAGATTGTCCTGTCTCAGAGTCCCGCTATCCTGTCAGCAAGCCCTGGGGAGAAGGTGACCATGACATGCCGAG |
| | | | CCAGCTCCTCTGTCAGCTACATCCACTGGTTCCAGCAGAAGCCAGGCAGTTCACCTAAACCATGGATCTACGC |
| | | | CACATCTAACCTGGCTAGTGGAGTGCCCGTCCGGTTTTCCGGCTCTGGGAGTGGAACATCATACAGCCTGACT |
| | | | ATTTCCAGAGTGGAGGCCGAAGACGCCGCTACCTACTATTGCCAGCAGTGGACCTCTAATCCCCCTACATTCG |
| | | | GCGGGGGAACTAAGCTGGAGATCAAAAGGACTGTGGCAGCCCCTTCTGTCTTCATTTTTCCACCCAGTGACGA |
| | | | ACAGCTGAAATCAGGAACCGCTTCCGTGGTCTGTCTGCTGAACAACTTCTACCCCCGCGAGGCAAAGGTGCAG |
| | | | TGGAAAGTCGATAACGCCCTGCAGTCCGGCAATTCTCAGGAGAGTGTGACCGAACAGGACTCAAAGGATAGCA |
| | | | CATATTCCCTGAGCTCCACTCTGACCCTGTCCAAAGCTGATTACGAAAAGCATAAAGTGTATGCATGTGAGGT |
| | | | CACCCACCAGGGGCTGAGTAGTCCCGTCACAAAGAGTTTCAATAGAGGAGAGTGT |
| 92. | 1335 | VL | CAGATTGTCCTGTCTCAGAGTCCCGCTATCCTGTCAGCAAGCCCTGGGGAGAAGGTGACCATGACATGCCGAG |
| | | | CCAGCTCCTCTGTCAGCTACATCCACTGGTTCCAGCAGAAGCCAGGCAGTTCACCTAAACCATGGATCTACGC |
| | | | CACATCTAACCTGGCTAGTGGAGTGCCCGTCCGGTTTTCCGGCTCTGGGAGTGGAACATCATACAGCCTGACT |
| | | | ATTTCCAGAGTGGAGGCCGAAGACGCCGCTACCTACTATTGCCAGCAGTGGACCTCTAATCCCCCTACATTCG |
| | | | GCGGGGGAACTAAGCTGGAGATCAAA |
| 93. | 1335 | CL | AGGACTGTGGCAGCCCCTTCTGTCTTCATTTTTCCACCCAGTGACGAACAGCTGAAATCAGGAACCGCTTCCG |
| | | | TGGTCTGTCTGCTGAACAACTTCTACCCCCGCGAGGCAAAGGTGCAGTGGAAAGTCGATAACGCCCTGCAGTC |
| | | | CGGCAATTCTCAGGAGAGTGTGACCGAACAGGACTCAAAGGATAGCACATATTCCCTGAGCTCCACTCTGACC |
| | | | CTGTCCAAAGCTGATTACGAAAAGCATAAAGTGTATGCATGTGAGGTCACCCACCAGGGGCTGAGTAGTCCCG |
| | | | TCACAAAGAGTTTCAATAGAGGAGAGTGT |
| 94. | 1342 | Full | CAGGTCCAGCTGCAGCAGCCCGGAGCTGAACTGGTCAAACCTGGCGCATCCGTGAAAATGTCTTGCAAGGCTA |
| | | | GTGGCTACACATTCACTTCCTATAACATGCACTGGGTGAAGCAGACACCAGGACGAGGACTGGAGTGGATCGG |
| | | | AGCAATCTACCCTGGAAACGGCGACACTTCTTATAATCAGAAGTTTAAAGGCAAGGCCACCCTGACAGCTGAT |
| | | | AAGAGCTCCTCTACCGCCTACATGCAGCTGAGTTCACTGACAAGTGAAGACTCAGCAGTGTACTATTGCGCCA |
| | | | GAAGCACCTACTATGGCGGGGATTGGTACTTCAACGTGTGGGGGGCAGGAACCACAGTCACCGTGAGCGCCGC |
| | | | TTCCACAAAAGGACCAAGCGTGTTTCCACTGGCACCAAGCTCCAAGTCAACCAGCGGAGGAACAGCAGCCCTG |
| | | | GGATGTCTGGTGAAGGACTACTTCCCAGAGCCCGTCACCGTGTCTTGGAACAGTGGCGCCCTGACAAGCGGGG |
| | | | TCCATACTTTTCCCGCTGTGCTGCAGTCTAGTGGCCTGTACAGCCTGTCAAGCGTGGTCACCGTCCCTTCCTC |
| | | | TAGTCTGGGGACTCAGACCTATATCTGCAACGTGAATCACAAACCTTCTAATACAAAGGTCGACAAGAAAGTG |
| | | | GAACCAAAAAGTTGTGATAAGACACATACTTGCCCACCTTGTCCTGCACCAGAGCTGCTGGGAGGACCATCCG |
| | | | TGTTCCTGTTTCCACCCAAACCCAAGGACACTCTGATGATTAGCCGGACTCCTGAAGTCACCTGCGTGGTCGT |
| | | | GGACGTGAGCCACGAGGACCCCGAAGTCAAATTCAACTGGTACGTGGATGGCGTCGAGGTGCATAATGCCAAA |
| | | | ACAAAGCCCCGGGAGGAACAGTACAACTCAACATATAGAGTCGTGAGCGTCCTGACTGTGCTGCACCAGGACT |
| | | | GGCTGAACGGCAAGGAGTATAAATGCAAGGTGTCCAACAAGGCCCTGCCCGCACCTATCGAGAAGACTATTTC |
| | | | TAAAGCCAAGGGCCAGCCTAGGGAACCACAGGTGTACGTGCTGCCTCCAAGCCGCGACGAGCTGACTAAAAAC |
| | | | CAGGTCTCCCTGCTGTGTCTGGTGAAGGGGTTCTATCCAAGTGATATCGCTGTGGAGTGGGAATCAAATGGAC |
| | | | AGCCCGAGAACAATTACCTGACTTGGCCCCCTGTGCTGGACTCAGATGGGAGCTTCTTTCTGTATTCCAAACT |
| | | | GACCGTGGATAAGTCTCGGTGGCAGCAGGGAAATGTCTTTTCCTGTTCTGTGATGCACGAAGCACTGCACAAT |
| | | | CACTACACCCAGAAGTCCCTGAGCCTGTCACCCGGCAAA |
| 95. | 1342 | VH | CAGGTCCAGCTGCAGCAGCCCGGAGCTGAACTGGTCAAACCTGGCGCATCCGTGAAAATGTCTTGCAAGGCTA |
| | | | GTGGCTACACATTCACTTCCTATAACATGCACTGGGTGAAGCAGACACCAGGACGAGGACTGGAGTGGATCGG |
| | | | AGCAATCTACCCTGGAAACGGCGACACTTCTTATAATCAGAAGTTTAAAGGCAAGGCCACCCTGACAGCTGAT |
| | | | AAGAGCTCCTCTACCGCCTACATGCAGCTGAGTTCACTGACAAGTGAAGACTCAGCAGTGTACTATTGCGCCA |
| | | | GAAGCACCTACTATGGCGGGGATTGGTACTTCAACGTGTGGGGGGCAGGAACCACAGTCACCGTGAGCGCC |
| 96. | 1342 | CH1 | GCTTCCACAAAAGGACCAAGCGTGTTTCCACTGGCACCAAGCTCCAAGTCAACCAGCGGAGGAACAGCAGCCC |
| | | | TGGGATGTCTGGTGAAGGACTACTTCCCAGAGCCCGTCACCGTGTCTTGGAACAGTGGCGCCCTGACAAGCGG |
| | | | GGTCCATACTTTTCCCGCTGTGCTGCAGTCTAGTGGCCTGTACAGCCTGTCAAGCGTGGTCACCGTCCCTTCC |
| | | | TCTAGTCTGGGGACTCAGACCTATATCTGCAACGTGAATCACAAACCTTCTAATACAAAGGTCGACAAGAAAG |
| | | | TG |
| 97. | 1342 | CH2 | GCACCAGAGCTGCTGGGAGGACCATCCGTGTTCCTGTTTCCACCCAAACCCAAGGACACTCTGATGATTAGCC |
| | | | GGACTCCTGAAGTCACCTGCGTGGTCGTGGACGTGAGCCACGAGGACCCCGAAGTCAAATTCAACTGGTACGT |
| | | | GGATGGCGTCGAGGTGCATAATGCCAAAACAAAGCCCCGGGAGGAACAGTACAACTCAACATATAGAGTCGTG |
| | | | AGCGTCCTGACTGTGCTGCACCAGGACTGGCTGAACGGCAAGGAGTATAAATGCAAGGTGTCCAACAAGGCCC |
| | | | TGCCCGCACCTATCGAGAAGACTATTTCTAAAGCCAAG |
| 98. | 1342 | CH3 | GGCCAGCCTAGGGAACCACAGGTGTACGTGCTGCCTCCAAGCCGCGACGAGCTGACTAAAAACCAGGTCTCCC |
| | | | TGCTGTGTCTGGTGAAGGGGTTCTATCCAAGTGATATCGCTGTGGAGTGGGAATCAAATGGACAGCCCGAGAA |
| | | | CAATTACCTGACTTGGCCCCCTGTGCTGGACTCAGATGGGAGCTTCTTTCTGTATTCCAAACTGACCGTGGAT |
| | | | AAGTCTCGGTGGCAGCAGGGAAATGTCTTTTCCTGTTCTGTGATGCACGAAGCACTGCACAATCACTACACCC |
| | | | AGAAGTCCCTGAGCCTGTCACCCGGC |
| 99. | 5239 | Full | CAGGTCCAGCTGGTCCAGTCCGGAGGAGGAGTGGTCCAGCCAGGACGGTCACTGAGACTGAGCTGCAAGGCTT |
| | | | CCGGGTACACTTTCACCCGATATACCATGCACTGGGTGCGGCAGGCACCAGGGAAAGGACTGGAATGGATCGG |
| | | | GTACATTAACCCTAGCAGGGGATACACAAACTATAATCAGAAGGTGAAAGACAGGTTCACTATCTCTCGCGAT |
| | | | AACAGTAAGAATACCGCCTTTCTGCAGATGGACAGCCTGCGCCCCGAGGATACAGGCGTGTATTTCTGCGCTC |
| | | | GATACTATGACGATCACTACTGTCTGGACTATTGGGGCCAGGGGACTCCAGTCACCGTGAGCTCCGCATCAAC |
| | | | TAAGGGACCCAGCGTGTTTCCACTGGCCCCCTCTAGTAAATCCACATCTGGAGGAACTGCAGCTCTGGGATGC |
| | | | CTGGTGAAGGATTACTTCCCAGAGCCCGTCACCGTGAGCTGGAACTCCGGAGCCCTGACTTCCGGCGTCCATA |
| | | | CCTTTCCCGCTGTGCTGCAGTCAAGCGGGCTGTACTCTCTGTCCTCTGTGGTCACAGTGCCTAGTTCAAGCCT |
| | | | GGGAACACAGACTTATATCTGCAACGTGAATCACAAGCCTAGCAATACTAAAGTCGACAAGAAAGTGGAACCA |
| | | | AAGAGCTGTGATAAAACCCATACATGCCCCCCTTGTCCTGCACCAGAGGCAGCAGGAGGACCAAGCGTGTTCC |
| | | | TGTTTCCACCCAAGCCTAAAGACACCCTGATGATTAGCCGGACCCCTGAAGTGACATGTGTGGTCGTGAGTGT |
| | | | GTCACACGAGGACCCAGAAGTCAAGTTCAACTGGTACGTGGATGGCGTCGAGGTGCATAATGCCAAGACAAAA |
| | | | CCTAGAGAGGAACAGTACAATTCCACCTATAGGGTCGTGTCTGTCCTGACAGTGCTGCACCAGGATTGGCTGA |
| | | | ACGGGAAAGAGTATAAGTGCAAAGTGTCCAATAAGGCTCTGCCCGCACCTATCGAGAAAACCATTTCTAAGGC |
| | | | TAAAGGCCAGCCTAGGGAACCACAGGTCTACGTGTATCCTCCATCTCGCGACGAGCTGACAAAGAACCAGGTC |
| | | | AGTCTGACTTGTCTGGTGAAAGGATTTTACCCAAGCGATATTGCCGTGGAGTGGGAATCCAATGGCCAGCCCG |
| | | | AAAACAATTATAAGACCACACCCCCTGTGCTGGACTCTGATGGCAGTTTCGCACTGGTCAGTAAGCTGACTGT |
| | | | GGACAAATCAAGATGGCAGCAGGGGAACGTCTTTAGCTGTTCCGTGATGCATGAGGCCCTGCACAATCATTAC |
| | | | ACCCAGAAGTCTCTGAGTCTGTCACCCGGC |
| 100. | 5239 | VH | CAGGTCCAGCTGGTCCAGTCCGGAGGAGGAGTGGTCCAGCCAGGACGGTCACTGAGACTGAGCTGCAAGGCTT |
| | | | CCGGGTACACTTTCACCCGATATACCATGCACTGGGTGCGGCAGGCACCAGGGAAAGGACTGGAATGGATCGG |
| | | | GTACATTAACCCTAGCAGGGGATACACAAACTATAATCAGAAGGTGAAAGACAGGTTCACTATCTCTCGCGAT |
| | | | AACAGTAAGAATACCGCCTTTCTGCAGATGGACAGCCTGCGCCCCGAGGATACAGGCGTGTATTTCTGCGCTC |
| | | | GATACTATGACGATCACTACTGTCTGGACTATTGGGGCCAGGGGACTCCAGTCACCGTGAGCTCC |
| 101. | 5239 | CH1 | GCATCAACTAAGGGACCCAGCGTGTTTCCACTGGCCCCCTCTAGTAAATCCACATCTGGAGGAACTGCAGCTC |
| | | | TGGGATGCCTGGTGAAGGATTACTTCCCAGAGCCCGTCACCGTGAGCTGGAACTCCGGAGCCCTGACTTCCGG |
| | | | CGTCCATACCTTTCCCGCTGTGCTGCAGTCAAGCGGGCTGTACTCTCTGTCCTCTGTGGTCACAGTGCCTAGT |
| | | | TCAAGCCTGGGAACACAGACTTATATCTGCAACGTGAATCACAAGCCTAGCAATACTAAAGTCGACAAGAAAG |
| | | | TG |
| 102. | 5239 | CH2 | GCACCAGAGGCAGCAGGAGGACCAAGCGTGTTCCTGTTTCCACCCAAGCCTAAAGACACCCTGATGATTAGCC |
| | | | GGACCCCTGAAGTGACATGTGTGGTCGTGAGTGTGTCACACGAGGACCCAGAAGTCAAGTTCAACTGGTACGT |
| | | | GGATGGCGTCGAGGTGCATAATGCCAAGACAAAACCTAGAGAGGAACAGTACAATTCCACCTATAGGGTCGTG |
| | | | TCTGTCCTGACAGTGCTGCACCAGGATTGGCTGAACGGGAAAGAGTATAAGTGCAAAGTGTCCAATAAGGCTC |
| | | | TGCCCGCACCTATCGAGAAAACCATTTCTAAGGCTAAA |
| 103. | 5239 | CH3 | GGCCAGCCTAGGGAACCACAGGTCTACGTGTATCCTCCATCTCGCGACGAGCTGACAAAGAACCAGGTCAGTC |
| | | | TGACTTGTCTGGTGAAAGGATTTTACCCAAGCGATATTGCCGTGGAGTGGGAATCCAATGGCCAGCCCGAAAA |
| | | | CAATTATAAGACCACACCCCCTGTGCTGGACTCTGATGGCAGTTTCGCACTGGTCAGTAAGCTGACTGTGGAC |
| | | | AAATCAAGATGGCAGCAGGGGAACGTCTTTAGCTGTTCCGTGATGCATGAGGCCCTGCACAATCATTACACCC |
| | | | AGAAGTCTCTGAGTCTGTCACCCGGC |
| 104. | 3916 | Full | GAAGTCCAGCTGGTCGAGAGCGGAGGAGGACTGGTGCAGCCAGGACGGTCCCTGAGACTGTCTTGCGCCGCTA |
| | | | GTGGGTTCACCTTTAACGACTATGCCATGCACTGGGTCCGACAGGCTCCAGGAAAGGGACTGGAATGGGTGTC |
| | | | TACCATCAGTTGGAATAGTGGATCAATTGGCTATGCTGACTCCGTGAAAGGCAGGTTCACAATCTCACGCGAT |
| | | | AACGCAAAGAAAAGCCTGTACCTGCAGATGAACAGCCTGCGCGCCGAGGACACAGCTCTGTACTATTGCGCCA |
| | | | AGGATATTCAGTACGGGAACTACTATTACGGAATGGACGTGTGGGGGCAGGGAACCACAGTCACTGTGAGCTC |
| | | | CGGCGGGGGAGGCTCAGGAGGAGGAGGGAGCGGAGGAGGAGGCAGCGAAATCGTGCTGACTCAGAGCCCTGCA |
| | | | ACCCTGAGCCTGTCCCCAGGAGAGCGAGCTACACTGAGCTGTCGGGCATCTCAGAGTGTGTCTAGTTATCTGG |
| | | | CATGGTACCAGCAGAAGCCAGGGCAGGCCCCCAGACTGCTGATCTACGATGCATCCAACAGAGCCACTGGCAT |
| | | | CCCCGCAAGGTTCTCAGGCAGCGGGTCCGGAACCGACTTTACTCTGACCATCTCAAGCCTGGAGCCCGAAGAT |
| | | | TTCGCTGTGTATTACTGCCAGCAGAGGTCTAATTGGCCTATCACATTTGGCCAGGGGACTCGCCTGGAGATTA |
| | | | AGGCAGCCGAACCAAAGTCCTCTGACAAAACACACACTTGCCCCCCTTGTCCAGCACCAGAACTGCTGGGAGG |
| | | | ACCAAGCGTGTTCCTGTTTCCACCCAAGCCTAAAGATACCCTGATGATTAGTAGGACCCCTGAGGTCACATGT |
| | | | GTGGTCGTGGACGTGAGCCACGAGGACCCCGAAGTCAAGTTTAACTGGTACGTGGACGGCGTCGAAGTGCATA |
| | | | ATGCCAAGACAAAACCCCGCGAGGAACAGTATAATTCTACCTACCGAGTCGTGAGTGTCCTGACAGTGCTGCA |
| | | | TCAGGATTGGCTGAACGGAAAAGAGTACAAGTGCAAAGTGTCCAATAAGGCTCTGCCTGCACCAATCGAGAAA |
| | | | ACTATTTCTAAGGCAAAAGGGCAGCCCCGGGAACCTCAGGTCTATGTGCTGCCTCCATCCAGAGACGAGCTGA |
| | | | CCAAGAACCAGGTCTCTCTGCTGTGTCTGGTGAAAGGATTCTACCCATCAGATATCGCTGTGGAGTGGGAAAG |
| | | | CAATGGCCAGCCCGAGAACAATTATCTGACATGGCCCCCTGTGCTGGACTCAGATGGCAGCTTCTTTCTGTAC |
| | | | TCTAAGCTGACTGTGGATAAAAGTCGGTGGCAGCAGGGGAACGTCTTTTCTTGTAGTGTGATGCATGAGGCCC |
| | | | TGCACAATCATTACACCCAGAAGTCACTGAGCCTGTCCCCTGGCAAA |
| 105. | 3916 | VH | GAAGTCCAGCTGGTCGAGAGCGGAGGAGGACTGGTGCAGCCAGGACGGTCCCTGAGACTGTCTTGCGCCGCTA |
| | | | GTGGGTTCACCTTTAACGACTATGCCATGCACTGGGTCCGACAGGCTCCAGGAAAGGGACTGGAATGGGTGTC |
| | | | TACCATCAGTTGGAATAGTGGATCAATTGGCTATGCTGACTCCGTGAAAGGCAGGTTCACAATCTCACGCGAT |
| | | | AACGCAAAGAAAAGCCTGTACCTGCAGATGAACAGCCTGCGCGCCGAGGACACAGCTCTGTACTATTGCGCCA |
| | | | AGGATATTCAGTACGGGAACTACTATTACGGAATGGACGTGTGGGGGCAGGGAACCACAGTCACTGTGAGCTC |
| | | | C |
| 106. | 3916 | VL | GAAATCGTGCTGACTCAGAGCCCTGCAACCCTGAGCCTGTCCCCAGGAGAGCGAGCTACACTGAGCTGTCGGG |
| | | | CATCTCAGAGTGTGTCTAGTTATCTGGCATGGTACCAGCAGAAGCCAGGGCAGGCCCCCAGACTGCTGATCTA |
| | | | CGATGCATCCAACAGAGCCACTGGCATCCCCGCAAGGTTCTCAGGCAGCGGGTCCGGAACCGACTTTACTCTG |
| | | | ACCATCTCAAGCCTGGAGCCCGAAGATTTCGCTGTGTATTACTGCCAGCAGAGGTCTAATTGGCCTATCACAT |
| | | | TTGGCCAGGGGACTCGCCTGGAGATTAAG |
| 107. | 3916 | CH2 | GCACCAGAACTGCTGGGAGGACCAAGCGTGTTCCTGTTTCCACCCAAGCCTAAAGATACCCTGATGATTAGTA |
| | | | GGACCCCTGAGGTCACATGTGTGGTCGTGGACGTGAGCCACGAGGACCCCGAAGTCAAGTTTAACTGGTACGT |
| | | | GGACGGCGTCGAAGTGCATAATGCCAAGACAAAACCCCGCGAGGAACAGTATAATTCTACCTACCGAGTCGTG |
| | | | AGTGTCCTGACAGTGCTGCATCAGGATTGGCTGAACGGAAAAGAGTACAAGTGCAAAGTGTCCAATAAGGCTC |
| | | | TGCCTGCACCAATCGAGAAAACTATTTCTAAGGCAAAA |
| 108. | 3916 | CH3 | GGGCAGCCCCGGGAACCTCAGGTCTATGTGCTGCCTCCATCCAGAGACGAGCTGACCAAGAACCAGGTCTCTC |
| | | | TGCTGTGTCTGGTGAAAGGATTCTACCCATCAGATATCGCTGTGGAGTGGGAAAGCAATGGCCAGCCCGAGAA |
| | | | CAATTATCTGACATGGCCCCCTGTGCTGGACTCAGATGGCAGCTTCTTTCTGTACTCTAAGCTGACTGTGGAT |
| | | | AAAAGTCGGTGGCAGCAGGGGAACGTCTTTTCTTGTAGTGTGATGCATGAGGCCCTGCACAATCATTACACCC |
| | | | AGAAGTCACTGAGCCTGTCCCCTGGC |
| 109. | 2185 | Full | GATATTCAGCTGACCCAGAGTCCTGCATCACTGGCTGTGAGCCTGGGACAGCGAGCAACAATCTCCTGCAAAG |
| | | | CCAGTCAGTCAGTGGACTATGATGGCGACTCCTATCTGAACTGGTACCAGCAGATCCCAGGGCAGCCCCCTAA |
| | | | GCTGCTGATCTACGACGCTTCAAATCTGGTGAGCGGCATCCCACCACGATTCAGCGGCAGCGGCTCTGGAACC |
| | | | GATTTTACACTGAACATTCACCCAGTCGAGAAGGTGGACGCCGCTACCTACCATTGCCAGCAGTCTACAGAGG |
| | | | ACCCCTGGACTTTCGGCGGGGGAACCAAACTGGAAATCAAGGGAGGAGGAGGCAGTGGCGGAGGAGGGTCAGG |
| | | | AGGAGGAGGAAGCCAGGTGCAGCTGCAGCAGAGCGGAGCAGAGCTGGTCAGACCAGGAAGCTCCGTGAAAATT |
| | | | TCCTGTAAGGCTTCTGGCTATGCATTTTCTAGTTACTGGATGAATTGGGTGAAGCAGAGGCCAGGACAGGGCC |
| | | | TGGAATGGATCGGGCAGATTTGGCCCGGGGATGGAGACACAAACTATAATGGAAAGTTCAAAGGCAAGGCCAC |
| | | | TCTGACCGCTGACGAGTCAAGCTCCACTGCTTATATGCAGCTGTCTAGTCTGGCAAGCGAGGATTCCGCCGTC |
| | | | TACTTTTGCGCTCGGAGAGAAACCACAACTGTGGGCAGGTACTATTACGCAATGGACTACTGGGGCCAGGGGA |
| | | | CCACAGTCACCGTGTCAAGCGCAGCCGAACCCAAATCCTCTGATAAGACACACACTTGCCCTCCATGTCCAGC |
| | | | ACCTGAGGCTGCAGGAGGACCAAGCGTGTTCCTGTTTCCCCCTAAACCTAAGGACACTCTGATGATCTCTCGG |
| | | | ACTCCCGAAGTCACCTGTGTGGTCGTGAGCGTGAGCCACGAGGACCCTGAAGTCAAATTCAACTGGTACGTGG |
| | | | ATGGCGTCGAGGTGCATAATGCCAAAACAAAGCCTAGGGAGGAACAGTATAACTCCACATACCGCGTCGTGTC |
| | | | TGTCCTGACTGTGCTGCATCAGGACTGGCTGAACGGAAAGGAGTACAAATGCAAGGTGAGCAACAAGGCACTG |
| | | | CCAGCCCCCATCGAGAAGACCATTTCCAAAGCCAAGGGCCAGCCTCGAGAACCACAGGTCTATGTGCTGCCAC |
| | | | CCAGCCGGGACGAGCTGACAAAAAACCAGGTCTCCCTGCTGTGTCTGGTGAAGGGATTCTACCCTTCTGATAT |
| | | | TGCTGTGGAGTGGGAAAGTAATGGCCAGCCAGAAAACAATTATCTGACTTGGCCTCCAGTGCTGGATTCTGAC |
| | | | GGGAGTTTCTTTCTGTACAGTAAACTGACCGTGGATAAGTCACGGTGGCAGCAGGGAAACGTCTTTAGTTGTT |
| | | | CAGTGATGCACGAGGCCCTGCACAATCATTACACCCAGAAAAGCCTGTCCCTGTCTCCCGGCAAG |
| 110. | 2185 | VL | GATATTCAGCTGACCCAGAGTCCTGCATCACTGGCTGTGAGCCTGGGACAGCGAGCAACAATCTCCTGCAAAG |
| | | | CCAGTCAGTCAGTGGACTATGATGGCGACTCCTATCTGAACTGGTACCAGCAGATCCCAGGGCAGCCCCCTAA |
| | | | GCTGCTGATCTACGACGCTTCAAATCTGGTGAGCGGCATCCCACCACGATTCAGCGGCAGCGGCTCTGGAACC |
| | | | GATTTTACACTGAACATTCACCCAGTCGAGAAGGTGGACGCCGCTACCTACCATTGCCAGCAGTCTACAGAGG |
| | | | ACCCCTGGACTTTCGGCGGGGGAACCAAACTGGAAATCAAG |
| 111. | 2185 | VH | CAGGTGCAGCTGCAGCAGAGCGGAGCAGAGCTGGTCAGACCAGGAAGCTCCGTGAAAATTTCCTGTAAGGCTT |
| | | | CTGGCTATGCATTTTCTAGTTACTGGATGAATTGGGTGAAGCAGAGGCCAGGACAGGGCCTGGAATGGATCGG |
| | | | GCAGATTTGGCCCGGGGATGGAGACACAAACTATAATGGAAAGTTCAAAGGCAAGGCCACTCTGACCGCTGAC |
| | | | GAGTCAAGCTCCACTGCTTATATGCAGCTGTCTAGTCTGGCAAGCGAGGATTCCGCCGTCTACTTTTGCGCTC |
| | | | GGAGAGAAACCACAACTGTGGGCAGGTACTATTACGCAATGGACTACTGGGGCCAGGGGACCACAGTCACCGT |
| | | | GTCAAGC |
| 112. | 2185 | CH2 | GCACCTGAGGCTGCAGGAGGACCAAGCGTGTTCCTGTTTCCCCCTAAACCTAAGGACACTCTGATGATCTCTC |
| | | | GGACTCCCGAAGTCACCTGTGTGGTCGTGAGCGTGAGCCACGAGGACCCTGAAGTCAAATTCAACTGGTACGT |
| | | | GGATGGCGTCGAGGTGCATAATGCCAAAACAAAGCCTAGGGAGGAACAGTATAACTCCACATACCGCGTCGTG |
| | | | TCTGTCCTGACTGTGCTGCATCAGGACTGGCTGAACGGAAAGGAGTACAAATGCAAGGTGAGCAACAAGGCAC |
| | | | TGCCAGCCCCCATCGAGAAGACCATTTCCAAAGCCAAG |
| 113. | 2185 | CH3 | GGCCAGCCTCGAGAACCACAGGTCTATGTGCTGCCACCCAGCCGGGACGAGCTGACAAAAAACCAGGTCTCCC |
| | | | TGCTGTGTCTGGTGAAGGGATTCTACCCTTCTGATATTGCTGTGGAGTGGGAAAGTAATGGCCAGCCAGAAAA |
| | | | CAATTATCTGACTTGGCCTCCAGTGCTGGATTCTGACGGGAGTTTCTTTCTGTACAGTAAACTGACCGTGGAT |
| | | | AAGTCACGGTGGCAGCAGGGAAACGTCTTTAGTTGTTCAGTGATGCACGAGGCCCTGCACAATCATTACACCC |
| | | | AGAAAAGCCTGTCCCTGTCTCCCGGC |
| 114. | 5242 | Full | CAGGTCCAGCTGCAGCAGTCCGGAGCCGAACTGGTCAGACCCGGCAGCTCCGTGAAAATCAGCTGCAAGGCCT |
| | | | CCGGCTATGCTTTCTCTAGTTACTGGATGAACTGGGTGAAGCAGAGGCCTGGGCAGGGACTGGAATGGATCGG |
| | | | GCAGATTTGGCCAGGCGACGGGGATACAAACTATAATGGGAAGTTCAAAGGAAAGGCAACACTGACTGCCGAC |
| | | | GAGTCAAGCTCCACTGCTTATATGCAGCTGTCTAGTCTGGCTTCAGAGGATAGCGCAGTGTACTTTTGCGCCC |
| | | | GGAGAGAAACCACAACTGTGGGCCGCTACTATTACGCAATGGACTATTGGGGACAGGGCACCACAGTCACAGT |
| | | | GTCAAGCGCCTCTACTAAAGGGCCTAGTGTGTTTCCACTGGCTCCCTCCTCTAAGAGCACATCCGGAGGAACT |
| | | | GCAGCTCTGGGATGTCTGGTGAAGGATTACTTCCCAGAGCCCGTCACAGTGTCCTGGAACTCTGGCGCTCTGA |
| | | | CTAGCGGGGTCCACACCTTTCCTGCAGTGCTGCAGAGTTCAGGCCTGTATAGCCTGAGCTCCGTGGTCACCGT |
| | | | GCCATCTAGTTCACTGGGGACCCAGACATACATCTGCAACGTGAATCACAAACCAAGCAATACAAAGGTCGAC |
| | | | AAGAAAGTGGAACCCAAAAGCTGTGATAAGACTCATACCTGCCCCCCTTGTCCTGCACCAGAGGCAGCAGGAG |
| | | | GACCAAGCGTGTTCCTGTTTCCACCCAAACCTAAGGACACACTGATGATTTCCCGAACCCCAGAAGTGACATG |
| | | | CGTGGTCGTGTCTGTGAGTCACGAGGACCCCGAAGTCAAATTCAACTGGTACGTGGATGGGGTCGAGGTGCAT |
| | | | AATGCCAAAACCAAGCCCAGGGAGGAACAGTATAATTCAACTTACCGCGTCGTGAGCGTCCTGACCGTGCTGC |
| | | | ACCAGGATTGGCTGAACGGAAAGGAGTACAAATGCAAGGTGTCCAACAAGGCTCTGCCCGCACCTATCGAGAA |
| | | | GACCATTTCTAAAGCTAAGGGCCAGCCTCGAGAACCACAGGTCTATGTGTACCCTCCATCCCGGGACGAGCTG |
| | | | ACCAAAAACCAGGTCTCTCTGACATGTCTGGTGAAGGGGTTTTATCCCAGTGATATTGCCGTGGAGTGGGAAA |
| | | | GCAATGGACAGCCTGAAAACAATTACAAGACTACCCCCCCTGTGCTGGACAGTGATGGATCATTCGCACTGGT |
| | | | CTCCAAACTGACTGTGGACAAGTCTAGGTGGCAGCAGGGCAACGTCTTTTCATGTAGCGTGATGCATGAGGCC |
| | | | CTGCACAATCATTACACCCAGAAGTCCCTGTCTCTGAGTCCCGGC |
| 115. | 5242 | VH | CAGGTCCAGCTGCAGCAGTCCGGAGCCGAACTGGTCAGACCCGGCAGCTCCGTGAAAATCAGCTGCAAGGCCT |
| | | | CCGGCTATGCTTTCTCTAGTTACTGGATGAACTGGGTGAAGCAGAGGCCTGGGCAGGGACTGGAATGGATCGG |
| | | | GCAGATTTGGCCAGGCGACGGGGATACAAACTATAATGGGAAGTTCAAAGGAAAGGCAACACTGACTGCCGAC |
| | | | GAGTCAAGCTCCACTGCTTATATGCAGCTGTCTAGTCTGGCTTCAGAGGATAGCGCAGTGTACTTTTGCGCCC |
| | | | GGAGAGAAACCACAACTGTGGGCCGCTACTATTACGCAATGGACTATTGGGGACAGGGCACCACAGTCACAGT |
| | | | GTCAAGC |
| 116. | 5242 | CH1 | GCCTCTACTAAAGGGCCTAGTGTGTTTCCACTGGCTCCCTCCTCTAAGAGCACATCCGGAGGAACTGCAGCTC |
| | | | TGGGATGTCTGGTGAAGGATTACTTCCCAGAGCCCGTCACAGTGTCCTGGAACTCTGGCGCTCTGACTAGCGG |
| | | | GGTCCACACCTTTCCTGCAGTGCTGCAGAGTTCAGGCCTGTATAGCCTGAGCTCCGTGGTCACCGTGCCATCT |
| | | | AGTTCACTGGGGACCCAGACATACATCTGCAACGTGAATCACAAACCAAGCAATACAAAGGTCGACAAGAAAG |
| | | | TG |
| 117. | 5242 | CH2 | GCACCAGAGGCAGCAGGAGGACCAAGCGTGTTCCTGTTTCCACCCAAACCTAAGGACACACTGATGATTTCCC |
| | | | GAACCCCAGAAGTGACATGCGTGGTCGTGTCTGTGAGTCACGAGGACCCCGAAGTCAAATTCAACTGGTACGT |
| | | | GGATGGGGTCGAGGTGCATAATGCCAAAACCAAGCCCAGGGAGGAACAGTATAATTCAACTTACCGCGTCGTG |
| | | | AGCGTCCTGACCGTGCTGCACCAGGATTGGCTGAACGGAAAGGAGTACAAATGCAAGGTGTCCAACAAGGCTC |
| | | | TGCCCGCACCTATCGAGAAGACCATTTCTAAAGCTAAG |
| 118. | 5242 | CH3 | GGCCAGCCTCGAGAACCACAGGTCTATGTGTACCCTCCATCCCGGGACGAGCTGACCAAAAACCAGGTCTCTC |
| | | | TGACATGTCTGGTGAAGGGGTTTTATCCCAGTGATATTGCCGTGGAGTGGGAAAGCAATGGACAGCCTGAAAA |
| | | | CAATTACAAGACTACCCCCCCTGTGCTGGACAGTGATGGATCATTCGCACTGGTCTCCAAACTGACTGTGGAC |
| | | | AAGTCTAGGTGGCAGCAGGGCAACGTCTTTTCATGTAGCGTGATGCATGAGGCCCTGCACAATCATTACACCC |
| | | | AGAAGTCCCTGTCTCTGAGTCCCGGC |
| 119. | 2171 | Full | CAGATCGTCCTGACACAGAGCCCAGCAATCATGTCAGCCAGCCCCGGCGAGAAAGTCACCATGACATGCTCAG |
| | | | CCAGCTCCTCTGTGAGCTACATGAACTGGTATCAGCAGAAAAGCGGAACATCCCCCAAGAGATGGATCTACGA |
| | | | CACTTCCAAGCTGGCTTCTGGAGTGCCTGCACACTTCAGGGGCAGCGGCTCTGGGACTAGTTATTCACTGACC |
| | | | ATTTCCGGCATGGAGGCCGAAGATGCCGCTACCTACTATTGCCAGCAGTGGAGTTCAAACCCATTCACATTTG |
| | | | GATCTGGCACTAAGCTGGAAATTAATGGCGGAGGAGGCTCCGGAGGAGGAGGGTCTGGAGGAGGAGGAAGTCA |
| | | | GGTCCAGCTGCAGCAGAGCGGAGCTGAGCTGGCACGACCAGGAGCAAGTGTGAAAATGTCCTGTAAGGCCAGC |
| | | | GGCTACACTTTCACCCGGTATACCATGCATTGGGTGAAACAGAGACCCGGGCAGGGACTGGAATGGATCGGGT |
| | | | ACATTAATCCTTCCCGAGGATACACAAACTACAACCAGAAGTTTAAAGACAAGGCTACCCTGACCACAGATAA |
| | | | GAGCTCCTCTACAGCATATATGCAGCTGAGTTCACTGACTTCTGAGGACAGTGCCGTGTACTATTGCGCTAGG |
| | | | TACTATGACGATCACTACTCCCTGGATTATTGGGGCCAGGGGACTACCCTGACCGTGAGCTCCGCAGCCGAAC |
| | | | CTAAATCTAGTGACAAGACACATACTTGCCCACCTTGTCCAGCACCAGAGCTGCTGGGAGGACCTAGCGTGTT |
| | | | CCTGTTTCCACCCAAACCAAAGGATACACTGATGATCTCCCGGACCCCTGAAGTCACATGTGTGGTCGTGGAC |
| | | | GTGTCTCACGAGGACCCCGAAGTCAAGTTCAACTGGTACGTGGACGGCGTCGAGGTGCATAATGCCAAAACTA |
| | | | AGCCCAGGGAGGAACAGTACAACTCCACTTATCGCGTCGTGTCTGTCCTGACCGTGCTGCACCAGGATTGGCT |
| | | | GAACGGCAAGGAGTACAAATGCAAGGTGAGCAACAAGGCCCTGCCTGCTCCAATCGAGAAGACCATTAGCAAA |
| | | | GCAAAGGGGCAGCCCCGAGAACCTCAGGTCTACGTGTATCCTCCATCTCGGGACGAGCTGACCAAAAACCAGG |
| | | | TCAGTCTGACATGTCTGGTGAAGGGCTTTTACCCAAGCGATATTGCTGTGGAGTGGGAATCCAATGGGCAGCC |
| | | | CGAAAACAATTATAAGACAACTCCCCCTGTGCTGGACTCAGATGGGAGCTTCGCCCTGGTCAGTAAACTGACT |
| | | | GTGGACAAGTCACGGTGGCAGCAGGGAAACGTCTTTAGCTGTTCCGTGATGCATGAGGCTCTGCACAATCATT |
| | | | ACACCCAGAAATCTCTGAGTCTGTCACCCGGCAAG |
| 120. | 2171 | VL | CAGATCGTCCTGACACAGAGCCCAGCAATCATGTCAGCCAGCCCCGGCGAGAAAGTCACCATGACATGCTCAG |
| | | | CCAGCTCCTCTGTGAGCTACATGAACTGGTATCAGCAGAAAAGCGGAACATCCCCCAAGAGATGGATCTACGA |
| | | | CACTTCCAAGCTGGCTTCTGGAGTGCCTGCACACTTCAGGGGCAGCGGCTCTGGGACTAGTTATTCACTGACC |
| | | | ATTTCCGGCATGGAGGCCGAAGATGCCGCTACCTACTATTGCCAGCAGTGGAGTTCAAACCCATTCACATTTG |
| | | | GATCTGGCACTAAGCTGGAAATTAAT |
| 121. | 2171 | VH | CAGGTCCAGCTGCAGCAGAGCGGAGCTGAGCTGGCACGACCAGGAGCAAGTGTGAAAATGTCCTGTAAGGCCA |
| | | | GCGGCTACACTTTCACCCGGTATACCATGCATTGGGTGAAACAGAGACCCGGGCAGGGACTGGAATGGATCGG |
| | | | GTACATTAATCCTTCCCGAGGATACACAAACTACAACCAGAAGTTTAAAGACAAGGCTACCCTGACCACAGAT |
| | | | AAGAGCTCCTCTACAGCATATATGCAGCTGAGTTCACTGACTTCTGAGGACAGTGCCGTGTACTATTGCGCTA |
| | | | GGTACTATGACGATCACTACTCCCTGGATTATTGGGGCCAGGGGACTACCCTGACCGTGAGCTCC |
| 122. | 2171 | CH2 | GCACCAGAGCTGCTGGGAGGACCTAGCGTGTTCCTGTTTCCACCCAAACCAAAGGATACACTGATGATCTCCC |
| | | | GGACCCCTGAAGTCACATGTGTGGTCGTGGACGTGTCTCACGAGGACCCCGAAGTCAAGTTCAACTGGTACGT |
| | | | GGACGGCGTCGAGGTGCATAATGCCAAAACTAAGCCCAGGGAGGAACAGTACAACTCCACTTATCGCGTCGTG |
| | | | TCTGTCCTGACCGTGCTGCACCAGGATTGGCTGAACGGCAAGGAGTACAAATGCAAGGTGAGCAACAAGGCCC |
| | | | TGCCTGCTCCAATCGAGAAGACCATTAGCAAAGCAAAG |
| 123. | 2171 | CH3 | GGGCAGCCCCGAGAACCTCAGGTCTACGTGTATCCTCCATCTCGGGACGAGCTGACCAAAAACCAGGTCAGTC |
| | | | TGACATGTCTGGTGAAGGGCTTTTACCCAAGCGATATTGCTGTGGAGTGGGAATCCAATGGGCAGCCCGAAAA |
| | | | CAATTATAAGACAACTCCCCCTGTGCTGGACTCAGATGGGAGCTTCGCCCTGGTCAGTAAACTGACTGTGGAC |
| | | | AAGTCACGGTGGCAGCAGGGAAACGTCTTTAGCTGTTCCGTGATGCATGAGGCTCTGCACAATCATTACACCC |
| | | | AGAAATCTCTGAGTCTGTCACCCGGC |
| 124. | 2177 | Full | CAGATCGTCCTGACACAGAGCCCAGCTATCATGTCAGCAAGCCCCGGCGAGAAAGTCACAATGACTTGCTCAG |
| | | | CCAGCTCCTCTGTGAGCTACATGAACTGGTATCAGCAGAAAAGCGGAACCTCCCCCAAGAGATGGATCTACGA |
| | | | CACATCCAAGCTGGCCTCTGGAGTGCCTGCTCACTTCAGGGGCAGCGGCTCTGGGACCAGTTATTCACTGACA |
| | | | ATTTCCGGCATGGAGGCCGAAGATGCCGCTACCTACTATTGCCAGCAGTGGAGTTCAAACCCATTCACTTTTG |
| | | | GATCTGGCACCAAGCTGGAAATTAATGGCGGAGGAGGCTCCGGAGGAGGAGGGTCTGGAGGAGGAGGAAGTCA |
| | | | GGTGCAGCTGCAGCAGAGCGGAGCAGAGCTGGCTCGACCAGGAGCTAGTGTGAAAATGTCCTGTAAGGCAAGC |
| | | | GGCTACACCTTCACACGGTATACCATGCATTGGGTGAAACAGAGACCCGGGCAGGGACTGGAATGGATCGGGT |
| | | | ACATTAATCCTTCCCGAGGATACACAAACTACAACCAGAAGTTTAAAGACAAGGCCACTCTGACCACAGATAA |
| | | | GAGCTCCTCTACCGCTTATATGCAGCTGAGTTCACTGACATCTGAGGACAGTGCAGTGTACTATTGCGCCAGG |
| | | | TACTATGACGATCACTACTCCCTGGATTATTGGGGCCAGGGGACTACCCTGACAGTGAGCTCCGCAGCCGAAC |
| | | | CTAAATCTAGTGACAAGACTCATACCTGCCCCCCTTGTCCAGCACCAGAGGCTGCAGGAGGACCTAGCGTGTT |
| | | | CCTGTTTCCACCCAAACCAAAGGATACTCTGATGATCTCCCGGACACCTGAAGTCACTTGTGTGGTCGTGAGC |
| | | | GTGTCTCACGAGGACCCCGAAGTCAAGTTTAACTGGTACGTGGACGGCGTCGAGGTGCATAATGCCAAAACCA |
| | | | AGCCCAGGGAGGAACAGTACAACTCCACATATCGCGTCGTGTCTGTCCTGACTGTGCTGCACCAGGATTGGCT |
| | | | GAACGGCAAGGAGTACAAATGCAAGGTGAGCAACAAGGCACTGCCTGCCCCAATCGAGAAGACAATTAGCAAA |
| | | | GCAAAGGGGCAGCCCCGAGAACCTCAGGTCTACGTGCTGCCTCCATCTCGGGACGAGCTGACTAAAAACCAGG |
| | | | TCAGTCTGCTGTGTCTGGTGAAGGGCTTCTATCCAAGCGATATTGCTGTGGAGTGGGAATCCAATGGGCAGCC |
| | | | CGAAAACAATTACCTGACTTGGCCCCCTGTCCTGGACTCAGATGGGAGCTTCTTTCTGTATAGTAAACTGACC |
| | | | GTGGACAAGTCACGGTGGCAGCAGGGAAACGTCTTTAGCTGTTCCGTGATGCATGAGGCCCTGCACAATCATT |
| | | | ACACCCAGAAATCTCTGAGTCTGTCACCCGGCAAG |
| 125. | 2177 | VL | CAGATCGTCCTGACACAGAGCCCAGCTATCATGTCAGCAAGCCCCGGCGAGAAAGTCACAATGACTTGCTCAG |
| | | | CCAGCTCCTCTGTGAGCTACATGAACTGGTATCAGCAGAAAAGCGGAACCTCCCCCAAGAGATGGATCTACGA |
| | | | CACATCCAAGCTGGCCTCTGGAGTGCCTGCTCACTTCAGGGGCAGCGGCTCTGGGACCAGTTATTCACTGACA |
| | | | ATTTCCGGCATGGAGGCCGAAGATGCCGCTACCTACTATTGCCAGCAGTGGAGTTCAAACCCATTCACTTTTG |
| | | | GATCTGGCACCAAGCTGGAAATTAAT |
| 126. | 2177 | VH | CAGGTGCAGCTGCAGCAGAGCGGAGCAGAGCTGGCTCGACCAGGAGCTAGTGTGAAAATGTCCTGTAAGGCAA |
| | | | GCGGCTACACCTTCACACGGTATACCATGCATTGGGTGAAACAGAGACCCGGGCAGGGACTGGAATGGATCGG |
| | | | GTACATTAATCCTTCCCGAGGATACACAAACTACAACCAGAAGTTTAAAGACAAGGCCACTCTGACCACAGAT |
| | | | AAGAGCTCCTCTACCGCTTATATGCAGCTGAGTTCACTGACATCTGAGGACAGTGCAGTGTACTATTGCGCCA |
| | | | GGTACTATGACGATCACTACTCCCTGGATTATTGGGGCCAGGGGACTACCCTGACAGTGAGCTCC |
| 127. | 2177 | CH2 | GCACCAGAGGCTGCAGGAGGACCTAGCGTGTTCCTGTTTCCACCCAAACCAAAGGATACTCTGATGATCTCCC |
| | | | GGACACCTGAAGTCACTTGTGTGGTCGTGAGCGTGTCTCACGAGGACCCCGAAGTCAAGTTTAACTGGTACGT |
| | | | GGACGGCGTCGAGGTGCATAATGCCAAAACCAAGCCCAGGGAGGAACAGTACAACTCCACATATCGCGTCGTG |
| | | | TCTGTCCTGACTGTGCTGCACCAGGATTGGCTGAACGGCAAGGAGTACAAATGCAAGGTGAGCAACAAGGCAC |
| | | | TGCCTGCCCCAATCGAGAAGACAATTAGCAAAGCAAAG |
| 128. | 2177 | CH3 | GGGCAGCCCCGAGAACCTCAGGTCTACGTGCTGCCTCCATCTCGGGACGAGCTGACTAAAAACCAGGTCAGTC |
| | | | TGCTGTGTCTGGTGAAGGGCTTCTATCCAAGCGATATTGCTGTGGAGTGGGAATCCAATGGGCAGCCCGAAAA |
| | | | CAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCC |
| | | | AGAAATCTCTGAGTCTGTCACCCGGC |
| 129. | 2305 | Full | CAGGTCCAGCTGCAGCAGAGCGGAGCCGAACTGGTCAGACCCGGCAGCTCCGTGAAAATCAGTTGCAAGGCTT |
| | | | CAGGCTATGCATTCTCTAGTTACTGGATGAACTGGGTGAAGCAGAGGCCTGGGCAGGGACTGGAATGGATCGG |
| | | | GCAGATTTGGCCAGGCGACGGGGATACTAACTATAATGGGAAGTTCAAAGGAAAGGCCACTCTGACCGCTGAC |
| | | | GAGTCAAGCTCCACCGCCTATATGCAGCTGTCTAGTCTGGCATCTGAGGATAGTGCCGTGTACTTTTGCGCTC |
| | | | GGAGAGAAACCACAACTGTGGGCCGCTACTATTACGCTATGGACTATTGGGGACAGGGCACCACAGTCACTGT |
| | | | GTCAAGCGCTAGCACCAAAGGGCCTTCCGTGTTTCCACTGGCACCCTCCTCTAAGAGCACTTCCGGAGGAACC |
| | | | GCAGCTCTGGGATGTCTGGTGAAGGATTACTTCCCAGAGCCCGTCACAGTGTCATGGAACAGCGGAGCACTGA |
| | | | CCAGCGGAGTCCACACATTTCCTGCCGTGCTGCAGAGTTCAGGCCTGTATTCCCTGAGCTCCGTGGTCACAGT |
| | | | GCCATCTAGTTCACTGGGGACACAGACTTACATCTGCAACGTGAATCACAAACCATCCAATACTAAGGTCGAC |
| | | | AAGAAAGTGGAACCCAAATCTTGTGATAAGACCCATACATGCCCCCCTTGTCCTGCTCCAGAGCTGCTGGGAG |
| | | | GACCAAGCGTGTTCCTGTTTCCACCCAAACCTAAGGACACTCTGATGATTAGCCGAACACCAGAAGTCACTTG |
| | | | CGTGGTCGTGGACGTGAGCCACGAGGACCCCGAAGTCAAGTTCAACTGGTACGTGGATGGGGTCGAGGTGCAT |
| | | | AATGCCAAAACCAAGCCCAGGGAGGAACAGTATAATTCTACATACCGCGTCGTGAGTGTCCTGACTGTGCTGC |
| | | | ACCAGGACTGGCTGAACGGAAAGGAGTACAAATGCAAGGTGTCCAACAAGGCACTGCCCGCCCCTATCGAGAA |
| | | | GACCATTTCTAAAGCAAAGGGCCAGCCTCGAGAACCACAGGTCTATGTGCTGCCTCCAAGTCGGGACGAGCTG |
| | | | ACAAAAAACCAGGTCAGCCTGCTGTGTCTGGTGAAGGGGTTCTACCCCTCCGATATTGCCGTGGAGTGGGAAT |
| | | | CTAATGGACAGCCTGAAAACAATTATCTGACCTGGCCCCCTGTGCTGGACTCCGATGGATCTTTCTTTCTGTA |
| | | | CTCAAAACTGACAGTGGATAAGAGCAGGTGGCAGCAGGGCAACGTCTTTTCTTGTAGTGTGATGCATGAGGCC |
| | | | CTGCACAATCATTACACCCAGAAATCACTGAGCCTGTCCCCCGGCAAG |
| 130. | 2305 | VH | CAGGTCCAGCTGCAGCAGAGCGGAGCCGAACTGGTCAGACCCGGCAGCTCCGTGAAAATCAGTTGCAAGGCTT |
| | | | CAGGCTATGCATTCTCTAGTTACTGGATGAACTGGGTGAAGCAGAGGCCTGGGCAGGGACTGGAATGGATCGG |
| | | | GCAGATTTGGCCAGGCGACGGGGATACTAACTATAATGGGAAGTTCAAAGGAAAGGCCACTCTGACCGCTGAC |
| | | | GAGTCAAGCTCCACCGCCTATATGCAGCTGTCTAGTCTGGCATCTGAGGATAGTGCCGTGTACTTTTGCGCTC |
| | | | GGAGAGAAACCACAACTGTGGGCCGCTACTATTACGCTATGGACTATTGGGGACAGGGCACCACAGTCACTGT |
| | | | GTCAAGC |
| 131. | 2305 | CH1 | GCTAGCACCAAAGGGCCTTCCGTGTTTCCACTGGCACCCTCCTCTAAGAGCACTTCCGGAGGAACCGCAGCTC |
| | | | TGGGATGTCTGGTGAAGGATTACTTCCCAGAGCCCGTCACAGTGTCATGGAACAGCGGAGCACTGACCAGCGG |
| | | | AGTCCACACATTTCCTGCCGTGCTGCAGAGTTCAGGCCTGTATTCCCTGAGCTCCGTGGTCACAGTGCCATCT |
| | | | AGTTCACTGGGGACACAGACTTACATCTGCAACGTGAATCACAAACCATCCAATACTAAGGTCGACAAGAAAG |
| | | | TG |
| 132. | 2305 | CH2 | GCTCCAGAGCTGCTGGGAGGACCAAGCGTGTTCCTGTTTCCACCCAAACCTAAGGACACTCTGATGATTAGCC |
| | | | GAACACCAGAAGTCACTTGCGTGGTCGTGGACGTGAGCCACGAGGACCCCGAAGTCAAGTTCAACTGGTACGT |
| | | | GGATGGGGTCGAGGTGCATAATGCCAAAACCAAGCCCAGGGAGGAACAGTATAATTCTACATACCGCGTCGTG |
| | | | AGTGTCCTGACTGTGCTGCACCAGGACTGGCTGAACGGAAAGGAGTACAAATGCAAGGTGTCCAACAAGGCAC |
| | | | TGCCCGCCCCTATCGAGAAGACCATTTCTAAAGCAAAG |
| 133. | 2305 | CH3 | GGCCAGCCTCGAGAACCACAGGTCTATGTGCTGCCTCCAAGTCGGGACGAGCTGACAAAAAACCAGGTCAGCC |
| | | | TGCTGTGTCTGGTGAAGGGGTTCTACCCCTCCGATATTGCCGTGGAGTGGGAATCTAATGGACAGCCTGAAAA |
| | | | CAATTATCTGACCTGGCCCCCTGTGCTGGACTCCGATGGATCTTTCTTTCTGTACTCAAAACTGACAGTGGAT |
| | | | AAGAGCAGGTGGCAGCAGGGCAACGTCTTTTCTTGTAGTGTGATGCATGAGGCCCTGCACAATCATTACACCC |
| | | | AGAAATCACTGAGCCTGTCCCCCGGC |
| 134. | 5238 | Full | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACATACTATTGCCAGCAGTGGAGTTCAAACCCATTCACTTTTG |
| | | | GATCTGGCACCAAGCTGGAAATTAATGGCGGAGGAGGCTCCGGAGGAGGAGGGTCTGGAGGAGGAGGAAGTCA |
| | | | GGTGCAGCTGCAGCAGTCCGGAGCTGAGCTGGCACGACCAGGAGCAAGTGTGAAAATGTCCTGTAAGGCCAGC |
| | | | GGCTACACCTTCACACGGTATACCATGCATTGGGTGAAACAGAGACCCGGGCAGGGACTGGAATGGATCGGGT |
| | | | ACATTAATCCTAGCCGAGGATACACAAACTACAACCAGAAGTTTAAAGACAAGGCTACTCTGACCACAGATAA |
| | | | GAGCTCCTCTACCGCATATATGCAGCTGAGTTCACTGACATCTGAGGACAGTGCCGTGTACTATTGCGCTAGG |
| | | | TACTATGACGATCACTACTGTCTGGATTATTGGGGCCAGGGGACTACCCTGACCGTGAGCTCCGCAGCCGAAC |
| | | | CTAAATCTAGTGACAAGACTCATACCTGCCCCCCTTGTCCAGCACCAGAGCTGCTGGGAGGACCTTCCGTGTT |
| | | | CCTGTTTCCACCCAAACCAAAGGATACTCTGATGATCTCCCGGACACCTGAAGTCACTTGCGTGGTCGTGGAC |
| | | | GTGTCTCACGAGGACCCCGAAGTCAAGTTCAACTGGTACGTGGACGGCGTCGAGGTGCATAATGCCAAAACCA |
| | | | AGCCCAGGGAGGAACAGTACAACTCCACATATCGCGTCGTGTCTGTCCTGACTGTGCTGCACCAGGATTGGCT |
| | | | GAACGGCAAGGAGTACAAATGCAAGGTGAGCAACAAGGCCCTGCCTGCTCCAATCGAGAAGACAATTAGCAAA |
| | | | GCCAAGGGGCAGCCCCGAGAACCTCAGGTCTACGTGCTGCCTCCATCTCGGGACGAGCTGACTAAAAACCAGG |
| | | | TCAGTCTGCTGTGTCTGGTGAAGGGCTTCTATCCAAGCGATATTGCTGTGGAGTGGGAATCCAATGGGCAGCC |
| | | | CGAAAACAATTACCTGACTTGGCCCCCTGTCCTGGACTCAGATGGGAGCTTCTTTCTGTATAGTAAACTGACC |
| | | | GTGGACAAGTCACGGTGGCAGCAGGGAAACGTCTTTAGCTGTTCCGTGATGCATGAGGCCCTGCACAATCATT |
| | | | ACACCCAGAAGTCTCTGAGTCTGTCACCCGGC |
| 135. | 5238 | VL | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACATACTATTGCCAGCAGTGGAGTTCAAACCCATTCACTTTTG |
| | | | GATCTGGCACCAAGCTGGAAATTAAT |
| 136. | 5238 | VH | CAGGTGCAGCTGCAGCAGTCCGGAGCTGAGCTGGCACGACCAGGAGCAAGTGTGAAAATGTCCTGTAAGGCCA |
| | | | GCGGCTACACCTTCACACGGTATACCATGCATTGGGTGAAACAGAGACCCGGGCAGGGACTGGAATGGATCGG |
| | | | GTACATTAATCCTAGCCGAGGATACACAAACTACAACCAGAAGTTTAAAGACAAGGCTACTCTGACCACAGAT |
| | | | AAGAGCTCCTCTACCGCATATATGCAGCTGAGTTCACTGACATCTGAGGACAGTGCCGTGTACTATTGCGCTA |
| | | | GGTACTATGACGATCACTACTGTCTGGATTATTGGGGCCAGGGGACTACCCTGACCGTGAGCTCC |
| 137. | 5238 | CH2 | GCACCAGAGCTGCTGGGAGGACCTTCCGTGTTCCTGTTTCCACCCAAACCAAAGGATACTCTGATGATCTCCC |
| | | | GGACACCTGAAGTCACTTGCGTGGTCGTGGACGTGTCTCACGAGGACCCCGAAGTCAAGTTCAACTGGTACGT |
| | | | GGACGGCGTCGAGGTGCATAATGCCAAAACCAAGCCCAGGGAGGAACAGTACAACTCCACATATCGCGTCGTG |
| | | | TCTGTCCTGACTGTGCTGCACCAGGATTGGCTGAACGGCAAGGAGTACAAATGCAAGGTGAGCAACAAGGCCC |
| | | | TGCCTGCTCCAATCGAGAAGACAATTAGCAAAGCCAAG |
| 138. | 5238 | CH3 | GGGCAGCCCCGAGAACCTCAGGTCTACGTGCTGCCTCCATCTCGGGACGAGCTGACTAAAAACCAGGTCAGTC |
| | | | TGCTGTGTCTGGTGAAGGGCTTCTATCCAAGCGATATTGCTGTGGAGTGGGAATCCAATGGGCAGCCCGAAAA |
| | | | CAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCC |
| | | | AGAAGTCTCTGAGTCTGTCACCCGGC |
| 139. | 2167 | Full | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACCTACTATTGCCAGCAGTGGAGTTCAAACCCATTCACTTTTG |
| | | | GATCTGGCACCAAGCTGGAAATTAATGGCGGAGGAGGCTCCGGAGGAGGAGGGTCTGGAGGAGGAGGAAGTCA |
| | | | GGTGCAGCTGCAGCAGAGCGGAGCTGAGCTGGCACGACCAGGAGCAAGTGTGAAAATGTCCTGTAAGGCCAGC |
| | | | GGCTACACCTTCACACGGTATACCATGCATTGGGTGAAACAGAGACCCGGGCAGGGACTGGAATGGATCGGGT |
| | | | ACATTAATCCTTCCCGAGGATACACAAACTACAACCAGAAGTTTAAAGACAAGGCTACTCTGACCACAGATAA |
| | | | GAGCTCCTCTACCGCATATATGCAGCTGAGTTCACTGACATCTGAGGACAGTGCCGTGTACTATTGCGCTAGG |
| | | | TACTATGACGATCACTACTCCCTGGATTATTGGGGCCAGGGGACTACCCTGACAGTGAGCTCCGCAGCCGAAC |
| | | | CTAAATCTAGTGACAAGACTCATACCTGCCCCCCTTGTCCAGCACCAGAGCTGCTGGGAGGACCTAGCGTGTT |
| | | | CCTGTTTCCACCCAAACCAAAGGATACTCTGATGATCTCCCGGACACCTGAAGTCACTTGTGTGGTCGTGGAC |
| | | | GTGTCTCACGAGGACCCCGAAGTCAAGTTTAACTGGTACGTGGACGGCGTCGAGGTGCATAATGCCAAAACCA |
| | | | AGCCCAGGGAGGAACAGTACAACTCCACATATCGCGTCGTGTCTGTCCTGACTGTGCTGCACCAGGATTGGCT |
| | | | GAACGGCAAGGAGTACAAATGCAAGGTGAGCAACAAGGCCCTGCCTGCTCCAATCGAGAAGACAATTAGCAAA |
| | | | GCCAAGGGGCAGCCCCGAGAACCTCAGGTCTACGTGCTGCCTCCATCTCGGGACGAGCTGACTAAAAACCAGG |
| | | | TCAGTCTGCTGTGTCTGGTGAAGGGCTTCTATCCAAGCGATATTGCTGTGGAGTGGGAATCCAATGGGCAGCC |
| | | | CGAAAACAATTACCTGACTTGGCCCCCTGTCCTGGACTCAGATGGGAGCTTCTTTCTGTATAGTAAACTGACC |
| | | | GTGGACAAGTCACGGTGGCAGCAGGGAAACGTCTTTAGCTGTTCCGTGATGCATGAGGCCCTGCACAATCATT |
| | | | ACACCCAGAAATCTCTGAGTCTGTCACCCGGCAAG |
| 140. | 2167 | VL | CAGATCGTCCTGACACAGAGCCCAGCAATCATGTCAGCCAGCCCCGGCGAGAAAGTCACAATGACTTGCTCAG |
| | | | CAAGCTCCTCTGTGAGCTACATGAACTGGTATCAGCAGAAAAGCGGAACCTCCCCCAAGAGATGGATCTACGA |
| | | | CACATCCAAGCTGGCTTCTGGAGTGCCTGCACACTTCAGGGGCAGCGGCTCTGGGACCAGTTATTCACTGACA |
| | | | ATTTCCGGCATGGAGGCTGAAGATGCCGCTACCTACTATTGCCAGCAGTGGAGTTCAAACCCATTCACTTTTG |
| | | | GATCTGGCACCAAGCTGGAAATTAAT |
| 141. | 2167 | VH | CAGGTGCAGCTGCAGCAGAGCGGAGCTGAGCTGGCACGACCAGGAGCAAGTGTGAAAATGTCCTGTAAGGCCA |
| | | | GCGGCTACACCTTCACACGGTATACCATGCATTGGGTGAAACAGAGACCCGGGCAGGGACTGGAATGGATCGG |
| | | | GTACATTAATCCTTCCCGAGGATACACAAACTACAACCAGAAGTTTAAAGACAAGGCTACTCTGACCACAGAT |
| | | | AAGAGCTCCTCTACCGCATATATGCAGCTGAGTTCACTGACATCTGAGGACAGTGCCGTGTACTATTGCGCTA |
| | | | GGTACTATGACGATCACTACTCCCTGGATTATTGGGGCCAGGGGACTACCCTGACAGTGAGCTCC |
| 142. | 2167 | CH2 | GCACCAGAGCTGCTGGGAGGACCTAGCGTGTTCCTGTTTCCACCCAAACCAAAGGATACTCTGATGATCTCCC |
| | | | GGACACCTGAAGTCACTTGTGTGGTCGTGGACGTGTCTCACGAGGACCCCGAAGTCAAGTTTAACTGGTACGT |
| | | | GGACGGCGTCGAGGTGCATAATGCCAAAACCAAGCCCAGGGAGGAACAGTACAACTCCACATATCGCGTCGTG |
| | | | TCTGTCCTGACTGTGCTGCACCAGGATTGGCTGAACGGCAAGGAGTACAAATGCAAGGTGAGCAACAAGGCCC |
| | | | TGCCTGCTCCAATCGAGAAGACAATTAGCAAAGCCAAG |
| 143. | 2167 | CH3 | GGGCAGCCCCGAGAACCTCAGGTCTACGTGCTGCCTCCATCTCGGGACGAGCTGACTAAAAACCAGGTCAGTC |
| | | | TGCTGTGTCTGGTGAAGGGCTTCTATCCAAGCGATATTGCTGTGGAGTGGGAATCCAATGGGCAGCCCGAAAA |
| | | | CAATTACCTGACTTGGCCCCCTGTCCTGGACTCAGATGGGAGCTTCTTTCTGTATAGTAAACTGACCGTGGAC |
| | | | AAGTCACGGTGGCAGCAGGGAAACGTCTTTAGCTGTTCCGTGATGCATGAGGCCCTGCACAATCATTACACCC |
| | | | AGAAATCTCTGAGTCTGTCACCCGGC |
| 144. | 3320 | Full | GAAGTCCAGCTGGTCGAGTCCGGAGGAGGACTGGTGCAGCCAGGAGGGTCACTGAAACTGAGCTGCGCCGCTT |
| | | | CCGGCTTCACTTTTAACAAGTATGCAATGAATTGGGTGCGGCAGGCACCAGGGAAGGGACTGGAATGGGTGGC |
| | | | CCGGATCAGATCTAAGTACAACAACTACGCTACCTACTATGCAGACAGTGTGAAGGATAGGTTCACAATTTCT |
| | | | CGCGACGATAGTAAAAACACTGCTTACCTGCAGATGAACAATCTGAAGACAGAGGACACTGCAGTCTACTATT |
| | | | GCGTGAGACACGGAAACTTTGGCAATAGCTACATCTCCTATTGGGCATACTGGGGACAGGGAACCCTGGTCAC |
| | | | AGTGAGCTCCGGAGGAGGAGGCAGCGGAGGAGGAGGCTCTGGGGGAGGCGGGAGTCAGACTGTGGTCACCCAG |
| | | | GAGCCCTCACTGACAGTCAGCCCTGGAGGCACTGTGACCCTGACATGTGGGTCTAGTACCGGAGCCGTGACAT |
| | | | CTGGCAACTATCCCAATTGGGTGCAGCAGAAACCTGGACAGGCTCCACGAGGACTGATTGGAGGAACAAAGTT |
| | | | CCTGGCCCCCGGAACTCCTGCTCGATTTTCCGGCTCTCTGCTGGGAGGGAAAGCAGCACTGACCCTGAGCGGA |
| | | | GTGCAGCCTGAGGATGAAGCCGAGTACTATTGCGTGCTGTGGTACAGCAACAGATGGGTGTTCGGAGGCGGGA |
| | | | CAAAGCTGACTGTGCTGGCTGCAGAGCCAAAGTCAAGCGACAAAACTCACACCTGCCCACCTTGTCCAGCTCC |
| | | | AGAAGCAGCTGGAGGACCATCCGTGTTCCTGTTTCCACCCAAGCCCAAAGATACACTGATGATCTCTCGCACT |
| | | | CCCGAGGTCACCTGTGTGGTCGTGAGTGTGTCACACGAAGACCCTGAGGTCAAGTTTAACTGGTACGTGGATG |
| | | | GCGTCGAAGTGCATAATGCCAAGACCAAACCTCGAGAGGAACAGTATAATTCAACTTACCGGGTCGTGAGCGT |
| | | | CCTGACCGTGCTGCATCAGGACTGGCTGAACGGAAAGGAGTACAAGTGCAAAGTGAGCAATAAGGCACTGCCT |
| | | | GCCCCAATCGAAAAAACCATTAGCAAGGCTAAAGGGCAGCCAAGAGAGCCCCAGGTCTACGTGTATCCTCCAA |
| | | | GCAGGGACGAACTGACCAAGAACCAGGTCTCCCTGACATGTCTGGTGAAAGGGTTCTATCCTAGTGATATTGC |
| | | | AGTGGAATGGGAGTCAAATGGACAGCCAGAGAACAATTACAAGACCACACCCCCTGTGCTGGACTCTGATGGC |
| | | | AGTTTCGCACTGGTCTCCAAGCTGACCGTGGATAAATCTAGGTGGCAGCAGGGGAACGTCTTTAGCTGTTCCG |
| | | | TGATGCATGAAGCCCTGCACAATCATTACACACAGAAGTCTCTGAGTCTGTCACCCGGCAAA |
| 145. | 3320 | VH | GAAGTCCAGCTGGTCGAGTCCGGAGGAGGACTGGTGCAGCCAGGAGGGTCACTGAAACTGAGCTGCGCCGCTT |
| | | | CCGGCTTCACTTTTAACAAGTATGCAATGAATTGGGTGCGGCAGGCACCAGGGAAGGGACTGGAATGGGTGGC |
| | | | CCGGATCAGATCTAAGTACAACAACTACGCTACCTACTATGCAGACAGTGTGAAGGATAGGTTCACAATTTCT |
| | | | CGCGACGATAGTAAAAACACTGCTTACCTGCAGATGAACAATCTGAAGACAGAGGACACTGCAGTCTACTATT |
| | | | GCGTGAGACACGGAAACTTTGGCAATAGCTACATCTCCTATTGGGCATACTGGGGACAGGGAACCCTGGTCAC |
| | | | AGTGAGCTCC |
| 146. | 3320 | VL | CAGACTGTGGTCACCCAGGAGCCCTCACTGACAGTCAGCCCTGGAGGCACTGTGACCCTGACATGTGGGTCTA |
| | | | GTACCGGAGCCGTGACATCTGGCAACTATCCCAATTGGGTGCAGCAGAAACCTGGACAGGCTCCACGAGGACT |
| | | | GATTGGAGGAACAAAGTTCCTGGCCCCCGGAACTCCTGCTCGATTTTCCGGCTCTCTGCTGGGAGGGAAAGCA |
| | | | GCACTGACCCTGAGCGGAGTGCAGCCTGAGGATGAAGCCGAGTACTATTGCGTGCTGTGGTACAGCAACAGAT |
| | | | GGGTGTTCGGAGGCGGGACAAAGCTGACTGTGCTG |
| 147. | 3320 | CH2 | GCTCCAGAAGCAGCTGGAGGACCATCCGTGTTCCTGTTTCCACCCAAGCCCAAAGATACACTGATGATCTCTC |
| | | | GCACTCCCGAGGTCACCTGTGTGGTCGTGAGTGTGTCACACGAAGACCCTGAGGTCAAGTTTAACTGGTACGT |
| | | | GGATGGCGTCGAAGTGCATAATGCCAAGACCAAACCTCGAGAGGAACAGTATAATTCAACTTACCGGGTCGTG |
| | | | AGCGTCCTGACCGTGCTGCATCAGGACTGGCTGAACGGAAAGGAGTACAAGTGCAAAGTGAGCAATAAGGCAC |
| | | | TGCCTGCCCCAATCGAAAAAACCATTAGCAAGGCTAAA |
| 148. | 3320 | CH3 | GGGCAGCCAAGAGAGCCCCAGGTCTACGTGTATCCTCCAAGCAGGGACGAACTGACCAAGAACCAGGTCTCCC |
| | | | TGACATGTCTGGTGAAAGGGTTCTATCCTAGTGATATTGCAGTGGAATGGGAGTCAAATGGACAGCCAGAGAA |
| | | | CAATTACAAGACCACACCCCCTGTGCTGGACTCTGATGGCAGTTTCGCACTGGTCTCCAAGCTGACCGTGGAT |
| | | | AAATCTAGGTGGCAGCAGGGGAACGTCTTTAGCTGTTCCGTGATGCATGAAGCCCTGCACAATCATTACACAC |
| | | | AGAAGTCTCTGAGTCTGTCACCCGGC |
| 149. | 5241 | Full | CAGGTCCAGCTGCAGCAGAGCGGAGCCGAACTGGTCAGACCCGGCAGCTCCGTGAAAATCAGTTGCAAGGCTT |
| | | | CAGGCTATGCATTCTCTAGTTACTGGATGAACTGGGTGAAGCAGAGGCCTGGGCAGGGACTGGAATGGATCGG |
| | | | GCAGATTTGGCCAGGCGACGGGGATACAAACTATAATGGGAAGTTCAAAGGAAAGGCCACACTGACTGCTGAC |
| | | | GAGTCAAGCTCCACTGCATATATGCAGCTGTCTAGTCTGGCATCTGAGGATAGTGCCGTGTACTTTTGCGCTC |
| | | | GGAGAGAAACCACAACTGTGGGCCGCTACTATTACGCCATGGACTATTGGGGACAGGGCACCACAGTCACAGT |
| | | | GTCAAGCGCTAGCACTAAAGGGCCTTCCGTGTTTCCACTGGCACCCTCCTCTAAGAGCACATCCGGAGGAACT |
| | | | GCAGCTCTGGGATGTCTGGTGAAGGATTACTTCCCAGAGCCCGTCACAGTGTCATGGAACAGCGGCGCACTGA |
| | | | CTAGCGGGGTCCACACCTTTCCTGCCGTGCTGCAGAGTTCAGGCCTGTATTCCCTGAGCTCCGTGGTCACCGT |
| | | | GCCATCTAGTTCACTGGGGACCCAGACATACATCTGCAACGTGAATCACAAACCATCCAATACAAAGGTCGAC |
| | | | AAGAAAGTGGAACCCAAATCTTGTGATAAGACTCATACCTGCCCCCCTTGTCCTGCTCCAGAGCTGCTGGGAG |
| | | | GACCAAGCGTGTTCCTGTTTCCACCCAAACCTAAGGACACACTGATGATTAGCCGAACCCCAGAAGTGACATG |
| | | | CGTGGTCGTGGACGTGAGCCACGAGGACCCCGAAGTCAAATTCAACTGGTACGTGGATGGGGTCGAGGTGCAT |
| | | | AATGCCAAAACCAAGCCCAGGGAGGAACAGTATAATTCTACTTACCGCGTCGTGAGTGTCCTGACCGTGCTGC |
| | | | ACCAGGACTGGCTGAACGGAAAGGAGTACAAATGCAAGGTGTCCAACAAGGCACTGCCCGCCCCTATCGAGAA |
| | | | GACCATTTCTAAAGCTAAGGGCCAGCCTCGAGAACCACAGGTCTATGTGTACCCTCCAAGTCGGGACGAGCTG |
| | | | ACCAAAAACCAGGTCAGCCTGACATGTCTGGTGAAGGGGTTTTATCCCTCCGATATTGCAGTGGAGTGGGAAT |
| | | | CTAATGGACAGCCTGAAAACAATTACAAGACTACCCCCCCTGTGCTGGACTCCGATGGATCTTTCGCCCTGGT |
| | | | CTCAAAACTGACTGTGGATAAGAGCAGGTGGCAGCAGGGCAACGTCTTTTCTTGTAGTGTGATGCATGAGGCT |
| | | | CTGCACAATCATTACACCCAGAAGTCACTGAGCCTGTCCCCCGGC |
| 150. | 5241 | VH | CAGGTCCAGCTGCAGCAGAGCGGAGCCGAACTGGTCAGACCCGGCAGCTCCGTGAAAATCAGTTGCAAGGCTT |
| | | | CAGGCTATGCATTCTCTAGTTACTGGATGAACTGGGTGAAGCAGAGGCCTGGGCAGGGACTGGAATGGATCGG |
| | | | GCAGATTTGGCCAGGCGACGGGGATACAAACTATAATGGGAAGTTCAAAGGAAAGGCCACACTGACTGCTGAC |
| | | | GAGTCAAGCTCCACTGCATATATGCAGCTGTCTAGTCTGGCATCTGAGGATAGTGCCGTGTACTTTTGCGCTC |
| | | | GGAGAGAAACCACAACTGTGGGCCGCTACTATTACGCCATGGACTATTGGGGACAGGGCACCACAGTCACAGT |
| | | | GTCAAGC |
| 151. | 5241 | CH1 | GCTAGCACTAAAGGGCCTTCCGTGTTTCCACTGGCACCCTCCTCTAAGAGCACATCCGGAGGAACTGCAGCTC |
| | | | TGGGATGTCTGGTGAAGGATTACTTCCCAGAGCCCGTCACAGTGTCATGGAACAGCGGCGCACTGACTAGCGG |
| | | | GGTCCACACCTTTCCTGCCGTGCTGCAGAGTTCAGGCCTGTATTCCCTGAGCTCCGTGGTCACCGTGCCATCT |
| | | | AGTTCACTGGGGACCCAGACATACATCTGCAACGTGAATCACAAACCATCCAATACAAAGGTCGACAAGAAAG |
| | | | TG |
| 152. | 5241 | CH2 | GCTCCAGAGCTGCTGGGAGGACCAAGCGTGTTCCTGTTTCCACCCAAACCTAAGGACACACTGATGATTAGCC |
| | | | GAACCCCAGAAGTGACATGCGTGGTCGTGGACGTGAGCCACGAGGACCCCGAAGTCAAATTCAACTGGTACGT |
| | | | GGATGGGGTCGAGGTGCATAATGCCAAAACCAAGCCCAGGGAGGAACAGTATAATTCTACTTACCGCGTCGTG |
| | | | AGTGTCCTGACCGTGCTGCACCAGGACTGGCTGAACGGAAAGGAGTACAAATGCAAGGTGTCCAACAAGGCAC |
| | | | TGCCCGCCCCTATCGAGAAGACCATTTCTAAAGCTAAG |
| 153. | 5241 | CH3 | GGCCAGCCTCGAGAACCACAGGTCTATGTGTACCCTCCAAGTCGGGACGAGCTGACCAAAAACCAGGTCAGCC |
| | | | TGACATGTCTGGTGAAGGGGTTTTATCCCTCCGATATTGCAGTGGAGTGGGAATCTAATGGACAGCCTGAAAA |
| | | | CAATTACAAGACTACCCCCCCTGTGCTGGACTCCGATGGATCTTTCGCCCTGGTCTCAAAACTGACTGTGGAT |
| | | | AAGAGCAGGTGGCAGCAGGGCAACGTCTTTTCTTGTAGTGTGATGCATGAGGCTCTGCACAATCATTACACCC |
| | | | AGAAGTCACTGAGCCTGTCCCCCGGC |
| 154. | 3322 | Full | GAAGTCCAGCTGGTCGAGTCTGGAGGAGGACTGGTGAAGCCAGGAGGGAGTCTGAAACTGTCATGCGCCGCTA |
| | | | GCGGGTATACCTTCACAAGCTACGTCATGCACTGGGTGAGGCAGGCACCAGGGAAGGGACTGGAATGGATCGG |
| | | | CTATATTAATCCCTACAACGACGGGACTAAGTATAATGAGAAATTTCAGGGCAGGGTGACCATCAGCTCCGAT |
| | | | AAGTCTATTAGTACAGCCTACATGGAGCTGTCTAGTCTGCGCAGCGAAGACACAGCAATGTACTATTGCGCCA |
| | | | GGGGGACATACTATTACGGAACTCGCGTGTTCGATTACTGGGGCCAGGGGACCCTGGTCACAGTGTCAAGCGG |
| | | | AGGCGGGGGAAGTGGAGGAGGAGGCTCAGGAGGAGGAGGGAGCGACATCGTGATGACCCAGTCCCCTGCTACA |
| | | | CTGTCACTGAGCCCAGGCGAGCGGGCAACTCTGTCCTGTAGATCCTCTAAGTCTCTGCAGAACGTGAATGGAA |
| | | | ACACCTATCTGTACTGGTTTCAGCAGAAACCAGGCCAGAGCCCCCAGCTGCTGATCTATAGAATGTCCAATCT |
| | | | GAACTCTGGCGTGCCTGATAGGTTCTCCGGATCTGGCAGTGGGACCGAGTTCACCCTGACCATTAGTTCACTG |
| | | | GAGCCAGAAGACTTCGCCGTGTATTACTGCATGCAGCACCTGGAGTACCCCATCACTTTTGGAGCTGGCACCA |
| | | | AGCTGGAGATCAAGGCAGCCGAACCAAAGAGCTCCGATAAAACACATACTTGCCCACCTTGTCCAGCACCAGA |
| | | | AGCTGCAGGAGGACCAAGCGTGTTCCTGTTTCCACCCAAGCCTAAAGACACCCTGATGATCTCCCGGACTCCC |
| | | | GAGGTCACCTGTGTGGTCGTGTCAGTGAGCCACGAGGACCCTGAAGTCAAGTTCAATTGGTACGTGGATGGCG |
| | | | TCGAAGTGCATAACGCTAAGACAAAACCCCGAGAGGAACAGTATAACAGTACATACCGGGTCGTGTCAGTGCT |
| | | | GACCGTCCTGCACCAGGATTGGCTGAATGGAAAGGAGTACAAGTGCAAAGTGTCTAACAAGGCCCTGCCTGCT |
| | | | CCAATCGAGAAAACCATTAGCAAGGCTAAAGGCCAGCCCCGCGAACCTCAGGTCTATGTGCTGCCTCCAAGCC |
| | | | GAGATGAGCTGACAAAGAATCAGGTCTCCCTGCTGTGTCTGGTGAAAGGGTTCTACCCTTCTGACATTGCAGT |
| | | | GGAGTGGGAAAGTAACGGACAGCCAGAGAACAATTATCTGACATGGCCCCCTGTCCTGGACTCCGATGGCTCT |
| | | | TTCTTTCTGTACAGCAAGCTGACTGTGGACAAATCCAGATGGCAGCAGGGGAATGTCTTTTCCTGTTCTGTGA |
| | | | TGCATGAAGCCCTGCACAACCATTACACCCAGAAGAGTCTGTCACTGAGCCCTGGCAAA |
| 155. | 3322 | VH | GAAGTCCAGCTGGTCGAGTCTGGAGGAGGACTGGTGAAGCCAGGAGGGAGTCTGAAACTGTCATGCGCCGCTA |
| | | | GCGGGTATACCTTCACAAGCTACGTCATGCACTGGGTGAGGCAGGCACCAGGGAAGGGACTGGAATGGATCGG |
| | | | CTATATTAATCCCTACAACGACGGGACTAAGTATAATGAGAAATTTCAGGGCAGGGTGACCATCAGCTCCGAT |
| | | | AAGTCTATTAGTACAGCCTACATGGAGCTGTCTAGTCTGCGCAGCGAAGACACAGCAATGTACTATTGCGCCA |
| | | | GGGGGACATACTATTACGGAACTCGCGTGTTCGATTACTGGGGCCAGGGGACCCTGGTCACAGTGTCAAGC |
| 156. | 3322 | VL | GACATCGTGATGACCCAGTCCCCTGCTACACTGTCACTGAGCCCAGGCGAGCGGGCAACTCTGTCCTGTAGAT |
| | | | CCTCTAAGTCTCTGCAGAACGTGAATGGAAACACCTATCTGTACTGGTTTCAGCAGAAACCAGGCCAGAGCCC |
| | | | CCAGCTGCTGATCTATAGAATGTCCAATCTGAACTCTGGCGTGCCTGATAGGTTCTCCGGATCTGGCAGTGGG |
| | | | ACCGAGTTCACCCTGACCATTAGTTCACTGGAGCCAGAAGACTTCGCCGTGTATTACTGCATGCAGCACCTGG |
| | | | AGTACCCCATCACTTTTGGAGCTGGCACCAAGCTGGAGATCAAG |
| 157. | 3322 | CH2 | GCACCAGAAGCTGCAGGAGGACCAAGCGTGTTCCTGTTTCCACCCAAGCCTAAAGACACCCTGATGATCTCCC |
| | | | GGACTCCCGAGGTCACCTGTGTGGTCGTGTCAGTGAGCCACGAGGACCCTGAAGTCAAGTTCAATTGGTACGT |
| | | | GGATGGCGTCGAAGTGCATAACGCTAAGACAAAACCCCGAGAGGAACAGTATAACAGTACATACCGGGTCGTG |
| | | | TCAGTGCTGACCGTCCTGCACCAGGATTGGCTGAATGGAAAGGAGTACAAGTGCAAAGTGTCTAACAAGGCCC |
| | | | TGCCTGCTCCAATCGAGAAAACCATTAGCAAGGCTAAA |
| 158. | 3322 | CH3 | GGCCAGCCCCGCGAACCTCAGGTCTATGTGCTGCCTCCAAGCCGAGATGAGCTGACAAAGAATCAGGTCTCCC |
| | | | TGCTGTGTCTGGTGAAAGGGTTCTACCCTTCTGACATTGCAGTGGAGTGGGAAAGTAACGGACAGCCAGAGAA |
| | | | CAATTATCTGACATGGCCCCCTGTCCTGGACTCCGATGGCTCTTTCTTTCTGTACAGCAAGCTGACTGTGGAC |
| | | | AAATCCAGATGGCAGCAGGGGAATGTCTTTTCCTGTTCTGTGATGCATGAAGCCCTGCACAACCATTACACCC |
| | | | AGAAGAGTCTGTCACTGAGCCCTGGC |
| 159. | 2175 | Full | GACATTCAGCTGACCCAGAGTCCTGCTTCACTGGCAGTGAGCCTGGGACAGCGAGCAACAATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGAACC |
| | | | GATTTTACACTGAACATTCACCCAGTCGAGAAGGTGGACGCCGCTACCTACCATTGCCAGCAGTCTACAGAGG |
| | | | ACCCCTGGACTTTCGGCGGGGGAACCAAACTGGAAATCAAGGGAGGAGGAGGCAGTGGCGGAGGAGGGTCAGG |
| | | | AGGAGGAGGAAGCCAGGTGCAGCTGCAGCAGAGCGGAGCAGAGCTGGTCAGACCAGGAAGCTCCGTGAAAATT |
| | | | TCCTGTAAGGCATCTGGCTATGCCTTTTCTAGTTACTGGATGAATTGGGTGAAGCAGAGGCCAGGACAGGGCC |
| | | | TGGAATGGATCGGGCAGATTTGGCCCGGGGATGGAGACACAAACTATAATGGAAAGTTCAAAGGCAAGGCTAC |
| | | | TCTGACCGCAGACGAGTCAAGCTCCACTGCATATATGCAGCTGTCTAGTCTGGCCAGCGAGGATTCCGCTGTC |
| | | | TACTTTTGCGCACGGAGAGAAACCACAACTGTGGGCAGGTACTATTACGCCATGGACTACTGGGGCCAGGGGA |
| | | | CCACAGTCACCGTGTCAAGCGCAGCCGAACCCAAATCCTCTGATAAGACACACACTTGCCCTCCATGTCCAGC |
| | | | TCCTGAGCTGCTGGGAGGACCAAGCGTGTTCCTGTTTCCACCTAAACCTAAGGACACTCTGATGATCTCTCGG |
| | | | ACTCCCGAAGTCACCTGTGTGGTCGTGGATGTGAGCCACGAGGACCCTGAAGTCAAATTCAACTGGTACGTGG |
| | | | ATGGCGTCGAGGTGCATAATGCCAAAACAAAGCCTAGGGAGGAACAGTATAACTCCACATACCGCGTCGTGTC |
| | | | TGTCCTGACTGTGCTGCATCAGGACTGGCTGAACGGAAAGGAGTACAAATGCAAGGTGAGCAACAAGGCCCTG |
| | | | CCAGCTCCCATCGAGAAGACCATTTCCAAAGCTAAGGGCCAGCCTCGAGAACCACAGGTCTATGTGCTGCCAC |
| | | | CCAGCCGGGACGAGCTGACAAAAAACCAGGTCTCCCTGCTGTGTCTGGTGAAGGGATTCTACCCTTCTGATAT |
| | | | TGCAGTGGAGTGGGAAAGTAATGGCCAGCCAGAAAACAATTATCTGACTTGGCCTCCAGTGCTGGATTCTGAC |
| | | | GGGAGTTTCTTTCTGTACAGTAAACTGACCGTGGATAAGTCACGGTGGCAGCAGGGAAACGTCTTTAGTTGTT |
| | | | CAGTGATGCACGAGGCCCTGCACAATCATTACACCCAGAAAAGCCTGTCCCTGTCTCCCGGCAAG |
| 160. | 2175 | VL | GACATTCAGCTGACCCAGAGTCCTGCTTCACTGGCAGTGAGCCTGGGACAGCGAGCAACAATCTCCTGCAAAG |
| | | | CTAGTCAGTCAGTGGACTATGATGGCGACTCCTATCTGAACTGGTACCAGCAGATCCCAGGGCAGCCCCCTAA |
| | | | GCTGCTGATCTACGACGCCTCAAATCTGGTGAGCGGCATCCCACCACGATTCAGCGGCAGCGGCTCTGGAACC |
| | | | GATTTTACACTGAACATTCACCCAGTCGAGAAGGTGGACGCCGCTACCTACCATTGCCAGCAGTCTACAGAGG |
| | | | ACCCCTGGACTTTCGGCGGGGGAACCAAACTGGAAATCAAG |
| 161. | 2175 | VH | CAGGTGCAGCTGCAGCAGAGCGGAGCAGAGCTGGTCAGACCAGGAAGCTCCGTGAAAATTTCCTGTAAGGCAT |
| | | | CTGGCTATGCCTTTTCTAGTTACTGGATGAATTGGGTGAAGCAGAGGCCAGGACAGGGCCTGGAATGGATCGG |
| | | | GCAGATTTGGCCCGGGGATGGAGACACAAACTATAATGGAAAGTTCAAAGGCAAGGCTACTCTGACCGCAGAC |
| | | | GAGTCAAGCTCCACTGCATATATGCAGCTGTCTAGTCTGGCCAGCGAGGATTCCGCTGTCTACTTTTGCGCAC |
| | | | GGAGAGAAACCACAACTGTGGGCAGGTACTATTACGCCATGGACTACTGGGGCCAGGGGACCACAGTCACCGT |
| | | | GTCAAGC |
| 162. | 2175 | CH2 | GCTCCTGAGCTGCTGGGAGGACCAAGCGTGTTCCTGTTTCCACCTAAACCTAAGGACACTCTGATGATCTCTC |
| | | | GGACTCCCGAAGTCACCTGTGTGGTCGTGGATGTGAGCCACGAGGACCCTGAAGTCAAATTCAACTGGTACGT |
| | | | GGATGGCGTCGAGGTGCATAATGCCAAAACAAAGCCTAGGGAGGAACAGTATAACTCCACATACCGCGTCGTG |
| | | | TCTGTCCTGACTGTGCTGCATCAGGACTGGCTGAACGGAAAGGAGTACAAATGCAAGGTGAGCAACAAGGCCC |
| | | | TGCCAGCTCCCATCGAGAAGACCATTTCCAAAGCTAAG |
| 163. | 2175 | CH3 | GGCCAGCCTCGAGAACCACAGGTCTATGTGCTGCCACCCAGCCGGGACGAGCTGACAAAAAACCAGGTCTCCC |
| | | | TGCTGTGTCTGGTGAAGGGATTCTACCCTTCTGATATTGCAGTGGAGTGGGAAAGTAATGGCCAGCCAGAAAA |
| | | | CAATTATCTGACTTGGCCTCCAGTGCTGGATTCTGACGGGAGTTTCTTTCTGTACAGTAAACTGACCGTGGAT |
| | | | AAGTCACGGTGGCAGCAGGGAAACGTCTTTAGTTGTTCAGTGATGCACGAGGCCCTGCACAATCATTACACCC |
| | | | AGAAAAGCCTGTCCCTGTCTCCCGGC |
| 164. | 2303 | Full | CAGGTCCAGCTGGTGCAGTCCGGAGGAGGAGTGGTCCAGCCAGGACGGTCCCTGAGACTGTCTTGCAAGGCTA |
| | | | GTGGGTATACTTTCACCTCTTACACCATGCACTGGGTGCGCCAGGCACCAGGGAAGGGACTGGAATGGATCGG |
| | | | GTATATTAACCCTAGCTCCGGATACACAAAGTACAACCAGAAGTTCAAAGACCGGTTCACCATCTCCGCTGAT |
| | | | AAGAGTAAATCAACCGCATTCCTGCAGATGGACTCTCTGCGACCCGAGGATACAGGCGTGTACTTCTGCGCCC |
| | | | GGTGGCAGGACTACGATGTGTATTTTGACTACTGGGGCCAGGGGACTCCAGTCACCGTGTCTAGTGCATCAAC |
| | | | TAAGGGACCCAGCGTGTTTCCACTGGCCCCCTCAAGCAAAAGCACATCCGGAGGAACTGCAGCTCTGGGATGT |
| | | | CTGGTGAAGGATTATTTCCCAGAGCCCGTCACCGTGTCTTGGAACAGTGGAGCCCTGACTAGCGGCGTCCATA |
| | | | CCTTTCCCGCTGTGCTGCAGTCCTCTGGGCTGTATAGCCTGAGTTCAGTGGTCACAGTGCCTAGCTCCTCTCT |
| | | | GGGAACACAGACTTACATCTGCAACGTGAATCACAAGCCTTCAAATACTAAAGTCGACAAGAAAGTGGAACCA |
| | | | AAGAGCTGTGATAAAACCCATACATGCCCACCTTGTCCTGCACCAGAGCTGCTGGGAGGACCAAGCGTGTTCC |
| | | | TGTTTCCACCCAAGCCTAAAGACACCCTGATGATTTCCAGGACCCCTGAAGTCACATGCGTGGTCGTGGACGT |
| | | | GTCTCACGAGGACCCCGAAGTCAAGTTCAACTGGTACGTGGATGGCGTCGAGGTGCATAATGCCAAGACAAAA |
| | | | CCTAGGGAGGAACAGTATAACTCCACCTACCGCGTCGTGTCTGTCCTGACAGTGCTGCACCAGGACTGGCTGA |
| | | | ACGGGAAGGAGTACAAGTGCAAAGTGAGTAATAAGGCACTGCCCGCCCCTATCGAGAAAACCATTAGCAAGGC |
| | | | AAAAGGCCAGCCTAGAGAACCACAGGTCTACGTGTATCCTCCATCTAGGGACGAGCTGACAAAGAACCAGGTC |
| | | | AGTCTGACTTGTCTGGTGAAAGGATTTTATCCAAGCGATATTGCTGTGGAGTGGGAATCCAATGGCCAGCCCG |
| | | | AAAACAATTACAAGACCACACCCCCTGTGCTGGACTCAGATGGCAGCTTCGCCCTGGTCAGTAAGCTGACTGT |
| | | | GGATAAATCACGGTGGCAGCAGGGGAACGTCTTTTCTTGTAGTGTGATGCATGAGGCTCTGCACAATCATTAC |
| | | | ACCCAGAAGTCACTGAGCCTGTCCCCCGGCAAA |
| 165. | 2303 | VH | CAGGTCCAGCTGGTGCAGTCCGGAGGAGGAGTGGTCCAGCCAGGACGGTCCCTGAGACTGTCTTGCAAGGCTA |
| | | | GTGGGTATACTTTCACCTCTTACACCATGCACTGGGTGCGCCAGGCACCAGGGAAGGGACTGGAATGGATCGG |
| | | | GTATATTAACCCTAGCTCCGGATACACAAAGTACAACCAGAAGTTCAAAGACCGGTTCACCATCTCCGCTGAT |
| | | | AAGAGTAAATCAACCGCATTCCTGCAGATGGACTCTCTGCGACCCGAGGATACAGGCGTGTACTTCTGCGCCC |
| | | | GGTGGCAGGACTACGATGTGTATTTTGACTACTGGGGCCAGGGGACTCCAGTCACCGTGTCTAGT |
| 166. | 2303 | CH1 | GCATCAACTAAGGGACCCAGCGTGTTTCCACTGGCCCCCTCAAGCAAAAGCACATCCGGAGGAACTGCAGCTC |
| | | | TGGGATGTCTGGTGAAGGATTATTTCCCAGAGCCCGTCACCGTGTCTTGGAACAGTGGAGCCCTGACTAGCGG |
| | | | CGTCCATACCTTTCCCGCTGTGCTGCAGTCCTCTGGGCTGTATAGCCTGAGTTCAGTGGTCACAGTGCCTAGC |
| | | | TCCTCTCTGGGAACACAGACTTACATCTGCAACGTGAATCACAAGCCTTCAAATACTAAAGTCGACAAGAAAG |
| | | | TG |
| 167. | 2303 | CH2 | GCACCAGAGCTGCTGGGAGGACCAAGCGTGTTCCTGTTTCCACCCAAGCCTAAAGACACCCTGATGATTTCCA |
| | | | GGACCCCTGAAGTCACATGCGTGGTCGTGGACGTGTCTCACGAGGACCCCGAAGTCAAGTTCAACTGGTACGT |
| | | | GGATGGCGTCGAGGTGCATAATGCCAAGACAAAACCTAGGGAGGAACAGTATAACTCCACCTACCGCGTCGTG |
| | | | TCTGTCCTGACAGTGCTGCACCAGGACTGGCTGAACGGGAAGGAGTACAAGTGCAAAGTGAGTAATAAGGCAC |
| | | | TGCCCGCCCCTATCGAGAAAACCATTAGCAAGGCAAAA |
| 168. | 2303 | CH3 | GGCCAGCCTAGAGAACCACAGGTCTACGTGTATCCTCCATCTAGGGACGAGCTGACAAAGAACCAGGTCAGTC |
| | | | TGACTTGTCTGGTGAAAGGATTTTATCCAAGCGATATTGCTGTGGAGTGGGAATCCAATGGCCAGCCCGAAAA |
| | | | CAATTACAAGACCACACCCCCTGTGCTGGACTCAGATGGCAGCTTCGCCCTGGTCAGTAAGCTGACTGTGGAT |
| | | | AAATCACGGTGGCAGCAGGGGAACGTCTTTTCTTGTAGTGTGATGCATGAGGCTCTGCACAATCATTACACCC |
| | | | AGAAGTCACTGAGCCTGTCCCCCGGC |

TABLE YY2
Polypeptide sequences of clones described in Table YY.

| SEQ | | | |
|---|---|---|---|
| ID | | | |
| No. | Clone | Desc | Polypeptide Sequence |
| 169. | 6690 | VH | QVQLQQSGAELARPGASVKMSCKASGYTFTRYTMHWVKQRPGQCLEWIGYINPSRGYTNYNQKFKDKATLTTD |
| | | | KSSSTAYMQLSSLTSEDSAVYYCARYYDDHYSLDYWGQGTTLTVSS |
| 170. | 6690 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 171. | 6690 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 172. | 6691 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGCGTKLEIKGGGGSGGGGSGGGGSQVQLQQSGAELVRPGSSVKI |
| | | | SCKASGYAFSSYWMNWVKQRPGQCLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPEAAGGPSVFLFPPKPKDTLMISR |
| | | | TPEVTCVVVSVSHEDPEVKFNWYVDGVEVHNAKTKPREEQYNSTYRVVSVLTVLHQDWLNGKEYKCKVSNKAL |
| | | | PAPIEKTISKAKGQPREPQVYVLPPSRDELTKNQVSLLCLVKGFYPSDIAVEWESNGQPENNYLTWPPVLDSD |
| | | | GSFFLYSKLTVDKSRWQQGNVFSCSVMHEALHNHYTQKSLSLSPG |
| 173. | 6691 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGCGTKLEIK |
| 174. | 6691 | VH | QVQLQQSGAELVRPGSSVKISCKASGYAFSSYWMNWVKQRPGQCLEWIGQIWPGDGDTNYNGKFKGKATLTAD |
| | | | ESSSTAYMQLSSLASEDSAVYFCARRETTTVGRYYYAMDYWGQGTTVTVSS |
| 175. | 6691 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 176. | 6691 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 177. | 1064 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLNGKEYKCKVSNKAL |
| | | | PAPIEKTISKAKGQPREPQVYTYPPSRDELTKNQVSLTCLVKGFYPSDIAVEWESNGQPENNYKTTPPVLDSD |
| | | | GSFALVSKLTVDKSRWQQGNVFSCSVMHEALHNHYTQKSLSLSPGK |
| 178. | 1064 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 179. | 1064 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 180. | 1064 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 181. | 1064 | CH3 | GQPREPQVYTYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 182. | 1065 | Full | DIKLQQSGAELARPGASVKMSCKTSGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSSVEGGSGGSGGSGGSGGVDDIQLTQSPA |
| | | | IMSASPGEKVTMTCRASSSVSYMNWYQQKSGTSPKRWIYDTSKVASGVPYRFSGSGSGTSYSLTISSMEAEDA |
| | | | ATYYCQQWSSNPLTFGAGTKLELKAAEPKSSDKTHTCPPCPAPELLGGPSVFLFPPKPKDTLMISRTPEVTCV |
| | | | VVDVSHEDPEVKFNWYVDGVEVHNAKTKPREEQYNSTYRVVSVLTVLHQDWLNGKEYKCKVSNKALPAPIEKT |
| | | | ISKAKGQPREPQVYTLPPSRDELTKNQVSLLCLVKGFYPSDIAVEWESNGQPENNYMTWPPVLDSDGSFFLYS |
| | | | KLTVDKSRWQQGNVFSCSVMHEALHNHYTQKSLSLSPGK |
| 183. | 1065 | VH | DIKLQQSGAELARPGASVKMSCKTSGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSS |
| 184. | 1065 | VL | DIQLTQSPAIMSASPGEKVTMTCRASSSVSYMNWYQQKSGTSPKRWIYDTSKVASGVPYRFSGSGSGTSYSLT |
| | | | ISSMEAEDAATYYCQQWSSNPLTFGAGTKLELK |
| 185. | 1065 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 186. | 1065 | CH3 | GQPREPQVYTLPPSRDELTKNQVSLLCLVKGFYPSDIAVEWESNGQPENNYMTWPPVLDSDGSFFLYSKLTVD |
| | | | KSRWQQGNVFSCSVMHEALHNHYTQKSLSLSPG |
| 187. | 1067 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYCLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYTLPPSRDELTKNQVSLLCLVKGFYPSDIAVEWESNGQPENNYMTWPPVLDSDGSFFLYSKLT |
| | | | VDKSRWQQGNVFSCSVMHEALHNHYTQKSLSLSPGK |
| 188. | 1067 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 189. | 1067 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSS |
| 190. | 1067 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 191. | 1067 | CH3 | GQPREPQVYTLPPSRDELTKNQVSLLCLVKGFYPSDIAVEWESNGQPENNYMTWPPVLDSDGSFFLYSKLTVD |
| | | | KSRWQQGNVFSCSVMHEALHNHYTQKSLSLSPG |
| 192. | 1842 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLNGKEYKCKVSNKAL |
| | | | PAPIEKTISKAKGQPREPQVYVYPPSRDELTKNQVSLTCLVKGFYPSDIAVEWESNGQPENNYKTTPPVLDSD |
| | | | GSFALVSKLTVDKSRWQQGNVFSCSVMHEALHNHYTQKSLSLSPGK |
| 193. | 1842 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 194. | 1842 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 195. | 1842 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 196. | 1842 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 197. | 1335 | Full | QIVLSQSPAILSASPGEKVTMTCRASSSVSYIHWFQQKPGSSPKPWIYATSNLASGVPVRFSGSGSGTSYSLT |
| | | | ISRVEAEDAATYYCQQWTSNPPTFGGGTKLEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYACEVTHQGLSSPVTKSFNRGEC |
| 198. | 1335 | VL | QIVLSQSPAILSASPGEKVTMTCRASSSVSYIHWFQQKPGSSPKPWIYATSNLASGVPVRFSGSGSGTSYSLT |
| | | | ISRVEAEDAATYYCQQWTSNPPTFGGGTKLEIK |
| 199. | 1335 | CL | RTVAAPSVFIFPPSDEQLKSGTASVVCLLNNFYPREAKVQWKVDNALQSGNSQESVTEQDSKDSTYSLSSTLT |
| | | | LSKADYEKHKVYACEVTHQGLSSPVTKSFNRGEC |
| 200. | 1342 | Full | QVQLQQPGAELVKPGASVKMSCKASGYTFTSYNMHWVKQTPGRGLEWIGAIYPGNGDTSYNQKFKGKATLTAD |
| | | | KSSSTAYMQLSSLTSEDSAVYYCARSTYYGGDWYFNVWGAGTTVTVSAASTKGPSVFPLAPSSKSTSGGTAAL |
| | | | GCLVKDYFPEPVTVSWNSGALTSGVHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYVLPPSRDELTKN |
| | | | QVSLLCLVKGFYPSDIAVEWESNGQPENNYLTWPPVLDSDGSFFLYSKLTVDKSRWQQGNVFSCSVMHEALHN |
| | | | HYTQKSLSLSPGK |
| 201. | 1342 | VH | QVQLQQPGAELVKPGASVKMSCKASGYTFTSYNMHWVKQTPGRGLEWIGAIYPGNGDTSYNQKFKGKATLTAD |
| | | | KSSSTAYMQLSSLTSEDSAVYYCARSTYYGGDWYFNVWGAGTTVTVSA |
| 202. | 1342 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 203. | 1342 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 204. | 1342 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 205. | 5239 | Full | QVQLVQSGGGVVQPGRSLRLSCKASGYTFTRYTMHWVRQAPGKGLEWIGYINPSRGYTNYNQKVKDRFTISRD |
| | | | NSKNTAFLQMDSLRPEDTGVYFCARYYDDHYCLDYWGQGTPVTVSSASTKGPSVFPLAPSSKSTSGGTAALGC |
| | | | LVKDYFPEPVTVSWNSGALTSGVHTFPAVLQSSGLYSLSSVVTVPSSSLGTQTYICNVNHKPSNTKVDKKVEP |
| | | | KSCDKTHTCPPCPAPEAAGGPSVFLFPPKPKDTLMISRTPEVTCVVVSVSHEDPEVKFNWYVDGVEVHNAKTK |
| | | | PREEQYNSTYRVVSVLTVLHQDWLNGKEYKCKVSNKALPAPIEKTISKAKGQPREPQVYVYPPSRDELTKNQV |
| | | | SLTCLVKGFYPSDIAVEWESNGQPENNYKTTPPVLDSDGSFALVSKLTVDKSRWQQGNVFSCSVMHEALHNHY |
| | | | TQKSLSLSPG |
| 206. | 5239 | VH | QVQLVQSGGGVVQPGRSLRLSCKASGYTFTRYTMHWVRQAPGKGLEWIGYINPSRGYTNYNQKVKDRFTISRD |
| | | | NSKNTAFLQMDSLRPEDTGVYFCARYYDDHYCLDYWGQGTPVTVSS |
| 207. | 5239 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 208. | 5239 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 209. | 5239 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 210. | 3916 | Full | EVQLVESGGGLVQPGRSLRLSCAASGFTFNDYAMHWVRQAPGKGLEWVSTISWNSGSIGYADSVKGRFTISRD |
| | | | NAKKSLYLQMNSLRAEDTALYYCAKDIQYGNYYYGMDVWGQGTTVTVSSGGGGSGGGGSGGGGSEIVLTQSPA |
| | | | TLSLSPGERATLSCRASQSVSSYLAWYQQKPGQAPRLLIYDASNRATGIPARFSGSGSGTDFTLTISSLEPED |
| | | | FAVYYCQQRSNWPITFGQGTRLEIKAAEPKSSDKTHTCPPCPAPELLGGPSVFLFPPKPKDTLMISRTPEVTC |
| | | | VVVDVSHEDPEVKFNWYVDGVEVHNAKTKPREEQYNSTYRVVSVLTVLHQDWLNGKEYKCKVSNKALPAPIEK |
| | | | TISKAKGQPREPQVYVLPPSRDELTKNQVSLLCLVKGFYPSDIAVEWESNGQPENNYLTWPPVLDSDGSFFLY |
| | | | SKLTVDKSRWQQGNVFSCSVMHEALHNHYTQKSLSLSPGK |
| 211. | 3916 | VH | EVQLVESGGGLVQPGRSLRLSCAASGFTFNDYAMHWVRQAPGKGLEWVSTISWNSGSIGYADSVKGRFTISRD |
| | | | NAKKSLYLQMNSLRAEDTALYYCAKDIQYGNYYYGMDVWGQGTTVTVSS |
| 212. | 3916 | VL | EIVLTQSPATLSLSPGERATLSCRASQSVSSYLAWYQQKPGQAPRLLIYDASNRATGIPARFSGSGSGTDFTL |
| | | | TISSLEPEDFAVYYCQQRSNWPITFGQGTRLEIK |
| 213. | 3916 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 214. | 3916 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 215. | 2185 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPEAAGGPSVFLFPPKPKDTLMISR |
| | | | TPEVTCVVVSVSHEDPEVKFNWYVDGVEVHNAKTKPREEQYNSTYRVVSVLTVLHQDWLNGKEYKCKVSNKAL |
| | | | PAPIEKTISKAKGQPREPQVYVLPPSRDELTKNQVSLLCLVKGFYPSDIAVEWESNGQPENNYLTWPPVLDSD |
| | | | GSFFLYSKLTVDKSRWQQGNVFSCSVMHEALHNHYTQKSLSLSPGK |
| 216. | 2185 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 217. | 2185 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 218. | 2185 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 219. | 2185 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 220. | 5242 | Full | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSSASTKGPSVFPLAPSSKSTSGGT |
| | | | AALGCLVKDYFPEPVTVSWNSGALTSGVHTFPAVLQSSGLYSLSSVVTVPSSSLGTQTYICNVNHKPSNTKVD |
| | | | KKVEPKSCDKTHTCPPCPAPEAAGGPSVFLFPPKPKDTLMISRTPEVTCVVVSVSHEDPEVKFNWYVDGVEVH |
| | | | NAKTKPREEQYNSTYRVVSVLTVLHQDWLNGKEYKCKVSNKALPAPIEKTISKAKGQPREPQVYVYPPSRDEL |
| | | | TKNQVSLTCLVKGFYPSDIAVEWESNGQPENNYKTTPPVLDSDGSFALVSKLTVDKSRWQQGNVFSCSVMHEA |
| | | | LHNHYTQKSLSLSPG |
| 221. | 5242 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 222. | 5242 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 223. | 5242 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 224. | 5242 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 225. | 2171 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYSLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVYPPSRDELTKNQVSLTCLVKGFYPSDIAVEWESNGQPENNYKTTPPVLDSDGSFALVSKLT |
| | | | VDKSRWQQGNVFSCSVMHEALHNHYTQKSLSLSPGK |
| 226. | 2171 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 227. | 2171 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYSLDYWGQGTTLTVSS |
| 228. | 2171 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 229. | 2171 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 230. | 2177 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYSLDYWGQGTTLTVSSAAEPKSSDKTHTCPPCPAPEAAGGPSVFLFPPKPKDTLMISRTPEVTCVVVS |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVLPPSRDELTKNQVSLLCLVKGFYPSDIAVEWESNGQPENNYLTWPPVLDSDGSFFLYSKLT |
| | | | VDKSRWQQGNVFSCSVMHEALHNHYTQKSLSLSPGK |
| 231. | 2177 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 232. | 2177 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYSLDYWGQGTTLTVSS |
| 233. | 2177 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 234. | 2177 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 235. | 2305 | Full | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSSASTKGPSVFPLAPSSKSTSGGT |
| | | | AALGCLVKDYFPEPVTVSWNSGALTSGVHTFPAVLQSSGLYSLSSVVTVPSSSLGTQTYICNVNHKPSNTKVD |
| | | | KKVEPKSCDKTHTCPPCPAPELLGGPSVFLFPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPIEKTISKAKGQPREPQVYVLPPSRDEL |
| | | | TKNQVSLLCLVKGFYPSDIAVEWESNGQPENNYLTWPPVLDSDGSFFLYSKLTVDKSRWQQGNVFSCSVMHEA |
| | | | LHNHYTQKSLSLSPGK |
| 236. | 2305 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 237. | 2305 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 238. | 2305 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 239. | 2305 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 240. | 5238 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYCLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVLPPSRDELTKNQVSLLCLVKGFYPSDIAVEWESNGQPENNYLTWPPVLDSDGSFFLYSKLT |
| | | | VDKSRWQQGNVFSCSVMHEALHNHYTQKSLSLSPG |
| 241. | 5238 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 242. | 5238 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSS |
| 243. | 5238 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 244. | 5238 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 245. | 2167 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYSLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVLPPSRDELTKNQVSLLCLVKGFYPSDIAVEWESNGQPENNYLTWPPVLDSDGSFFLYSKLT |
| | | | VDKSRWQQGNVFSCSVMHEALHNHYTQKSLSLSPGK |
| 246. | 2167 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 247. | 2167 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYSLDYWGQGTTLTVSS |
| 248. | 2167 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 249. | 2167 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 250. | 3320 | Full | EVQLVESGGGLVQPGGSLKLSCAASGFTFNKYAMNWVRQAPGKGLEWVARIRSKYNNYATYYADSVKDRFTIS |
| | | | RDDSKNTAYLQMNNLKTEDTAVYYCVRHGNFGNSYISYWAYWGQGTLVTVSSGGGGSGGGGSGGGGSQTVVTQ |
| | | | EPSLTVSPGGTVTLTCGSSTGAVTSGNYPNWVQQKPGQAPRGLIGGTKFLAPGTPARFSGSLLGGKAALTLSG |
| | | | VQPEDEAEYYCVLWYSNRWVFGGGTKLTVLAAEPKSSDKTHTCPPCPAPEAAGGPSVFLFPPKPKDTLMISRT |
| | | | PEVTCVVVSVSHEDPEVKFNWYVDGVEVHNAKTKPREEQYNSTYRVVSVLTVLHQDWLNGKEYKCKVSNKALP |
| | | | APIEKTISKAKGQPREPQVYVYPPSRDELTKNQVSLTCLVKGFYPSDIAVEWESNGQPENNYKTTPPVLDSDG |
| | | | SFALVSKLTVDKSRWQQGNVFSCSVMHEALHNHYTQKSLSLSPGK |
| 251. | 3320 | VH | EVQLVESGGGLVQPGGSLKLSCAASGFTFNKYAMNWVRQAPGKGLEWVARIRSKYNNYATYYADSVKDRFTIS |
| | | | RDDSKNTAYLQMNNLKTEDTAVYYCVRHGNFGNSYISYWAYWGQGTLVTVSS |
| 252. | 3320 | VL | QTVVTQEPSLTVSPGGTVTLTCGSSTGAVTSGNYPNWVQQKPGQAPRGLIGGTKFLAPGTPARFSGSLLGGKA |
| | | | ALTLSGVQPEDEAEYYCVLWYSNRWVFGGGTKLTVL |
| 253. | 3320 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 254. | 3320 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 255. | 5241 | Full | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSSASTKGPSVFPLAPSSKSTSGGT |
| | | | AALGCLVKDYFPEPVTVSWNSGALTSGVHTFPAVLQSSGLYSLSSVVTVPSSSLGTQTYICNVNHKPSNTKVD |
| | | | KKVEPKSCDKTHTCPPCPAPELLGGPSVFLFPPKPKDTLMISRTPEVTCVVVDVSHEDPEVKFNWYVDGVEVH |
| | | | NAKTKPREEQYNSTYRVVSVLTVLHQDWLNGKEYKCKVSNKALPAPIEKTISKAKGQPREPQVYVYPPSRDEL |
| | | | TKNQVSLTCLVKGFYPSDIAVEWESNGQPENNYKTTPPVLDSDGSFALVSKLTVDKSRWQQGNVFSCSVMHEA |
| | | | LHNHYTQKSLSLSPG |
| 256. | 5241 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 257. | 5241 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 258. | 5241 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 259. | 5241 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 260. | 3322 | Full | EVQLVESGGGLVKPGGSLKLSCAASGYTFTSYVMHWVRQAPGKGLEWIGYINPYNDGTKYNEKFQGRVTISSD |
| | | | KSISTAYMELSSLRSEDTAMYYCARGTYYYGTRVFDYWGQGTLVTVSSGGGGSGGGGSGGGGSDIVMTQSPAT |
| | | | LSLSPGERATLSCRSSKSLQNVNGNTYLYWFQQKPGQSPQLLIYRMSNLNSGVPDRFSGSGSGTEFTLTISSL |
| | | | EPEDFAVYYCMQHLEYPITFGAGTKLEIKAAEPKSSDKTHTCPPCPAPEAAGGPSVFLFPPKPKDTLMISRTP |
| | | | EVTCVVVSVSHEDPEVKFNWYVDGVEVHNAKTKPREEQYNSTYRVVSVLTVLHQDWLNGKEYKCKVSNKALPA |
| | | | PIEKTISKAKGQPREPQVYVLPPSRDELTKNQVSLLCLVKGFYPSDIAVEWESNGQPENNYLTWPPVLDSDGS |
| | | | FFLYSKLTVDKSRWQQGNVFSCSVMHEALHNHYTQKSLSLSPGK |
| 261. | 3322 | VH | EVQLVESGGGLVKPGGSLKLSCAASGYTFTSYVMHWVRQAPGKGLEWIGYINPYNDGTKYNEKFQGRVTISSD |
| | | | KSISTAYMELSSLRSEDTAMYYCARGTYYYGTRVFDYWGQGTLVTVSS |
| 262. | 3322 | VL | DIVMTQSPATLSLSPGERATLSCRSSKSLQNVNGNTYLYWFQQKPGQSPQLLIYRMSNLNSGVPDRFSGSGSG |
| | | | TEFTLTISSLEPEDFAVYYCMQHLEYPITFGAGTKLEIK |
| 263. | 3322 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 264. | 3322 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 265. | 2175 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLNGKEYKCKVSNKAL |
| | | | PAPIEKTISKAKGQPREPQVYVLPPSRDELTKNQVSLLCLVKGFYPSDIAVEWESNGQPENNYLTWPPVLDSD |
| | | | GSFFLYSKLTVDKSRWQQGNVFSCSVMHEALHNHYTQKSLSLSPGK |
| 266. | 2175 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 267. | 2175 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 268. | 2175 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 269. | 2175 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 270. | 2303 | Full | QVQLVQSGGGVVQPGRSLRLSCKASGYTFTSYTMHWVRQAPGKGLEWIGYINPSSGYTKYNQKFKDRFTISAD |
| | | | KSKSTAFLQMDSLRPEDTGVYFCARWQDYDVYFDYWGQGTPVTVSSASTKGPSVFPLAPSSKSTSGGTAALGC |
| | | | LVKDYFPEPVTVSWNSGALTSGVHTFPAVLQSSGLYSLSSVVTVPSSSLGTQTYICNVNHKPSNTKVDKKVEP |
| | | | KSCDKTHTCPPCPAPELLGGPSVFLFPPKPKDTLMISRTPEVTCVVVDVSHEDPEVKFNWYVDGVEVHNAKTK |
| | | | PREEQYNSTYRVVSVLTVLHQDWLNGKEYKCKVSNKALPAPIEKTISKAKGQPREPQVYVYPPSRDELTKNQV |
| | | | SLTCLVKGFYPSDIAVEWESNGQPENNYKTTPPVLDSDGSFALVSKLTVDKSRWQQGNVFSCSVMHEALHNHY |
| | | | TQKSLSLSPGK |
| 271. | 2303 | VH | QVQLVQSGGGVVQPGRSLRLSCKASGYTFTSYTMHWVRQAPGKGLEWIGYINPSSGYTKYNQKFKDRFTISAD |
| | | | KSKSTAFLQMDSLRPEDTGVYFCARWQDYDVYFDYWGQGTPVTVSS |
| 272. | 2303 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 273. | 2303 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 274. | 2303 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 275. | 6690 | VH | QVQLQQSGAELARPGASVKMSCKASGYTFTRYTMHWVKQRPGQCLEWIGYINPSRGYTNYNQKFKDKATLTTD |
| | | | KSSSTAYMQLSSLTSEDSAVYYCARYYDDHYSLDYWGQGTTLTVSS |
| 276. | 6690 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 277. | 6690 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 278. | 6691 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGCGTKLEIKGGGGSGGGGSGGGGSQVQLQQSGAELVRPGSSVKI |
| | | | SCKASGYAFSSYWMNWVKQRPGQCLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPEAAGGPSVFLFPPKPKDTLMISR |
| | | | TPEVTCVVVSVSHEDPEVKFNWYVDGVEVHNAKTKPREEQYNSTYRVVSVLTVLHQDWLNGKEYKCKVSNKAL |
| | | | PAPIEKTISKAKGQPREPQVYVLPPSRDELTKNQVSLLCLVKGFYPSDIAVEWESNGQPENNYLTWPPVLDSD |
| | | | GSFFLYSKLTVDKSRWQQGNVFSCSVMHEALHNHYTQKSLSLSPG |
| 279. | 6691 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGCGTKLEIK |
| 280. | 6691 | VH | QVQLQQSGAELVRPGSSVKISCKASGYAFSSYWMNWVKQRPGQCLEWIGQIWPGDGDTNYNGKFKGKATLTAD |
| | | | ESSSTAYMQLSSLASEDSAVYFCARRETTTVGRYYYAMDYWGQGTTVTVSS |
| 281. | 6691 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 282. | 6691 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 283. | 1064 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLNGKEYKCKVSNKAL |
| | | | PAPIEKTISKAKGQPREPQVYTYPPSRDELTKNQVSLTCLVKGFYPSDIAVEWESNGQPENNYKTTPPVLDSD |
| | | | GSFALVSKLTVDKSRWQQGNVFSCSVMHEALHNHYTQKSLSLSPGK |
| 284. | 1064 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 285. | 1064 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 286. | 1064 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 287. | 1064 | CH3 | GQPREPQVYTYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 288. | 1065 | Full | DIKLQQSGAELARPGASVKMSCKTSGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSSVEGGSGGSGGSGGSGGVDDIQLTQSPA |
| | | | IMSASPGEKVTMTCRASSSVSYMNWYQQKSGTSPKRWIYDTSKVASGVPYRFSGSGSGTSYSLTISSMEAEDA |
| | | | ATYYCQQWSSNPLTFGAGTKLELKAAEPKSSDKTHTCPPCPAPELLGGPSVFLFPPKPKDTLMISRTPEVTCV |
| | | | VVDVSHEDPEVKFNWYVDGVEVHNAKTKPREEQYNSTYRVVSVLTVLHQDWLNGKEYKCKVSNKALPAPIEKT |
| | | | ISKAKGQPREPQVYTLPPSRDELTKNQVSLLCLVKGFYPSDIAVEWESNGQPENNYMTWPPVLDSDGSFFLYS |
| | | | KLTVDKSRWQQGNVFSCSVMHEALHNHYTQKSLSLSPGK |
| 289. | 1065 | VH | DIKLQQSGAELARPGASVKMSCKTSGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSS |
| 290. | 1065 | VL | DIQLTQSPAIMSASPGEKVTMTCRASSSVSYMNWYQQKSGTSPKRWIYDTSKVASGVPYRFSGSGSGTSYSLT |
| | | | ISSMEAEDAATYYCQQWSSNPLTFGAGTKLELK |
| 291. | 1065 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 292. | 1065 | CH3 | GQPREPQVYTLPPSRDELTKNQVSLLCLVKGFYPSDIAVEWESNGQPENNYMTWPPVLDSDGSFFLYSKLTVD |
| | | | KSRWQQGNVFSCSVMHEALHNHYTQKSLSLSPG |
| 293. | 1067 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYCLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYTLPPSRDELTKNQVSLLCLVKGFYPSDIAVEWESNGQPENNYMTWPPVLDSDGSFFLYSKLT |
| | | | VDKSRWQQGNVFSCSVMHEALHNHYTQKSLSLSPGK |
| 294. | 1067 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 295. | 1067 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYCLDYWGQGTTLTVSS |
| 296. | 1067 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 297. | 1067 | CH3 | GQPREPQVYTLPPSRDELTKNQVSLLCLVKGFYPSDIAVEWESNGQPENNYMTWPPVLDSDGSFFLYSKLTVD |
| | | | KSRWQQGNVFSCSVMHEALHNHYTQKSLSLSPG |
| 298. | 1842 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLNGKEYKCKVSNKAL |
| | | | PAPIEKTISKAKGQPREPQVYVYPPSRDELTKNQVSLTCLVKGFYPSDIAVEWESNGQPENNYKTTPPVLDSD |
| | | | GSFALVSKLTVDKSRWQQGNVFSCSVMHEALHNHYTQKSLSLSPGK |
| 299. | 1842 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 300. | 1842 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 301. | 1842 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 302. | 1842 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 303. | 1335 | Full | QIVLSQSPAILSASPGEKVTMTCRASSSVSYIHWFQQKPGSSPKPWIYATSNLASGVPVRFSGSGSGTSYSLT |
| | | | ISRVEAEDAATYYCQQWTSNPPTFGGGTKLEIKRTVAAPSVFIFPPSDEQLKSGTASVVCLLNNFYPREAKVQ |
| | | | WKVDNALQSGNSQESVTEQDSKDSTYSLSSTLTLSKADYEKHKVYACEVTHQGLSSPVTKSFNRGEC |
| 304. | 1335 | VL | QIVLSQSPAILSASPGEKVTMTCRASSSVSYIHWFQQKPGSSPKPWIYATSNLASGVPVRFSGSGSGTSYSLT |
| | | | ISRVEAEDAATYYCQQWTSNPPTFGGGTKLEIK |
| 305. | 1335 | CL | RTVAAPSVFIFPPSDEQLKSGTASVVCLLNNFYPREAKVQWKVDNALQSGNSQESVTEQDSKDSTYSLSSTLT |
| | | | LSKADYEKHKVYACEVTHQGLSSPVTKSFNRGEC |
| 306. | 1342 | Full | QVQLQQPGAELVKPGASVKMSCKASGYTFTSYNMHWVKQTPGRGLEWIGAIYPGNGDTSYNQKFKGKATLTAD |
| | | | KSSSTAYMQLSSLTSEDSAVYYCARSTYYGGDWYFNVWGAGTTVTVSAASTKGPSVFPLAPSSKSTSGGTAAL |
| | | | GCLVKDYFPEPVTVSWNSGALTSGVHTFPAVLQSSGLYSLSSVVTVPSSSLGTQTYICNVNHKPSNTKVDKKV |
| | | | EPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYVLPPSRDELTKN |
| | | | QVSLLCLVKGFYPSDIAVEWESNGQPENNYLTWPPVLDSDGSFFLYSKLTVDKSRWQQGNVFSCSVMHEALHN |
| | | | HYTQKSLSLSPGK |
| 307. | 1342 | VH | QVQLQQPGAELVKPGASVKMSCKASGYTFTSYNMHWVKQTPGRGLEWIGAIYPGNGDTSYNQKFKGKATLTAD |
| | | | KSSSTAYMQLSSLTSEDSAVYYCARSTYYGGDWYFNVWGAGTTVTVSA |
| 308. | 1342 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 309. | 1342 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 310. | 1342 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 311. | 5239 | Full | QVQLVQSGGGVVQPGRSLRLSCKASGYTFTRYTMHWVRQAPGKGLEWIGYINPSRGYTNYNQKVKDRFTISRD |
| | | | NSKNTAFLQMDSLRPEDTGVYFCARYYDDHYCLDYWGQGTPVTVSSASTKGPSVFPLAPSSKSTSGGTAALGC |
| | | | LVKDYFPEPVTVSWNSGALTSGVHTFPAVLQSSGLYSLSSVVTVPSSSLGTQTYICNVNHKPSNTKVDKKVEP |
| | | | KSCDKTHTCPPCPAPEAAGGPSVFLFPPKPKDTLMISRTPEVTCVVVSVSHEDPEVKFNWYVDGVEVHNAKTK |
| | | | PREEQYNSTYRVVSVLTVLHQDWLNGKEYKCKVSNKALPAPIEKTISKAKGQPREPQVYVYPPSRDELTKNQV |
| | | | SLTCLVKGFYPSDIAVEWESNGQPENNYKTTPPVLDSDGSFALVSKLTVDKSRWQQGNVFSCSVMHEALHNHY |
| | | | TQKSLSLSPG |
| 312. | 5239 | VH | QVQLVQSGGGVVQPGRSLRLSCKASGYTFTRYTMHWVRQAPGKGLEWIGYINPSRGYTNYNQKVKDRFTISRD |
| | | | NSKNTAFLQMDSLRPEDTGVYFCARYYDDHYCLDYWGQGTPVTVSS |
| 313. | 5239 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 314. | 5239 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 315. | 5239 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 316. | 3916 | Full | EVQLVESGGGLVQPGRSLRLSCAASGFTFNDYAMHWVRQAPGKGLEWVSTISWNSGSIGYADSVKGRFTISRD |
| | | | NAKKSLYLQMNSLRAEDTALYYCAKDIQYGNYYYGMDVWGQGTTVTVSSGGGGSGGGGSGGGGSEIVLTQSPA |
| | | | TLSLSPGERATLSCRASQSVSSYLAWYQQKPGQAPRLLIYDASNRATGIPARFSGSGSGTDFTLTISSLEPED |
| | | | FAVYYCQQRSNWPITFGQGTRLEIKAAEPKSSDKTHTCPPCPAPELLGGPSVFLFPPKPKDTLMISRTPEVTC |
| | | | VVVDVSHEDPEVKFNWYVDGVEVHNAKTKPREEQYNSTYRVVSVLTVLHQDWLNGKEYKCKVSNKALPAPIEK |
| | | | TISKAKGQPREPQVYVLPPSRDELTKNQVSLLCLVKGFYPSDIAVEWESNGQPENNYLTWPPVLDSDGSFFLY |
| | | | SKLTVDKSRWQQGNVFSCSVMHEALHNHYTQKSLSLSPGK |
| 317. | 3916 | VH | EVQLVESGGGLVQPGRSLRLSCAASGFTFNDYAMHWVRQAPGKGLEWVSTISWNSGSIGYADSVKGRFTISRD |
| | | | NAKKSLYLQMNSLRAEDTALYYCAKDIQYGNYYYGMDVWGQGTTVTVSS |
| 318. | 3916 | VL | EIVLTQSPATLSLSPGERATLSCRASQSVSSYLAWYQQKPGQAPRLLIYDASNRATGIPARFSGSGSGTDFTL |
| | | | TISSLEPEDFAVYYCQQRSNWPITFGQGTRLEIK |
| 319. | 3916 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 320. | 3916 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 321. | 2185 | Full | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIKGGGGSGGGGSGGGGSQVQLQQSGAELVRPGSSVKI |
| | | | SCKASGYAFSSYWMNWVKQRPGQGLEWIGQIWPGDGDTNYNGKFKGKATLTADESSSTAYMQLSSLASEDSAV |
| | | | YFCARRETTTVGRYYYAMDYWGQGTTVTVSSAAEPKSSDKTHTCPPCPAPEAAGGPSVFLFPPKPKDTLMISR |
| | | | TPEVTCVVVSVSHEDPEVKFNWYVDGVEVHNAKTKPREEQYNSTYRVVSVLTVLHQDWLNGKEYKCKVSNKAL |
| | | | PAPIEKTISKAKGQPREPQVYVLPPSRDELTKNQVSLLCLVKGFYPSDIAVEWESNGQPENNYLTWPPVLDSD |
| | | | GSFFLYSKLTVDKSRWQQGNVFSCSVMHEALHNHYTQKSLSLSPGK |
| 322. | 2185 | VL | DIQLTQSPASLAVSLGQRATISCKASQSVDYDGDSYLNWYQQIPGQPPKLLIYDASNLVSGIPPRFSGSGSGT |
| | | | DFTLNIHPVEKVDAATYHCQQSTEDPWTFGGGTKLEIK |
| 323. | 2185 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 324. | 2185 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 325. | 2185 | CH3 | GQPREPQVYVLPPSRDELTKNQVSLLCLVKGFYPSDIAVEWESNGQPENNYLTWPPVLDSDGSFFLYSKLTVD |
| | | | KSRWQQGNVFSCSVMHEALHNHYTQKSLSLSPG |
| 326. | 5242 | Full | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSSASTKGPSVFPLAPSSKSTSGGT |
| | | | AALGCLVKDYFPEPVTVSWNSGALTSGVHTFPAVLQSSGLYSLSSVVTVPSSSLGTQTYICNVNHKPSNTKVD |
| | | | KKVEPKSCDKTHTCPPCPAPEAAGGPSVFLFPPKPKDTLMISRTPEVTCVVVSVSHEDPEVKFNWYVDGVEVH |
| | | | NAKTKPREEQYNSTYRVVSVLTVLHQDWLNGKEYKCKVSNKALPAPIEKTISKAKGQPREPQVYVYPPSRDEL |
| | | | TKNQVSLTCLVKGFYPSDIAVEWESNGQPENNYKTTPPVLDSDGSFALVSKLTVDKSRWQQGNVFSCSVMHEA |
| | | | LHNHYTQKSLSLSPG |
| 327. | 5242 | VH | QVQLQQSGAELVRPGSSVKISCKASGYAFSSYWMNWVKQRPGQGLEWIGQIWPGDGDTNYNGKFKGKATLTAD |
| | | | ESSSTAYMQLSSLASEDSAVYFCARRETTTVGRYYYAMDYWGQGTTVTVSS |
| 328. | 5242 | CH1 | ASTKGPSVFPLAPSSKSTSGGTAALGCLVKDYFPEPVTVSWNSGALTSGVHTFPAVLQSSGLYSLSSVVTVPS |
| | | | SSLGTQTYICNVNHKPSNTKVDKKV |
| 329. | 5242 | CH2 | APEAAGGPSVFLFPPKPKDTLMISRTPEVTCVVVSVSHEDPEVKFNWYVDGVEVHNAKTKPREEQYNSTYRVV |
| | | | SVLTVLHQDWLNGKEYKCKVSNKALPAPIEKTISKAK |
| 330. | 5242 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 331. | 2171 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYSLDYWGQGTTLTVSSAAEPKSSDKTHTCPPCPAPELLGGPSVFLFPPKPKDTLMISRTPEVTCVVVD |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVYPPSRDELTKNQVSLTCLVKGFYPSDIAVEWESNGQPENNYKTTPPVLDSDGSFALVSKLT |
| | | | VDKSRWQQGNVFSCSVMHEALHNHYTQKSLSLSPGK |
| 332. | 2171 | VL | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEIN |
| 333. | 2171 | VH | QVQLQQSGAELARPGASVKMSCKASGYTFTRYTMHWVKQRPGQGLEWIGYINPSRGYTNYNQKFKDKATLTTD |
| | | | KSSSTAYMQLSSLTSEDSAVYYCARYYDDHYSLDYWGQGTTLTVSS |
| 334. | 2171 | CH2 | APELLGGPSVFLFPPKPKDTLMISRTPEVTCVVVDVSHEDPEVKFNWYVDGVEVHNAKTKPREEQYNSTYRVV |
| | | | SVLTVLHQDWLNGKEYKCKVSNKALPAPIEKTISKAK |
| 335. | 2171 | CH3 | GQPREPQVYVYPPSRDELTKNQVSLTCLVKGFYPSDIAVEWESNGQPENNYKTTPPVLDSDGSFALVSKLTVD |
| | | | KSRWQQGNVFSCSVMHEALHNHYTQKSLSLSPG |
| 336. | 2177 | Full | QIVLTQSPAIMSASPGEKVTMTCSASSSVSYMNWYQQKSGTSPKRWIYDTSKLASGVPAHFRGSGSGTSYSLT |
| | | | ISGMEAEDAATYYCQQWSSNPFTFGSGTKLEINGGGGSGGGGSGGGGSQVQLQQSGAELARPGASVKMSCKAS |
| | | | GYTFTRYTMHWVKQRPGQGLEWIGYINPSRGYTNYNQKFKDKATLTTDKSSSTAYMQLSSLTSEDSAVYYCAR |
| | | | YYDDHYSLDYWGQGTTLTVSSAAEPKSSDKTHTCPPCPAPEAAGGPSVFLFPPKPKDTLMISRTPEVTCVVVS |
| | | | VSHEDPEVKFNWYVDGVEVHNAKTKPREEQYNSTYRVVSVLTVLHQDWLNGKEYKCKVSNKALPAPIEKTISK |
| | | | AKGQPREPQVYVLPPSRDELTKNQVSLLCLVKGFYPSDIAVEWESNGQPENNYLTWPPVLDSDGSFFLYSKLT |
| | | | VDKSRWQQGNVFSCSVMHEALHNHYTQKSLSLSPGK |

TABLE ZZ
Exemplary CDR sequences of antigen
binding polypeptide constructs

| CDR | SEQUENCE |
|---|---|
| Wild-type OKT3 | L1: SSVSY (SEQ ID NO: 337) |
| (CD3 binding) | L2: DTS (SEQ ID NO: 338) |
| | L3: QQWSSNP (SEQ ID NO: 339) |
| | H1: GYTFTRYT (SEQ ID NO: 340) |
| | H2: INPSRGYT (SEQ ID NO: 341) |
| | H3: ARYYDDHYCLDY (SEQ ID NO: 342) |
| Stabilized | L1: SSVSY (SEQ ID NO: 343) |
| VARIANT of OKT3 | L2: DTS (SEQ ID NO: 344) |
| (CD3 binding) | L3: QQWSSNP (SEQ ID NO: 345) |
| | H1: GYTFTRYT (SEQ ID NO: 346) |
| | H2: INPSRGYT (SEQ ID NO: 347) |
| | H3: ARYYDDHYSLDY (SEQ ID NO: 348) |
| HD37 | L1: QSVDYDGDSYL (SEQ ID NO: 348) |
| (CD19 binding) | L2: DAS (SEQ ID NO: 349) |
| | L3: QQSTEDPWT (SEQ ID NO: 350) |
| | H1: GYAFSSYW (SEQ ID NO: 351) |
| | H2: IWPGDGDT (SEQ ID NO: 352) |
| | H3: ARRETTTVGRYYYAMDY |
| | (SEQ ID NO: 353) |

1. An isolated bispecific antigen binding construct comprising a first antigen-binding polypeptide construct which monovalently and specifically binds a CD19 antigen and is a Fab;
a second antigen-binding polypeptide construct which monovalently and specifically binds a CD3 antigen and is an scFv; and
a heterodimeric Fc comprising first and second Fc polypeptides each comprising a modified CH3 domain, wherein each modified CH3 domain comprises asymmetric amino acid modifications that promote the formation of a heterodimeric Fc and the dimerized CH3 domains having a melting temperature (Tm) of about 68° C. or higher, wherein the first Fc polypeptide is linked to the first antigen-binding polypeptide construct, with or without a first linker, and the second monomeric Fc polypeptide is linked to the second antigen-binding polypeptide construct with or without a second linker.

2. (canceled)

3. The isolated bispecific antigen binding construct of claim 1, comprising at least three, at least six, or at least 12 CDRs of variant 6754, 6751, 1853, 10151, 6475, 6749, 10152, 10153, 6476 5850, 5851, 5852, or 6325.

4. The isolated bispecific antigen binding construct of claim 1, wherein at least one polypeptide comprises an amino acid sequence at least 80%, 90%, 95%, 96%, 97%, 98%, or 99% identical to at least one polypeptide of Variant 6754, 6751, 1853, 10151, 6475, 6749, 10152, 10153, 6476, 5850, 5851, 5852, or 6325.

5. The isolated bispecific antigen binding construct of claim 1, wherein
a. the first antigen-binding polypeptide construct comprises the antigen-binding polypeptide construct specific for CD19 derived from an antibody selected from the group consisting of 4G7; B4; B43; BU12; CLB-CD19; Leu-12; SJ25-C1; J4.119, B43, SJ25C1, FMC63 (IgG2a) HD237 (IgG2b), Mor-208, MEDI-551, and MDX-1342;
b. and the second antigen-binding polypeptide construct comprises the binding polypeptide construct specific for CD3 derived from an antibody selected from OKT3; Teplizumab™ (MGA031, Eli Lilly); Micromet, Blinatumomab™; UCHT1; NI0401; visilizumab; X35-3, VIT3, BMA030 (BW264/56), CLB-T3/3, CRIS7, YTH12.5, F111-409, CLB-T3.4.2, WT31, WT32, SPv-T3b, 11D8, XIII-141, XIII-46, XIII-87, 12F6, T3/RW2-8C8, T3/RW2-4B6, OKT3D, M-T301, SMC2 and F101.01;
c. and/or the antigen binding construct competes with an antibody described in a or b
d. and/or a humanized version thereof.

6.-7. (canceled)

8. The isolated bispecific antigen binding construct of claim 1, wherein at least one Fc polypeptide comprises an amino acid sequence at least 80%, 90%, 95%, 96%, 97%, 98%, or 99% identical to at least one Fc polypeptide of a heterodimeric Fc of Table A or variant 6754, 6751, 1853, 10151, 6475, 6749, 10152, 10153, 6476, 5850, 5851, 5852, or 6325.

9. The isolated bispecific antigen binding construct of claim 1, wherein the heterodimeric Fc
is a human Fc; and/or
is a human IgG1 Fc or IgG4 Fc; and/or
comprises one or more modifications in at least one of the CH3 domains; and/or
comprises one or more modifications in at least one of the CH3 domains that promote formation of a heterodimer with stability comparable to a wild-type homodimeric Fc; and/or
comprises one or more modifications in at least one of the CH3 domains as described in Table A;
further comprises at least one CH2 domain; and/or
further comprises at least one CH2 domain comprising one or more modifications; and/or
further comprises at least one CH2 domain comprising one or more modifications in at least one of the CH2 domains as described in Table B; and/or
comprises one or more modifications to promote selective binding of Fc-gamma receptors and/or complement.

10. The isolated bispecific antigen binding construct of claim 1, wherein the dimerized CH3 domains have a melting temperature (Tm) of 68, 69, 70, 71, 72, 73, 74, 75, 76, 77, 77.5, 78, 79, 80, 81, 82, 83, 84, or 85° C. or higher.

11. The isolated bispecific antigen binding construct of claim 1, wherein each heterodimeric Fc polypeptide is fused to each antigen-binding polypeptide construct by a linker, optionally wherein the linker is a polypeptide linker, or optionally wherein the linker comprises an IgG1 hinge region.

12.-13. (canceled)

14. The isolated bispecific antigen binding construct of claim 1, displaying reduced Fc gamma receptor binding and no associated immune-cell mediated effector activity.

15. The isolated bispecific antigen binding construct of claim 1, wherein the bispecific antigen binding construct
is capable of synapse formation and bridging between CD19+ Raji B-cells and Jurkat T-cells as assayed by FACS and/or microscopy; and/or
mediates T-cell directed killing of CD20+ B cells in human whole blood; and/or
displays improved biophysical properties compared to v875; and/or
displays improved yield compared to v875, e.g., expressed at >10 mg/L after SEC (size exclusion chromatography); and/or
displays 10-fold better yield of the desired homogeneous species under comparable expression conditions, and/or
displays heterodimer purity, e.g., >95%.

16. The isolated bispecific antigen binding construct of claim 1, wherein the antigen-binding construct is conjugated to a drug.

17. A pharmaceutical composition comprising the isolated bispecific antigen binding construct of claim 1 and a pharmaceutical carrier.

18.-20. (canceled)

21. A method of treating a cancer in a subject, the method comprising administering an effective amount of the isolated antigen-binding construct of claim 1 to the subject.

22.-23. (canceled)

24. A method of treating a condition in a subject, the method comprising administering an effective amount of the isolated antigen-binding construct of claim 1 to the subject, wherein the condition is an inflammatory condition, a proliferative disease, a minimal residual cancer, a tumorous disease, an inflammatory disease, an immunological disorder, an autoimmune disease, an infectious disease, a viral disease, an allergic reaction, parasitic reaction, a graft-versus-host disease or host-versus-graft disease or a cell malignancies, a disease associated with B cells, a disease not responsive to treatment with at least one of an anti-CD19 antibody and an anti-CD20 antibody.

25. (canceled)

26. A method of producing the bispecific antigen binding construct of claim 1 comprising culturing a host cell under conditions suitable for expressing the bispecific antigen binding construct wherein the host cell comprises a polynucleotide encoding the isolated bispecific antigen binding construct of claim 1, and purifying the bispecific antigen binding construct.

27. A method of detecting or measuring CD3 and/or CD19 in a sample comprising contacting the sample with the bispecific antigen binding construct of claim 1 and detecting or measuring the bound complex.

28. A method of inhibiting, reducing or blocking CD3 and/or CD19 signaling in a cell comprising administering an effective amount of the bispecific antigen binding construct of claim 1 to the cell, and optional administering small molecule or a second antibody

29. An isolated polynucleotide or set of isolated polynucleotides comprising at least one nucleic acid sequence that encodes at least one polypeptide of the isolated bispecific antigen binding construct of claim 1.

30.-31. (canceled)

32. A vector or set of vectors comprising one or more of the polynucleotides or sets of polynucleotides according to claim 29.

33. (canceled)

34. An isolated cell comprising a polynucleotide or set of polynucleotides according to claim 29.

35.-36. (canceled)
